(19) United States
(12) Patent Application Publication
(10) Pub. No.: US 2025/0154494 A1
(43) Pub. Date: May 15, 2025

(54) Microfluidic Co-Encapsulation Device and System and Methods for Identifying T-Cell Receptor Ligands

(71) Applicant: JANSSEN BIOTECH, INC., Horsham, PA (US)

(72) Inventors: Nathan Felix, Spring House, PA (US); John M. Lindner, Heidelberg (DE); Kristina Kromer, Heidelberg (DE); Constantin Diekmann, Heidelberg (DE); Yonatan Herzig, Heidelberg (DE); Veronica Pinamonti, Heidelberg (DE); Miguel A. Hernandez, Heidelberg (DE); Miray Cetin, Heidelberg (DE); Jing Zhang, Heidelberg (DE); Laura Fisch, Heidelberg (DE)

(21) Appl. No.: 18/708,079

(22) PCT Filed: Nov. 8, 2022
(86) PCT No.: PCT/US2022/079461

§ 371 Date: May 7, 2024

Related U.S. Application Data
(60) Provisional application No. 63/277,347, filed on Nov. 9, 2021.
(60) Provisional application No. 63/277,311, filed on Nov. 9, 2021.

(51) Int. Cl. C12N 15/10; C07K 16/28; C12M 1/00; C12M 1/12; C12M 3/06; C12N 5/0781; C12N 5/0783; G01N 33/566

(52) U.S. Cl. CPC C12N 15/1037; C07K 16/2803; C07K 16/2887; C12M 23/16; C12M 25/16; C12M 29/06; C12N 5/0635; C12N 5/0636; C12N 15/1086; G01N 33/566; C07K 2317/31; C07K 2317/56; C07K 2317/622; G01N 2333/7051; G01N 2500/10

(57) ABSTRACT

The present invention provides a platform for co-culturing T cells with APC cells expressing a library of antigenic sequences is disclosed, as well as compositions for use in the system, a co-culturing device and methods of use of the system for identifying novel T cell receptor:antigen interactions. The present invention also provides microfluidic co-encapsulation devices configured to generate longitudinal flows of individual particles (such as individual cells) from fluid particle suspensions, combining two or more individual particle flows into a single stream of individual particles, and segmenting the single stream using an isolation fluid, resulting in a suspension of co-encapsulated individual particles from each fluid particle suspension. The present invention also provides methods of using the microfluidic co-encapsulation devices and methods of modifying fluid particle suspensions to promote individual particle separation.

### CROSS REFERENCE TO RELATED APPLICATIONS

This application claims priority to U.S. Provisional Application No. 63/277,347; filed Nov. 9, 2021 and to U.S. Provisional Application No. 63/277,311; filed Nov. 9, 2021 each of which is hereby incorporated by reference herein in its entirety.

### BACKGROUND OF THE INVENTION

T lymphocytes comprise approximately 20% of all leukocytes in human peripheral blood and are a major component of the adaptive immune system. A unique feature of each newly formed T cell is its T-cell receptor (TCR), which is formed during T cell genesis in the bone marrow and thymus via somatic recombination of genomic elements. As such, each TCR has a unique ligand-binding site, forming a receptor repertoire with the potential to bind up to 10²² distinct ligands. In order to aid in the diagnosis, prevention, and treatment of diseases and disorders such as diseases associated with pathogen infection, autoimmunity disorders, and cancer, it is crucial to determine functional relationships between TCRs and their ligands.

In droplet-based microfluidics, cells can be physically isolated from each other in aqueous droplets emulsified in immiscible oil. This offers the major advantage of culture conditions with a controlled extracellular environment and the prevention of cross-contamination on a microscale level. Droplet-based microfluidics have been applied to a variety of biological experimental systems, including mammalian cells.

In high-throughput cellular interaction screening, microfluidic co-encapsulation can be used to bring together small numbers of cells and screen in parallel for millions of potential interactions, owing to the high frequency of droplet generation, enabling the formation of millions of droplets per hour. This is performed in a controlled fashion, e.g. one-to-one droplet encapsulation of T cells and antigen-presenting cells (APCs), such as B cells. For the generation of monodisperse droplets with two cell types in a two-phase system (aqueous phase droplets in an oil phase), microfluidic designs have been described in the literature (Mazutis L et al., Nature protocols. 2013 May; 8(5):870-91). These generally consist of two cell inlets containing filter regions and a fluid resistor area, both of which converge with a fluorinated oil-based detergent phase at a T-junction nozzle. This facilitates the formation of micelle-based, cell-containing droplets.

Being able to perform high-throughput screening for millions of potential cell interactions necessitates a microfluidic device that allows droplet encapsulation for several hours. Using existing designs, droplet encapsulation of T cells with APCs could only be maintained for up to 15 min, as the filters of the aqueous inlets clog due to the tendency of suspension immune cells to aggregate together and form clumps, even when in motion. Additionally, droplet encapsulation of cells is a random process; that is, the discrete cellular encapsulation frequencies are distributed according to the Poisson distribution. Even when cells are optimized for single cell conditions, working with an average cell density of one cell per droplet volume, this results in a single-cell encapsulation rate of approximately 30% (for each cell type, when applicable).

There is thus a need in the art for art for improved microfluidic devices that reliably co-encapsulate single cells in an evenly distributed manner for prolonged periods of time without clogging as well as for a platform to rapidly deconvolute antigen:TCR interactions. The present invention addresses these unmet needs in the art.

### SUMMARY OF THE INVENTION

In one embodiment, the invention relates to a system for identifying an antigen as a ligand for binding by a TCR comprising:
a) a device for co-culturing at least one cell and at least one APC;
b) at least one isolated cell comprising at least one TCR and further comprising a nucleic acid molecule comprising a TCR responsive promoter operably linked to a nucleic acid molecule encoding an antibody or fragment thereof specific for binding to an APC marker; and
c) at least one APC of an APC library comprising a plurality of APCs, wherein each APC comprises a nucleic acid molecule of a minigene library comprising a plurality of nucleic acid molecules wherein each nucleic acid molecule comprises a nucleotide sequence encoding at least one antigenic polypeptide for presentation, wherein the sequence encoding the at least one antigenic polypeptide is of a predetermined length; wherein binding of the TCR of the T cell to at least one antigenic polypeptide of the APC induces expression of the nucleic acid molecule encoding an antibody or fragment thereof specific for binding to an APC marker, whereby the APC is tagged. In one embodiment the sequence encoding the at least one antigenic polypeptide is operably linked to a unique nucleotide barcode sequence encoding the same amino acid sequence which is shared by each member of the minigene library.

In one embodiment, the device comprises a microfluidic co-culture device comprising two inlets.

In one embodiment, the APC library is in a solution comprising EDTA.

In one embodiment, the at least one cell is in a solution comprising Ca²⁺ In one embodiment, the at least one cell is a T cell.

In one embodiment, the invention relates to a method of identifying an antigen as a ligand for binding by a TCR, the method comprising:
a) co-culturing at least one cell comprising at least one TCR and further comprising a nucleic acid molecule comprising a TCR responsive promoter operably linked to a nucleic acid molecule encoding an antibody or fragment thereof specific for binding to an APC marker with one or more APC of an APC library comprising a plurality of APCs, wherein each APC comprises a nucleic acid molecule of a minigene library comprising a plurality of nucleic acid molecules wherein each nucleic acid molecule comprises: a nucleotide sequence encoding at least one antigenic polypeptide for presentation, wherein the sequence encoding the at least one antigenic polypeptide is of a predetermined length; wherein binding of the TCR to at least one antigenic polypeptide of the APC induces expression of the nucleic acid molecule encoding an antibody or fragment thereof specific for binding to an APC marker, whereby the APC is tagged;
b) isolating the tagged APC cell; and
c) sequencing the nucleic acid molecule of the minigene library of the APC cell to identify the antigenic polypeptide as a ligand for binding by the TCR.

In one embodiment, the nucleotide sequence encoding at least one antigenic polypeptide for presentation further comprises a unique nucleotide barcode sequence encoding the same amino acid sequence which is shared by each member of the minigene library.

In one embodiment, the method of co-culturing comprises applying the at least one cell comprising at least one TCR to a first inlet of a microfluidic co-culture device comprising two inlets and applying the APC library to a second inlet of the microfluidic co-culture device.

In one embodiment, the APC library is applied to the microfluidic co-culture device in a solution comprising EDTA.

In one embodiment, the at least one cell comprising at least one TCR is applied to the microfluidic co-culture device in a solution comprising Ca²⁺.

In one embodiment, the TCR responsive promoter is activated by NFAT.

In one embodiment, the TCR responsive promoter comprises a sequence selected from the group consisting of SEQ ID NO:50-SEQ ID NO:74. In one embodiment, the expression cassette is under the control of at least one, at least two, at least three, at least four, or more than four copies of the TCR responsive promoter comprising a sequence as set forth in SEQ ID NO:50 (NBV promoter).

In one embodiment, the expression cassette comprises a nucleotide sequence encoding at least one protein selected from the group consisting of a fluorescent marker and an antibody, or fragment thereof, specific for binding to an APC marker. In one embodiment, the APC is a B cell, and wherein the APC marker is selected from the group consisting of CD19, CD20, CD38, CD40, CD45R, CD79a or CD79b. In one embodiment, the sequence encoding the antibody or fragment thereof specific for binding to the APC marker further comprises at least one selected from the group consisting of a linker sequence, a leader sequence and a tag.

In one embodiment, the TCR responsive promoter is operably linked to a nucleic acid molecule encoding at least two tandem scFv molecules for binding to an APC marker, wherein each of the at least two tandem scFv molecules is separated by a linker sequence. In one embodiment, the cell comprising the TCR promoter further comprises a nucleic acid molecule comprising a nucleotide sequence encoding an NBV transcription factor for inducing transcription at an NBV promoter comprising the nucleotide sequence of SEQ ID NO:50, wherein the NBV transcription factor comprises a fusion of
a) the cytoplasmic retention and DNA-binding domains from the N′-terminus of the nuclear factor in activated T cells (NFAT),
b) the octamer motif (‘ATGCAAAT’)-binding domain from the transcriptional co-activator Bob1, and
c) the C′-terminal transactivation domain (TAD) from the herpesvirus VP16 protein.

In one embodiment, the NBV transcription factor comprises an amino acid sequence as set forth in SEQ ID NO:76. In one embodiment, the nucleotide sequence is set forth in SEQ ID NO:75.

In one embodiment, the sequence encoding the NBV transcription factor is operably linked to a TCR responsive promoter. In one embodiment, the sequence encoding the NBV transcription factor is further regulated by at least one insulator element, an enhancer, or a combination thereof.

In one embodiment, the cell comprising the TCR responsive promoter further comprises at least one nucleotide sequence encoding a T cell co-stimulatory molecule. In one embodiment, the T cell co-stimulatory molecule is selected from the group consisting of CD2, CD226, CD40L, ICOS, OX40 and 41BB.

In one embodiment, the nucleic acid molecule of the minigene library further comprises a sequence encoding at least one transmembrane domain, or fragment thereof. In one embodiment, the transmembrane domain is SEQ ID NO:114, SEQ ID NO:116, SEQ ID NO:118, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO: 128, SEQ ID NO:129, SEQ ID NO:130, SEQ ID NO:131, SEQ ID NO:132, SEQ ID NO:133, SEQ ID NO:134, or SEQ ID NO:135.

In one embodiment, the nucleic acid molecule of the minigene library further comprises a nucleotide sequence encoding an 6X flexible Flag construct. In one embodiment, the nucleic acid molecule further comprises a nucleotide sequence of SEQ ID NO:42.

In one embodiment, the nucleic acid molecule of the minigene library further comprises a nucleotide sequence encoding at least one T cell co-stimulatory molecule. In one embodiment, the T cell co-stimulatory molecule is selected from the group consisting of CD40, CD58, CD80, CD83, CD86, OX40L, 4-1BBL, and a combination thereof.

In one embodiment, the invention relates to a nucleic acid molecule encoding an antibody or fragment thereof specific for binding to an antigen presenting cell (APC) marker. In some embodiments, the APC marker is CD19 or CD20.

In some embodiments, the nucleic acid molecule comprises a nucleotide sequence encoding a heavy chain variable region of an anti-CD19 synthetic antibody. In some embodiments, the nucleotide sequence encoding the heavy chain variable region of the anti-CD19 synthetic antibody is SEQ ID NO:4 or SEQ ID NO:12.

In some embodiments, the nucleic acid molecule comprises a nucleotide sequence encoding a light chain variable region of an anti-CD19 synthetic antibody. In some embodiments, the nucleotide sequence encoding the light chain variable region of the anti-CD19 synthetic antibody is SEQ ID NO:8 or SEQ ID NO:16.

In some embodiments, the nucleic acid molecule comprises a nucleotide sequence encoding a heavy chain variable region of an anti-CD20 synthetic antibody. In some embodiments, the nucleotide sequence encoding the heavy chain variable region of the anti-CD20 synthetic antibody is SEQ ID NO:20 or SEQ ID NO:28.

In some embodiments, the nucleic acid molecule comprises a nucleotide sequence encoding a light chain variable region of an anti-CD20 synthetic antibody. In some embodiments, the nucleotide sequence encoding the light chain variable region of the anti-CD20 synthetic antibody is SEQ ID NO:24 or SEQ ID NO:32.

In some embodiments, the nucleic acid molecule encodes an scFV antibody fragment. In some embodiments, the nucleic acid molecule comprises a nucleotide sequence encoding a heavy chain variable region of an anti-CD19 synthetic antibody comprising SEQ ID NO:4 and a light chain variable region of an anti-CD19 synthetic antibody comprising SEQ ID NO:8. In some embodiments, the nucleic acid molecule comprises a nucleotide sequence encoding a heavy chain variable region of an anti-CD19 synthetic antibody comprising SEQ ID NO:12 and a light chain variable region of an anti-CD19 synthetic antibody comprising SEQ ID NO:16. In some embodiments, the nucleic acid molecule comprises a nucleotide sequence encoding a heavy chain variable region of an anti-CD20 synthetic antibody comprising SEQ ID NO:20 and a light chain variable region of an anti-CD20 synthetic antibody comprising SEQ ID NO:24. In some embodiments, the nucleic acid molecule comprises a nucleotide sequence encoding a heavy chain variable region of an anti-CD20 synthetic antibody comprising SEQ ID NO:28 and a light chain variable region of an anti-CD20 synthetic antibody comprising SEQ ID NO:32.

In some embodiments, the sequence encoding the antibody or fragment thereof specific for binding to an antigen presenting cell marker further comprises a linker sequence, a leader sequence, a tag, or a combination thereof.

In some embodiments, the nucleic acid molecule comprises a nucleotide sequence encoding at least two tandem scFv molecules separated by a linker sequence.

In one embodiment, the invention relates to a nucleic acid molecule comprising an expression cassette for expression under the control of a T-cell receptor (TCR) responsive promoter that is activated by a molecule that is expressed upon binding of a TCR to its ligand. In some embodiments, the ligand is a polypeptide:major histocompatibility complex (MHC) complex. In some embodiments, the ligand is a polypeptide:human leukocyte antigen (HLA) complex.

In one embodiment, the TCR responsive promoter is activated by NFAT. In one embodiment, the TCR responsive promoter comprises a sequence of SEQ ID NO:50-SEQ ID NO:74. In one embodiment, the TCR responsive promoter comprises a promoter comprising the sequence of SEQ ID NO:50, which is referred to elsewhere hereinafter as the “NBV promoter”.

In one embodiment, the nucleic acid molecule comprising an expression cassette for expression under the control of a TCR responsive promoter that is activated by a molecule that is expressed, or activated, upon binding of a TCR to an antigenic polypeptide, comprises a TCR responsive promoter operably linked to a nucleic acid molecule encoding an antibody or fragment thereof specific for binding to an APC marker.

In one embodiment, the APC is a B cell, dendritic cell (DC), monocyte, macrophage, or engineered APC. In one embodiment, the APC is a B cell. In one embodiment, the APC marker is CD19, CD20, CD38, CD40, CD45R, CD79a or CD79b.

In one embodiment, the nucleic acid molecule comprises an NBV promoter comprising a sequence as set forth in SEQ ID NO:50 operably linked to a nucleotide sequence encoding a heavy chain variable region of an anti-CD19 synthetic antibody. In some embodiments, the nucleotide sequence encoding the heavy chain variable region of the anti-CD19 synthetic antibody is SEQ ID NO:4 or SEQ ID NO:12.

In one embodiment, the nucleic acid molecule comprises an NBV promoter comprising a sequence as set forth in SEQ ID NO:50 operably linked to a nucleotide sequence encoding a light chain variable region of an anti-CD19 synthetic antibody. In some embodiments, the nucleotide sequence encoding the light chain variable region of the anti-CD19 synthetic antibody is SEQ ID NO:8 or SEQ ID NO:16.

In one embodiment, the nucleic acid molecule comprises an NBV promoter comprising a sequence as set forth in SEQ ID NO:50 operably linked to a nucleotide sequence encoding a heavy chain variable region of an anti-CD20 synthetic antibody. In some embodiments, the nucleotide sequence encoding the heavy chain variable region of the anti-CD20 synthetic antibody is SEQ ID NO:20 or SEQ ID NO:28.

In one embodiment, the nucleic acid molecule comprises an NBV promoter comprising a sequence as set forth in SEQ ID NO:50 operably linked to a nucleotide sequence encoding a light chain variable region of an anti-CD20 synthetic antibody. In some embodiments, the nucleotide sequence encoding the light chain variable region of the anti-CD20 synthetic antibody is SEQ ID NO:24 or SEQ ID NO:32.

In one embodiment, the nucleic acid molecule comprises an NBV promoter comprising a sequence as set forth in SEQ ID NO:50 operably linked to a nucleotide sequence encoding a heavy chain variable region of an anti-CD19 synthetic antibody comprising SEQ ID NO:4 and a light chain variable region of an anti-CD19 synthetic antibody comprising SEQ ID NO:8.

In one embodiment, the nucleic acid molecule comprises an NBV promoter comprising a sequence as set forth in SEQ ID NO:50 operably linked to a nucleotide sequence encoding a heavy chain variable region of an anti-CD19 synthetic antibody comprising SEQ ID NO:12 and a light chain variable region of an anti-CD19 synthetic antibody comprising SEQ ID NO:16.

In one embodiment, the nucleic acid molecule comprises an NBV promoter comprising a sequence as set forth in SEQ ID NO:50 operably linked to a nucleotide sequence encoding a heavy chain variable region of an anti-CD20 synthetic antibody comprising SEQ ID NO:20 and a light chain variable region of an anti-CD20 synthetic antibody comprising SEQ ID NO:24.

In one embodiment, the nucleic acid molecule comprises an NBV promoter comprising a sequence as set forth in SEQ ID NO:50 operably linked to a nucleotide sequence encoding a heavy chain variable region of an anti-CD20 synthetic antibody comprising SEQ ID NO:28 and a light chain variable region of an anti-CD20 synthetic antibody comprising SEQ ID NO:32.

In one embodiment, the sequence encoding the antibody or fragment thereof specific for binding to the APC marker further comprises at least one of a linker sequence, a leader sequence, a tag, or a combination thereof.

In one embodiment, the nucleic acid molecule comprises a TCR responsive promoter operably linked to a nucleic acid molecule encoding at least two tandem scFv molecules for binding to an APC marker, wherein each of the at least two tandem scFv molecules is separated by a linker sequence.

In one embodiment, the invention relates to a nucleic acid molecule comprising a nucleotide sequence encoding an NBV transcription factor for inducing transcription at an NBV promoter, wherein the NBV transcription factor comprises a fusion of a) the cytoplasmic retention and DNA-binding domains from the N′-terminus of the nuclear factor in activated T cells (NFAT), b) the octamer motif (‘ATGCAAAT’)-binding domain from the transcriptional co-activator Bob1, and c) the C′-terminal transactivation domain (TAD) from the herpesvirus VP16 protein.

In one embodiment, the NBV transcription factor comprises an amino acid sequence as set forth in SEQ ID NO:76.

In one embodiment, the nucleic acid molecule encoding an NBV transcription factor for inducing transcription at an NBV promoter comprises a nucleotide sequence as set forth in SEQ ID NO:75. In one embodiment, the sequence encoding the NBV transcription factor is operably linked to a TCR responsive promoter. In one embodiment, the sequence encoding the NBV transcription factor is further regulated by at least one insulator element, an enhancer, or a combination thereof.

In one embodiment, the invention relates to a cell comprising at least one nucleic acid molecule comprising an expression cassette for expression under the control of a T cell receptor (TCR) responsive promoter that is activated by a molecule that is expressed upon binding of a TCR to an antigenic polypeptide. In one embodiment, the cell further comprises at least one nucleic acid molecule comprising a nucleotide sequence encoding an NBV transcription factor for inducing transcription at an NBV promoter, wherein the NBV transcription factor comprises a fusion of a) the cytoplasmic retention and DNA-binding domains from the N′-terminus of the nuclear factor in activated T cells (NFAT), b) the octamer motif (‘ATGCAAAT’)-binding domain from the transcriptional co-activator Bob1, and c) the C′-terminal transactivation domain (TAD) from the herpesvirus VP16 protein.

In one embodiment, the cell further comprises at least one nucleotide sequence encoding a co-stimulatory molecule. In one embodiment, the co-stimulatory molecule is CD2, CD226, CD40L, ICOS, OX40 or 41BB, or any combination thereof.

In one embodiment, the invention relates to a minigene library comprising a plurality of nucleic acid molecules wherein each nucleic acid molecule comprises a nucleotide sequence encoding at least one antigenic polypeptide for presentation, wherein the sequence encoding the at least one antigenic polypeptide is of a predetermined length.

In one embodiment, each nucleic acid molecule further comprises a unique nucleotide barcode sequence encoding the same amino acid sequence which is shared by each member of the minigene library.

In some embodiments, the nucleic acid molecule encoding the minigene antigen expression cassette further comprises a sequence encoding at least one transmembrane domain for class II antigen presentation, or fragment thereof. In some embodiments, the nucleic acid molecule encoding the minigene antigen expression cassette further comprises SEQ ID NO:114, SEQ ID NO:116, SEQ ID NO:118, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, or SEQ ID NO:135.

In one embodiment, the nucleic acid molecule further comprises a nucleotide sequence encoding a 6× flexible Flag construct. In one embodiment, each nucleic acid molecule further comprises a nucleotide sequence of SEQ ID NO:42.

In one embodiment, each nucleic acid molecule further comprises a nucleotide sequence encoding at least one co-stimulatory molecule. In one embodiment, the co-stimulatory molecule is CD40, CD58, CD80, CD83, CD86, OX40L, 4-1BBL, or a combination thereof. In one embodiment, each nucleic acid molecule comprises a nucleotide sequence of SEQ ID NO:43-SEQ ID NO:49.

In one embodiment, the invention relates to an APC library comprising a plurality of APCs, wherein each APC comprises a nucleic acid molecule of a minigene library comprising a plurality of nucleic acid molecules wherein each nucleic acid molecule comprises a nucleotide sequence encoding at least one antigenic polypeptide for presentation, wherein the sequence encoding the at least one antigenic polypeptide is of a predetermined length. In one embodiment, each nucleic acid molecule further comprises a unique nucleotide barcode sequence encoding the same amino acid sequence which is shared by each member of the minigene library.

In one embodiment, the APC is a B cell, dendritic cell (DC), monocyte or macrophage. In one embodiment, the APCs are B cells. In one embodiment, the APCs are immortalized patient-derived B cells.

In one aspect, the present invention relates to a microfluidic device for co-encapsulation of individual cells, comprising: two or more proximal inlets fluidly connected to one or more distal outlets; a filter positioned downstream from each proximal inlet; a series of asymmetrical focusing loops positioned downstream from each filter; a nozzle positioned upstream from the one or more distal outlets; and one or more isolation fluid inlets connected to the nozzle; wherein each of the series of asymmetrical focusing loops comprise arcuate channel lengths curving in alternating directions about a central axis, such that arcuate channel lengths on a first side of the central axis are larger than arcuate channel lengths on an opposing second side of the central axis; and wherein each of the series of asymmetrical focusing loops converge into a single microchannel fluidly connected to the nozzle.

In one embodiment, a channel fluidly connecting each proximal inlet to a filter has a width that gradually expands to a width between about 10 μm and 10000 μm. In one embodiment, a channel fluidly connecting each proximal inlet to a filter comprises one or more stream separators, wherein each stream separator is an elongated barrier substantially in parallel alignment with the channel and is configured to evenly distribute fluid flow from each proximal inlet.

In one embodiment, the filter comprises a plurality of pillars spaced apart by a distance between about 50 μm and 5000 μm. In one embodiment, increasing spacing between pillars is configured to filter particles while minimizing clogging occurrences. In one embodiment, each pillar has a cross-sectional shape selected from the group consisting of: a circle, an oval, a triangle, a square, a rectangle, a diamond, a polygon, a V-shape, and a U-shape. In one embodiment, each pillar has a width between about 1 μm and 100 μm.

In one embodiment, each of the series of asymmetrical focusing loops has an overall wave-like shape. In one embodiment, each of the series of asymmetrical focusing loops is configured to encourage a flow of particles to evenly space apart both laterally and longitudinally within a flow of fluid.

In one embodiment, the arcuate channel lengths on the first side and the second side have equal widths. In one embodiment, the arcuate channel lengths on the first side have a width between about 50 μm and 10000 μm. In one embodiment, the arcuate channel lengths on the second side have a width between about 20 μm and 5000 μm. In one embodiment, the arcuate channel lengths on the first side and the second side comprise a curvature defined by a degree of a substantially circular path. In one embodiment, the degree is between about 5° and 355°. In one embodiment, the degree is about 180°. In one embodiment, the arcuate channel lengths on the first side and the second side comprise a curvature defined by a diameter of a substantially circular path. In one embodiment, the diameter is between about 20 μm and 1000 μm. In one embodiment, the diameter is about 110 μm for the arcuate channel lengths on the first side. In one embodiment, the diameter is about 50 μm for the arcuate channel lengths on the second side.

In one embodiment, a channel fluidly connects each of the series of asymmetrical focusing loops to the single microchannel fluidly connected to the nozzle. In one embodiment, the channel has a tapered width. In one embodiment, a channel fluidly connects the nozzle with each of the one or more outlets. In one embodiment, the channel has an expanded width. In one embodiment, the channel is aligned in-line with the single microchannel. In one embodiment, two channels fluidly connect each of the isolation fluid inlets to the nozzle. In one embodiment, the two channels fluidly connect on opposing sides of the nozzle.

In one embodiment, the device further comprises one or more additional inlets fluidly connected directly to or to a position between one or more of the proximal inlets, filters, focusing loops, nozzle, and outlet. In one embodiment, the device further comprises one or more flow modulators positioned between one or more of the proximal inlets, filters, focusing loops, nozzle, and outlet, wherein the one or more flow modulators is selected from the group consisting of: valves, fluid resistors, expandable elements, contractible elements, pumps, and membranes.

In one aspect, the present invention relates to a method of forming co-encapsulated particles, comprising the steps of: providing a microfluidic device of the present invention; providing a first suspension of a particle providing at least one second suspension of a particle; flowing each of the suspensions through a proximal inlet of the device; and flowing an isolation fluid through an isolation fluid inlet of the device.

In one embodiment, the particle of the first suspension and the at least one second suspension is selected from the group consisting of: cells, viruses, bacteria, amoeba, protozoa, paramecium, microparticles, nanoparticles, beads, microorganisms, vesicles, nucleic acid oligonucleotides, proteins, polypeptides, carbohydrates, and fragments thereof.

In one embodiment, the first suspension and the at least one second suspension comprises a suspension fluid selected from the group consisting of: water, cell growth media, serum, plasma, and oil. In one embodiment, the isolation fluid is immiscible with the suspension fluid.

In one embodiment, the particle of the first suspension is a B cell. In one embodiment, the first suspension comprises one or more additives. In one embodiment, the one or more additives comprises a chelating agent. In one embodiment, the chelating agent is configured to inhibit B-cell clumping in a B cell suspension. In one embodiment, the chelating agent is selected from EDTA and EGTA.

In one embodiment, the particle of the at least one second suspension is a T cell. In one embodiment, the at least one second suspension comprises one or more additives. In one embodiment, the one or more additives comprises an ion additive. In one embodiment, the ion additive is configured to restore ion concentration balance in a B-cell and T-cell co-encapsulate, such that T-cell activation is rescued within the B-cell and T-cell co-encapsulate. In one embodiment, the ion additive is calcium.

In one embodiment, the invention relates to a nucleic acid molecule comprising a nucleotide sequence encoding at least one antigenic polypeptide for presentation at a cell surface, and further comprising at least one transmembrane sequence for enhanced presentation, or fragment thereof. In one embodiment, the transmembrane domain is SEQ ID NO:114, SEQ ID NO:116, SEQ ID NO:118, SEQ ID NO:120, SEQ ID NO:121, SEQ ID NO:122, SEQ ID NO:123, SEQ ID NO:124, SEQ ID NO: 125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, or SEQ ID NO:135.

In one embodiment, the antigenic polypeptide is a pathogenic peptide. In one embodiment, the antigenic polypeptide is a viral antigen.

In one embodiment, the invention relates to an immunogenic composition comprising at least one nucleic acid molecule comprising a nucleotide sequence encoding at least one antigenic polypeptide for presentation at a cell surface, and further comprising at least one transmembrane sequence for enhanced presentation. In one embodiment, the transmembrane domain is SEQ ID NO:114, SEQ ID NO:116, SEQ ID NO:118, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO: 129, SEQ ID NO:130, SEQ ID NO:131, SEQ ID NO:132, SEQ ID NO:133, SEQ ID NO:134, or SEQ ID NO:135.

In one embodiment, the antigenic polypeptide is a pathogenic peptide. In one embodiment, the antigenic polypeptide is a viral antigen.

In one embodiment, the composition comprises a vaccine or a vaccine adjuvant.

In one embodiment, the invention relates to a method of inducing an immune response, the method comprising administering an immunogenic composition comprising at least one nucleic acid molecule comprising a nucleotide sequence encoding at least one antigenic polypeptide for presentation at a cell surface, and further comprising at least one transmembrane sequence for enhanced presentation to a subject in need thereof. In one embodiment, the transmembrane domain is SEQ ID NO:114, SEQ ID NO:116, SEQ ID NO:118, SEQ ID NO:120, SEQ ID NO:121, SEQ ID NO:122, SEQ ID NO: 123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, or SEQ ID NO:135.

In one embodiment, the invention relates to a method of enhancing a vaccine response, the method comprising administering an immunogenic composition comprising at least one nucleic acid molecule comprising a nucleotide sequence encoding at least one antigenic polypeptide for presentation at a cell surface, and further comprising at least one transmembrane sequence for enhanced presentation to a subject in need thereof. In one embodiment, the transmembrane domain is SEQ ID NO:114, SEQ ID NO:116, SEQ ID NO:118, SEQ ID NO:120, SEQ ID NO:121, SEQ ID NO:122, SEQ ID NO: 123, SEQ ID NO:124, SEQ ID NO:125, SEQ ID NO:126, SEQ ID NO:127, SEQ ID NO: 128, SEQ ID NO:129, SEQ ID NO:130, SEQ ID NO:131, SEQ ID NO:132, SEQ ID NO:133, SEQ ID NO:134, or SEQ ID NO:135.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 depicts a flow diagram of the cellular interaction screening method.

FIG. 2 depicts a diagram of a T cell secreted αCD19 scFv as a B cell targeting reporter.

FIG. 3 depicts a diagram of a reporter T cells expressing the secreted αCD19 scFv under the control of a promoter activated upon interaction of a T cell receptor with an antigen.

FIG. 4 depicts a diagram of an immortalized B cell presenting an antigen as part of a minigene library.

FIG. 5 depicts a diagram of a universal antigen expression vector that has been established for minigene libraries from multiple sources.

FIG. 6 depicts a diagram of the method for microfluidic co-culture and antigen recovery.

FIG. 7 depicts a diagram of a microfluidic device design with structural modifications that lead to higher efficiency and stability in droplet formation.

FIG. 8A through FIG. 8D depict exemplary experimental data demonstrating that Ofatumumab as secreted αCD20-scFv can be used to label BOLETH cells. FIG. 8A depicts a schematic representation of the experimental layout. HEK cells are transiently transfected with scFv-containing CMV expression constructs. The supernatant (SN) is recovered 72 hours post-transfection and used to label BOLETH cells. FIG. 8B depicts a comparison of αCD20-V5, in the VH-VL configuration (left) and αCD19-HA (right) labeled BOLETH cells as representative flow cytometry histograms and quantified median fluorescence intensity (MFI) of signals from αCD19-HA and αCD20-V5. FIG. 8C depicts a comparison of tags on the αCD20-scFv in the VH-VL configuration, V5 (left) and HA (right). Representative flow cytometry histograms and quantified median fluorescence intensity (MFI) of V5 and HA signal from labeled BOLETH cells. FIG. 8D depicts a comparison of signal strength from BOLETH cells labeled with αCD20-scFv in the VH-linker-VL or VL-linker-VH configurations depicted as representative flow cytometry histograms and quantified median fluorescence intensity (MFI) of V5 signal from both αCD20 configurations. SN of sfGFP transfected HEK cells was used as negative control. Error bars show ±1 standard deviation (SD). Dots represent one experiment with 4 replicates, data was reliably reproduced across several experiments. ns, not significant; *, P<0.05; **, P<0.01; *** P<0.001 (unpaired Welch's t-test).

FIG. 9A through FIG. 9C depict exemplary experimental data demonstrating that tandem scFv-reporter constructs have reduced αCD20-scFv signal strength. FIG. 9A depicts a comparison of αCD19-HA and αCD20-V5 containing tandem constructs with changed expression order. Representative flow cytometry histograms of V5 (left) and HA (right) signal from both scFvs and quantified median fluorescence intensity (MFI) of the double constructs. FIG. 9B depicts a comparison of tandem constructs containing either αCD20-V5 in the VH-VL or VL-VH configuration.

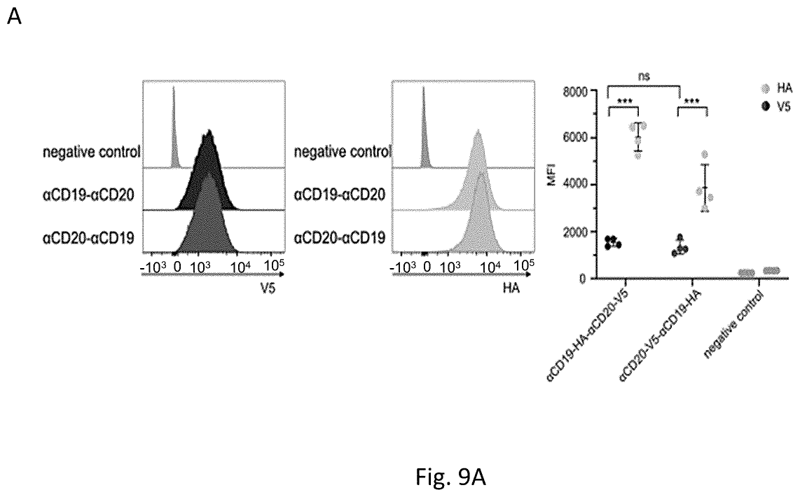

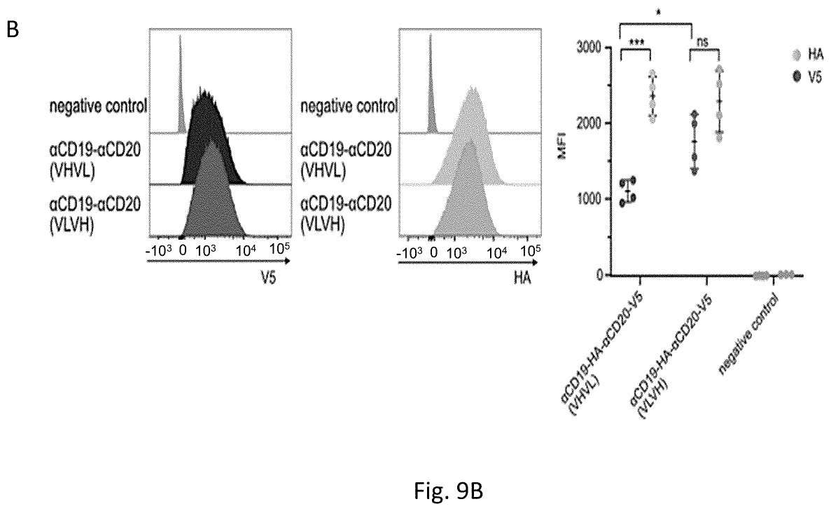

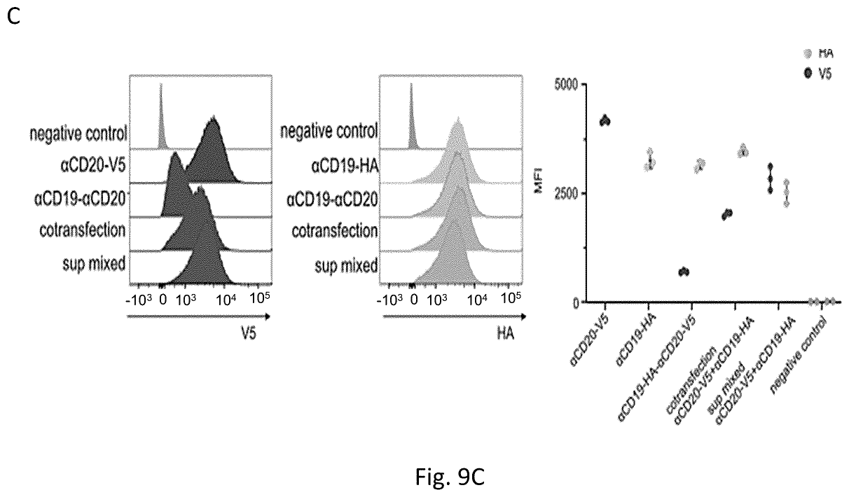

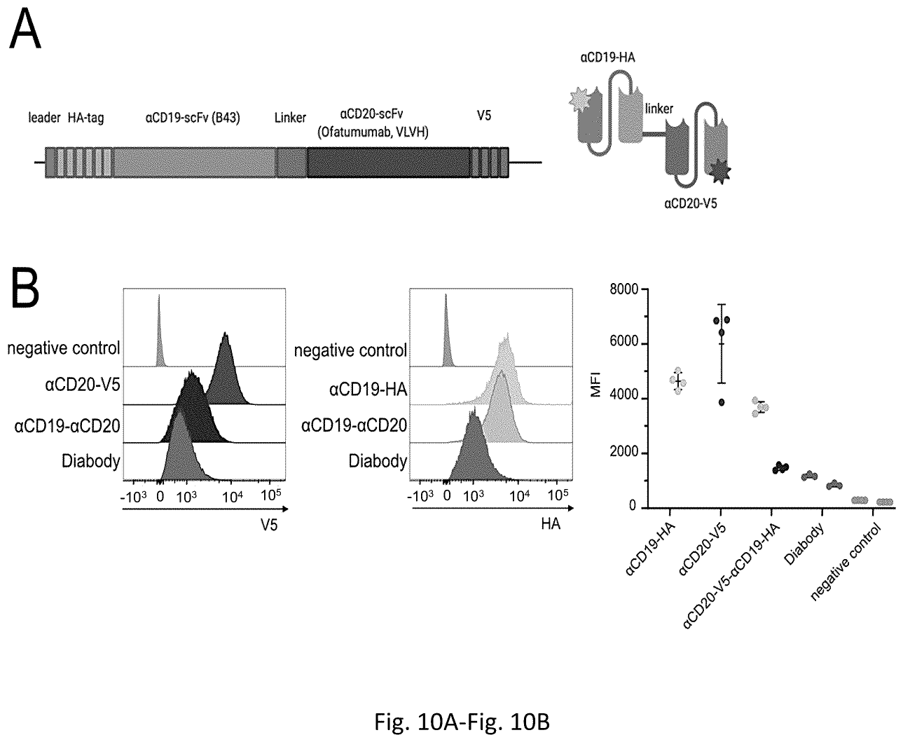

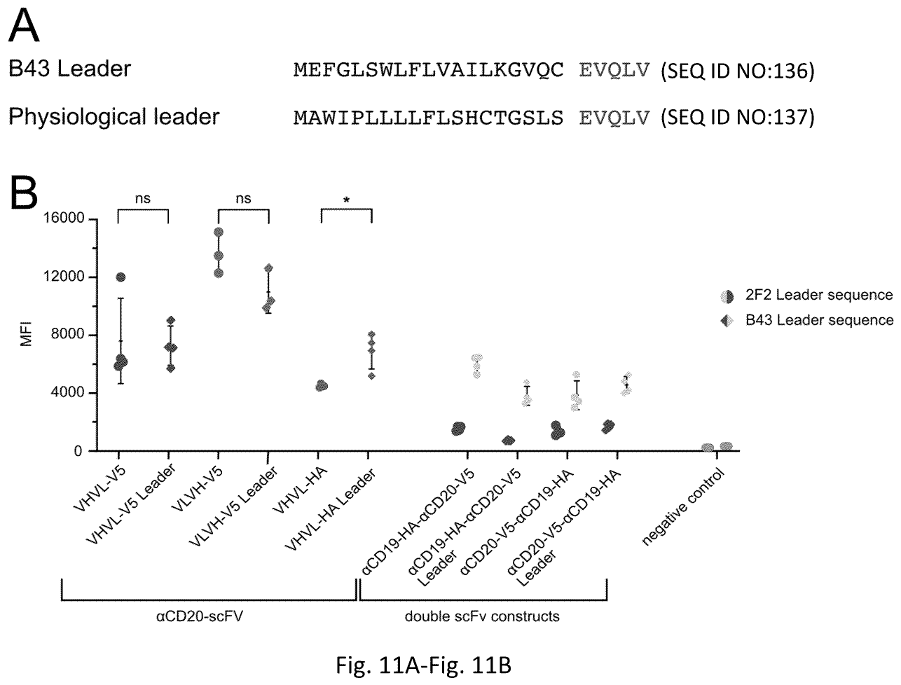

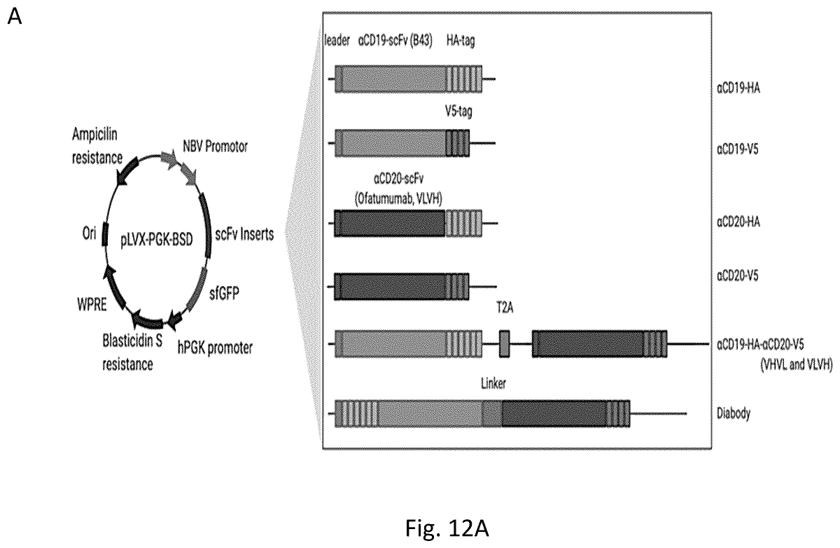

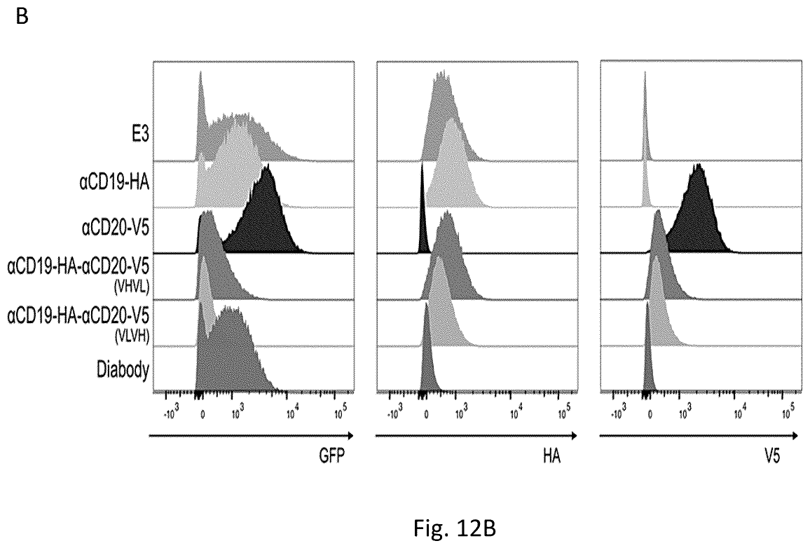

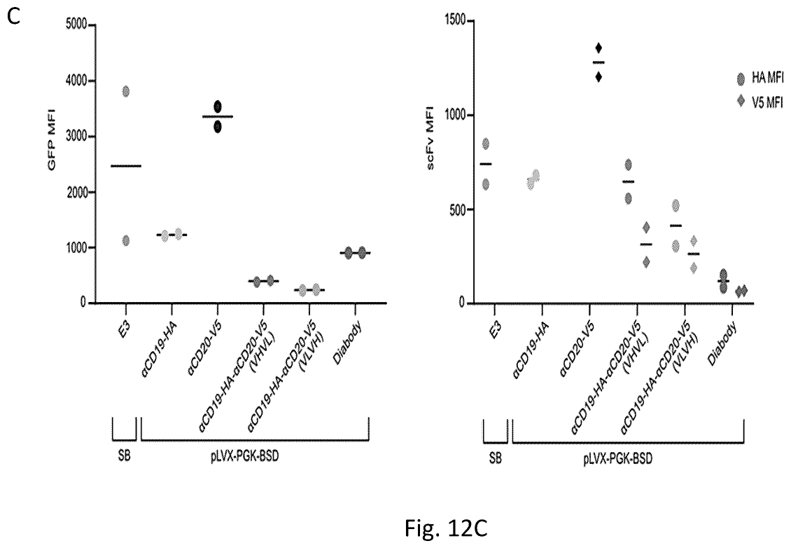

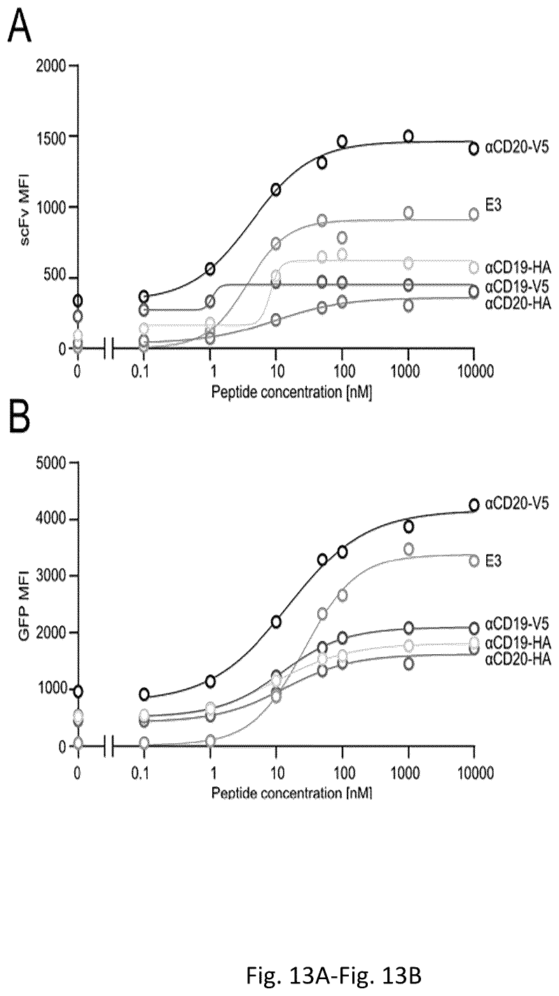

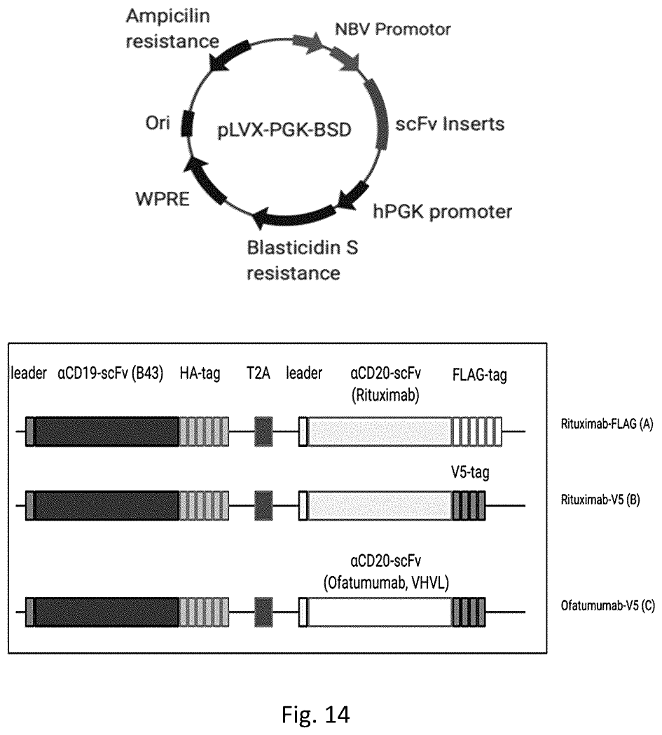

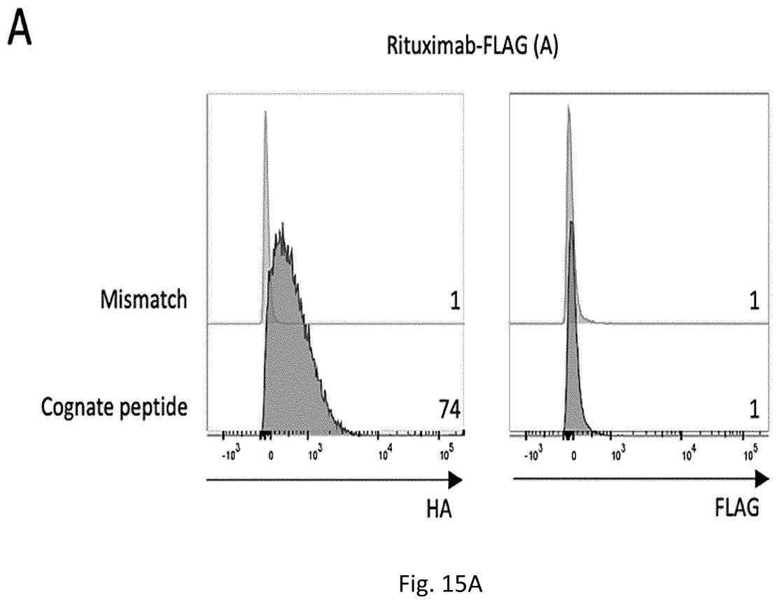

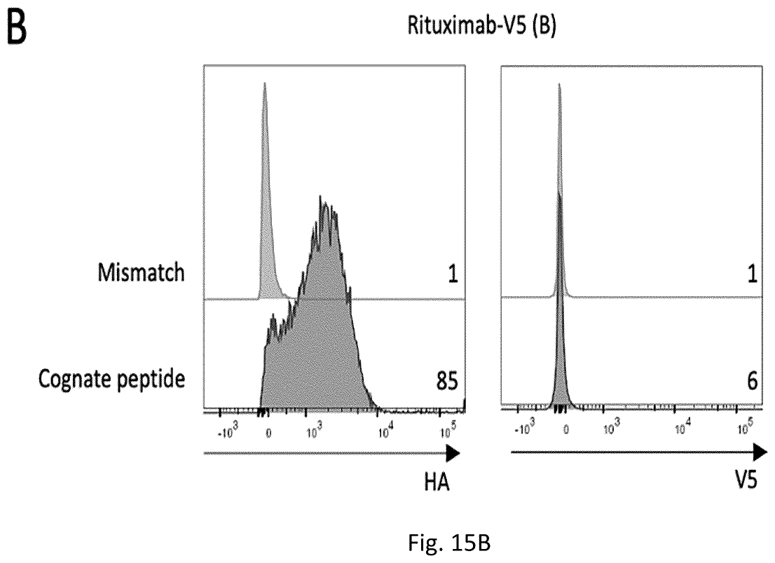

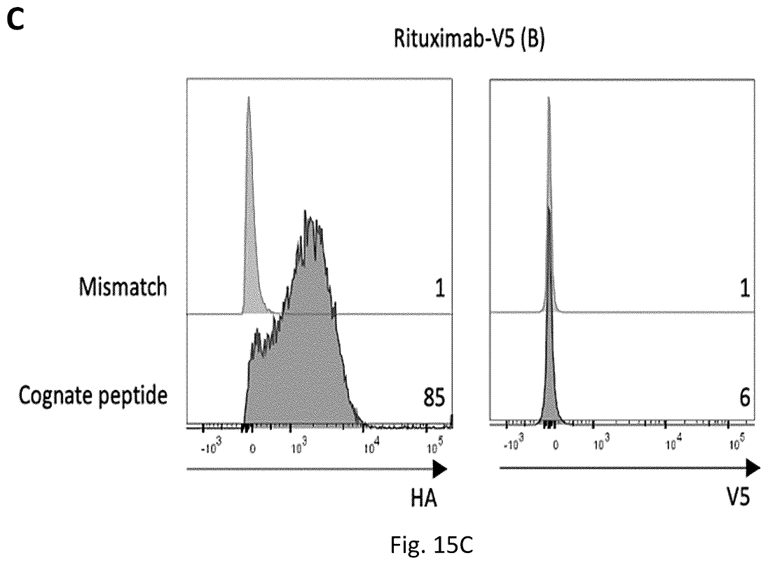

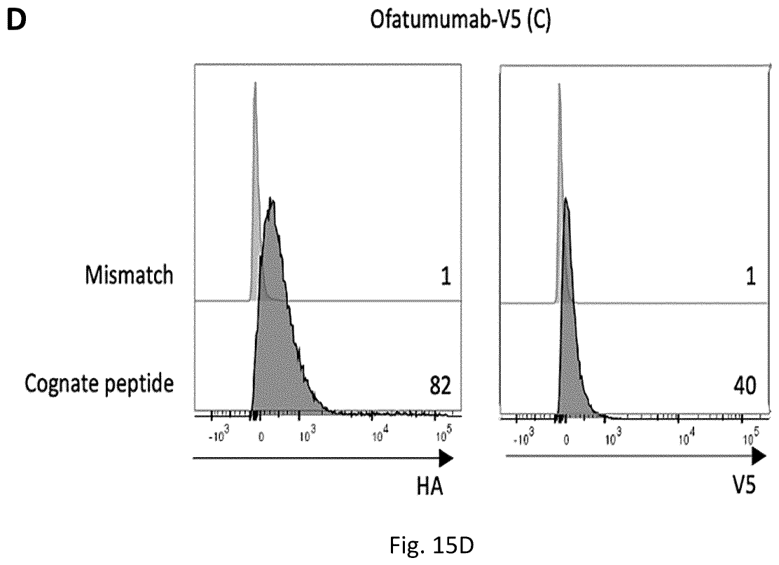

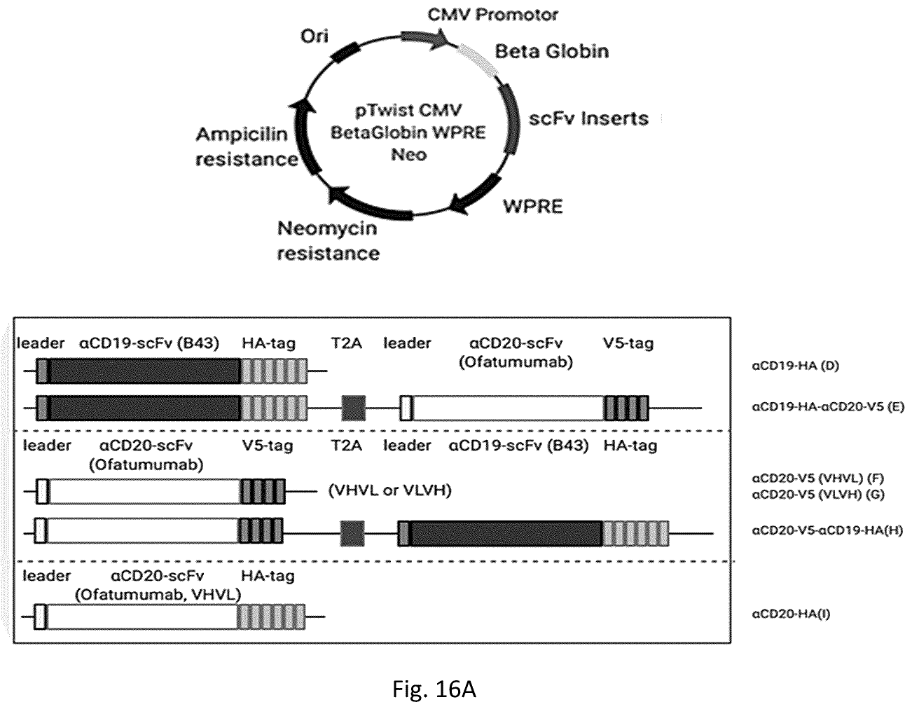

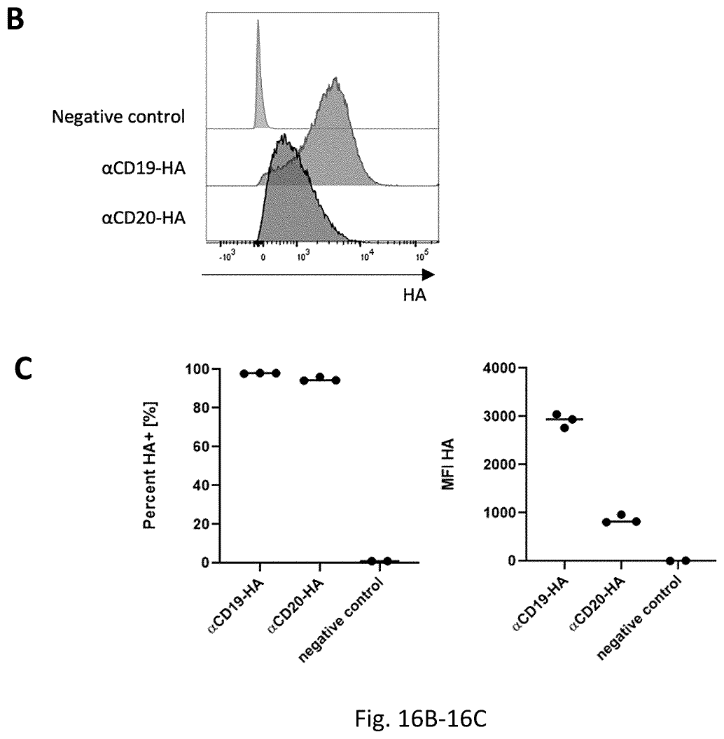

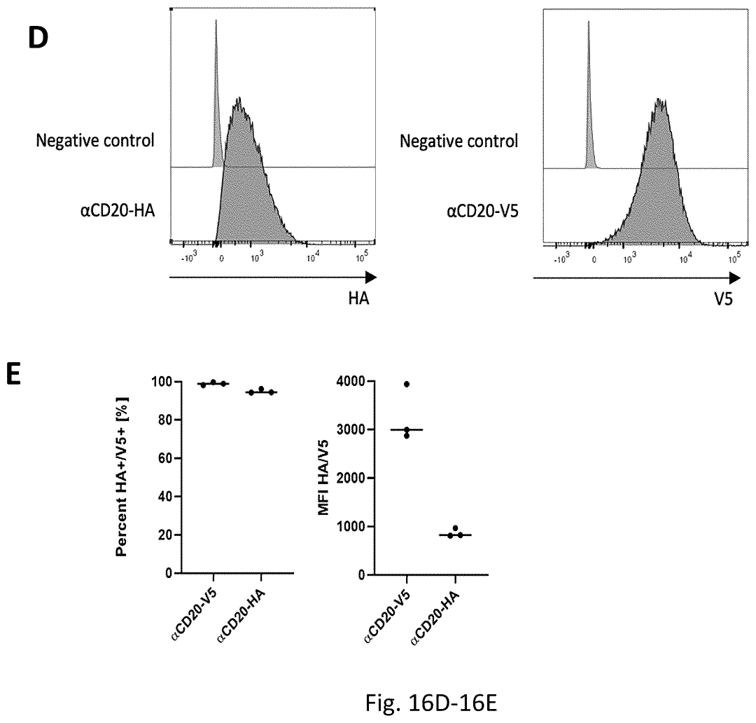

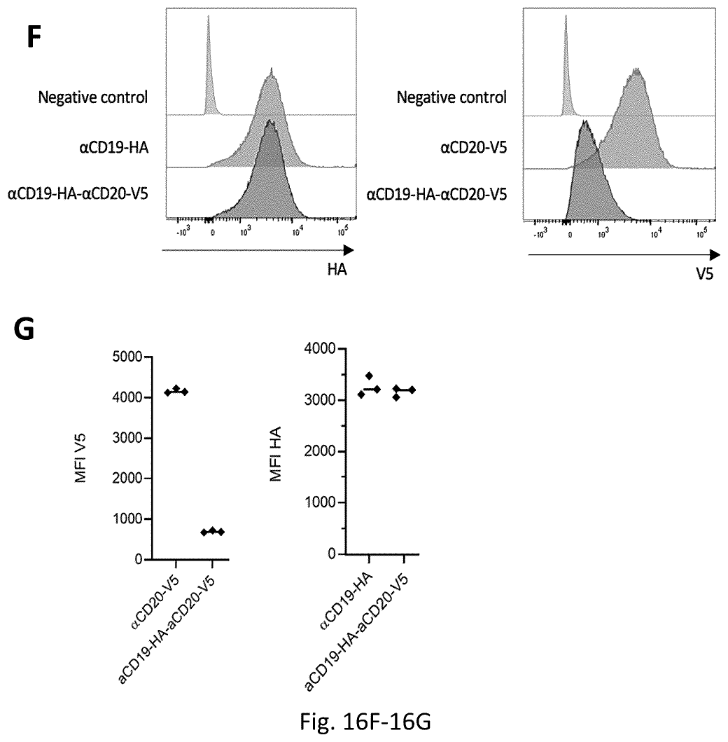

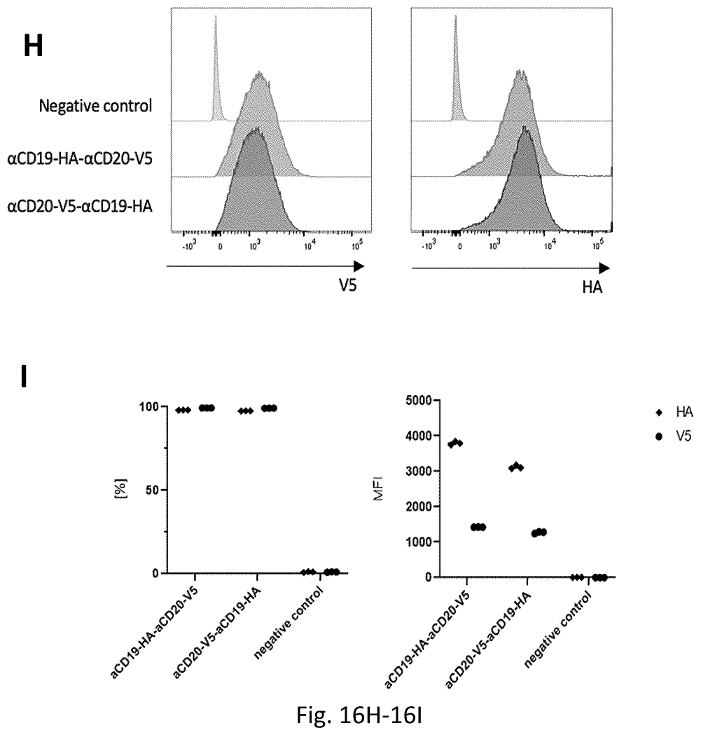

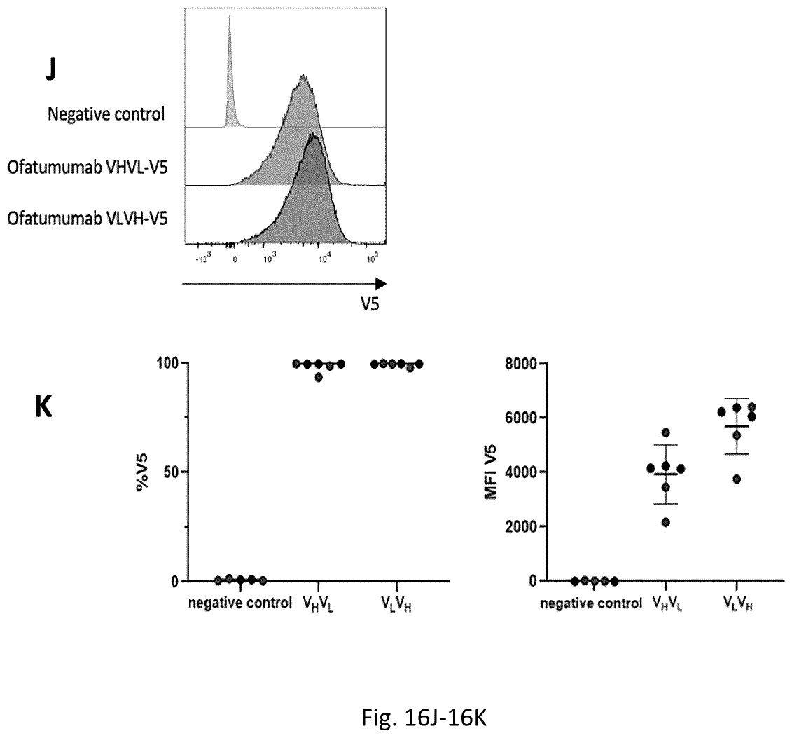

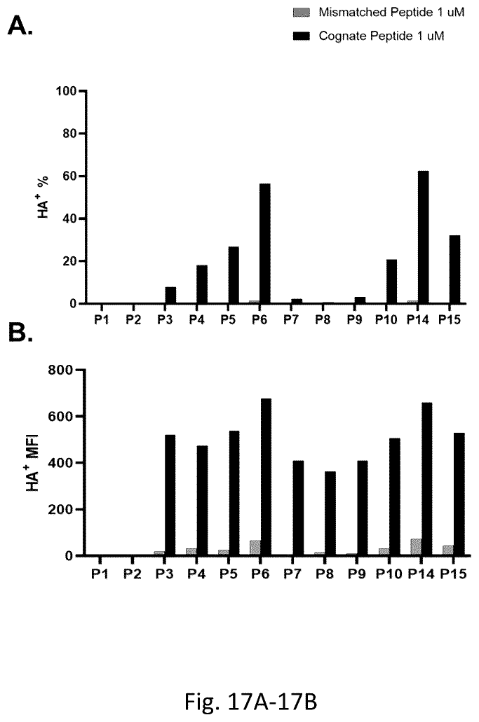

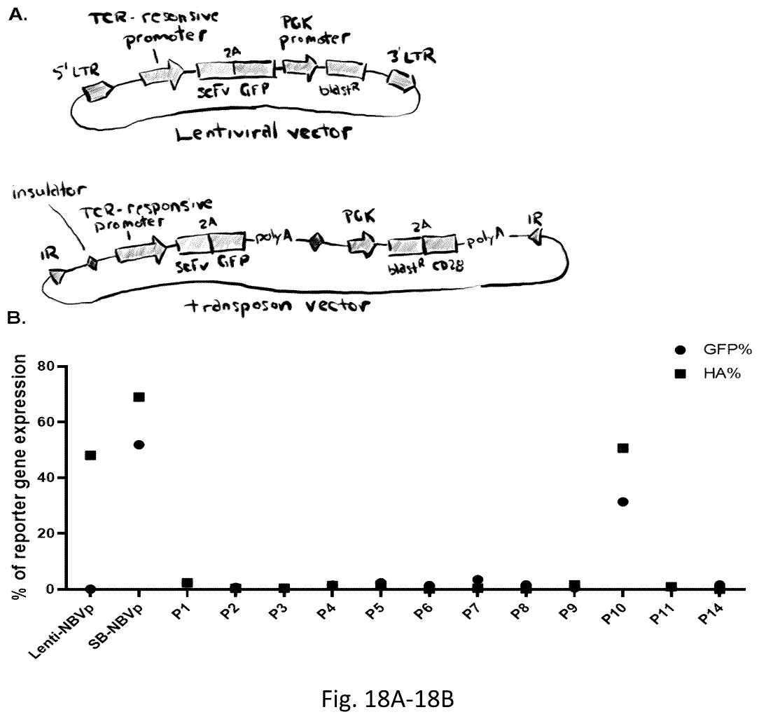

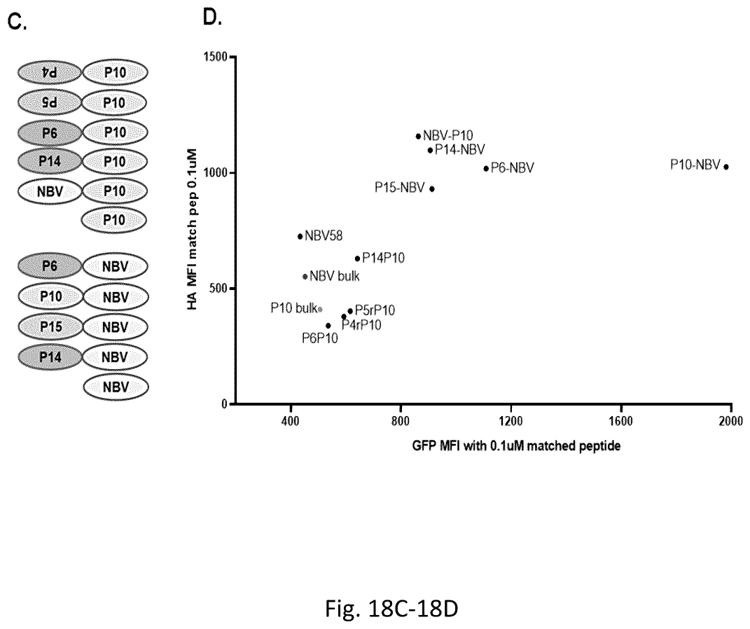

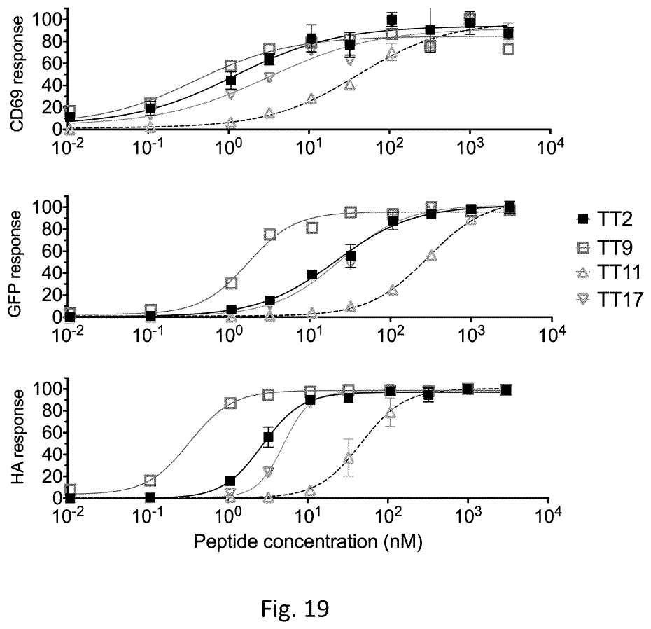

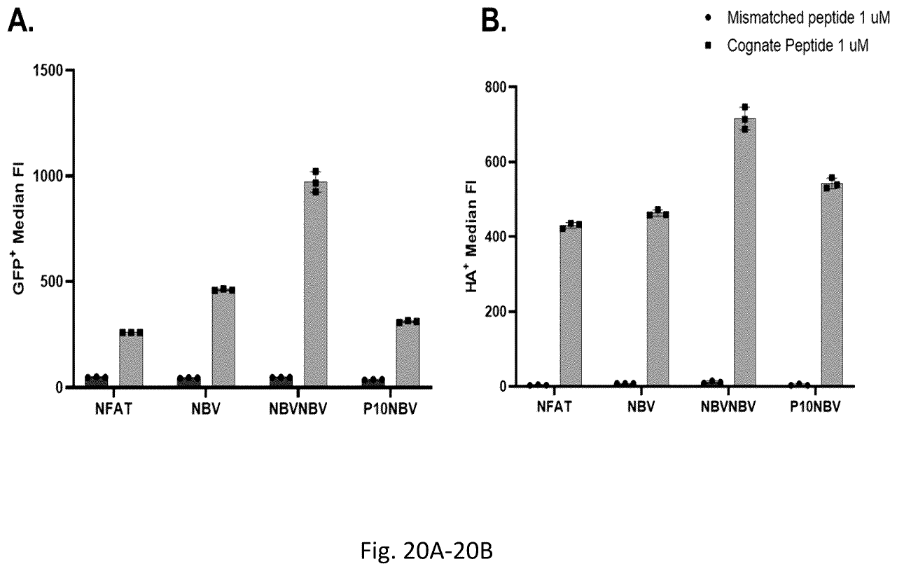

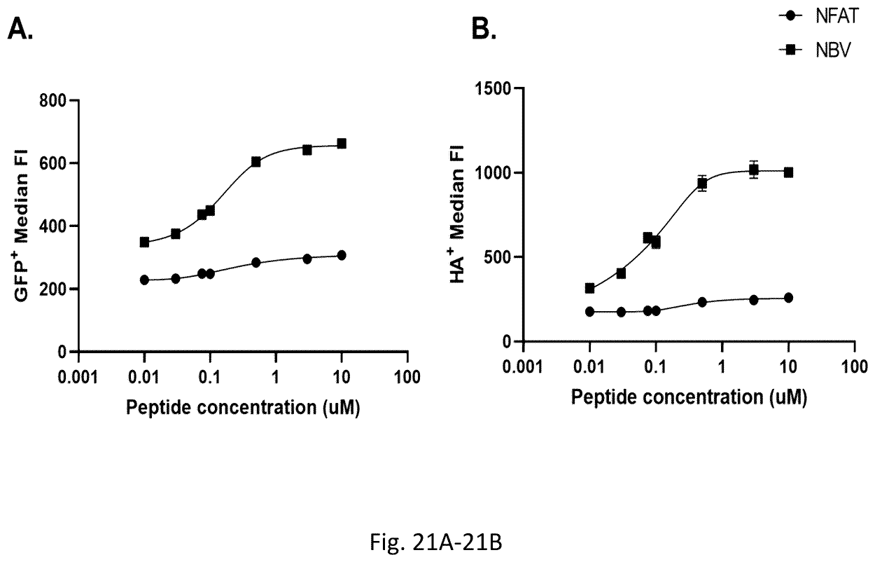

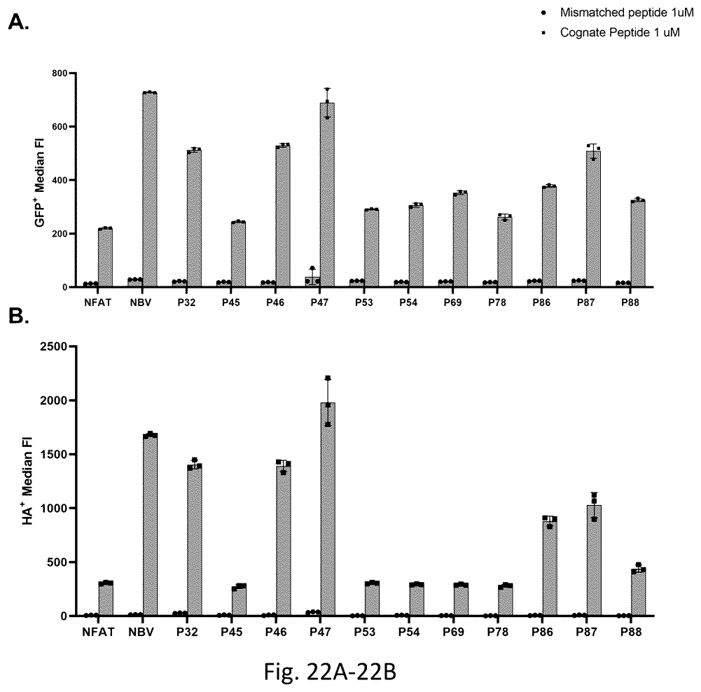

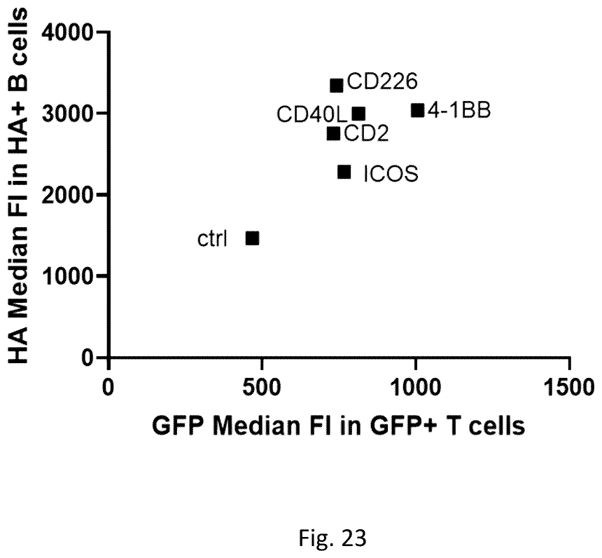

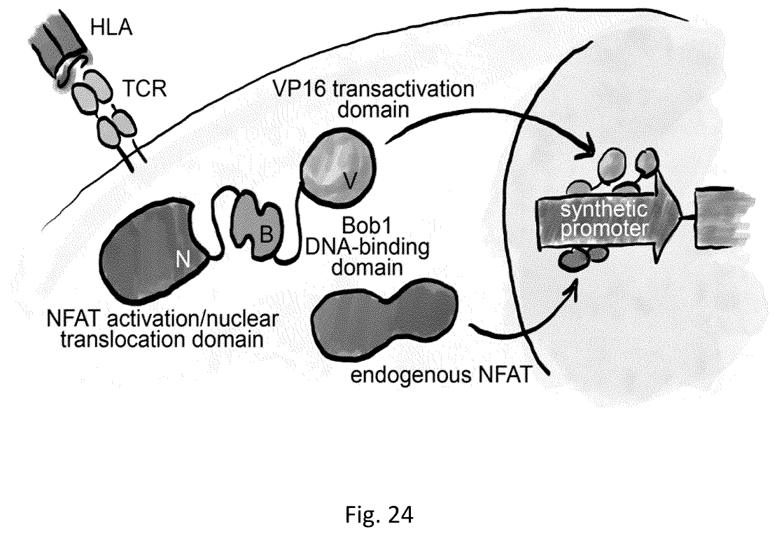

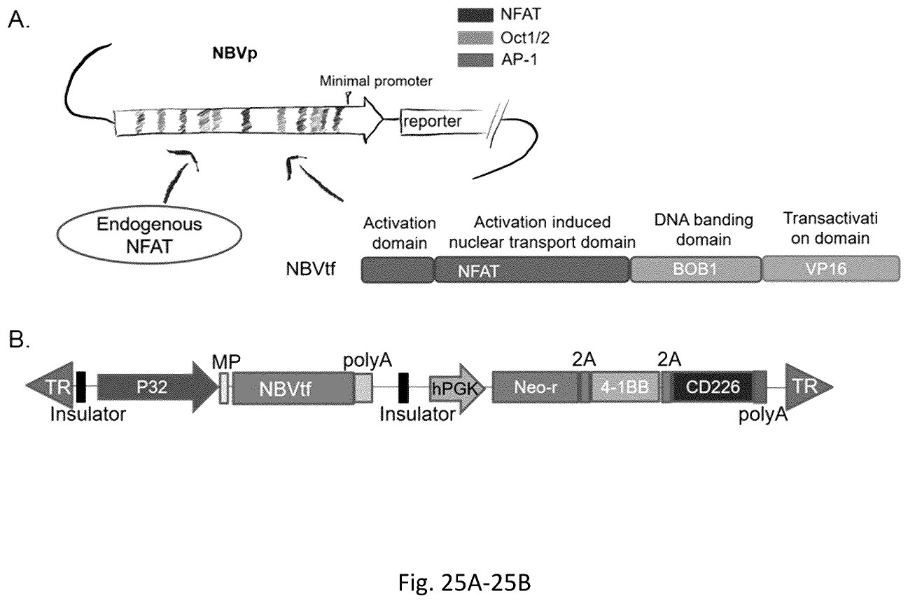

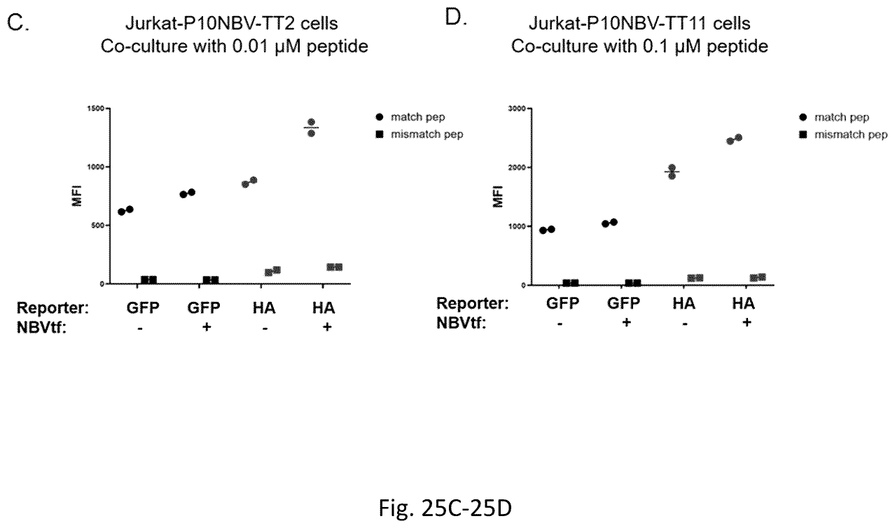

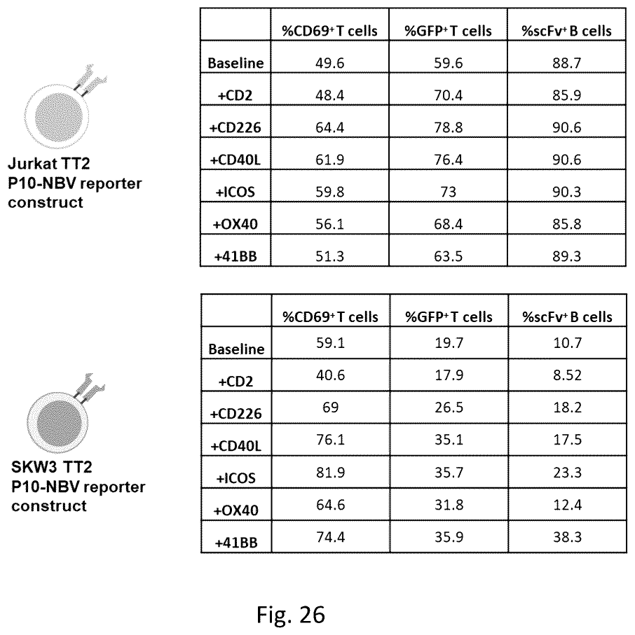

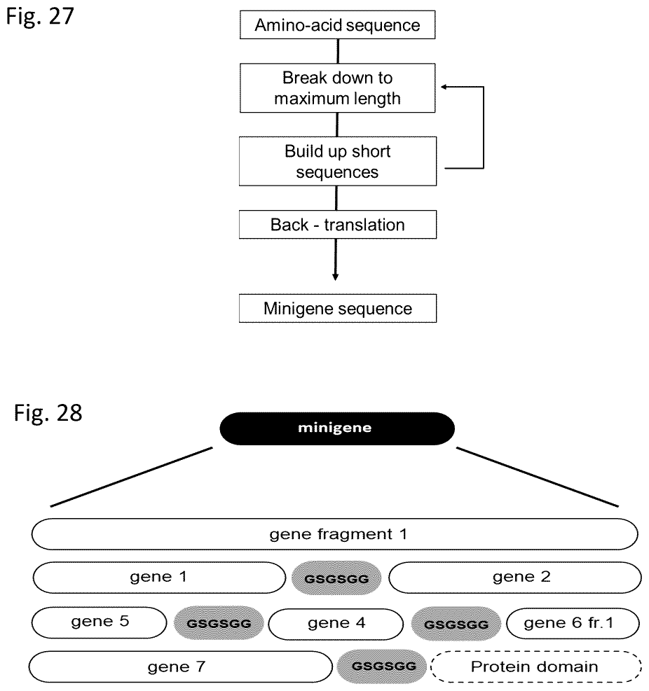

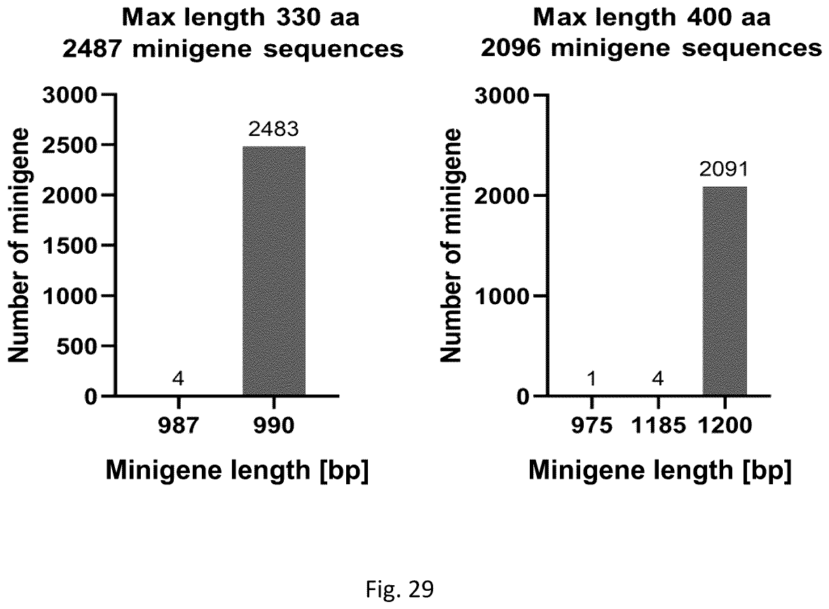

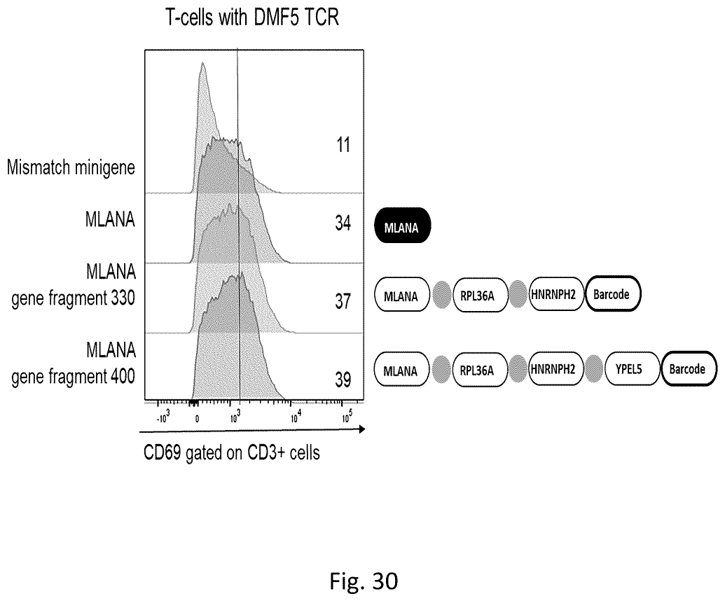

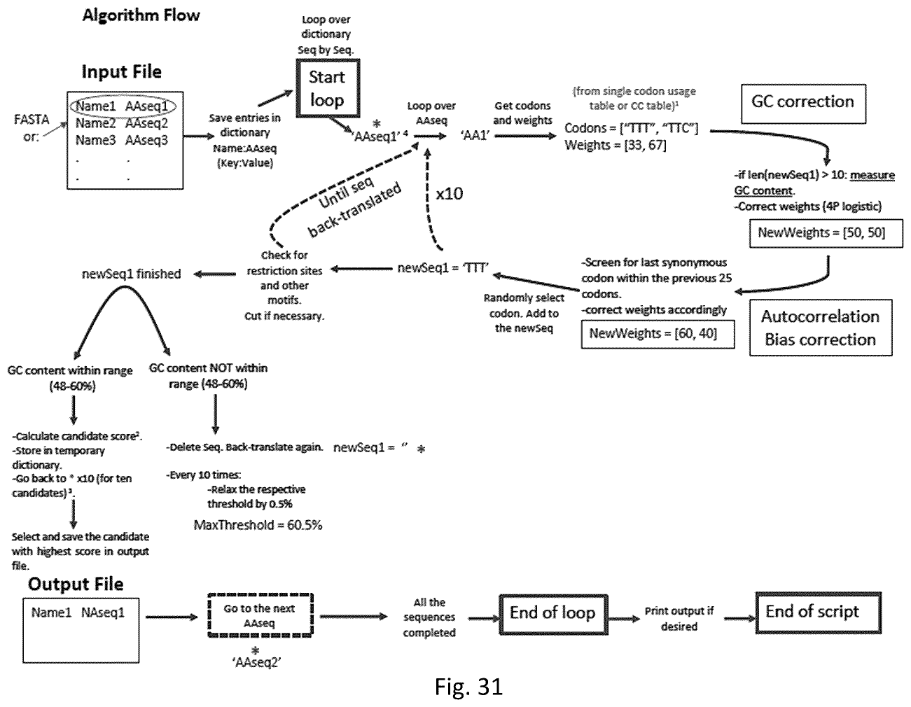

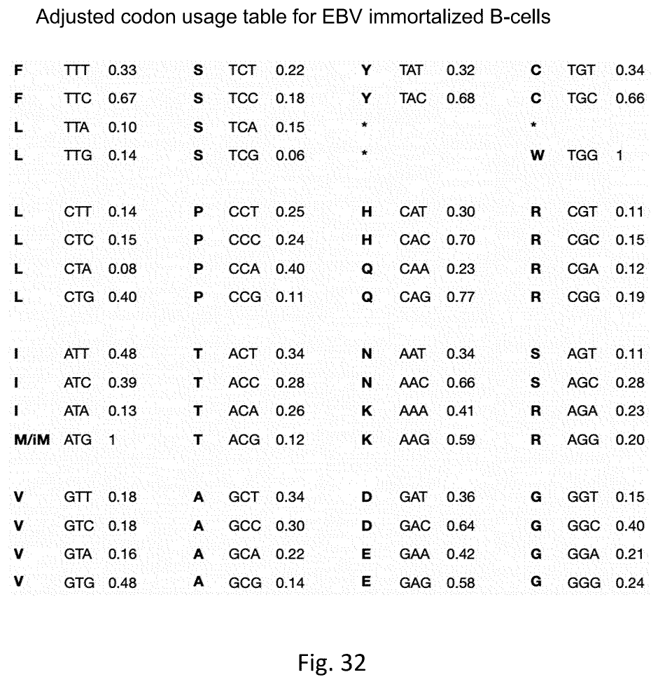

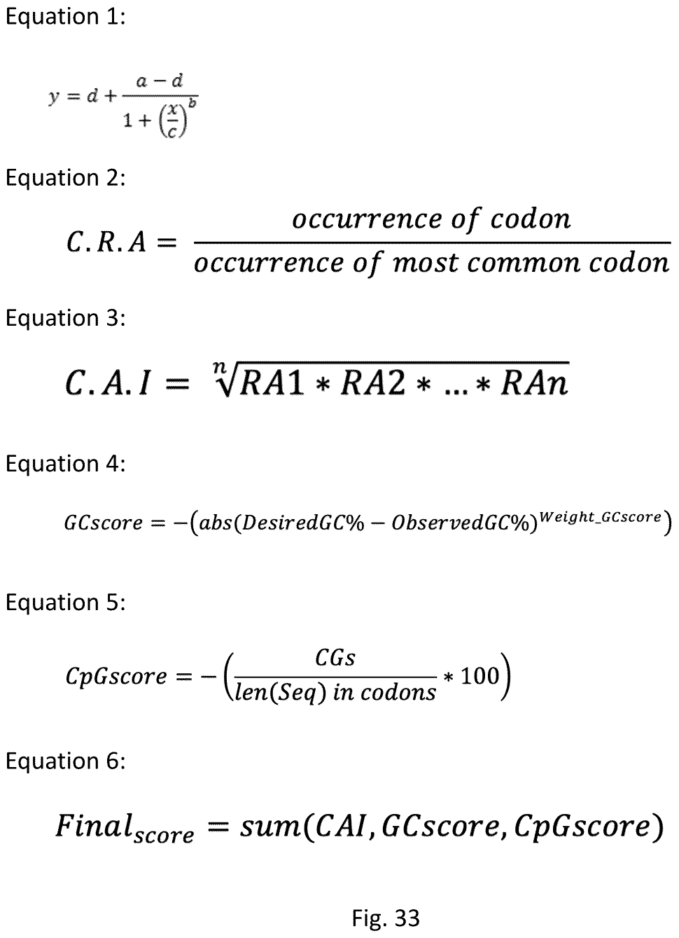

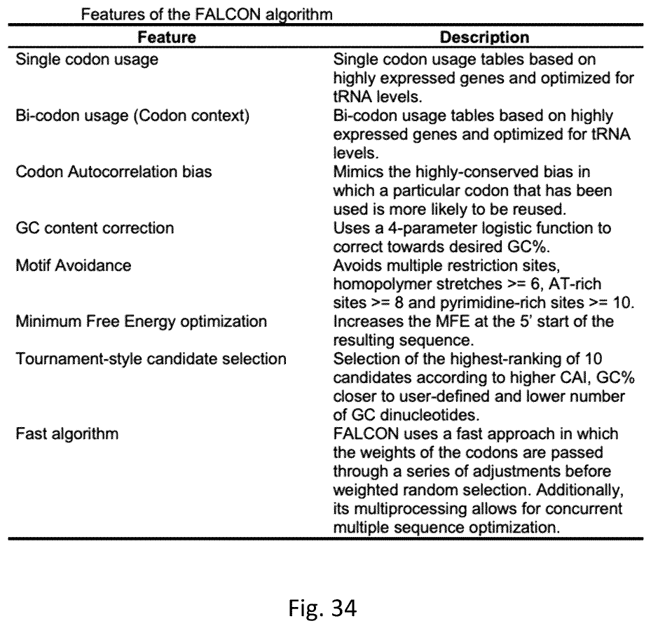

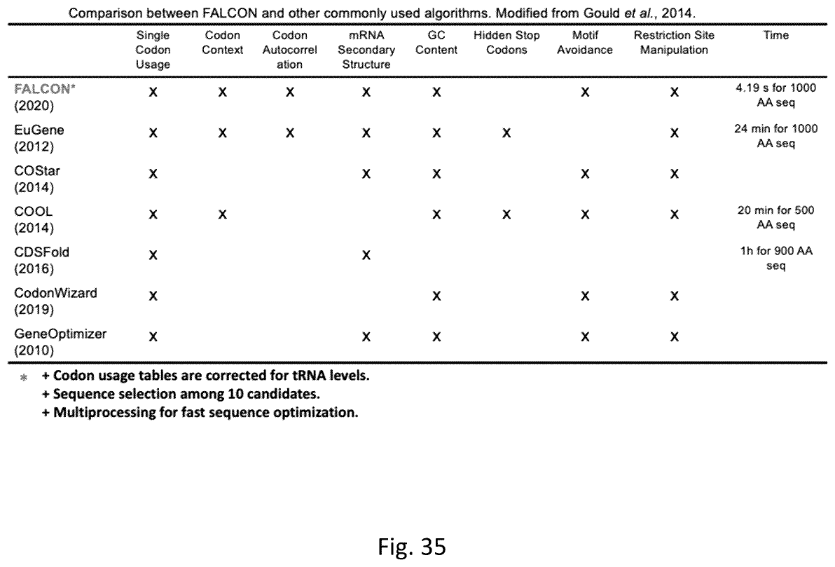

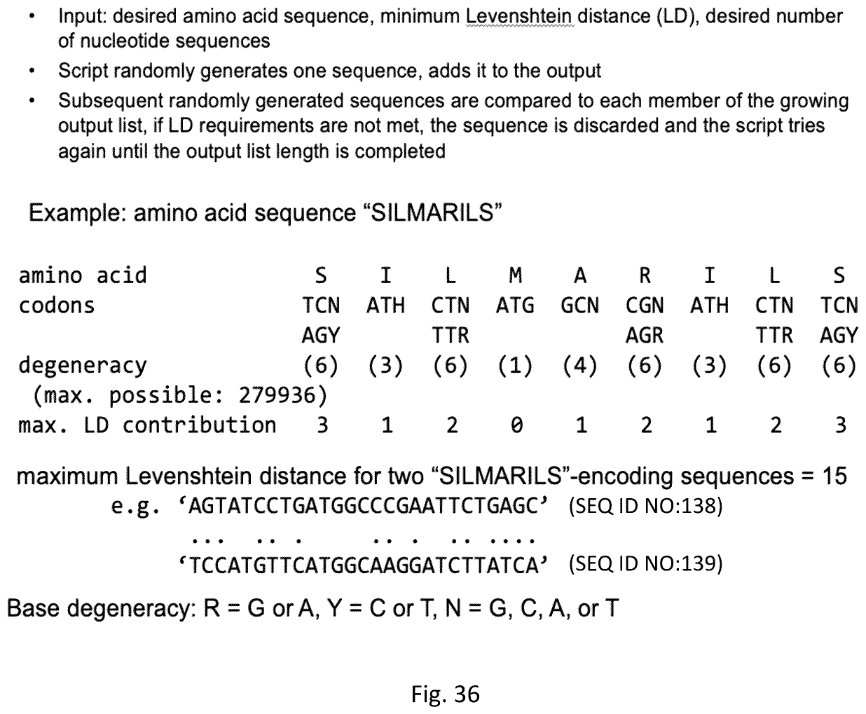

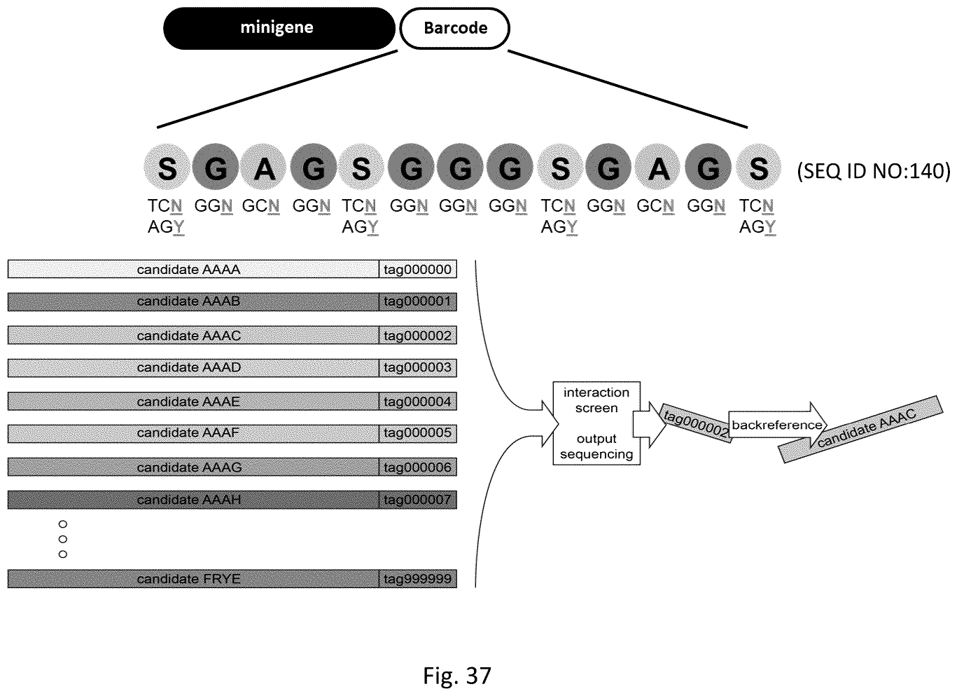

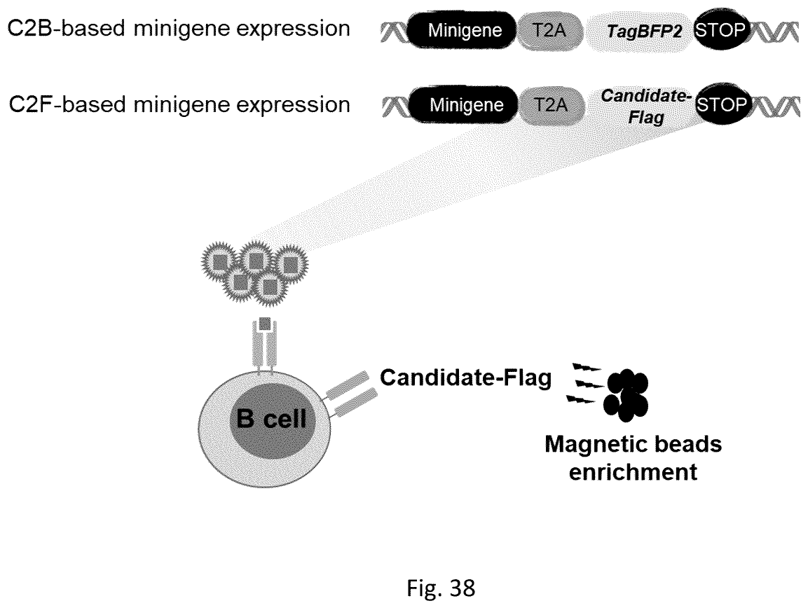

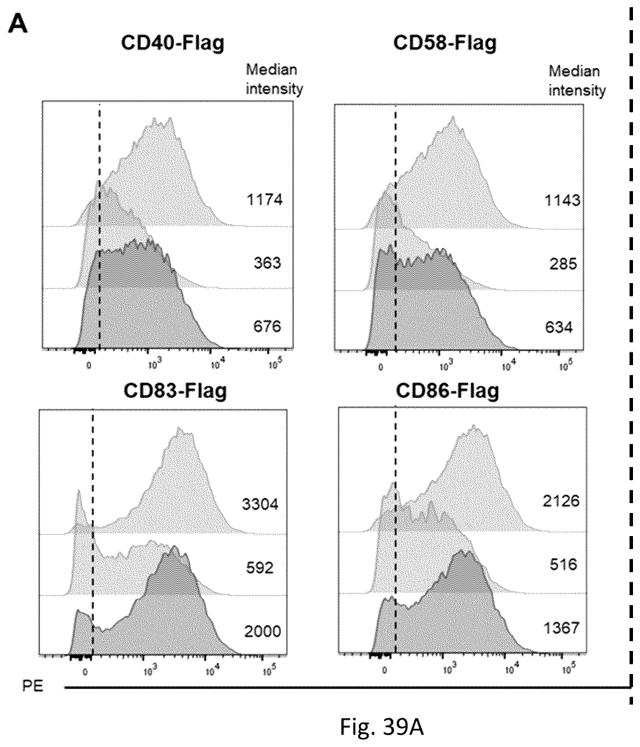

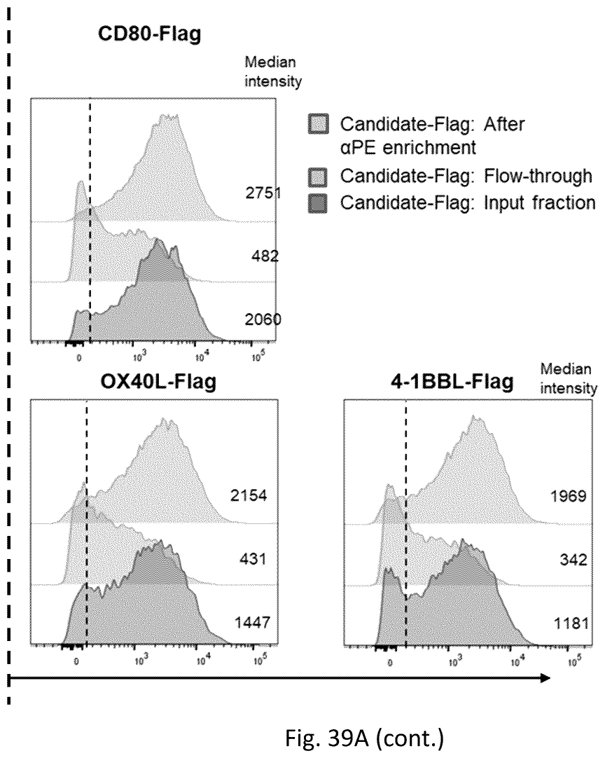

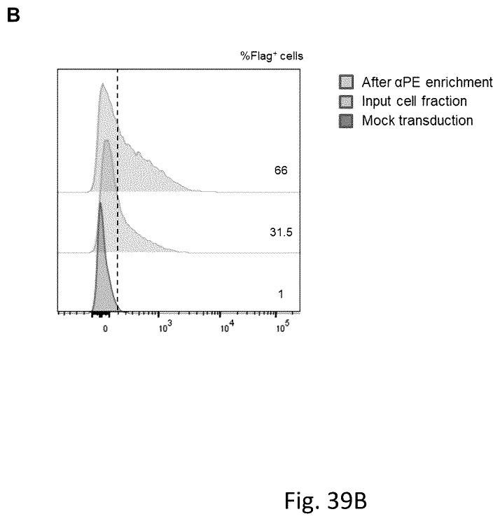

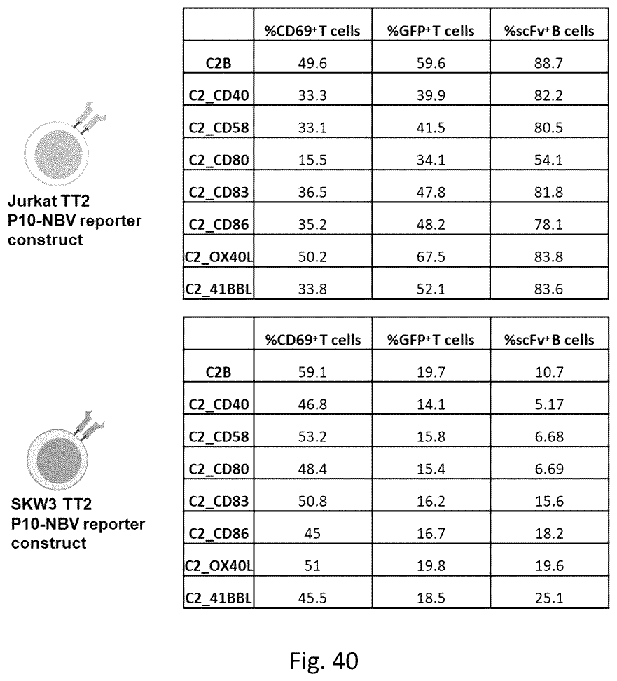

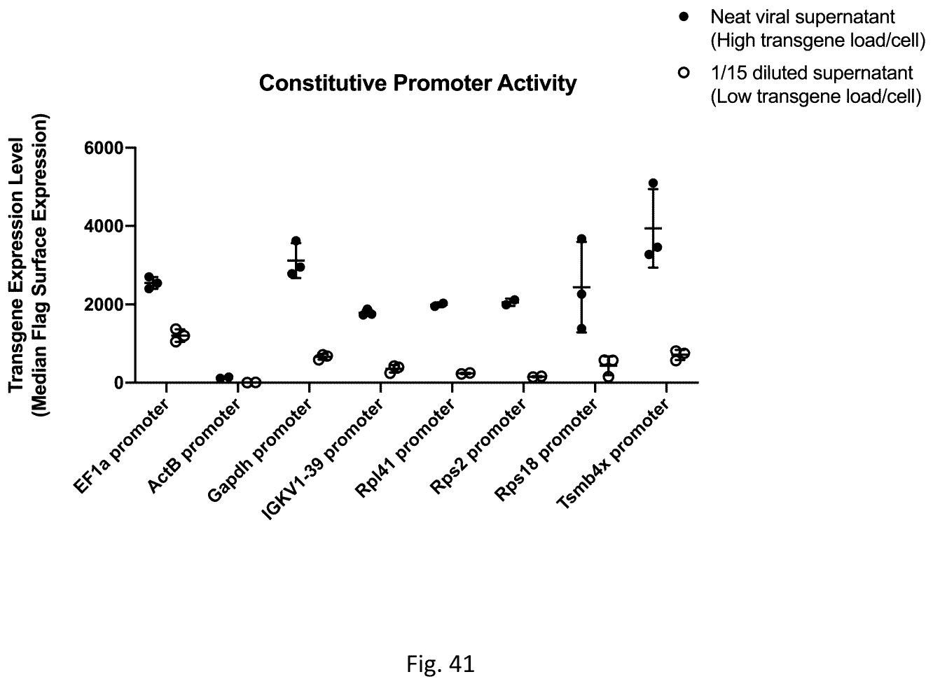

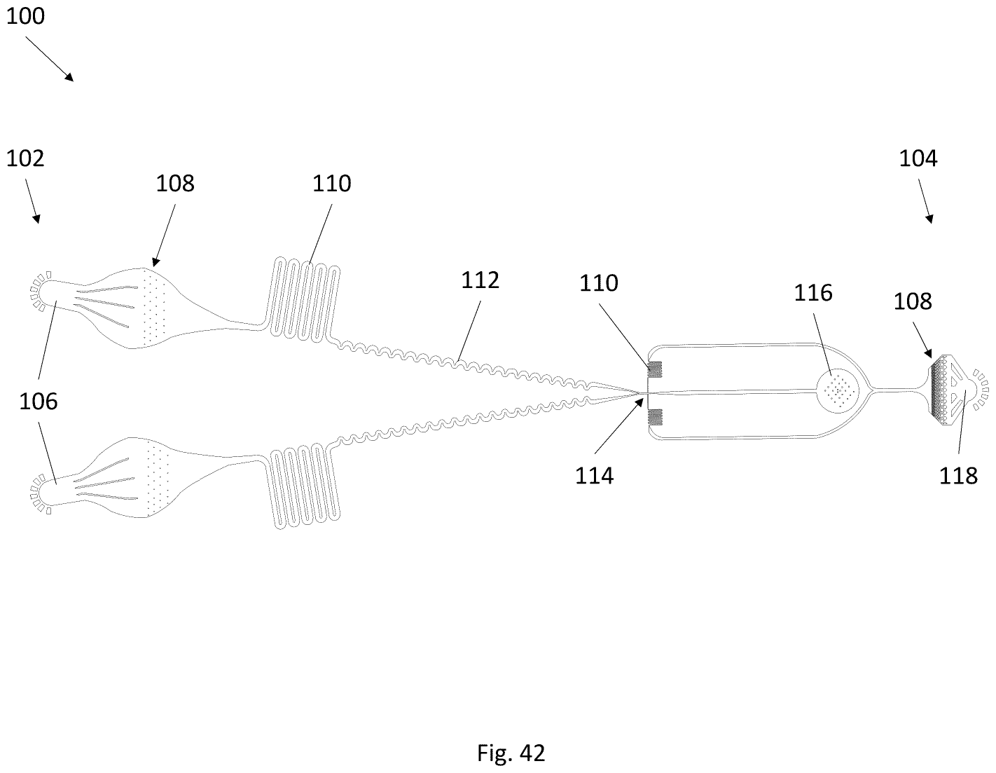

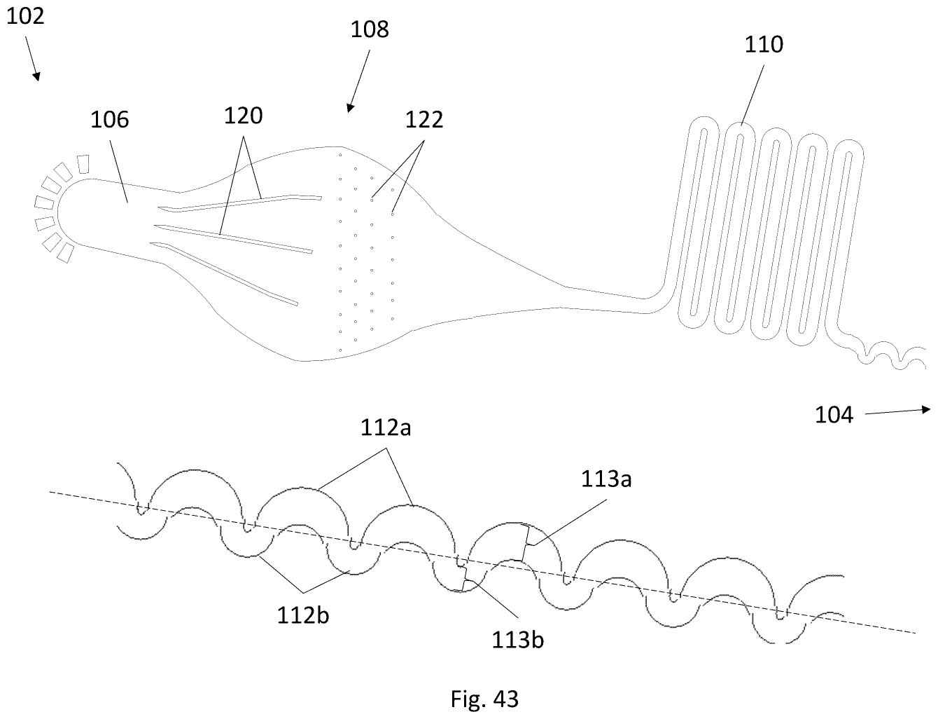

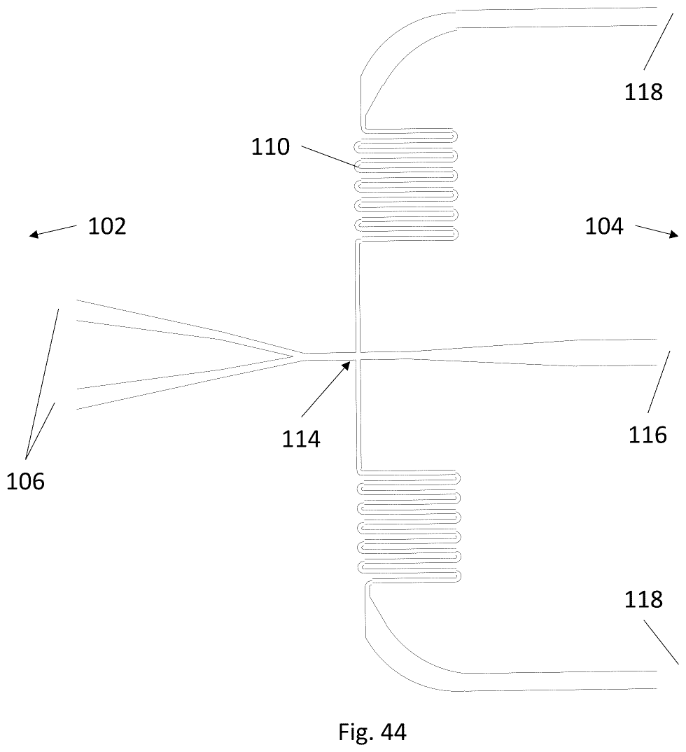

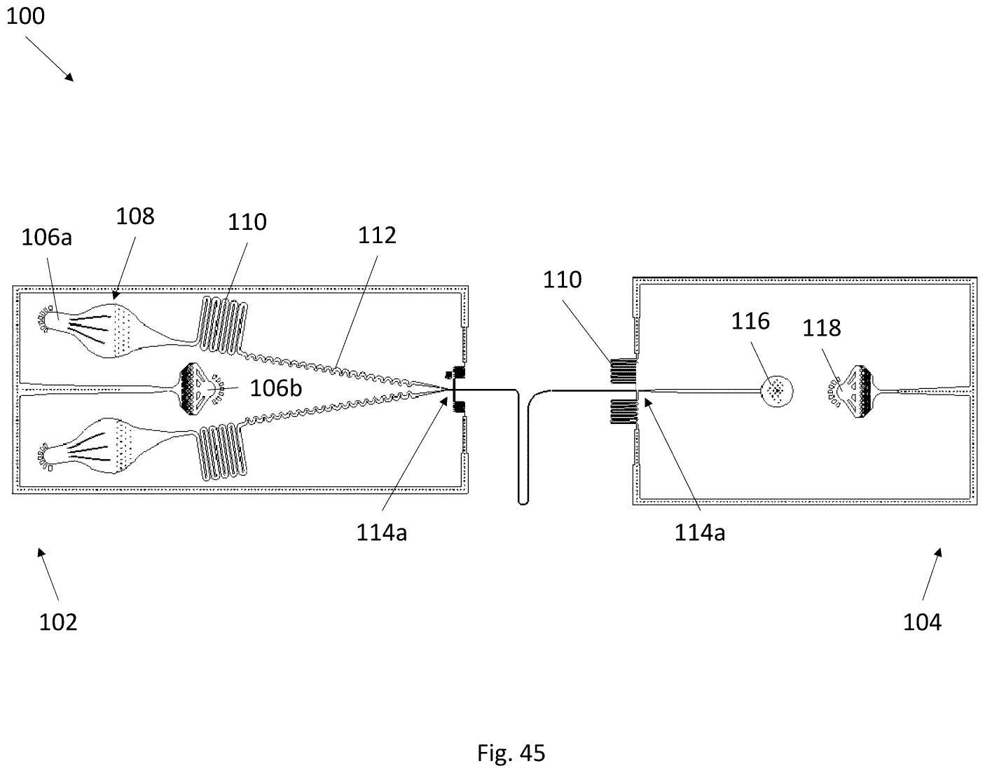

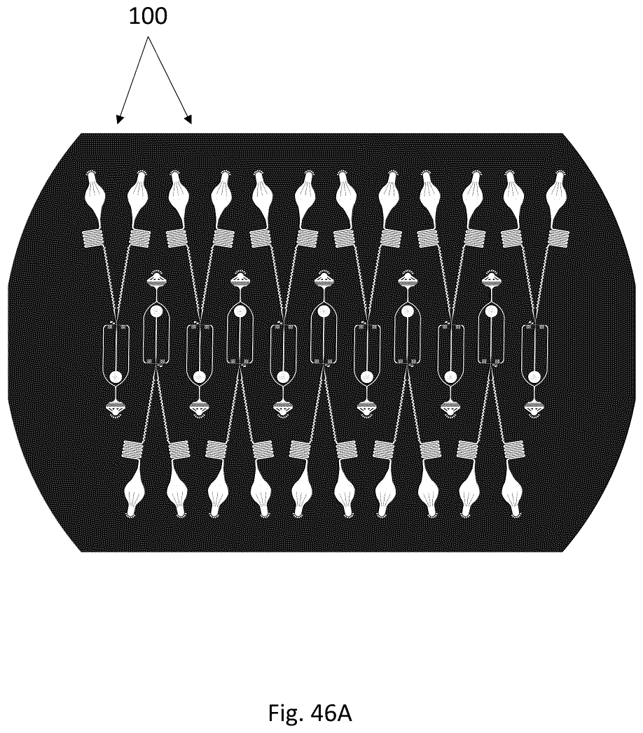

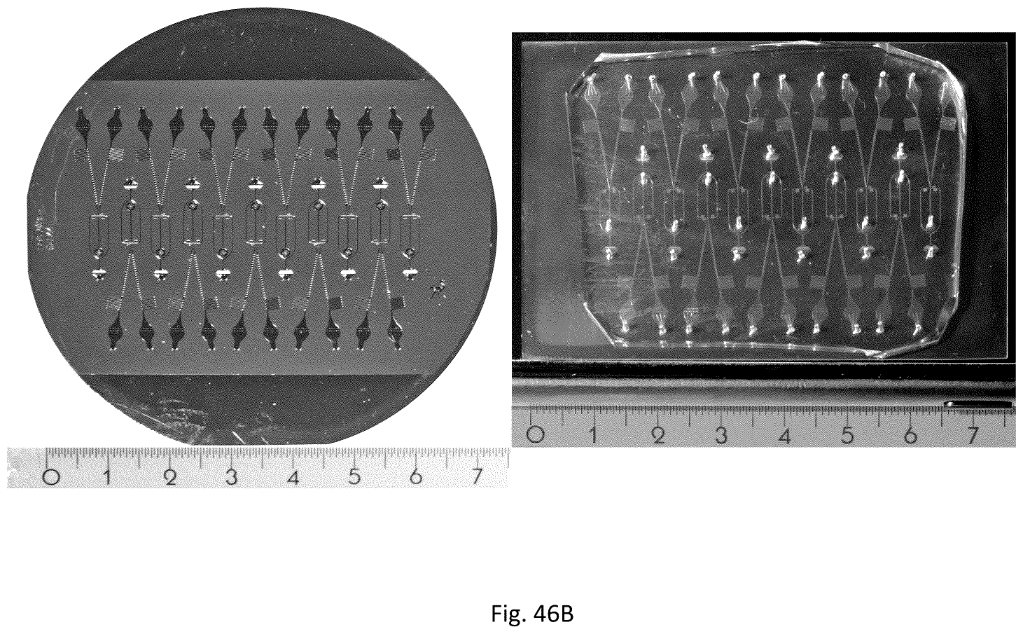

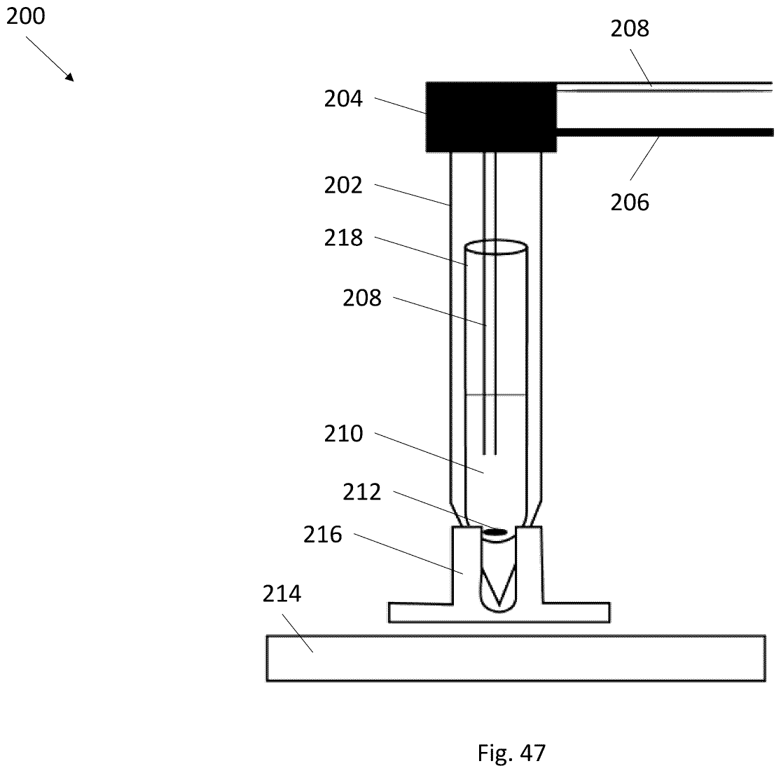

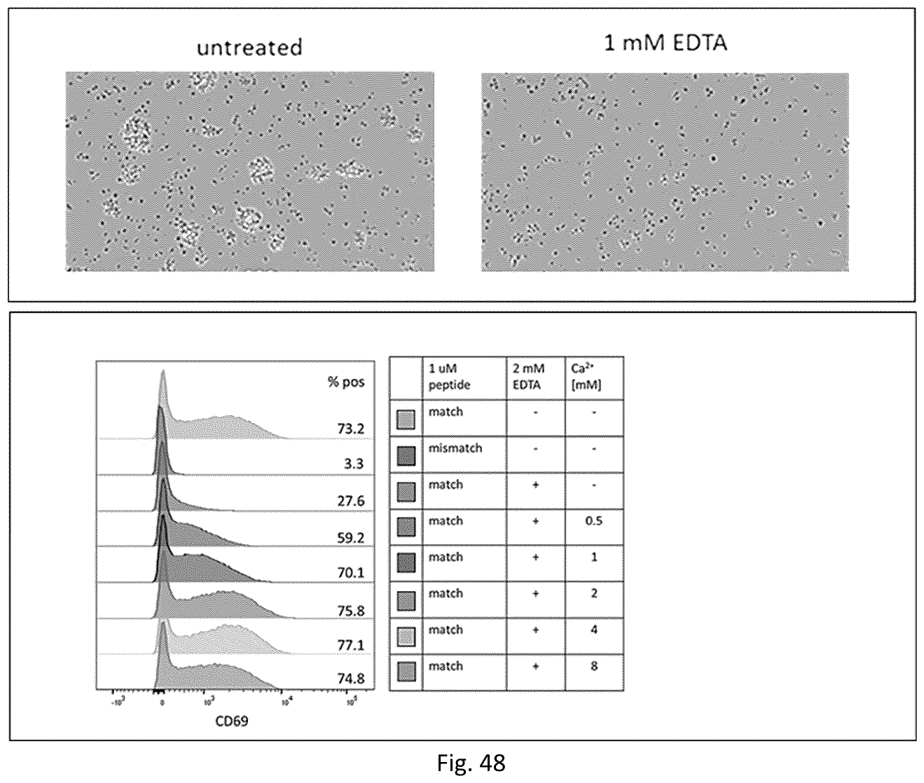

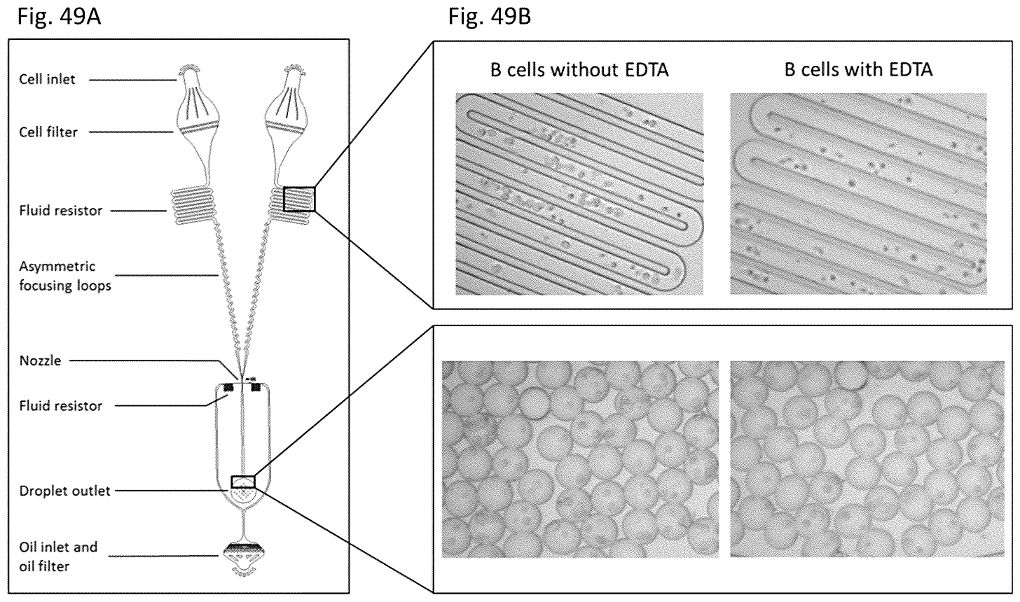

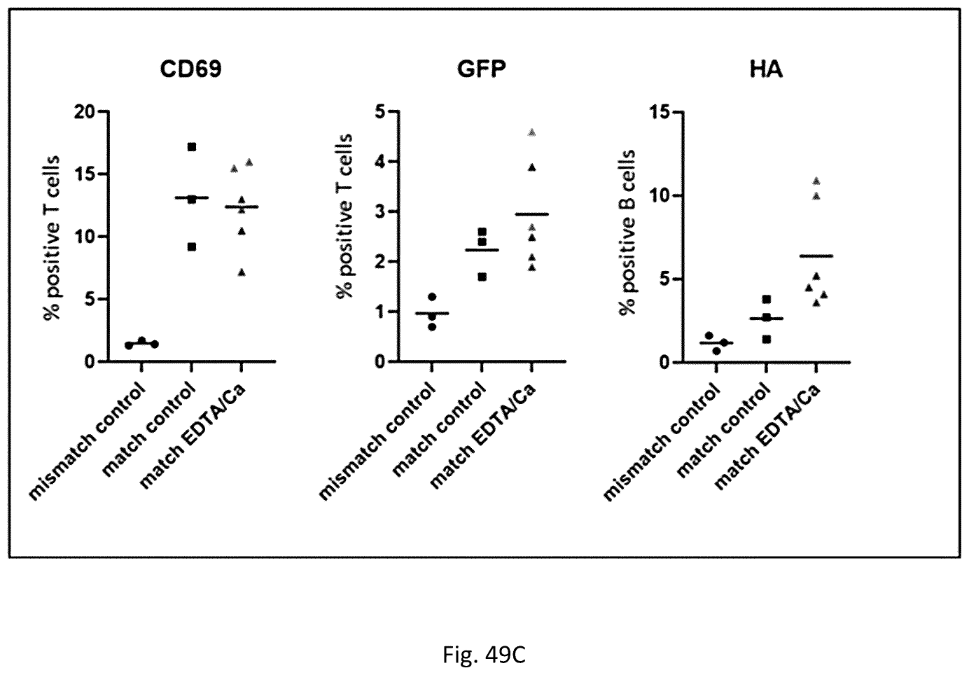

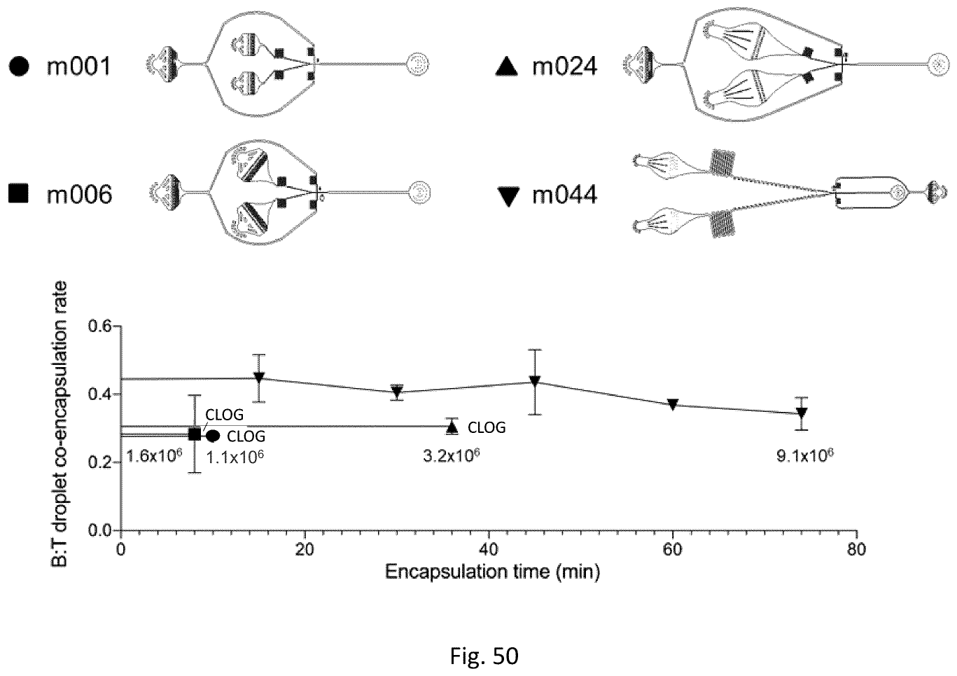

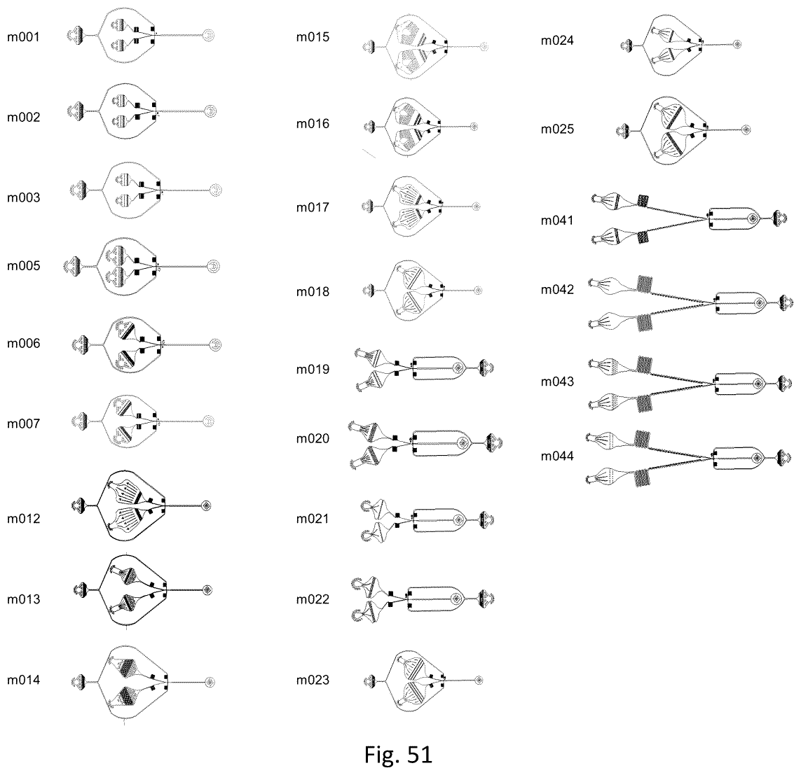

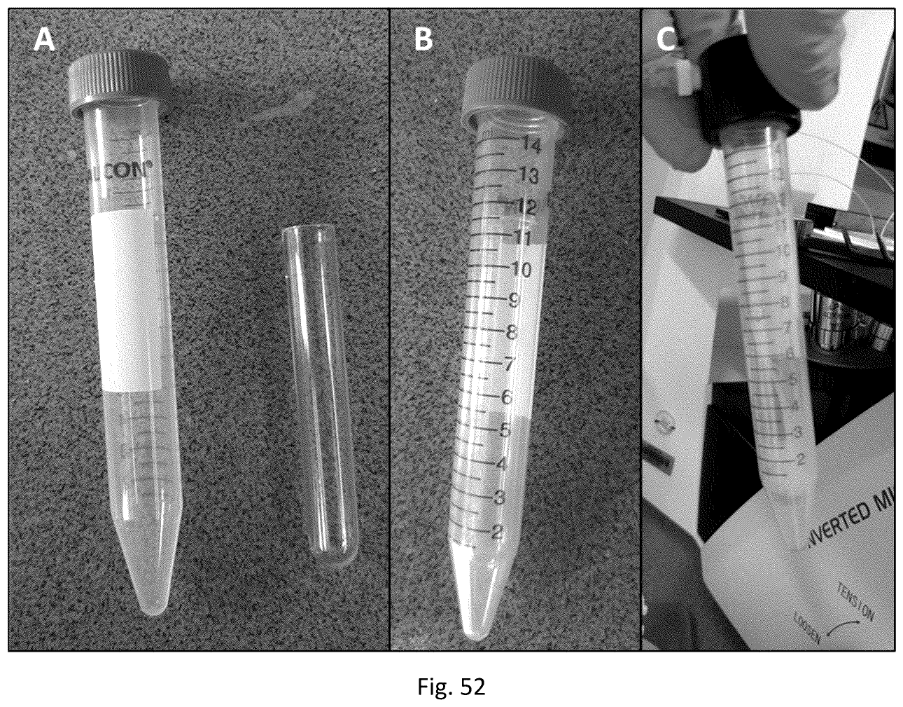

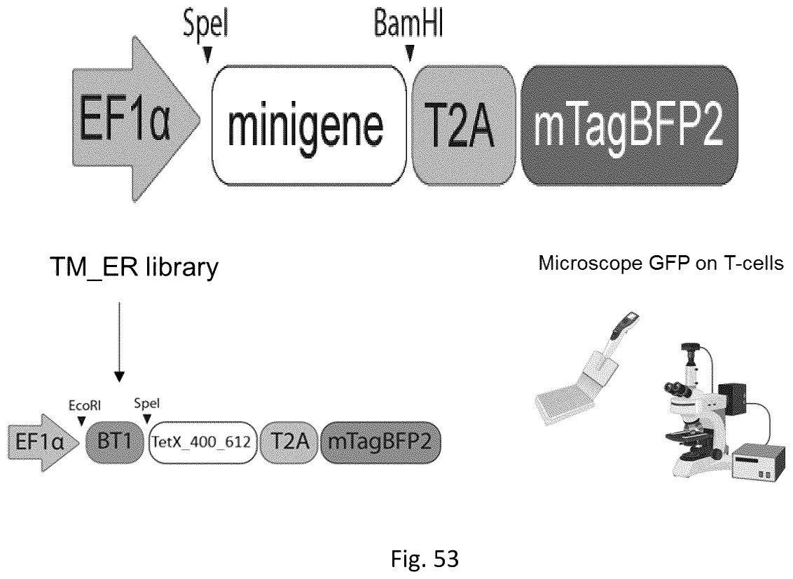

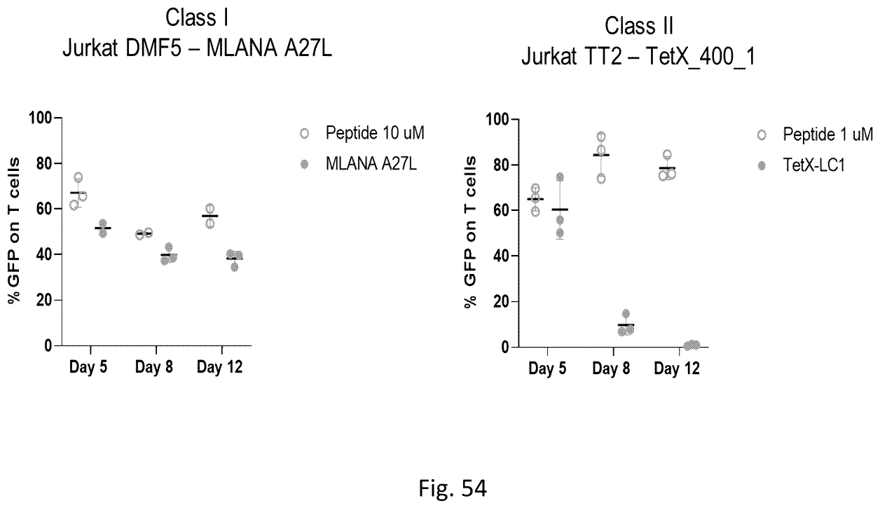

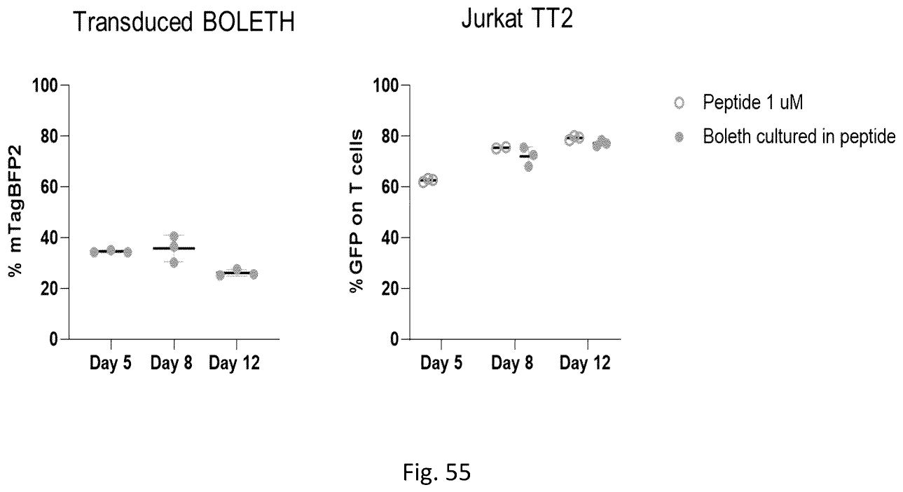

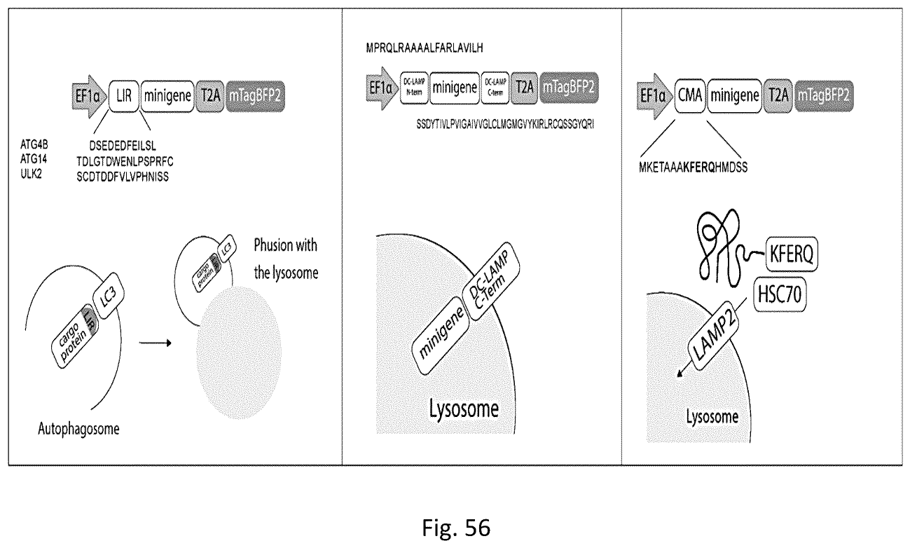

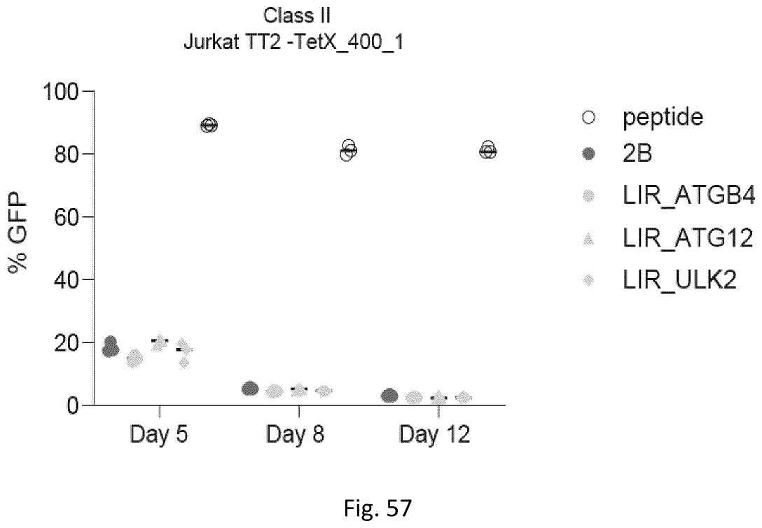

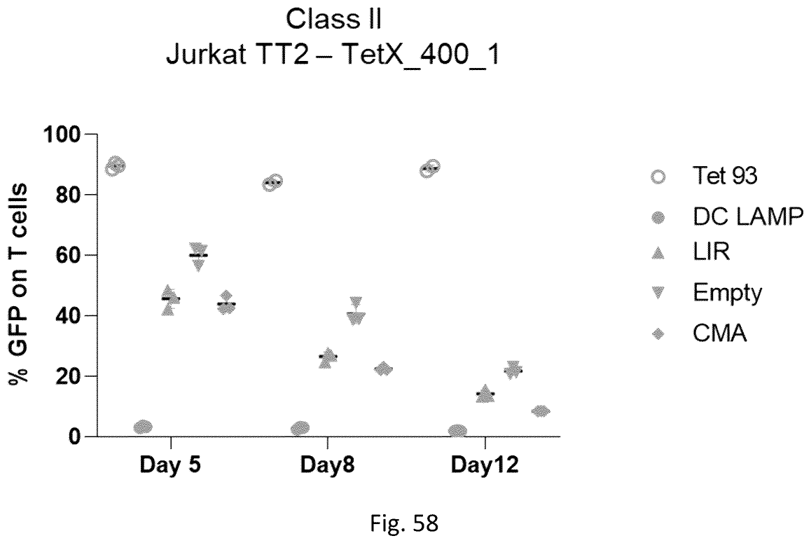

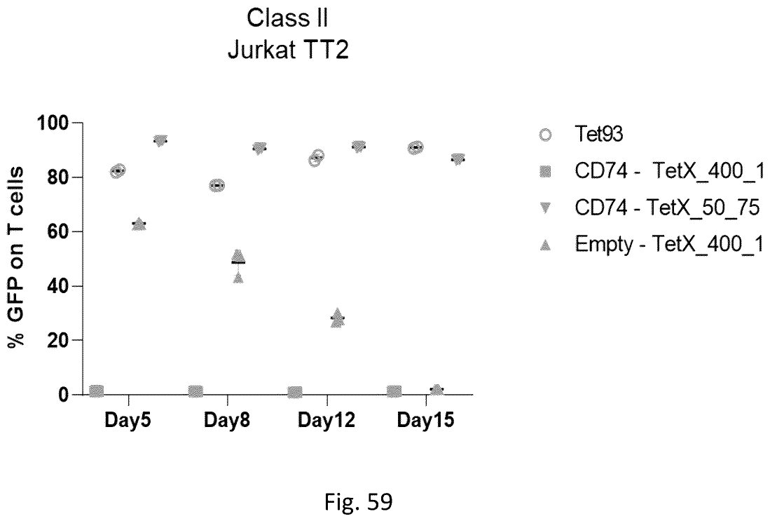

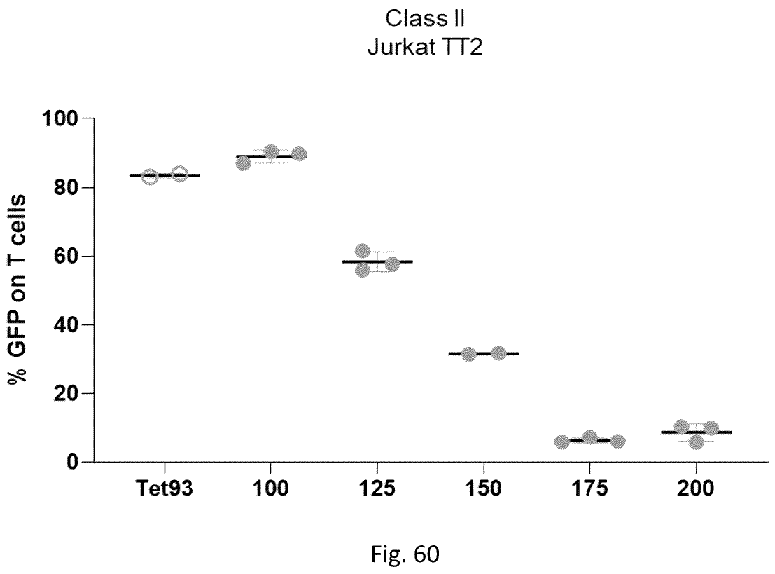

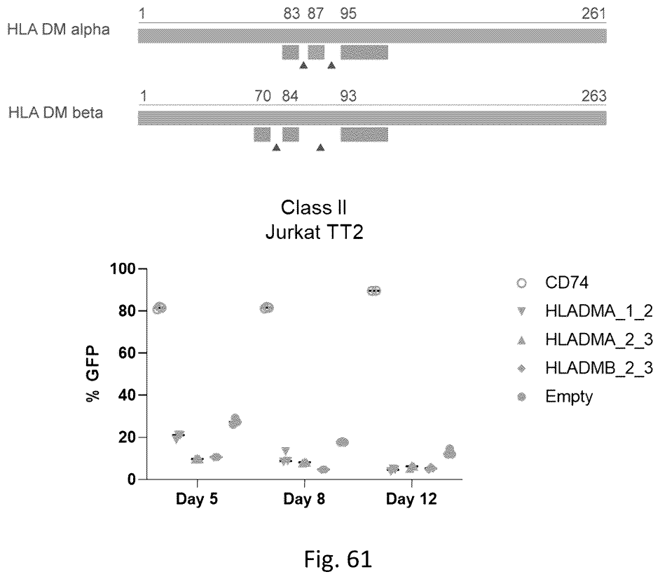

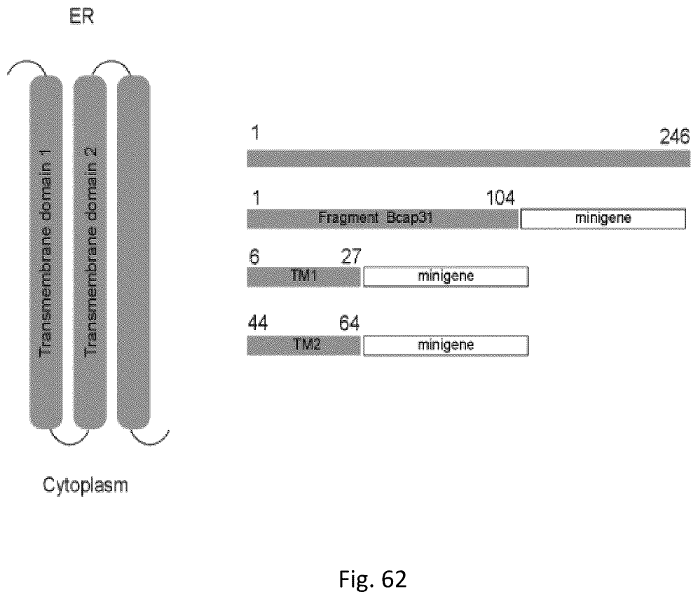

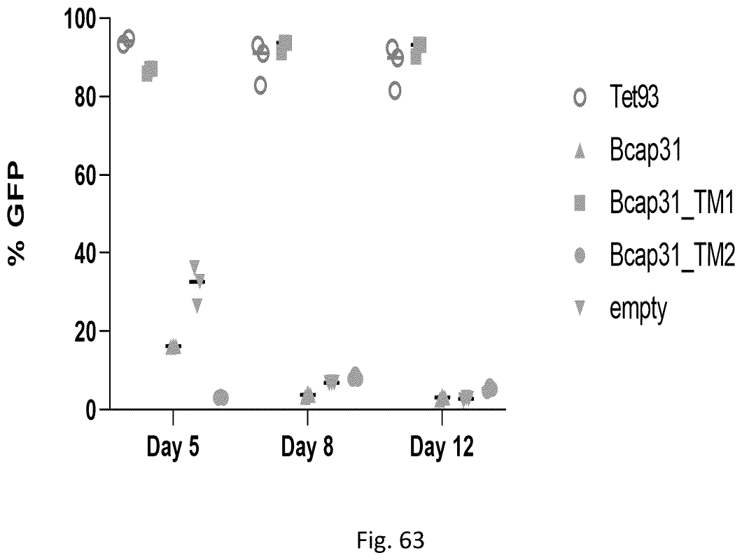

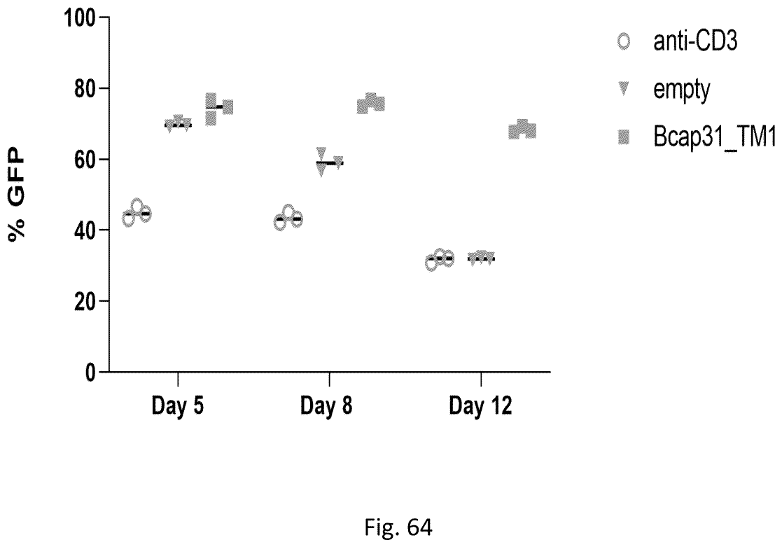

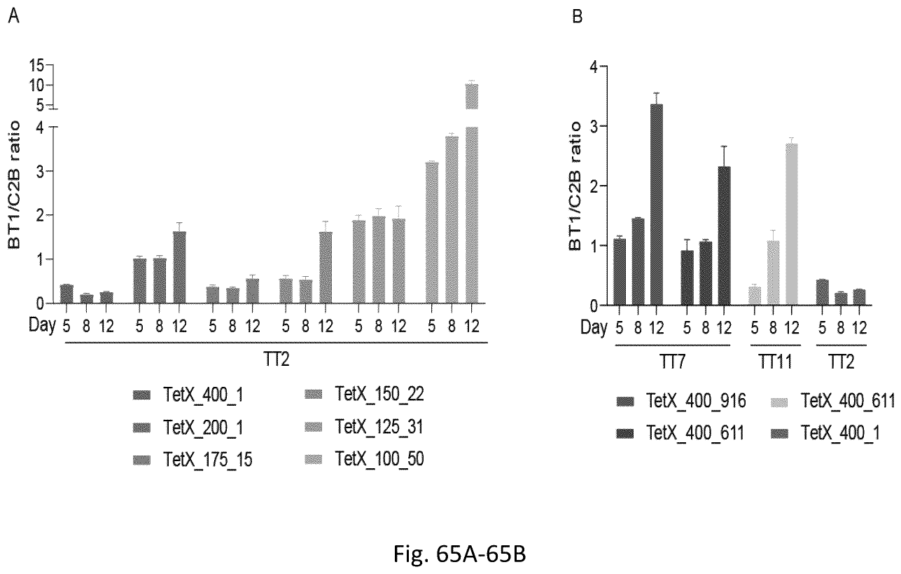

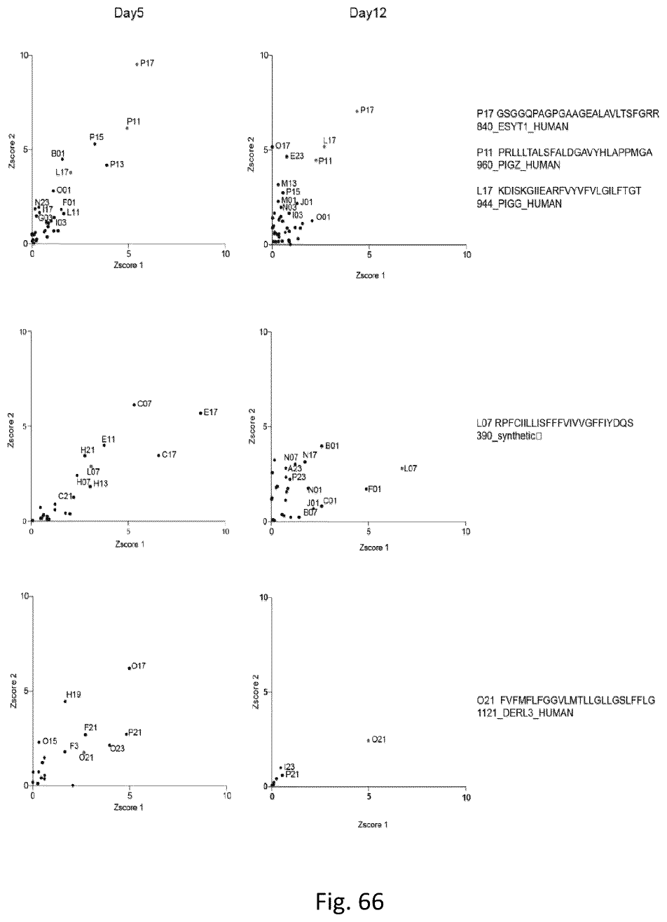

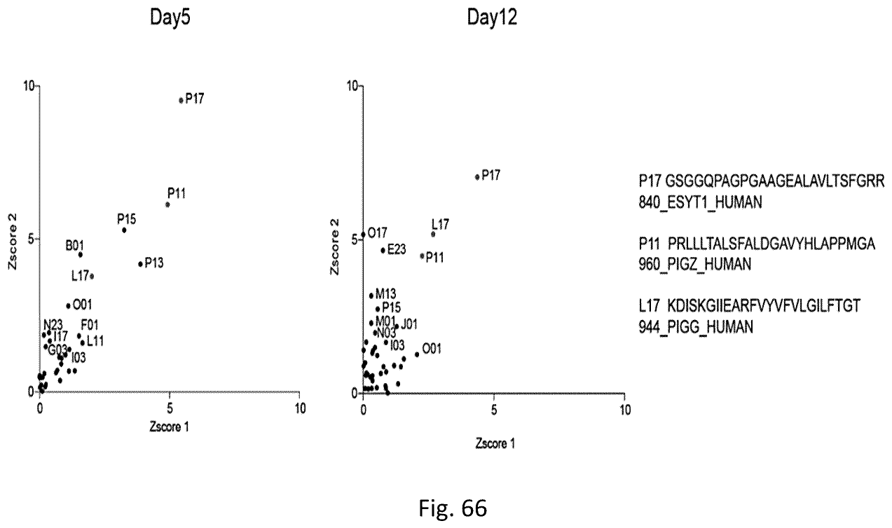

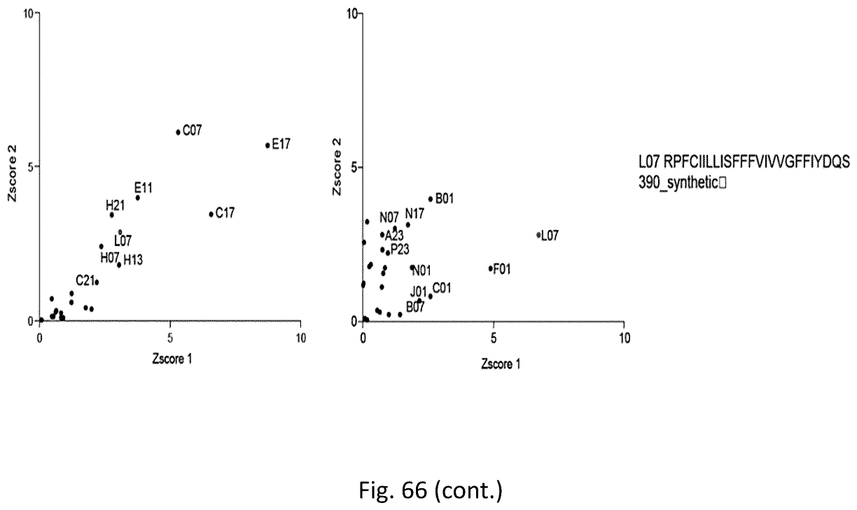

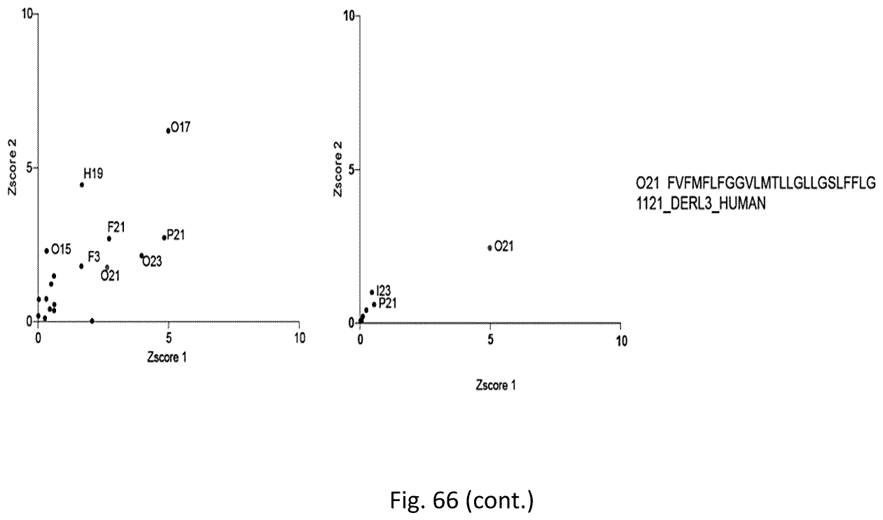

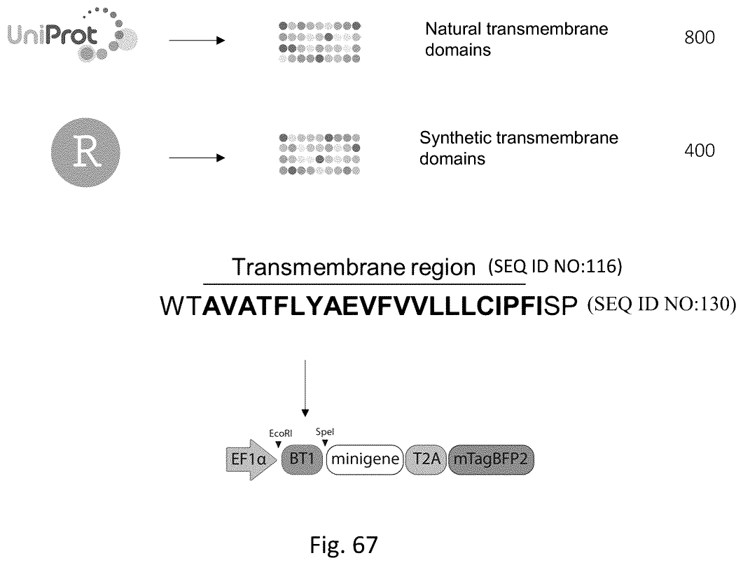

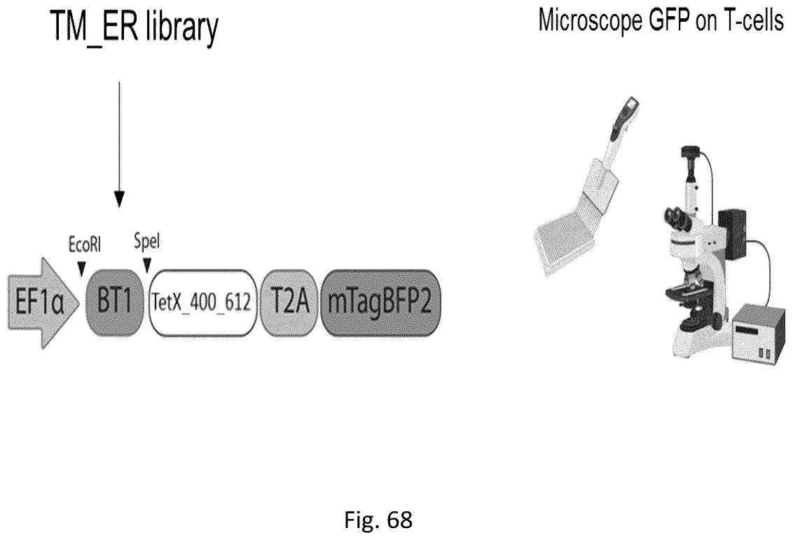

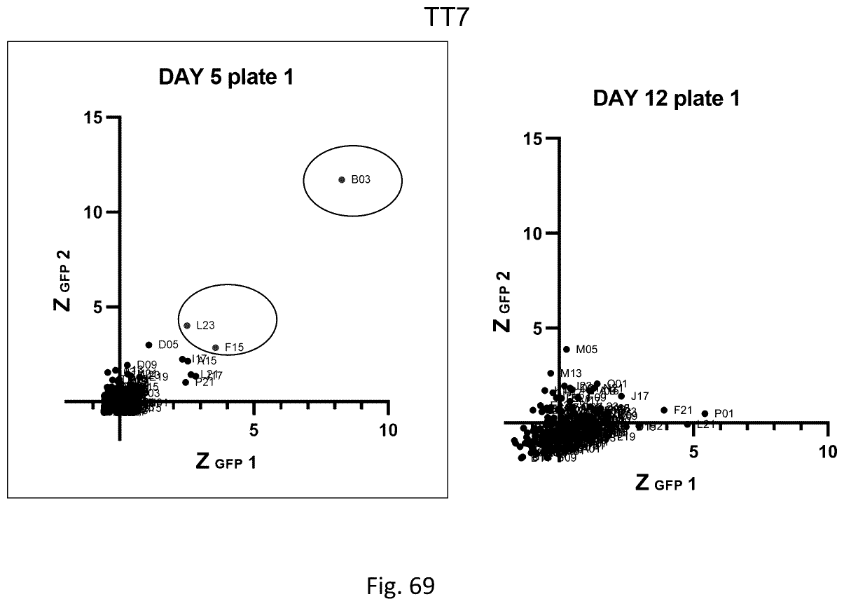

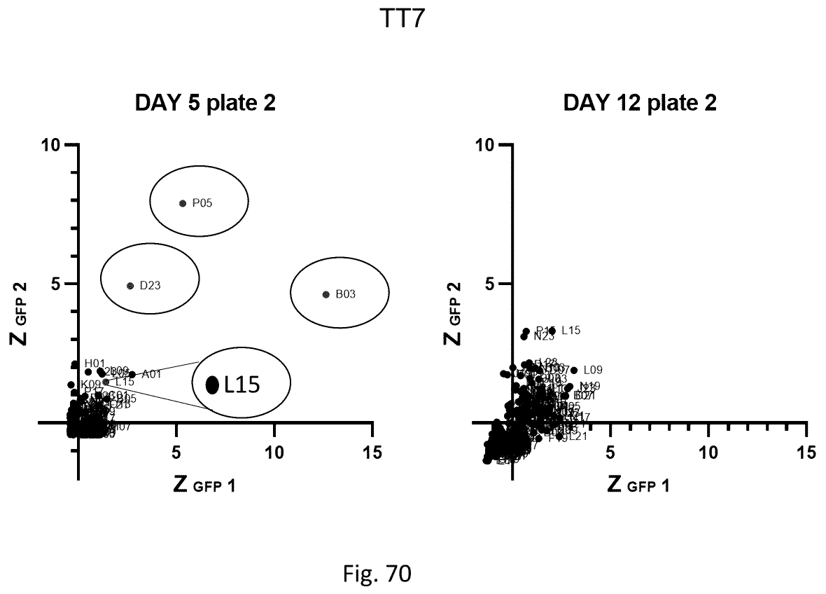

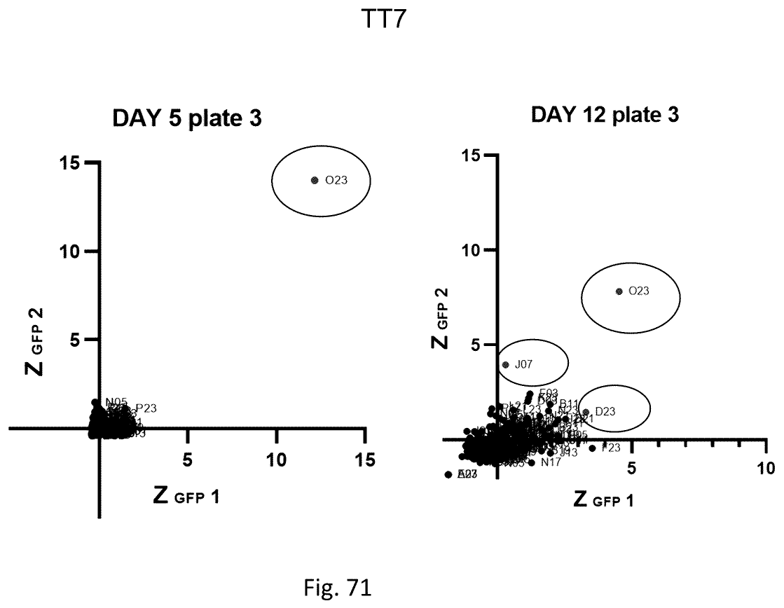

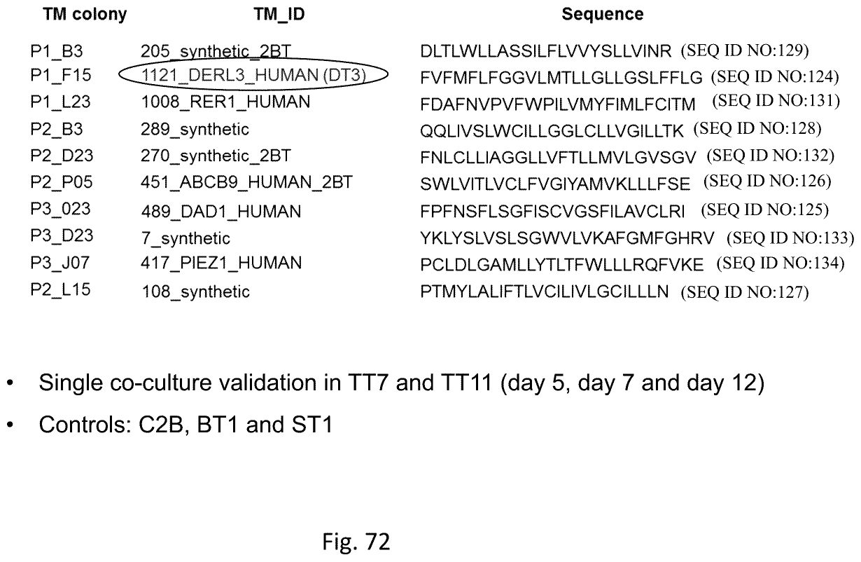

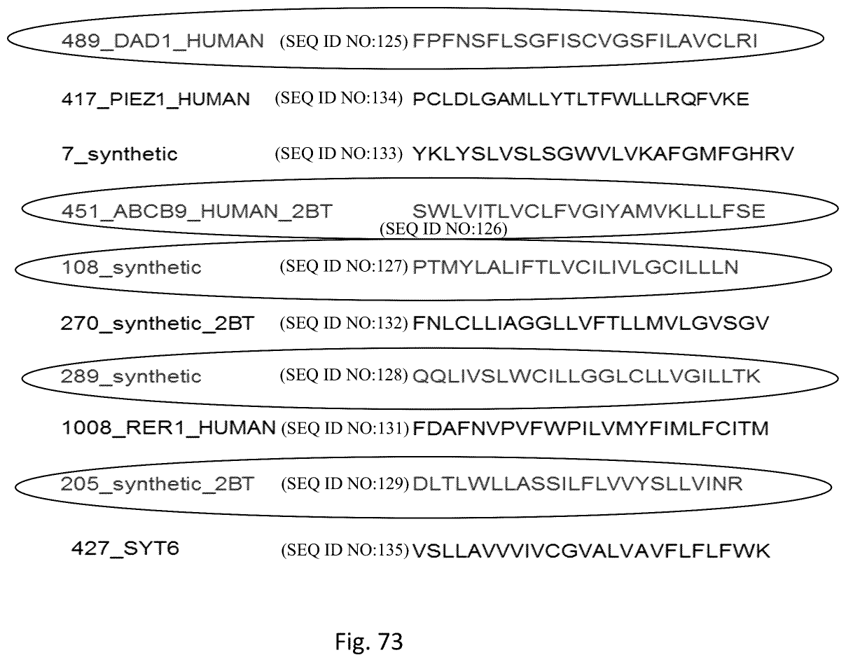

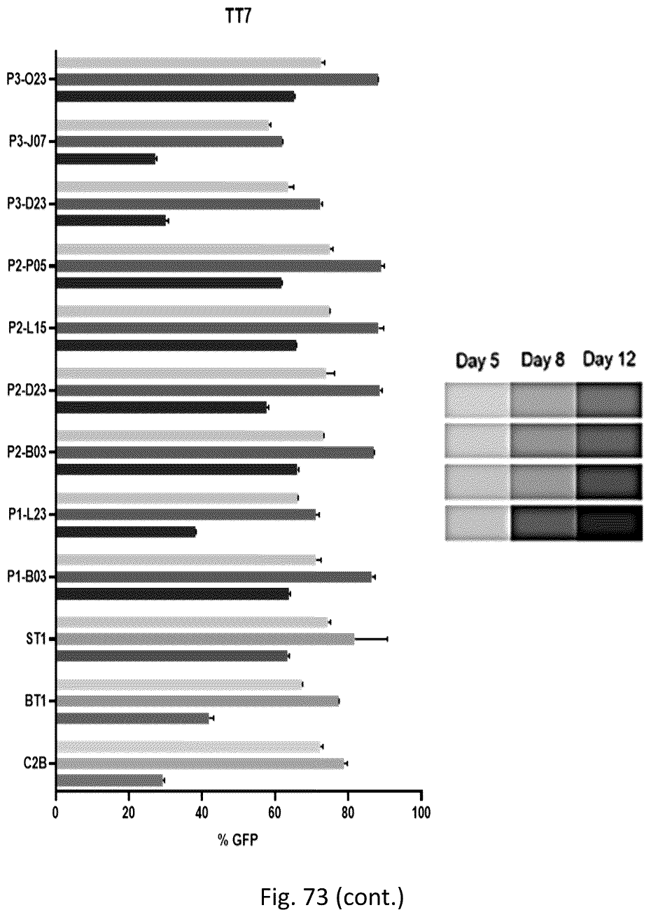

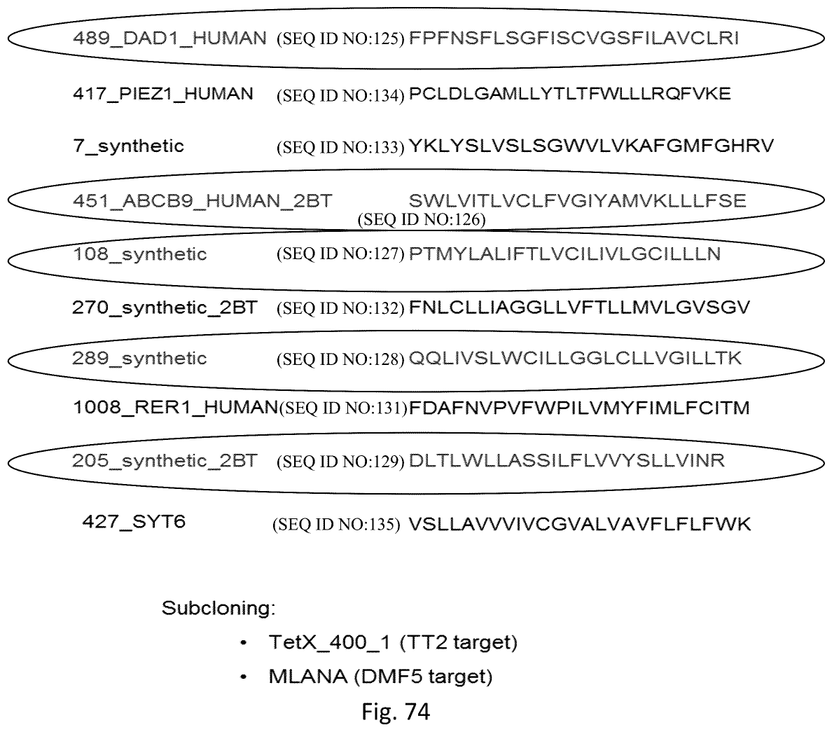

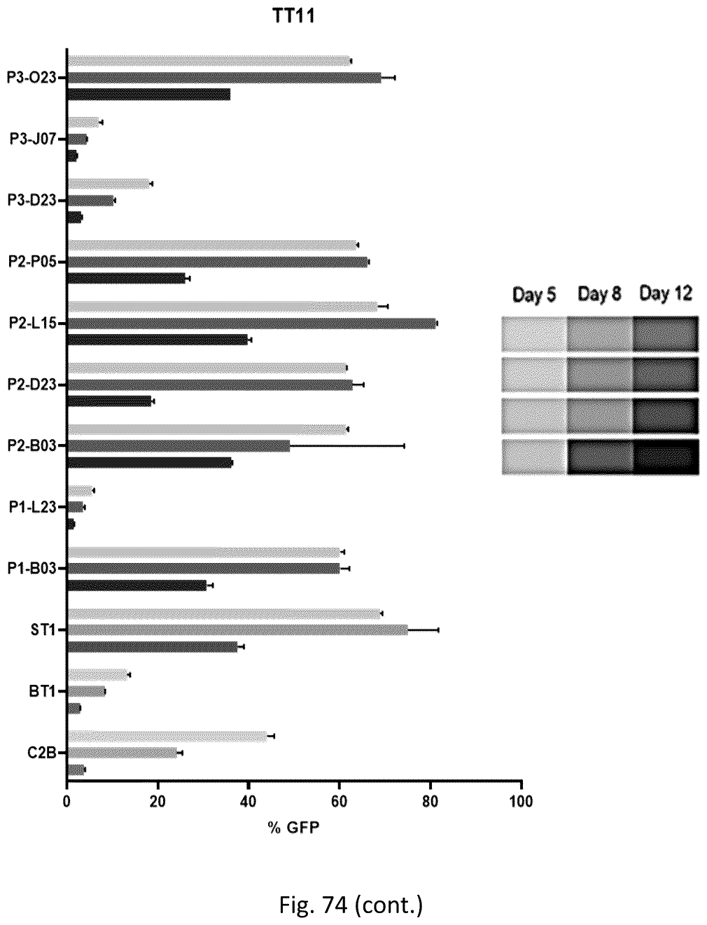

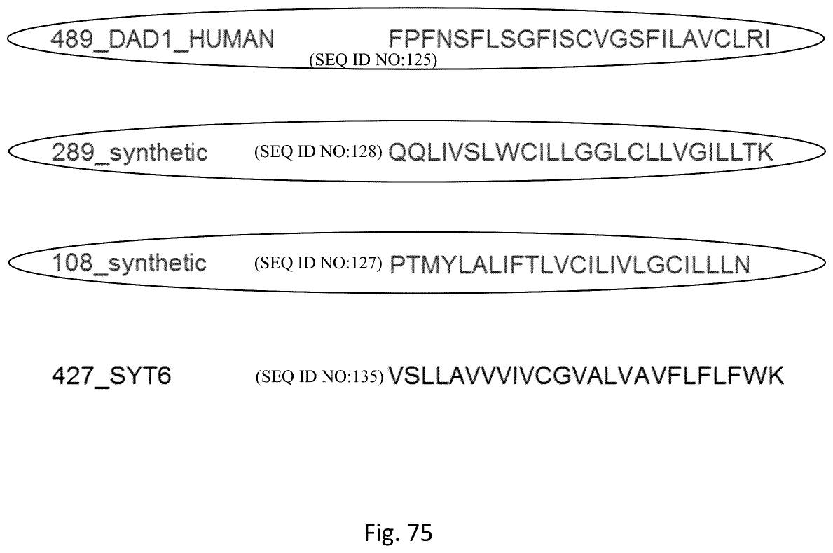

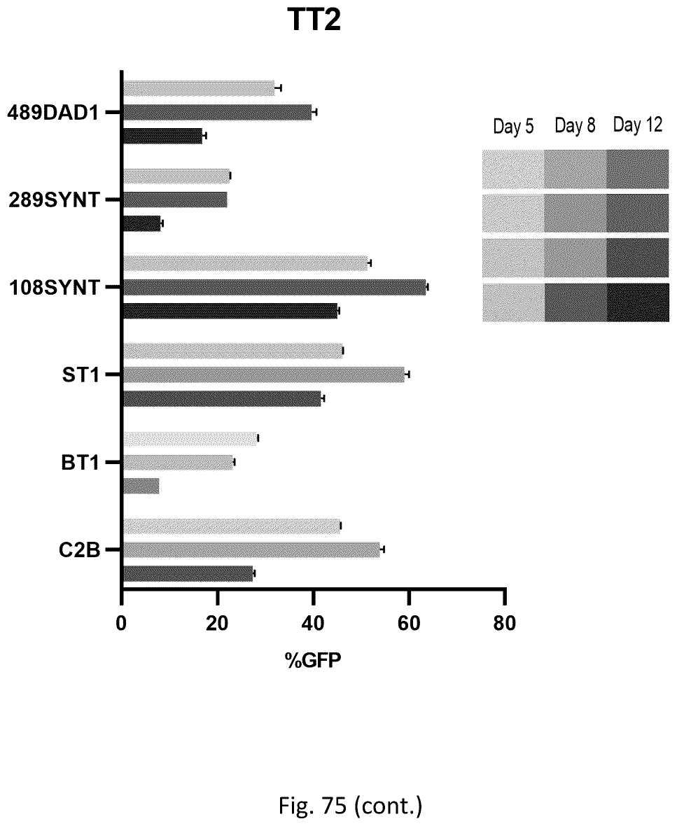

Representative flow cytometry histograms of V5 (left) and HA (right) signals from both scFvs and quantified median fluorescence intensity (MFI). FIG. 9C depicts the signal intensity resulting from single scFv constructs compared to tandem constructs as well as different transfections conditions (SN for BOLETH staining mixed or co-transfection of αCD19-HA and αCD20-V5. Representative flow cytometry histograms of V5 (left) and HA (right) signal from both scFvs and quantified median fluorescence intensity (MFI) of all tested conditions. SN of sfGFP transfected HEK cells was used as negative control. Error bars show ±1 SD. Dots represent one experiment with 4 replicates, data was reliably reproduced across several experiments. ns, not significant; *, P<0.05; **, P<0.01; ***, P<0.001 (unpaired Welch's t-test).

FIG. 10A and FIG. 10B depict exemplary experimental data demonstrating that an αCD19-αCD20-bispecific diabody does not robustly label B cells.

FIG. 10A depicts a schematic representation of diabody reporter DNA construct and of αCD19-αCD20-bispecific diabody protein. FIG. 10B depicts a comparison of single αCD19-HA and αCD20-V5, double αCD19-αCD20-diabody expression constructs. Representative flow cytometry histograms of V5 (left) and HA (right) signals from secreted scFvs and quantified median fluorescence intensity (MFI) of V5 and HA signals from the various scFvs. SN of sfGFP transfected HEK cells was used as a negative control. Error bars show ±1 SD. Dots represent one experiment with 4 replicates, data was reliably reproduced across several experiments.

FIG. 11A and FIG. 11B depict exemplary experimental data demonstrating that an alternative leader sequence does not improve signal strength of the tested scFvs. FIG. 11A depicts a sequence alignment of the optimized B43 leader sequence (SEQ ID NO:136) and the physiological ofatumumab leader sequence (SEQ ID NO:137). In purple, the first amino acids of ofatumumab-scFv in the VH-VL configuration are indicated. FIG. 11B depicts a comparison of HA and V5 median fluorescence intensity (MFI) from tagged scFvs with either a 2F2-leader sequence (dots) or an optimized B43 leader (diamonds). SN of sfGFP transfected HEK cells was used as negative control. Error bars show ±1 SD. Dots represent one experiment with 4 replicates, data was reliably reproduced across several experiments. ns, not significant; *, P<0.05; **, P<0.01; ***, P<0.001 (unpaired Welch's t-test).

FIG. 12A through FIG. 12C depict exemplary experimental data demonstrating that αCD20-scFv secreted by Jurkat reporter cells gives the highest MFI. FIG. 12A depicts a schematic representation of a T cell reporter in lentiviral constructs. Each scFv consists of a leader sequence, a heavy chain, a light chain, and a tag. The αCD19- and αCD20-scFv are tested with both a four-times V5 tag and a six-times HA-tag. Tandem scFv constructs are connected by a T2A sequence and contain αCD20-Ofatumumab scFv in either the VH-VL or VL-VH configuration. The diabody connects both αCD19- and αCD20-scFv with a flexible-rigid-flexible linker and contains a C-terminal HA and N-terminal V5 tag. Expression of scFvs is driven by a synthetic promoter called NBV and are connected via a T2A sequence to sfGFP expression upon engagement of the T cell receptor with the cognate antigen:HLA complex. All constructs were transduced into the TT2-TCR proof-of-principle T cell line. FIG. 12B depicts representative flow cytometry histograms of GFP (left), HA (middle), and V5 (right) signals from the mentioned reporter cell lines activated in presence of 1 μM cognate peptide. GFP signal from CD2 positive cells, HA and V5 signals from CD20-positive gated cells. E3 reporter cells contain αCD19-HA scFv in a sleeping beauty (SB) based transposon construct. FIG. 12C depicts a comparison of GFP (left) median fluorescence intensity (MFI) in Jurkat reporter cells and HA/V5-MFI (right) on B cells in the presence of 1 μM cognate peptide. Dots represent one experimental replicate.

FIG. 13A and FIG. 13B depict exemplary experimental data demonstrating that V5-tagged αCD20-scFv secreted by Jurkat TT2 cells produces the strongest GFP and V5 signals. FIG. 13A depicts a comparison of V5 and HA-tagged scFv median fluorescence intensity (MFI) from Jurkat TT2 reporter cells. Jurkat TT2 cells expressing the respective reporter were incubated with BOLETH cells in the presence of 0-10 μM cognate peptide. Gated on CD20 positive cells. E3 reporter cells contain αCD19-HA scFv-sfGFP reporter in a sleeping beauty transposon construct. MFI was determined using flow cytometry. FIG. 13B depicts a comparison of GFP median fluorescence intensity (MFI) from Jurkat TT2 reporter cells. Jurkat TT2 cells expressing the respective reporter were incubated with BOLETH cells in presence of 0-1 μM cognate peptide. Gated on CD2 positive cells. E3 reporter cells contain αCD19-HA scFv-sfGFP reporter in a sleeping beauty transposon construct. MFI was determined using flow cytometry. Dots represent N=2.

FIG. 14 depicts a schematic representation of the lentiviral constructs tested. scFv inserts αCD20-Rituximab (C2B8) and Ofatumumab (2F2) were cloned into pLVX-PGK-BSD containing αCD19-HA-scFv (B43) and were tested with two different tags (4×V5 and 6× FLAG).

FIG. 15A through FIG. 15D depict comparisons of the ability of scFv molecules to label B cells in a co-culture. FIG. 15A and FIG. 15B depict a comparison of Rituximab-FLAG(A) and Rituximab-V5(B) to label B cells in a co-culture. HA, FLAG, or V5 signal on B cells co-cultured with 1 μM cognate or mismatched peptide and Jurkat TT2 cells expressing (FIG. 15A) B43-HA and Rituximab-FLAG(A) or (FIG. 15B) B43-HA and Rituximab-V5(B) in lentiviral constructs. Gated on live CD20 positive cells. FIG. 15C and FIG. 15D depict a comparison of Rituximab-V5(B) and Ofatumumab-V5(C) to label B cells in a co-culture. HA and V5 signal on B cells co-cultured with 1 μM cognate or mismatched peptide and Jurkat TT2 cells expressing (FIG. 15C) B43-HA and Rituximab-V5(B) or (FIG. 15D) B43-HA and Ofatumumab-V5(C) in lentiviral constructs. Gated on live CD20 positive cells.

FIG. 16A through FIG. 16K depict data demonstrating optimizations of the scFv constructs. FIG. 16A depicts a schematic representation of transient expression plasmids tested for optimizing B cell scFv labeling. scFv inserts αCD20-Ofatumumab (2F2) or αCD19 (B43) were cloned into pTwist CMV BetaGlobin WPRE Neo. FIG. 16B and FIG. 16C depict a comparison between HA-tag on αCD19-scFv (B43) and αCD20-scFv (2F2). HA signal was evaluated on B cells stained with the supernatant of transfected HEK cells secreting the respective scFvs. FIG. 16B: A flow cytometry histogram of αCD19-HA (B43, cf. FIG. 16A, construct D) and αCD20-HA (2F2, cf FIG. 16A, construct I) labeling of BOLETH cells. FIG. 16C: The percentage and median fluorescence intensity (MFI) of HA signal from αCD19-HA (B43, cf. FIG. 16A, construct D) and αCD20-HA (2F2, cf FIG. 16A, construct I) scFvs. Supernatant of sfGFP transfected HEK cells was used as negative control. FIG. 16C and FIG. 16D depict a comparison between HA- and V5-tag on αCD20-scFv(2F2). HA and V5 signals were evaluated on B cells stained with the supernatant of transfected HEK cells secreting the respective scFvs. FIG. 16D: A flow cytometry histogram of αCD20-scFv(2F2) labeling of BOLETH cells tagged with either HA (cf FIG. 16A, construct I) or V5 (cf. FIG. 16A, construct F). FIG. 16E: The percentage and median fluorescence intensity (MFI) of HA and V5 signal from αCD20-scFv (2F2, cf. FIG. 16A, constructs F/I). Supernatant of sfGFP-transfected HEK cells was used as negative control. FIG. 16F and FIG. 16G depict a comparison between the intensity resulting from single scFv constructs and double scFv constructs. HA and V5 signals were evaluated on B cells stained with the supernatant of transfected HEK cells secreting the respective scFvs. FIG. 16F: A flow cytometry histogram of HA and V5 signal from single scFv constructs αCD20-V5(2F2, cf. FIG. 16A, construct F) and αCD19-HA (B43, cf FIG. 16A, construct D) and double scFv construct αCD19-HA-αCD20-V5 (cf. FIG. 16A, construct E). FIG. 16G: The median fluorescence intensity (MFI) of V5 and HA signal from the single scFv constructs (cf. FIG. 16A, constructs F/D) compared to the intensity resulting from the double scFv construct (cf FIG. 16A, construct E). Supernatant of sfGFP transfected HEK cells was used as negative control. FIG. 16H and FIG. 16I depict a comparison of locating the scFvs upstream or downstream of the T2A sequence. HA and V5 signals were evaluated on B cells stained with the supernatant of transfected HEK cells secreting the respective scFvs. FIG. 16H: A flow cytometry histogram of V5 and HA signal from double scFv constructs (cf. FIG. 16A, construct E/H) with either αCD19 (B43) or αCD20 (2F2) directly downstream of the NBV promoter. FIG. 16I: The percentage of HA and V5 labeled BOLETH cells and median fluorescence intensity (MFI) of V5 and HA signal from the double scFv constructs (cf. FIG. 16A, constructs E/H). Supernatant of sfGFP transfected HEK cells was used as negative control. FIG. 16J and FIG. 16K depict a comparison of αCD20-V5 scFv light and heavy chain arrangement. The V5 signal was evaluated on B cells stained with the supernatant transfected HEK cells secreting the respective scFvs. FIG. 16J: A flow cytometry histogram of V5 signal from αCD20-V5 with rearrange heavy and light chain order VHVL (cf. FIG. 16A, construct F) or VLVH (cf. FIG. 16A, construct G). FIG. 16K: The percentage of V5 labeled BOLETH cells and median fluorescence intensity (MFI) of V5 from αCD20-V5 VHVL (cf. FIG. 16A, construct F) or VLVH (cf. FIG. 16A, construct G). Supernatant of sfGFP transfected HEK cells was used as negative control.

FIG. 17A and FIG. 17B depict HA tag signal on B cells co-cultured with 1 μM cognate or mismatched peptide and Jurkat cells transgenically modified with synthetic promoters via lentiviral integration. HA tag signal on B cells co cultured with 1 μM cognate or mismatched peptide and T cells expressing different synthetic promoters in the lentiviral reporter. FIG. 17A:HA percentage gated on CD20 positive cells. FIG. 17B:HA median fluorescence intensity gated on CD20 positive cells.

FIG. 18A through FIG. 18D depict the design and sensitivity of sleeping beauty-based transposon constructs. FIG. 18A:Upper panel: Schematic diagram of transgenic reporter construct design. Lentiviral reporter construct with TCR inducible reporter genes cassettes upstream of a constitutively expressed blasticidin resistance gene. Lower panel: Sleeping beauty constructs with TCR signaling-inducible reporter gene cassette flanked by insulator sequences with a downstream, constitutively expressed puromycin resistance gene and the co-stimulatory molecule CD28. FIG. 18B: NBVp and the P10 promoter drive high GFP and HA reporter gene expression in sleeping beauty constructs. FIG. 18C: Cloning synthetic promoter sequences 5′ of P10 or NBV sequences. FIG. 18D: Synergy between synthetic promoter sequences. Adding additional synthetic promoter sequences upstream of P10 or NBV sequences further improves reporter sensitivity in co-culture of Jurkat-P10NBV-TT2 cells with BOLETH cells loaded with 0.1 μM cognate or negative control peptides.

FIG. 19A through FIG. 19C depict the sensitivity of the Jurkat-P10NBV reporter line with various TCRs targeting DR4-presented peptides from the tetanus toxin protein. FIG. 19A: (Reporter-independent) CD69 surface expression on T cells. FIG. 19B: GFP expression in T cells. FIG. 19C. HA labeling on BOLETH cells.

FIG. 20A and FIG. 20B depict the GFP and HA median intensities of B cells co cultured with 1 μM cognate or mismatched peptide and SKW-3 cells expressing sleeping beauty transposon system reporters. The different synthetic promoters compared to 4×NFAT promoter in the sleeping beauty transposon reporter. FIG. 20A: GFP median intensity gated on CD3 positive cells. FIG. 20B: HA median intensity gated on CD20 positive B cells.

FIG. 21A and FIG. 21B depict a 4×NFAT and NBV promoter comparison in sleeping beauty transposon system via co-culturing with B cells using different concentrations of cognate peptide. FIG. 21A: Comparison of GFP median fluorescence gated on CD2 positive cells. FIG. 21B: Comparison of HA median fluorescence intensity gated on CD20 positive cells.

FIG. 22A and FIG. 22B depict the GFP and HA median intensities of B cells co cultured with 1 μM cognate or mismatched peptide and Jurkat cells expressing sleeping beauty transposon system reporters. The 4×NFAT promoter in the sleeping beauty transposon reporter represents current standards in regular use. FIG. 22A: GFP median intensity gated on CD3 positive cells. FIG. 22B: HA median intensity gated on CD20 positive B cells.

FIG. 23 depicts the ectopic expression of co-stimulatory molecules from lentiviral constructs can further increase reporter gene expression in Jurkat-NBV58-TT11 cells.

FIG. 24 depicts a diagram depicting the principle of function for the NBV synthetic transcription factor.

FIG. 25A through FIG. 25D depict the TCR responsive promoter-driven synthetic transcription factor NBVtf can further increase reporter gene expression. FIG. 25A: The synthetic transcription factor NBVtf can bind to the synthetic NBV promoter and activate reporter gene expression. FIG. 25B: Schematic design of inducibly-expressed NBVtf in sleeping beauty constructs with P32-driven NBVtf expression and constitutively expressed neomycin resistance gene and co-stimulatory molecules CD226 and 4-1BB. FIG. 25C: Sleeping beauty-expressed NBVtf further improves reporter gene expression in plate co-culture of Jurkat-P10NBV-TT2 cells with BOLETH cells loaded with 0.01 μM cognate peptide. FIG. 25D: Sleeping beauty-expressed NBVtf further improves reporter gene expression in plate co-culture of Jurkat-P10NBV-TT11 cells with BOLETH cells loaded with. 0.1 μM cognate peptide.

FIG. 26 depicts the correlation of surface-expressed co-stimulatory proteins with potentiated minigene-based T cell activation. TT2-expressing P10-NBV reporter Jurkat and SKW3 co-culture experiment summary depicting CD69 and GFP upregulation in T cells and anti-CD19 scFv marking on cognate minigene-transduced B cells.

FIG. 27 depicts a scheme of the process for generation of minigene sequences.

FIG. 28 depicts a diagram showing exemplary minigene structures.

FIG. 29 depicts an analysis of the number of minigenes with different lengths in the two exemplary libraries.

FIG. 30 depicts exemplary data demonstrating that minigene processing does not affect the antigen presentation and T cell activation.

FIG. 31 depicts an overview of the FALCON algorithm.

FIG. 32 depicts an adjusted codon usage table for EBV immortalized B cells.

FIG. 33 depicts equations used for the development and optimization of the FALCON algorithm.

FIG. 34 depicts a table showing the features of the FALCON algorithm.

FIG. 35 depicts a table showing a comparison between FALCON and other commonly used algorithms.

FIG. 36 depicts the design principle of synonymous peptide barcodes.

FIG. 37 depicts a diagram demonstrating that the barcode generator ensures that a minimal Levenshtein distance between any two members of the library is maintained.

FIG. 38 depicts a scheme of CMA-Minigene-BFP (C2B) based constructs and the modified, surface-receptor Flag-conjugated constructs (C2F).

FIG. 39A and FIG. 39B depict evidence that flag-based surface receptor constructs can be used to successfully enrich for transduced B cells. FIG. 39A: B cells transduced with lentiviral-containing supernatants were subsequently enriched for Flag surface expression. Relative Flag surface expression was then determined using a flow cytometer. Shown are histograms depicting the median PE fluorescent intensity. FIG. 39B: B cells transduced with diluted supernatants driving expression of OX40L-Flag. Cells were then enriched for Flag surface expression. Relative Flag surface expression was then determined using a flow cytometer. Shown are histograms depicting Flag⁺ cell fractions.

FIG. 40 depicts data demonstrating that OX40L and 4-1BBL overexpression potentiates minigene-based T cell activation. TT2-expressing P10-NBV reporter Jurkat and SKW3 co-culture experiment summary depicting CD69 and GFP upregulation in T cells and anti-CD19 scFv marking on cognate minigene-transduced B cells.

FIG. 41 depicts data demonstrating the expression of a transgene from genome-derived regulatory elements which can serve as constitutively active promoters.

FIG. 42 depicts an exemplary microfluidic device.

FIG. 43 depicts magnified views of an inlet and filter (top) and focusing loops of an exemplary microfluidic device.

FIG. 44 depicts a magnified view of a nozzle of an exemplary microfluidic device.

FIG. 45 depicts an exemplary double emulsion microfluidic device.

FIG. 46A and FIG. 46B depict exemplary arrays of microfluidic devices.

FIG. 47 depicts an exemplary reservoir.

FIG. 48 depicts the results of a cell culture clumping study. (Top) B cells in normal plate culture start to form clumps quickly. Addition of at least 1 mM EDTA greatly reduces clumping. (Bottom) Bulk plate co-culture of TCR-expressing Jurkat T cells with B cells presenting their cognate peptide. Addition of 2 mM EDTA inhibits cell clumping but also impairs T cell activation, as measured by surface expression of the CD69 activation marker. Addition of Ca²⁺ to the medium in equimolar concentration as that of the EDTA in the medium fully restores T cell activation.

FIG. 49A through FIG. 49C depict the results of experiments demonstrating a microfluidic setup for co-encapsulation of B and T cells with EDTA to prevent B cell clumping and Ca²⁺ to preserve T cell activation. FIG. 49A: Illustration of the microfluidic PDMS chip used for encapsulation of B and T cells. Cells enter the chip at the inlets, flow through the cell filter, fluid resistor, and asymmetric focusing loops before they are co-encapsulated in droplets at the nozzle. Droplets then leave the device in the droplet outlet. FIG. 49B: Addition of 2 mM EDTA to the B cell suspension improves cell flow through the filter area and inhibits the clumping of B cells. Fewer cell clumps are observed in all areas of the chips. Shown is the fluid resistor areas. Fewer droplets contain clumps or large numbers of cells. FIG. 49C: Addition of 8 mM Ca²⁺ to the T cell suspension replaces the Ca²⁺ chelated by EDTA when the droplets are formed and thereby rescues Ca²⁺ dependent T cell activation measured by CD69 surface expression with flow cytometry. GFP expression in encapsulated reporter T cells and scFv-HA labeling of B cells as indicators for T cell activation in droplets are indicated.

FIG. 50 depicts the results of experiments demonstrating that structural modifications in microfluidic devices lead to higher efficiency and stability in droplet formation. (Top) Illustration of representative microfluidic PDMS chips used to test for B and T cell encapsulation efficiencies in correspondence to rational improvements to the overall designs. (Bottom) Total encapsulation time prior to device clogging in devices presented in the top illustration. Depicted on the x-axis is total encapsulation time (min), and on the y-axis, the ratio of droplets showing co-encapsulation of at least one B and T cell. Indicated for each data point is the total number of viable B cells recovered from the respective droplet emulsion.

FIG. 51 depicts a representative selection of the progression of microfluidic device designs. (m001) Original design from Mazutis et al. (m002) Devices in which the size of the entire design was increased ×1.3 overall or (m003) with maintaining the same filter density. (m005-m007) Devices in which the overall size of the aqueous inlets were modified without changing the overall initial design. (m005) Increase in both aqueous inlets. (m006) Same as m005 but with tilting of the aqueous inlets to be in line with the angle of the flow chambers. (m007) same as m006 but with ×1.25 increase in spacing between the filter units within the aqueous inlets. (m012-m025) Different modifications to the overall structure and size of the aqueous inlets, the shape of the filter units, the number of filter unit layers, the spacing between the filter units. (m041-m044) Repositioning of the detergent inlet as well as introduction of lateral focusing loops while utilizing the aqueous inlets used in design m024. (m041) Base design. (m042) Complete removal of filter units in the aqueous inlets. (m043) Addition of 4 layers of small features to prevent ceiling collapse in the aqueous inlets. (m044) Same as m043 but with two supporting layers.

FIG. 52 depicts a mechanical protocol for eliminating homotypic cellular aggregation and cellular settling during encapsulation. (Left) A centrifuge tube and round-bottom tube. (Center) Round-bottom tube placed inside centrifuge tube. (Right) The tubes connected to an OB-1 device during a microfluidic experiment.

FIG. 53 depicts a schematic representation of minigene lentiviral expression construct. Minigene expression is driven by EF1α promoter and with T2A to fluorescent marker mTagBFP2. Minigene is flanked by SpeI and BamHI restriction sites for synthetic library cloning.

FIG. 54 depicts, on the left, co-culture results with DMF5 TCR and MLANA A27L minigene (HLA Class I), T-cell activation measured as T-cell reporter (GFP) activation. As positive control cells were incubated with 10 μM of cognate peptide. On the right, co-culture results with TT2 TCR and TetX_400_1 minigene (HLA Class II), T-cell activation was measured as T-cell reporter (GFP) activation. As positive control cells were incubated with 1 μM of cognate peptide.

FIG. 55 depicts, on the left, mTagBFP2 expression level on BOLETH cells transduced with TetX_400_1 minigene. On the right, co-culture experiment with Jurkat TT2 and BOLETH freshly pulsed with cognate peptide or cultured with cognate peptide over time.

FIG. 56 depicts a schematic representation of lysosome targeting strategies. First panel LC3 Interaction domain (LIR), middle panel Lysosome localization with DC-LAMP, right panel Chaperone mediated autophagy (CMA).

FIG. 57 depicts the co-culture results comparing empty lentiviral vector and the selected three LIR sequences. LIR sequence from ATG14 was selected for further experiments.

FIG. 58 depicts co-culture results comparing empty lentiviral with vectors containing CMA sequence, DC LAMP or LIR ATG14. No constructs increased T-cell activation or prevent the antigen processing attenuation.

FIG. 59 depicts co-culture results with Jurkat TT2 and 100 AA TetX fragment cloned in CD74, and 400 AA TetX fragment cloned in both CD74 and empty constructs. CD74 construct can process short minigene fragments but not 400AA minigenes.

FIG. 60 depicts co-culture results with Jurkat TT2 T cells and cognate minigene cloned in the CD74 construct. Minigene lengths in AA are listed on the plot.

FIG. 61 depicts, top panel, schematic representation of HLA-DM alpha and beta chain molecules. The three alpha helix domains for each chain are highlighted. Minigene insert position is represent by the triangles. Lower panel, co-culture results using the HLA-DM constructs, as positive control were used CD74 construct containing the same minigene.

FIG. 62 depicts a schematic representation of the Bcap31 protein and the two transmembrane domains, Bcap31_1_104, Bcap31_6_21 and Bcap31_21_44, were cloned in the antigen-expressing vector.

FIG. 63 depicts co-culture results using TetX_100_50 minigene and TT2 expressing T-cells. Both Bcap311104 and Bcap31_21_44 did not improve the synthetic class II presentation, while when using Bcap31_216, the T-cell activation signal is as strong and stable as when stimulated with CD74 transduced BOLETH.

FIG. 64 depicts co-culture result with 400 AA minigene cloned in empty and Bcap31_TM1 constructs. Bcap31_TM1 construct induces a strong T-cell activation, and it is stable over time.

FIG. 65 depicts co-culture result with 400 AA minigene cloned in empty and Bcap31_TM1 constructs. A) The BT1 construct results were first validated with minigenes with increasing length. For the co-culture experiment were used TetX_400_1, TetX_2001, TetX_175_15, TetX 150_22, TetX_125_31 and TetX_100_50. The results for the three-time point are expressed as the ratio between the T cells activation induced with the BT1 construct over the one obtained with the 2B construct. The BT1 boost in Class II presentation is not affecting all the constructs, however, the activation is stronger or similar when BT1 is used. Only in case TetX_400_1 and TetX_175_15 the BT1 construct underperforms the 2B vector. (B) Comparison of BT1 constructs performance with 400 AA minigenes. The validation experiment was performed using TT7 TCR with TetX_400_916 and TetX_400_612, TT11 with TetX_400_612 and TT2 TetX_400_1. The BT1 construct can boost the synthetic class II presentation of 400 AA minigene.

FIG. 66 depicts TM_ER domains microscope plate-based screening. 576 potential transmembrane domains (single clones) were selected. Colonies were purified using a 96-well plate miniprep kit. Microscope plate-based screening protocol in 384-well plates. TT7 TCR and TetX_400_612—Measurement of GFP signal on day 5 and day 12 after transduction.

FIG. 67 depicts the design of a library of genomic and artificial transmembrane domains to be inserted N-terminally of a minigene for antigen processing.

FIG. 68 depicts the design of a functional plate-based screening to identify transmembrane domains for class II presentation.

FIG. 69 depicts TM_ER domains microscope plate-based screening results for plate 1—TT7 TCR on day 5 and day 12.

FIG. 70 depicts TM_ER domains microscope plate-based screening results for plate 2—TT7 TCR on day 5 and day 12.

FIG. 71 depicts TM_ER domains microscope plate-based screening results for plate 3—TT7 TCR on day 5 and day 12.

FIG. 72 depicts the validation of the TM_ER domains identified in plate-based screening.

FIG. 73 depicts TM_ER domain validation (control TCR TT7).

FIG. 74 depicts TM_ER domain validation (control TCR TT11).

FIG. 75 depicts TM_ER domain validation (control TCR TT2).

### DETAILED DESCRIPTION

The present invention relates generally to a system for the identification of target antigens for T cell receptors (TCRs), compositions for use in the system of the invention and methods of use of the system.

In one embodiment, the system comprises a modified T cell expressing a marker for tagging an antigen presenting cell (APC) upon binding of a TCR expressed by the T cell to an antigen expressed by the APC. In one embodiment, the marker for tagging an APC comprises an antibody or binding fragment thereof specific for binding to a receptor expressed on the surface of the APC. In one embodiment, the marker for tagging an APC comprises an scFv binding molecule specific for binding to a surface molecule of the APC. In some embodiments, the APC is a B cell. In some embodiments, the surface molecule of the APC (e.g., a B cell) is CD19 or CD20.

Therefore, in one embodiment, the invention provides compositions comprising an antibody or binding fragment thereof specific for binding to a receptor expressed on the surface of the APC, or a nucleic acid molecule encoding the same. In one embodiment, the invention relates to an scFv binding molecule specific for binding to a surface molecule of an APC, or a nucleic acid molecule encoding the same. In some embodiments, the APC is a B cell. In some embodiments, the surface molecule of the APC (e.g., a B cell) is CD19 or CD20.

In one embodiment, the invention provides T cells modified to express at least one antibody or binding fragment thereof specific for binding to a receptor expressed on the surface of the APC, or a T cell comprising a nucleic acid molecule encoding at least one antibody or binding fragment thereof specific for binding to a receptor expressed on the surface of the APC. In one embodiment, the invention relates to an scFv binding molecule specific for binding to a surface molecule of an APC, or a nucleic acid molecule encoding the same. In some embodiments, the APC is a B cell. In some embodiments, the surface molecule of the APC (e.g., a B cell) is CD19 or CD20.

In one embodiment, the marker for tagging an APC upon binding of a TCR expressed by the T cell to an antigen expressed by the APC is under the control of one or more regulatory elements that induce expression of the marker upon binding of a TCR expressed by the T cell to an antigen expressed by the APC. In such an embodiment, the expression of the marker for tagging the APC is restricted spatially and temporally so that only the interacting APC is tagged by the marker, which is expressed upon binding of the antigen expressed by the APC (e.g., a B cell) to the TCR expressed by the T cell.

Therefore, in some embodiments, the invention provides nucleic acid molecules comprising an expression cassette under the control of one or more inducible regulatory element(s), wherein the one or more inducible regulatory element(s) is/are induced upon binding of a TCR to an antigen. In some embodiments, one or more regulatory element(s) is/are an inducible promoter that is activated upon binding of a TCR to an antigen (referred to herein as a TCR responsive promoter). In one embodiment, one or more TCR responsive promoter(s) is/are inducible by NFAT. In one embodiment, the TCR responsive promoter is SEQ ID NO:50.

In some embodiments, the invention provides T cells comprising a nucleic acid molecule encoding a marker for tagging an APC upon binding of a TCR expressed by the T cell to an antigen expressed by the APC, wherein the sequence encoding the marker is under the control of one or more inducible regulatory element(s), wherein the one or more inducible regulatory element(s) is/are induced upon binding of a TCR to an antigen. In some embodiments, one or more regulatory element(s) is a TCR responsive promoter. In one embodiment, one or more TCR responsive promoter(s) is/are inducible by NFAT. In one embodiment, the TCR responsive promoter is SEQ ID NO:50.

In some embodiments, the nucleic acid molecules or cells of the invention comprise one or more additional nucleic acid sequences to further regulate the expression of the marker for tagging an APC. In some embodiments, one or more additional nucleic acid sequences include an NBV transcription factor for promoting transcription from an NBV TCR responsive promoter, and surface receptors known to be involved in T cell activation including, but not limited to, CD2, CD226, CD40L, ICOS, OX40, and 41BB. In some embodiments, the nucleic acid molecules comprise one or more insulator elements flanking the expression cassette to avoid spreading of activated chromatin from adjacent genetic elements or from a constitutively expressed downstream selection cassette. In some embodiments, the nucleic acid molecule comprises a 3′-untranslated region (UTR), derived from the human IgG1 heavy chain locus, to increase transcript stability and provide a dedicated polyadenylation sequence to the inducible cassette. In some embodiments, the nucleic acid molecules comprise one or more unique restriction enzyme sites for the incorporation of signal-responsive intron elements, which also serve to increase transcript stability, and downstream cis-regulatory elements (e.g., enhancers).

In one embodiment, the system of the invention employs APC cells expressing a library of antigens to identify new TCR:antigen interactions. In some embodiments, therefore, the invention relates to compositions comprising an APC cell library expressing a plurality of antigens and methods of screening the APC library for antigens that interact with a TCR.

In some embodiments, the antigen presenting cell library has been developed as a library of APCs wherein each APC comprises a nucleic acid molecule that is a member of a minigene library. In one embodiment, each nucleic acid molecule of the minigene library comprises a nucleotide sequence for expression of one or more antigen sequence(s) and further comprises a unique nucleotide sequence encoding the same amino acid tag sequence wherein the sequence encoding the amino acid tag serves as a barcode for identification of the expressed antigen(s). In one embodiment, each nucleotide sequence for expression of one or more antigen sequence(s) is designed to be a pre-determined length. In one embodiment, each nucleotide sequence for expression of one or more antigen sequence(s) comprises a single antigen sequence, or multiple antigen sequences separated by a linker sequence. In some embodiments, the nucleotide sequence for expression of one or more antigen sequence(s) is codon optimized for high-yield protein expression.

Therefore, in some embodiments, the invention relates to a plurality of nucleic acid molecules encoding an optimized minigene library, wherein each nucleic acid molecule comprises an antigen expression cassette (or minigene) comprising nucleotide sequence encoding one or more antigen sequence(s), or fragment thereof, and a unique nucleotide sequence encoding an amino acid tag. In some embodiments, each minigene sequence encodes a single antigen sequence, or multiple antigen sequences separated by linker sequences. In some embodiments, the minigene sequence is codon optimized for high-yield protein expression.

In some embodiments, the nucleic acid molecule encoding the minigene antigen expression cassette further encodes a flag-based surface receptor, a co-stimulatory molecule, or a combination thereof. In some embodiments, the co-stimulatory molecule is CD40, CD58, CD80, CD83, CD86, OX40L, 4-1BBL, or a combination thereof. In one embodiment the nucleotide sequence encoding the flag-based surface receptor comprises a sequence as set forth in SEQ ID NO:78, or a fragment or variant thereof. In one embodiment the nucleotide sequence encoding the co-stimulatory molecule is SEQ ID NO:79, which is a codon optimized sequence encoding CD40, or a fragment or variant thereof. In one embodiment the nucleotide sequence encoding the co-stimulatory molecule is SEQ ID NO:80, which is a codon optimized sequence encoding CD58, or a fragment or variant thereof. In one embodiment the nucleotide sequence encoding the co-stimulatory molecule is SEQ ID NO:81, which is a codon optimized sequence encoding CD80, or a fragment or variant thereof. In one embodiment the nucleotide sequence encoding the co-stimulatory molecule is SEQ ID NO:82, which is a codon optimized sequence encoding CD83, or a fragment or variant thereof. In one embodiment the nucleotide sequence encoding the co-stimulatory molecule is SEQ ID NO:83, which is a codon optimized sequence encoding CD86, or a fragment or variant thereof. In one embodiment the nucleotide sequence encoding the co-stimulatory molecule is SEQ ID NO:84, which is a codon optimized sequence encoding OX40L, or a fragment or variant thereof. In one embodiment the nucleotide sequence encoding the co-stimulatory molecule is SEQ ID NO:85, which is a codon optimized sequence encoding 41BBL, or a fragment or variant thereof. In various embodiments, the fragment of SEQ ID NO:79-85 comprises a sequence comprising at least 60%, 65%, 70%, 75%, 80%, 85%, 90%, 95%, 96%, 97%, 98%, 99% or more of the full-length sequence of SEQ ID NO:79-85. Specific fragments included in the invention may be a fragment lacking one or more codon at the 5′ or 3′ end of the sequence (e.g., a fragment lacking a start or stop codon.)

In some embodiments, the invention provides methods of generating a minigene library. In some embodiments, the invention provides method of optimizing a minigene library for high-yield, efficient expression of one or more antigen sequence(s).

In some embodiments, the invention relates to methods of co-culturing at least one APC of the invention and at least one T cell of the invention using a microfluidic co-culture system.

The present invention also provides microfluidic co-encapsulation devices configured to generate longitudinal flows of individual particles (such as individual cells) from fluid particle suspensions, combining two or more individual particle flows into a single stream of individual particles, and segmenting the single stream using an isolation fluid, resulting in a suspension of co-encapsulated individual particles from each fluid particle suspension. The present invention also provides methods of using the microfluidic co-encapsulation devices and methods of modifying fluid particle suspensions to promote individual particle separation.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“Activation”, as used herein, refers to the state of a T cell that has been sufficiently stimulated to induce detectable cytokine production, effector functions, and detectable signals associated with an activated state, such as transcription factors and surface proteins.

The term “antibody,” as used herein, refers to an immunoglobulin molecule which is able to specifically bind to a specific epitope of an antigen. Antibodies can be intact immunoglobulins derived from natural sources, or from recombinant sources and can be immunoreactive portions of intact immunoglobulins. The antibodies in the present invention may exist in a variety of forms including, for example, polyclonal antibodies, monoclonal antibodies, intracellular antibodies (“intrabodies”), Fv, Fab, Fab′, F(ab)2 and F(ab′)2, as well as single chain antibodies (scFv), heavy chain antibodies, such as camelid antibodies, and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, New York; Houston et al., 1988, Proc. Natl. Acad. Sci. USA 85:5879-5883; Bird et al., 1988, Science 242:423-426).

The term “antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, scFv antibodies, and multispecific antibodies formed from antibody fragments.

By the term “synthetic antibody” as used herein, is meant an antibody which is generated using recombinant DNA technology, such as, for example, an antibody expressed by a bacteriophage. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

A “humanized antibody” refers to a type of engineered antibody having its CDRs derived from a non-human donor immunoglobulin, the remaining immunoglobulin-derived parts of the molecule being derived from one (or more) human immunoglobulin(s). In addition, framework support residues may be altered to preserve binding affinity (see, e.g., 1989, Queen et al., Proc. Natl. Acad Sci USA, 86:10029-10032; 1991, Hodgson et al., Bio/Technology, 9:421). A suitable human acceptor antibody may be one selected from a conventional database, e.g., the KABAT database, Los Alamos database, and Swiss Protein database, by homology to the nucleotide and amino acid sequences of the donor antibody. A human antibody characterized by a homology to the framework regions of the donor antibody (on an amino acid basis) may be suitable to provide a heavy chain constant region and/or a heavy chain variable framework region for insertion of the donor CDRs. A suitable acceptor antibody capable of donating light chain constant or variable framework regions may be selected in a similar manner. It should be noted that the acceptor antibody heavy and light chains are not required to originate from the same acceptor antibody. Methods of producing humanized antibodies are known in the art.

By the term “recombinant antibody” as used herein, is meant an antibody which is generated using recombinant DNA technology, such as, for example, an antibody expressed by a bacteriophage or yeast cell expression system. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using recombinant DNA or amino acid sequence technology which is available and well known in the art.

An “antibody heavy chain,” as used herein, refers to the larger of the two types of polypeptide chains present in antibody molecules in their naturally occurring conformations, and which normally determines the class to which the antibody belongs.

An “antibody light chain,” as used herein, refers to the smaller of the two types of polypeptide chains present in antibody molecules in their naturally occurring conformations. Kappa (κ) and lambda (λ) light chains refer to the two major antibody light chain isotypes.

As used herein, “antigen-binding domain” means that part of the antibody, recombinant molecule, the fusion protein, or the immunoconjugate of the invention which recognizes the target or portions thereof.

The term “antigen” or “Ag” as used herein is defined as a molecule that provokes an adaptive immune response. This immune response may involve either antibody production, or the activation of specific immunogenically-competent cells, or both. The skilled artisan will understand that any macromolecule, including virtually all proteins or peptides, can serve as an antigen. Furthermore, antigens can be derived from recombinant or genomic DNA or RNA. A skilled artisan will understand that any DNA or RNA, which comprises a nucleotide sequence or a partial nucleotide sequence encoding a protein that elicits an adaptive immune response therefore encodes an “antigen” as that term is used herein. Furthermore, one skilled in the art will understand that an antigen need not be encoded solely by a full-length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, tumor sample, cell, biological fluid, body fluid, blood, serum, plasma, tissue, or any combination thereof.

As used herein, an “instructional material” includes a publication, a recording, a diagram, or any other medium of expression which can be used to communicate the usefulness of a compound, composition, vector, or delivery system of the invention in the kit for effecting alleviation of the various diseases or disorders recited herein. Optionally, or alternately, the instructional material can describe one or more methods of alleviating the diseases or disorders in a cell or a tissue of a mammal. The instructional material of the kit of the invention can, for example, be affixed to a container which contains the identified compound, composition, vector, or delivery system of the invention or be shipped together with a container which contains the identified compound, composition, vector, or delivery system. Alternatively, the instructional material can be shipped separately from the container with the intention that the instructional material and the compound be used cooperatively by the recipient.

“Operably linked,” “operatively linked,” or “under transcriptional control” as used herein may mean that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function.

The phrase “biological sample”, “sample” or “specimen” as used herein, is intended to include any sample comprising a cell, a tissue, or a bodily fluid in which expression of a nucleic acid or polypeptide can be detected. The biological sample may contain any biological material suitable for detecting the desired biomarkers, and may comprise cellular and/or non-cellular material obtained from the individual. Examples of such biological samples include but are not limited to blood, lymph, bone marrow, biopsies, and smears. Samples that are liquid in nature are referred to herein as “bodily fluids.” Biological samples may be obtained from a patient by a variety of techniques including, for example, by scraping or swabbing an area or by using a needle to obtain bodily fluids. Methods for collecting various body samples are well known in the art.

“CDRs” are defined as the complementarity determining region amino acid sequences of a TCR or TCR chain.

As used herein, an “immunoassay” refers to any binding assay that uses an antibody capable of binding specifically to a target molecule to detect and quantify the target molecule.

By the term “specifically binds,” as used herein with respect to a binding molecule (e.g., an antibody or TCR), is meant a binding molecule which recognizes a specific target molecule (e.g., an antigen or epitope), but does not substantially recognize or bind other molecules in a sample. For example, an antibody that specifically binds to an antigen from one species may also bind to that antigen from one or more other species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

A “coding region” of a mRNA molecule also consists of the nucleotide residues of the mRNA molecule which are matched with an anti-codon region of a transfer RNA molecule during translation of the mRNA molecule or which encode a stop codon. The coding region may thus include nucleotide residues comprising codons for amino acid residues which are not present in the mature protein encoded by the mRNA molecule (e.g., amino acid residues in a protein export signal sequence).

“Complementary” as used herein to refer to a nucleic acid, refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. In some embodiments, the first region comprises a first portion and the second region comprises a second portion, whereby, when the first and second portions are arranged in an antiparallel fashion, at least about 50%, and or at least about 75%, or at least about 90%, or at least about 95% of the nucleotide residues of the first portion are capable of base pairing with nucleotide residues in the second portion. In some embodiments, all nucleotide residues of the first portion are capable of base pairing with nucleotide residues in the second portion.

The term “DNA” as used herein is defined as deoxyribonucleic acid.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting there from. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

“Isolated” means altered or removed from the natural state. For example, a nucleic acid or a peptide naturally present in its normal context in a living subject is not “isolated,” but the same nucleic acid or peptide partially or completely separated from the coexisting materials of its natural context is “isolated.” An isolated nucleic acid or protein can exist in substantially purified form, or can exist in a non-native environment such as, for example, a host cell.

An “isolated nucleic acid” refers to a nucleic acid segment or fragment which has been separated from sequences which flank it in a naturally occurring state, i.e., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, i.e., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes a recombinant DNA which is part of a hybrid gene encoding additional polypeptide sequence.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “RNA” as used herein is defined as ribonucleic acid.

The term “recombinant DNA” as used herein is defined as DNA produced by joining pieces of DNA from different sources.

The term “recombinant polypeptide” as used herein is defined as a polypeptide produced by using recombinant DNA methods.

As used herein, “conjugated” refers to covalent attachment of one molecule to a second molecule.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared ×100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

“Variant” as the term is used herein, is a nucleic acid sequence or a peptide sequence that differs in sequence from a reference nucleic acid sequence, or peptide sequence respectively, but retains essential biological properties of the reference molecule. Changes in the sequence of a nucleic acid variant may not alter the amino acid sequence of a peptide encoded by the reference nucleic acid, or may result in amino acid substitutions, additions, deletions, fusions, and truncations. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring, such as an allelic variant, or can be a variant that is not known to occur naturally. Non-naturally occurring variants of nucleic acids and peptides may be made by mutagenesis techniques or by direct synthesis. In various embodiments, the variant sequence is at least 99%, at least 98%, at least 97%, at least 96%, at least 95%, at least 94%, at least 93%, at least 92%, at least 91%, at least 90%, at least 89%, at least 88%, at least 87%, at least 86%, at least 85% identical to the reference sequence.

The term “regulating” as used herein can mean any method of altering the level or activity of a substrate. Non-limiting examples of regulating with regard to a protein include affecting expression (including transcription and/or translation), affecting folding, affecting degradation or protein turnover, and affecting localization of a protein. Non-limiting examples of regulating with regard to an enzyme further include affecting the enzymatic activity. “Regulator” refers to a molecule whose activity includes affecting the level or activity of a substrate. A regulator can be direct or indirect. A regulator can function to activate or inhibit or otherwise modulate its substrate.

“Variant” with respect to a peptide or polypeptide that differs in amino acid sequence by the insertion, deletion, or conservative substitution of amino acids, but retain at least one biological activity. Variant may also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of 2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference. Substitution of amino acids having similar hydrophilicity values can result in peptides retaining biological activity, for example immunogenicity, as is understood in the art. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

“Vector” as used herein may mean a nucleic acid sequence containing an origin of replication. A vector may be a plasmid, bacteriophage, bacterial artificial chromosome or yeast artificial chromosome. A vector may be a DNA or RNA vector. A vector may be either a self-replicating extrachromosomal vector or a vector which integrates into a host genome.

As used herein, a “substantially purified” cell is a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are cultured in vitro. In other embodiments, the cells are not cultured in vitro.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### APC Tagging Molecules

In one embodiment, the invention relates to compositions comprising a cell expressing a T cell receptor (TCR) molecule and further expressing a secreted molecule specific for binding to a marker expressed on a surface of an antigen-presenting cell (APC), referred to herein as an APC tagging molecule.

In some embodiments, the cell is an immune cell. Exemplary immune cells that may comprise or express one or more TCRs include, but are not limited to, T cells (including killer T cells, helper T cells, regulatory T cells, gamma delta T cells, and laboratory-isolated or -generated T cell lines), engineered natural killer (NK) cells, NK T cells, or cells engineered to express a TCR. Therefore, in some embodiments, the invention provides a T cell expressing an APC tagging molecule. In some embodiments, the invention provides a T cell comprising a nucleic acid molecule comprising a nucleotide sequence encoding an APC tagging molecule.

In some embodiments, the TCR is an engineered TCR. In some embodiments, the nucleotide sequence encoding the TCR comprises a nucleotide sequence generated from a nucleotide sequence of a TCR isolated from a subject (i.e., a subject or patient derived TCR). In such an embodiment, the T cell of the invention comprises a deletion of the native TCR, and further comprises an exogenous nucleic acid sequence encoding a non-native TCR. In one embodiment, the non-native TCR is a subject or patient derived TCR. In one embodiment, the T cell further comprises a second exogenous nucleic acid molecule encoding an APC tagging molecule.

An APC is any cell expressing one or more major histocompatibility complex (MHC), or as they are specifically named in human cells, human leukocyte antigen (HLA), proteins on its surface. Exemplary APCs include, but are not limited to, dendritic cells (DCs), macrophages, Langerhans cells, B cells, and the like. In some embodiments, the APC is a cell engineered to express major histocompatibility complex (MHC), or human leukocyte antigen (HLA).

In one embodiment, the APC is a B cell and the APC tagging molecule is a molecule specific for binding to a B cell marker. Exemplary B cell markers include, but are not limited to CD19, CD20, CD38, CD40, CD45R, CD79a, and CD79b. Therefore, in some embodiments, the APC tagging molecule comprises a molecule specific for binding to CD19, CD20, CD38, CD40, CD45R, CD79a, or CD79b.

In one embodiment, the APC is a DC and the APC tagging molecule is a molecule specific for binding to a DC marker. Exemplary DC markers include, but are not limited to, CD11c and CD1a. Therefore, in some embodiments, the APC tagging molecule comprises a molecule specific for binding to CD11c or CD1a.

In some embodiments, the APC tagging molecule comprises an antibody, or fragment thereof, specific for binding to an APC cell marker. In some embodiments, the APC tagging molecule comprises an antibody, or fragment thereof, specific for binding to CD19, CD20, CD38, CD40, CD45R, CD79a, CD79b, CD11c or CD1a.

In various embodiments, the APC tagging molecule is an antibody, an antibody fragment, a peptide sequence, aptamer, a ligand, a gene component, or any combination thereof. Examples of APC tagging molecule include, but are not limited to antibodies, peptidomimetics, synthetic ligands, and the like which specifically bind desired target APCs. APC tagging molecules of particular interest include peptidomimetics, peptides, antibodies (e.g., monoclonal antibodies, polyclonal antibodies, recombinant antibodies, human antibodies, humanized antibodies, etc.) and antibody fragments (e.g., the Fab′ fragment).

The invention encompasses monoclonal, synthetic antibodies, and the like. One skilled in the art would understand, based upon the disclosure provided herein, that the crucial feature of the antibody of the invention is that the antibody binds specifically with an APC marker of interest. That is, the antibody of the invention recognizes a target of interest or a fragment thereof.

The skilled artisan would appreciate, based upon the disclosure provided herein, that present invention includes use of a single antibody recognizing a single antibody target but that the invention is not limited to use of a single antibody, or fragment thereof. Instead, the invention encompasses use of at least one antibody, or fragment thereof, where the antibodies can be directed to the same or different antibody targets.

Nucleic acid molecules encoding the binding molecule described herein may be cloned and sequenced using technology, which is available in the art and is described, for example, in Wright et al. (1992, Critical Rev. Immunol. 12:125-168), and the references cited therein. Further, the antibody of the invention may be “humanized” using the technology described in, for example, Wright et al., and in the references cited therein, and in Gu et al. (1997, Thrombosis and Hematocyst 77:755-759), and other methods of humanizing antibodies well-known in the art or to be developed.

In some embodiments, a non-human antibody is humanized, where specific sequences or regions of the antibody are modified to increase similarity to an antibody naturally produced in a human or fragment thereof. A humanized antibody can be produced using a variety of techniques known in the art, including but not limited to, CDR-grafting (see, e.g., European Patent No. EP 239,400; International Publication No. WO 91/09967; and U.S. Pat. Nos. 5,225,539, 5,530,101, and 5,585,089), veneering or resurfacing (see, e.g., European Patent Nos. EP 592, 106 and EP 519,596; Padlan, 1991, Molecular Immunology, 28(4/5):489-498; Studnicka et al., 1994, Protein Engineering, 7(6):805-814; and Roguska et al., 1994, PNAS, 91:969-973), chain shuffling (see, e.g., U.S. Pat. No. 5,565,332), and techniques disclosed in, e.g., U.S. Patent Application Publication No. US2005/0042664, U.S. Patent Application Publication No. US2005/0048617, U.S. Pat. Nos. 6,407,213, 5,766,886, International Publication No. WO 9317105, Tan et al., J. Immunol., 169:1119-25 (2002), Caldas et al., Protein Eng., 13(5):353-60 (2000), Morea et al., Methods, 20(3):267-79 (2000), Baca et al., J. Biol. Chem., 272(16):10678-84 (1997), Roguska et al., Protein Eng., 9(10):895-904 (1996), Couto et al., Cancer Res., 55 (23 Supp):5973s-5977s (1995), Couto et al., Cancer Res., 55(8):1717-22 (1995), Sandhu J S, Gene, 150(2):409-10 (1994), and Pedersen et al., J. Mol. Biol., 235(3):959-73 (1994). Often, framework residues in the framework regions will be substituted with the corresponding residue from the CDR donor antibody to alter, for example improve, antigen binding. These framework substitutions are identified by methods well-known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; and Riechmann et al., 1988, Nature, 332:323.)

In one embodiment, the antibody fragment provided herein is a single chain variable fragment (scFv). In various embodiments, the antibodies of the invention may exist in a variety of other forms including, for example, Fv and Fab, as well as bi-functional (i.e. bi-specific) hybrid antibodies (e.g., Lanzavecchia et al., Eur. J. Immunol. 17, 105 (1987)). In some embodiments, the antibodies and fragments thereof of the invention bind an APC. In some embodiments, the antibodies and fragments thereof of the invention bind a B cell. In some embodiments, the antibodies and fragments thereof of the invention bind a DC.

In one embodiment, the APC tagging molecule is specific for binding to CD19. In one embodiment, the APC tagging molecule specific for binding to a CD19B cell marker comprises an scFv antibody fragment comprising a heavy chain variable region comprising an amino acid sequence as set forth in SEQ NO:4, or a fragment or variant thereof, and a light chain variable region comprising an amino acid sequence as set forth in SEQ ID NO:8, or a fragment or variant thereof. A fragment or variant of SEQ ID NO:4 may comprise a fragment or variant of SEQ ID NO:4 in which the CDR regions of SEQ ID NO:1, SEQ ID NO:2 and SEQ ID NO:3 are retained, but non-CDR sequences are modified or varied. A fragment or variant of SEQ ID NO:8 may comprise a fragment or variant of SEQ ID NO:8 in which the CDR regions of SEQ ID NO:5, SEQ ID NO:6 and SEQ ID NO:7 are retained, but non-CDR sequences are modified or varied.

In one embodiment, the APC tagging molecule is an scFv antibody fragment specific for binding to CD19. In one embodiment, the APC tagging molecule specific for binding to a CD19 B cell marker comprises an scFv antibody fragment comprising a heavy chain variable region comprising an amino acid sequence as set forth in SEQ NO: 12, or a fragment or variant thereof, and a light chain variable region comprising an amino acid sequence as set forth in SEQ ID NO:16, or a fragment or variant thereof. A fragment or variant of SEQ ID NO:12 may comprise a fragment or variant of SEQ ID NO:12 in which the CDR regions of SEQ ID NO:9, SEQ ID NO:10 and SEQ ID NO:11 are retained, but non-CDR sequences are modified or varied. A fragment or variant of SEQ ID NO:16 may comprise a fragment or variant of SEQ ID NO:16 in which the CDR regions of SEQ ID NO:13, SEQ ID NO:14 and SEQ ID NO:15 are retained, but non-CDR sequences are modified or varied.

In one embodiment, the APC tagging molecule is specific for binding to CD20. In one embodiment, the APC tagging molecule specific for binding to a CD20B cell marker comprises an scFv antibody fragment comprising a heavy chain variable region comprising an amino acid sequence as set forth in SEQ NO:20, or a fragment or variant thereof, and a light chain variable region comprising an amino acid sequence as set forth in SEQ ID NO:24, or a fragment or variant thereof. A fragment or variant of SEQ ID NO:20 may comprise a fragment or variant of SEQ ID NO:20 in which the CDR regions of SEQ ID NO:17, SEQ ID NO:18 and SEQ ID NO:19 are retained, but non-CDR sequences are modified or varied. A fragment or variant of SEQ ID NO:24 may comprise a fragment or variant of SEQ ID NO:24 in which the CDR regions of SEQ ID NO:21, SEQ ID NO:22 and SEQ ID NO:23 are retained, but non-CDR sequences are modified or varied.

In one embodiment, the APC tagging molecule is an scFv antibody fragment specific for binding to CD20. In one embodiment, the APC tagging molecule specific for binding to a CD20 B cell marker comprises an scFv antibody fragment comprising a heavy chain variable region comprising an amino acid sequence as set forth in SEQ NO:28, or a fragment or variant thereof, and a light chain variable region comprising an amino acid sequence as set forth in SEQ ID NO:32, or a fragment or variant thereof. A fragment or variant of SEQ ID NO:28 may comprise a fragment or variant of SEQ ID NO:28 in which the CDR regions of SEQ ID NO:25, SEQ ID NO:26 and SEQ ID NO:27 are retained, but non-CDR sequences are modified or varied. A fragment or variant of SEQ ID NO:32 may comprise a fragment or variant of SEQ ID NO:32 in which the CDR regions of SEQ ID NO:29, SEQ ID NO:30 and SEQ ID NO:31 are retained, but non-CDR sequences are modified or varied.

A variant of an amino acid sequence may be an amino acid sequence that is substantially identical over the full-length of the amino acid sequence or fragment thereof to a parental amino acid sequence. The amino acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full-length of the amino acid sequence or a fragment thereof to a parental amino acid sequence. In one embodiment, the variant of SEQ ID NO:4 comprises a sequence 80%, 81%, 82%, 83%, 84%, 85%, 86%, 87%, 88%, 89%, 90%, 91%, 92%, 93%, 94%, 95%, 96%, 97%, 98%, 99%, or 100% identical over the full-length of the amino acid sequence to SEQ ID NO:4, but in which the sequence of the CDR sequences of SEQ ID NO:1, SEQ ID NO:2, and SEQ ID NO:3 are not varied (i.e. the CDR sequences are 100% identical to the CDR sequences of SEQ ID NO:1, SEQ ID NO:2, and SEQ ID NO:3.) In one embodiment, the variant of SEQ ID NO:4 comprises a sequence 80%, 81%, 82%, 83%, 84%, 85%, 86%, 87%, 88%, 89%, 90%, 91%, 92%, 93%, 94%, 95%, 96%, 97%, 98%, 99%, or 100% identical over the full-length of the amino acid sequence to SEQ ID NO:8, but in which the sequence of the CDR sequences of SEQ ID NO:5, SEQ ID NO:6, and SEQ ID NO:7 are not varied (i.e. the CDR sequences are 100% identical to the CDR sequences of SEQ ID NO:5, SEQ ID NO:6, and SEQ ID NO:7.) In one embodiment, the variant of SEQ ID NO:4 comprises a sequence 80%, 81%, 82%, 83%, 84%, 85%, 86%, 87%, 88%, 89%, 90%, 91%, 92%, 93%, 94%, 95%, 96%, 97%, 98%, 99%, or 100% identical over the full-length of the amino acid sequence to SEQ ID NO:12, but in which the sequence of the CDR sequences of SEQ ID NO:9, SEQ ID NO:10, and SEQ ID NO:11 are not varied (i.e. the CDR sequences are 100% identical to the CDR sequences of SEQ ID NO:9, SEQ ID NO:10, and SEQ ID NO:11.) In one embodiment, the variant of SEQ ID NO:16 comprises a sequence 80%, 81%, 82%, 83%, 84%, 85%, 86%, 87%, 88%, 89%, 90%, 91%, 92%, 93%, 94%, 95%, 96%, 97%, 98%, 99%, or 100% identical over the full-length of the amino acid sequence to SEQ ID NO:16, but in which the sequence of the CDR sequences of SEQ ID NO:13, SEQ ID NO:14, and SEQ ID NO:15 are not varied (i.e. the CDR sequences are 100% identical to the CDR sequences of SEQ ID NO:13, SEQ ID NO:14, and SEQ ID NO:15.) In one embodiment, the variant of SEQ ID NO:20 comprises a sequence 80%, 81%, 82%, 83%, 84%, 85%, 86%, 87%, 88%, 89%, 90%, 91%, 92%, 93%, 94%, 95%, 96%, 97%, 98%, 99%, or 100% identical over the full-length of the amino acid sequence to SEQ ID NO:20, but in which the sequence of the CDR sequences of SEQ ID NO:17, SEQ ID NO:18, and SEQ ID NO:19 are not varied (i.e. the CDR sequences are 100% identical to the CDR sequences of SEQ ID NO:17, SEQ ID NO:18, and SEQ ID NO:19.) In one embodiment, the variant of SEQ ID NO:24 comprises a sequence 80%, 81%, 82%, 83%, 84%, 85%, 86%, 87%, 88%, 89%, 90%, 91%, 92%, 93%, 94%, 95%, 96%, 97%, 98%, 99%, or 100% identical over the full-length of the amino acid sequence to SEQ ID NO:24, but in which the sequence of the CDR sequences of SEQ ID NO:21, SEQ ID NO:22, and SEQ ID NO:23 are not varied (i.e. the CDR sequences are 100% identical to the CDR sequences of SEQ ID NO:21, SEQ ID NO:22, and SEQ ID NO:23.) In one embodiment, the variant of SEQ ID NO:28 comprises a sequence 80%, 81%, 82%, 83%, 84%, 85%, 86%, 87%, 88%, 89%, 90%, 91%, 92%, 93%, 94%, 95%, 96%, 97%, 98%, 99%, or 100% identical over the full-length of the amino acid sequence to SEQ ID NO:28, but in which the sequence of the CDR sequences of SEQ ID NO:25, SEQ ID NO:26, and SEQ ID NO:27 are not varied (i.e. the CDR sequences are 100% identical to the CDR sequences of SEQ ID NO:25, SEQ ID NO:26, and SEQ ID NO:27.) In one embodiment, the variant of SEQ ID NO:32 comprises a sequence 80%, 81%, 82%, 83%, 84%, 85%, 86%, 87%, 88%, 89%, 90%, 91%, 92%, 93%, 94%, 95%, 96%, 97%, 98%, 99%, or 100% identical over the full-length of the amino acid sequence to SEQ ID NO:32, but in which the sequence of the CDR sequences of SEQ ID NO:29, SEQ ID NO:30, and SEQ ID NO:31 are not varied (i.e. the CDR sequences are 100% identical to the CDR sequences of SEQ ID NO:29, SEQ ID NO:30, and SEQ ID NO:31.)

ScFvs can be prepared according to method known in the art (see, for example, Bird et al., (1988) Science 242:423-426 and Huston et al., (1988) Proc. Natl. Acad. Sci. USA 85:5879-5883). ScFv molecules can be produced by linking VH and VL regions together using flexible polypeptide linkers. The scFv molecules comprise flexible polypeptide linker (e.g., a Ser-Gly linker) with an optimized length and/or amino acid composition. The flexible polypeptide linker length can greatly affect how the variable regions of an scFv fold and interact. In fact, if a short polypeptide linker is employed (e.g., between 5-10 amino acids, intrachain folding is prevented. Interchain folding is also required to bring the two variable regions together to form a functional epitope binding site. For examples of linker orientation and size see, e.g., Hollinger et al. 1993 Proc Natl Acad. Sci. U.S.A. 90:6444-6448, U.S. Patent Application Publication Nos. 2005/0100543, 2005/0175606, 2007/0014794, and PCT publication Nos. WO2006/020258 and WO2007/024715.

The scFv can comprise a polypeptide linker sequence of at least 1, 2, 3, 4, 5, 6, 7, 8, 9, 10, 11, 12, 13, 14, 15, 16, 17, 18, 19, 20, 25, 30, 35, 40, 45, 50, or more amino acid residues between its VL and VH regions. The flexible polypeptide linker sequence may comprise any naturally occurring amino acid. In some embodiments, the flexible polypeptide linker sequence comprises amino acids glycine and serine. In another embodiment, the flexible polypeptide linker sequence comprises sets of glycine and serine repeats such as (Gly4Ser)n, where n is a positive integer equal to or greater than 1. In one embodiment, the flexible polypeptide linkers include, but are not limited to, (Gly4Ser)4, (Gly4Ser)3, and SEQ ID NO:42. Variation in the flexible polypeptide linker length may retain or enhance activity, giving rise to superior efficacy.

In some embodiments, the scFv molecule comprises a polypeptide leader sequence of at least 1, 2, 3, 4, 5, 6, 7, 8, 9, 10, 11, 12, 13, 14, 15, 16, 17, 18, 19, 20, 25, 30, 35, 40, 45, 50, or more amino acid residues. In one embodiment, the leader sequence comprises a sequence as set forth in SEQ ID NO:43.

In some embodiments, the scFv molecule comprises a capture tag sequence which allows for isolation of the scFv and an associated “tagged” APC. Exemplary capture tags that can be included on an scFv of the invention include, but are not limited to, a 4×V5 tag (SEQ ID NO:44), a 3×HA tag (SEQ ID NO:45), a 6×HA tag (SEQ ID NO:46), a 6× FLAG tag (SEQ ID NO:47), or a 3× FLAG tag (the FLAG sequence is DYKDDDDK (SEQ ID NO:48).

In one embodiment, the APC tagging molecule comprises two or more tandem APC tagging molecules, separated by a linker sequence. In some embodiments, the linker sequence comprises SEQ ID NO:49. In some embodiments, the tandem APC tagging molecule comprises at least two, at least three, at least four, or more than four tandem APC molecules. In some embodiments, the tandem APC tagging molecule comprises at least two, at least three, at least four, or more than four tandem scFv molecules. In some embodiments, two or more tandem APC tagging molecules target the same APC marker. In some embodiments, two or more tandem APC tagging molecules target two or more different APC markers. An exemplary diabody comprising two tandem APC tagging molecules comprises the sequence as set forth in SEQ ID NO:33.

### Recombinant Nucleic Acid Sequence Construct

As described above, the composition can comprise a recombinant nucleic acid sequence. The recombinant nucleic acid sequence can encode the antibody, a fragment thereof, a variant thereof, or a combination thereof. The recombinant nucleic acid sequence can be a heterologous nucleic acid sequence. The recombinant nucleic acid sequence can include one or more heterologous nucleic acid sequences.

The recombinant nucleic acid sequence can be an optimized nucleic acid sequence. Such optimization can increase or alter the immunogenicity of the antibody. Optimization can also improve transcription and/or translation. Optimization can include one or more of the following: low GC content leader sequence to increase transcription; mRNA stability and codon optimization; addition of a Kozak sequence for increased translation; addition of an immunoglobulin (Ig) leader sequence encoding a signal peptide; addition of an internal IRES sequence and eliminating to the extent possible cis-acting sequence motifs (i.e., internal TATA boxes).

The recombinant nucleic acid sequence can include one or more recombinant nucleic acid sequence constructs. The recombinant nucleic acid sequence construct can include one or more components, which are described in more detail below.

The recombinant nucleic acid sequence construct can include a heterologous nucleic acid sequence that encodes a heavy chain polypeptide, a fragment thereof, a variant thereof, or a combination thereof. The recombinant nucleic acid sequence construct can include a heterologous nucleic acid sequence that encodes a light chain polypeptide, a fragment thereof, a variant thereof, or a combination thereof. The recombinant nucleic acid sequence construct can also include a heterologous nucleic acid sequence that encodes a protease or peptidase cleavage site. The recombinant nucleic acid sequence construct can also include a heterologous nucleic acid sequence that encodes an internal ribosome entry site (IRES). An IRES may be either a viral IRES or a eukaryotic IRES. The recombinant nucleic acid sequence construct can include one or more leader sequences, in which each leader sequence encodes a signal peptide. The recombinant nucleic acid sequence construct can include one or more promoters, one or more introns, one or more transcription termination regions, one or more initiation codons, one or more termination or stop codons, and/or one or more polyadenylation signals. The recombinant nucleic acid sequence construct can also include one or more linker or tag sequences, as described above.

The recombinant nucleic acid molecule construct can include the heterologous nucleic acid sequence encoding the heavy chain polypeptide, a fragment thereof, a variant thereof, or a combination thereof. The heavy chain polypeptide can include a variable heavy chain (VH) region and/or at least one constant heavy chain (CH) region. The at least one constant heavy chain region can include a constant heavy chain region 1 (CH1), a constant heavy chain region 2 (CH2), and a constant heavy chain region 3 (CH3), and/or a hinge region.

In some embodiments, the heavy chain polypeptide can include a VH region and a CH1 region. In other embodiments, the heavy chain polypeptide can include a VH region, a CH1 region, a hinge region, a CH2 region, and a CH3 region.

The heavy chain polypeptide can include a complementarity determining region (“CDR”) set. The CDR set can contain three hypervariable regions of the VH region. Proceeding from N-terminus of the heavy chain polypeptide, these CDRs are denoted “CDR1,” “CDR2,” and “CDR3,” respectively. CDR1, CDR2, and CDR3 of the heavy chain polypeptide can contribute to binding or recognition of the antigen.

The recombinant nucleic acid sequence construct can include the heterologous nucleic acid sequence encoding the light chain polypeptide, a fragment thereof, a variant thereof, or a combination thereof. The light chain polypeptide can include a variable light chain (VL) region and/or a constant light chain (CL) region.

The light chain polypeptide can include a complementarity determining region (“CDR”) set. The CDR set can contain three hypervariable regions of the VL region. Proceeding from N-terminus of the light chain polypeptide, these CDRs are denoted “CDR1,” “CDR2,” and “CDR3,” respectively. CDR1, CDR2, and CDR3 of the light chain polypeptide can contribute to binding or recognition of the antigen.

In one embodiment, the nucleic acid molecule encoding an APC tagging molecule specific for binding to a CD19 B cell marker comprises a nucleotide sequence encoding an scFv antibody fragment comprising a heavy chain variable region comprising an amino acid sequence as set forth in SEQ NO:4, or a fragment or variant thereof, and a light chain variable region comprising an amino acid sequence as set forth in SEQ ID NO:8, or a fragment or variant thereof. A fragment or variant of SEQ ID NO:4 or SEQ ID NO:8 may comprise a fragment or variant of SEQ ID NO:4 or SEQ ID NO:8 in which the CDR regions of SEQ ID NO:1-3 and SEQ ID NO:5-7 respectively are retained, but non-CDR sequences are modified or varied. In one embodiment, the nucleic acid molecule encoding an APC tagging molecule specific for binding to a CD20 B cell marker comprises a nucleotide sequence encoding SEQ ID NO:33, SEQ ID NO:39 or SEQ ID NO:41. In one embodiment, the nucleic acid molecule encoding an APC tagging molecule specific for binding to a CD20 B cell marker comprises a nucleotide sequence as set forth in SEQ ID NO:40 or SEQ ID NO:38.

In one embodiment, the nucleic acid molecule encoding an APC tagging molecule specific for binding to a CD19 B cell marker comprises a nucleotide sequence encoding an scFv antibody fragment comprising a heavy chain variable region comprising an amino acid sequence as set forth in SEQ NO:12, or a fragment or variant thereof, and a light chain variable region comprising an amino acid sequence as set forth in SEQ ID NO:16, or a fragment or variant thereof. A fragment or variant of SEQ ID NO:12 or SEQ ID NO:16 may comprise a fragment or variant of SEQ ID NO:12 or SEQ ID NO:16 in which the CDR regions of SEQ ID NO:9-11 and SEQ ID NO:13-15 respectively are retained, but non-CDR sequences are modified or varied.

In one embodiment, the nucleic acid molecule encoding an APC tagging molecule specific for binding to a CD20 B cell marker comprises a nucleotide sequence encoding an scFv antibody fragment comprising a heavy chain variable region comprising an amino acid sequence as set forth in SEQ NO:20, or a fragment or variant thereof, and a light chain variable region comprising an amino acid sequence as set forth in SEQ ID NO:24, or a fragment or variant thereof. A fragment or variant of SEQ ID NO:20 or SEQ ID NO:24 may comprise a fragment or variant of SEQ ID NO:20 or SEQ ID NO:24 in which the CDR regions of SEQ ID NO:17-19 and SEQ ID NO:21-23 respectively are retained, but non-CDR sequences are modified or varied. In one embodiment, the nucleic acid molecule encoding an APC tagging molecule specific for binding to a CD20 B cell marker comprises a nucleotide sequence encoding SEQ ID NO:33, SEQ ID NO:35 or SEQ ID NO:37. In one embodiment, the nucleic acid molecule encoding an APC tagging molecule specific for binding to a CD20 B cell marker comprises a nucleotide sequence as set forth in SEQ ID NO:34 or SEQ ID NO:36.

In one embodiment, the nucleic acid molecule encoding an APC tagging molecule specific for binding to a CD20 B cell marker comprises a nucleotide sequence encoding an scFv antibody fragment comprising a heavy chain variable region comprising an amino acid sequence as set forth in SEQ NO:28, or a fragment or variant thereof, and a light chain variable region comprising an amino acid sequence as set forth in SEQ ID NO:32, or a fragment or variant thereof. A fragment or variant of SEQ ID NO:28 or SEQ ID NO:32 may comprise a fragment or variant of SEQ ID NO:28 or SEQ ID NO:32 in which the CDR regions of SEQ ID NO:25-27 and SEQ ID NO:29-31 respectively are retained, but non-CDR sequences are modified or varied.

A variant may be a sequence that is substantially identical over the full-length to a parental sequence or a fragment thereof. A variant of a nucleic acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full-length of the gene sequence or a fragment thereof to a parental gene sequence.

The recombinant nucleic acid sequence construct can include one or more linker sequences as described above. The linker sequence can spatially separate or link the one or more components described herein. In some embodiments, the linker sequence can encode an amino acid sequence that spatially separates or links a heavy chain and a light chain of an scFv antibody fragment. In some embodiments, the linker sequence can encode an amino acid sequence that spatially separates or links two or more tandem scFv antibody fragments.

### Promoter

The recombinant nucleic acid sequence construct can include one or more promoters. The one or more promoters may be any promoter that is capable of driving gene expression and regulating gene expression. Such a promoter is a cis-acting sequence element required for transcription via a DNA dependent RNA polymerase. Selection of the promoter used to direct gene expression depends on the particular application. The promoter may be positioned about the same distance from the transcription start in the recombinant nucleic acid sequence construct as it is from the transcription start site in its natural setting. However, variation in this distance may be accommodated without loss of promoter function.

The promoter may be operably linked to the heterologous nucleic acid sequence encoding the heavy chain polypeptide and/or light chain polypeptide. The promoter may be a promoter shown effective for expression in eukaryotic cells. The promoter operably linked to the coding sequence may be a CMV promoter, a promoter from simian virus 40 (SV40), such as SV40 early promoter and SV40 later promoter, a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter may also be a promoter from a human gene such as human actin, human myosin, human hemoglobin, human muscle creatine, human polyhedrin, or human metallothionein. In various embodiments, the constitutive promoter is the EFlalpha promoter, the ActB promoter, the Gapdh promoter, the IGKV1-39 promoter, the Rp141 promoter, the Rps2 promoter, the Rps18 promoter or the Tsmb4× promoter. The promoter may also be a synthetic, human-designed nucleotide sequence intended to achieve the desired effect on expression. The promoter may also be comprised of fragments of promoters from any or all of the above examples.

The promoter can be a constitutive promoter or an inducible promoter, (e.g., a promoter which initiates transcription only when a TCR expressed by the T cell is bound to an antigen presented by an APC, as described in more detail below.)

The promoter can be associated with an enhancer. The enhancer can be located upstream or downstream of the coding sequence. Exemplary enhancers include, but are not limited to, those described in more detail below.

### TCR Responsive Promoters

In one embodiment, the invention relates to the development of a system comprising a T cell that expresses a target protein or peptide under the control of a TCR responsive promoter, wherein the target protein or peptide is expressed when a TCR is bound to an antigenic protein or peptide. In one embodiment, exemplary target proteins or peptides that can be expressed using the TCR responsive promoter of the invention include, but are not limited to, an antibody or fragment thereof, a detectable marker, a fluorescent protein, a transcription factor, and an enzyme. In one embodiment, the invention relates to the development of a system comprising a T cell that expresses an APC tagging molecule under the control of an inducible promoter such that the APC tagging molecule is expressed upon binding of a TCR of the T cell to an antigen presented by the APC. Therefore, in some embodiments, the invention provides compositions comprising TCR responsive promoters.

In some embodiments, the TCR responsive promoter comprises a sequence selected from SEQ ID NO:50-SEQ ID NO:74. In one embodiment, therefore, the invention provides nucleic acid molecules comprising a TCR responsive promoter of SEQ ID NO:50-SEQ ID NO:74 operably linked to a sequence encoding a B cell tagging molecule of the invention.

In some embodiments, the TCR responsive promoter of the invention is inducible by NFAT. In some embodiments, the TCR responsive promoter of the invention is an NBV promoter which is inducible by a NBV transcription factor comprising a fusion of the following elements: the cytoplasmic retention and DNA-binding domains from the N′-terminus of the nuclear factor in activated T cells (NFAT), the octamer motif (‘ATGCAAAT’)-binding domain from the transcriptional co-activator Bob1, and the C′-terminal transactivation domain (TAD) from the herpesvirus VP16 protein. In one embodiment, the NBV promoter comprises a sequence as set forth in SEQ ID NO:50, and the NBV transcription factor comprises a sequence as set forth in SEQ ID NO:76. In one embodiment, the NBV transcription factor is encoded by a sequence as set forth in SEQ ID NO:75.

### Modified Cells

In one embodiment, the invention provides a modified cell containing a nucleic acid molecule comprising a nucleotide sequence encoding a heterologous sequence for expression under the control of a TCR responsive promoter. In one embodiment, the heterologous sequence encodes an antibody or fragment thereof, a detectable marker, a fluorescent protein, a transcription factor, or an enzyme. For example, in one embodiment, the invention provides a modified T cell containing a nucleic acid molecule comprising a nucleotide sequence encoding an APC tagging molecule operably linked to a TCR responsive promoter.

In some embodiments, the T cell of the invention comprises a deletion of the native TCR and further comprises a nucleic acid molecule encoding a non-native TCR. In some embodiments, the non-native TCR is a subject or patient derived TCR.

In one embodiment, the invention provides a T cell comprising a nucleic acid molecule comprising a nucleotide sequence encoding a B cell tagging molecule under the control of an NBV promoter and further comprises a nucleic acid molecule encoding an NBV transcription factor under the control of a TCR responsive promoter. In one embodiment, the nucleic acid molecule encoding an NBV transcription factor is operably linked to a promoter having a sequence as set forth in SEQ ID NO:50-SEQ ID NO:74.

In some embodiments, the nucleic acid molecule encoding the NBV transcription factor further comprises one or more additional regulatory elements, such as an insulator element or an enhancer element, which functions to further regulate expression of the NBV transcription factor.

In some embodiments, the nucleic acid molecule encoding the NBV transcription factor further comprises one or more nucleotide sequence encoding at least one co-stimulatory molecule to amplify expression of NFAT, thus increasing expression of the B cell tagging molecule. In some embodiments, the co-stimulatory molecule is CD2, CD226, CD40L, ICOS, OX40, and 41BB, or a combination thereof. In one embodiment, one or more nucleotide sequence encoding at least one co-stimulatory molecule is operably linked to a constitutive promoter.

Any method known in the art for introducing nucleic acid sequences into cells can be used to generate the modified T cells of the invention. Exemplary methods of introducing nucleic acid molecules into cells include, but are not limited to, electroporation, cell squeezing, sonoporation, optical transfection, protoplast fusion, impalefection, hydrodynamic delivery, fusion, magnetofection, particle bombardment, nucleofection, heat shock, lipofection, viral transduction, nonviral transfection, lithium acetate/PEG chemical transformation, or any combination thereof.

### APC Library

In various embodiments, the invention relates to methods of screening an antigen-presenting cell (APC) library, wherein each antigen-presenting cell displays one or more antigenic sequence. In some embodiments, the APC library comprises a plurality of cells, wherein together the plurality of cells displays at least 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10,000, 20,000 or more than 20,000 different polypeptides on the surface of the cells.

In some embodiments, the polypeptides for display are fusion proteins with linker molecules that allow display of multiple linked antigenic sequences on an APC.

In one embodiment, nucleic acid molecules encoding one or more antigen for display can be cloned into a minigene vector. In some embodiments, the antigen encoded on the minigene vector is expressed intracellularly, processed into small fragments, and presented as an antigen-MHC complex on the outer surface of an APC cell.

In one embodiment, nucleic acid molecules encoding one or more antigen for display can be cloned into a vector is designed to express the one or more encoded antigen on the outer surface of an APC cell containing the vector. Thereafter, the APC library can be screened for TCR reactivities with the displayed antigen(s).

Thus, in various embodiments, the present invention also includes a vector in which a nucleotide sequence encoding a polypeptide for display of the present invention is inserted. The art is replete with suitable vectors that are useful in the present invention.

In one embodiment, the expression of a nucleotide construct is typically achieved by operably linking a nucleic acid sequence comprising a promoter to a nucleic acid sequence encoding at least one antigen or fragment thereof, and incorporating the construct into an expression vector. In one embodiment, the vectors to be used are suitable for replication and, optionally, integration in eukaryotic cells. Typical vectors contain transcription and translation terminators, initiation sequences, and other regulatory sequences useful for regulation of the expression of the desired nucleic acid sequence.

The recombinant nucleotide sequences encoding one or more antigen for display of the invention can be cloned into a number of types of vectors. For example, the nucleic acid can be cloned into a vector including, but not limited to a plasmid or a viral vector.

In one embodiment, the vector may be provided to a cell in the form of a viral vector. Viral vector technology is well known in the art and is described, for example, in Sambrook et al. (2012, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

For example, vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. In one embodiment, the composition includes a vector derived from an adeno-associated virus (AAV). Adeno-associated viral (AAV) vectors have become powerful gene delivery tools for the treatment of various disorders. AAV vectors possess a number of features that render them ideally suited for gene therapy, including a lack of pathogenicity, minimal immunogenicity, and the ability to transduce postmitotic cells in a stable and efficient manner. Expression of a particular gene contained within an AAV vector can be specifically targeted to one or more types of cells by choosing the appropriate combination of AAV serotype, promoter, and delivery method

In certain embodiments, the vector also includes conventional control elements which are operably linked to the encoded antigen sequence in a manner which permits its transcription, translation and/or expression in a cell transfected with the plasmid vector or infected with the virus produced by the invention. As used herein, “operably linked” sequences include both expression control sequences that are contiguous with the reporter molecule and expression control sequences that act in trans or at a distance to control the expression of the reporter molecule. Expression control sequences include appropriate transcription initiation, termination, and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. All of the above-described functional elements can be used in any combination to produce a suitable display vector. In one embodiment, a display vector comprises an origin of replication capable of initiating DNA synthesis in a suitable APC.

In one embodiment, a display vector comprises a selection marker gene to facilitate identification and selection of expressing cells from the population of cells sought to be transfected or infected through viral vectors. In other aspects, the selectable marker may be carried on a separate piece of DNA and used in a co-transfection procedure. Selectable marker genes may be flanked with appropriate regulatory sequences to enable expression in the host cells.

A selection marker sequence can be used to eliminate host cells in which the display vector has not been properly transfected. A selection marker sequence can be a positive selection marker or negative selection marker. Positive selection markers permit the selection for cells in which the gene product of the marker is expressed. This generally comprises contacting cells with an appropriate agent that, but for the expression of the positive selection marker, kills or otherwise selects against the cells.

Examples of selection markers also include, but are not limited to, proteins conferring resistance to compounds such as antibiotics, proteins conferring the ability to grow on selected substrates, proteins that produce detectable signals such as luminescence, catalytic RNAs and antisense RNAs. A wide variety of such markers are known and available, including, for example, a Zeocin™ resistance marker, a blasticidin resistance marker, a neomycin resistance (neo) marker (Southern & Berg, J. Mol. Appl. Genet. 1: 327-41 (1982)), a puromycin (puro) resistance marker; a hygromycin resistance (hyg) marker (Te Riele et al., Nature 348:649-651 (1990)), thymidine kinase (tk), hypoxanthine phosphoribosyltransferase (hprt), and the bacterial guanine/xanthine phosphoribosyltransferase (gpt), which permits growth on MAX (mycophenolic acid, adenine, and xanthine) medium. See Song et al., Proc. Nat'l Acad. Sci. U.S.A. 84:6820-6824 (1987). Other selection markers include histidinol-dehydrogenase, chloramphenicol-acetyl transferase (CAT), dihydrofolate reductase (DHFR), β-galactosyltransferase and fluorescent proteins such as GFP.

Expression of a fluorescent protein can be detected using a fluorescent activated cell sorter (FACS). Expression of β-galactosyltransferase also can be sorted by FACS, coupled with staining of living cells with a suitable substrate for β-galactosidase. A selection marker also may be a cell-substrate adhesion molecule, such as integrins, which normally are not expressed by the host cell. In one embodiment, the cell selection marker is of mammalian origin, for example, thymidine kinase, aminoglycoside phosphotransferase, asparagine synthetase, adenosine deaminase or metallothionien. In one embodiment, the cell selection marker can be neomycin phosphotransferase, hygromycin phosphotransferase or puromycin phosphotransferase, which confer resistance to G418, hygromycin and puromycin, respectively.

Suitable prokaryotic and/or bacterial selection markers include proteins providing resistance to antibiotics, such as kanamycin, tetracycline, and ampicillin. In one embodiment, a selection marker includes a protein capable of conferring selectable traits to both a prokaryotic host cell and a mammalian target cell.

In one embodiment, the invention provides a plurality of at least 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10,000, 20,000 or more than 20,000 recombinant nucleic acid molecules (i.e. a minigene library), wherein together the plurality of recombinant nucleic acid molecules encode at least 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10,000, 20,000 or more than 20,000 different antigenic sequences for presentation by an APC library.

In some embodiments, the antigen encoding region of each nucleic acid molecule in the minigene library is designed to be a predetermined length. For example, in one embodiment, the minigene library is designed to comprise antigen encoding regions of 900 nucleotides. In one embodiment, the minigene library is designed to comprise antigen encoding regions of 1050 nucleotides. In one embodiment, the minigene library is designed to comprise antigen encoding regions of 1200 nucleotides. In such embodiments, antigen encoding regions are designed to comprise a combination of nucleotide sequences encoding fragments of proteins, wherein each fragment has the predetermined length (e.g., 990 or 1200 nucleotides) and nucleotide sequences encoding multiple peptides linked by one or more linker sequences such that the full-length sequence comprises the predetermined length (e.g., 990 or 1200 nucleotides).

In one embodiment, each of the recombinant nucleic acid molecules in the plurality of recombinant nucleic acid molecules encodes one or more polypeptide sequence for presentation by an APC and further comprises a unique nucleotide sequence encoding a set or shared amino acid barcode sequence. The unique nucleotide sequence is then associated with the encoded antigen sequence(s). In various embodiments, the unique barcode sequence comprises a nucleotide sequence of at least 4, 5, 6, 7, 8, 9, 10, 11, 12, 13, 14, 15, 16, 17, 18, 19, 20 or more than 20 nucleotides which is non-redundant with other nucleotide sequences included in the library, but which encodes the same amino acid sequences as other nucleotide barcode sequences included in the library.

In some embodiments, the nucleic acid molecule encoding the minigene antigen expression cassette further comprises a sequence encoding at least one transmembrane domain for class II antigen presentation, or fragment thereof. Exemplary transmembrane domains that can be encoded include, but are not limited to, SEQ ID NO:114, SEQ ID NO:116, SEQ ID NO:118, SEQ ID NO:120, SEQ ID NO:121, SEQ ID NO: 122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, and SEQ ID NO:135.

In some embodiments, the nucleic acid molecule encoding the minigene antigen expression cassette further encodes a flag-based surface receptor, a ligand for a co-stimulatory molecule, or a combination thereof. In some embodiments, the co-stimulatory molecule is CD40, CD58, CD80, CD83, CD86, OX40L, 4-1BBL, or a combination thereof.

In some embodiments, the invention relates to methods of generating a minigene library for expression of a plurality of antigens on the surface of a plurality of APCs. In some embodiments, the method comprises obtaining or generating a library of barcoded nucleic acid molecules, wherein each nucleic acid molecule comprises a nucleotide sequence encoding one or more polypeptide for presentation on the surface of an APC; and introducing the plurality of recombinant nucleic acid molecules into APC cells for expression and/or display of the recombinant nucleic acid molecules. In one embodiment, the nucleotide sequence encoding one or more polypeptide for presentation on the surface of an APC is operably linked to a unique nucleotide barcode sequence encoding a predetermined or shared amino acid sequence.

Any method known in the art for introducing nucleic acid sequences into cells can be used to generate the APC library of the invention. Exemplary methods of introducing nucleic acid molecules into cells include, but are not limited to, electroporation, cell squeezing, sonoporation, optical transfection, protoplast fusion, impalefection, hydrodynamic delivery, fusion, magnetofection, particle bombardment, nucleofection, heat shock, lipofection, viral transduction, nonviral transfection, lithium acetate/PEG chemical transformation, or any combination thereof.

In one embodiment, the method comprises generating a library of cells for displaying polypeptides which function as antigens for TCR binding. Thus, in one embodiment, the method comprises generating a library of cells, wherein the library comprises cells comprising barcode-labeled nucleic acid sequences, wherein the barcode-labeled nucleic acid sequences encode polypeptides which function as antigens for TCR binding.

### Falcon

In one embodiment, the sequences of the recombinant nucleic acid molecules for inclusion in the minigene library of the invention, or sequences for expression by the APCs of the invention, are determined using the Fast ALgorithm for Codon OptimizatioN (herein referred to as FALCON), which optimizes nucleotide sequences according to multiple parameters for a high-yield protein expression. The FALCON codon optimization process starts with a list of amino acid sequences corresponding to the genes that will be optimized. The system in which the genes will be expressed is chosen, and the sequences are back-translated using a weighted random codon selection function. In the process of codon selection, a codon usage table specific for the expression system is used. In this table, each codon is stored along its particular weight or selection probability. These weights reflect the single and bi-codon usage pattern in highly expressed genes of the expression system.

In one embodiment, the codon usage table selected is one designed in which codons with a higher representation have higher weights. Additionally, the codon weights also reflect the abundance of their cognate tRNAs. Thus, in one embodiment, the more commonly used codons for which respective tRNAs abound are more likely to be selected. This supports efficient protein translation in cells, as it promotes the usage of tRNAs that are more readily available. In some embodiments, the codon selection process includes a series of adjustment layers that modifies the codon weights according to the GC content of the growing nucleotide sequence and codon autocorrelation (i.e., the bias that exists in a number of codons that are correlated with themselves and other codons). For example, during the back-translation process, if a codon was already used by FALCON within the last 25 nucleotide triplets, its likelihood to be selected again will be proportionally higher to the distance of its last occurrence (distance in codons).

In some embodiments, the minigene library is optimized for a desired GC content. In some embodiments, the GC content optimization is performed using a 4-parameter logistic function (4PL). In this function, the four parameters; A, B, C and D, and the independent variable ‘x’; are used to calculate the correction coefficient ‘y’, according to equation 1 (FIG. 33). The equation defines the change of the correction coefficient ‘y’ as a function of the GC content of the growing nucleotide sequence. The parameters are calibrated with the least squares method using the scipy.optimize library (Virtanen et al., 2020, Nature Methods, 17(3), 261-272), such that when ‘x’, the GC % of a growing sequence, is equal to the GC % specified by the user (GC-aim), the equation will result in a correction coefficient ‘y’=0 (i.e., no GC correction). Contrariwise, any deviation from the GC-aim is countered by the correction ratio. The higher the deviation, the stronger the correction. If the GC %>GC-aim, the weights of the codons containing an A/T at the wobble position will be increased, and the codons containing a G/C will be disfavored proportionally. If the GC %<GC-aim, the opposite applies.

In some embodiments, after 10 codons have been selected, the sequence is subjected to several motif avoidance steps. In some embodiments, restriction sites as well as other undesired patterns are eliminated.

In some embodiments, the process of back-translation and motif avoidance is repeated continuously until an optimized minigene library is obtained.

In some embodiments, to promote efficient translation initiation, FALCON optimizes the 5′ start of the first 20 codons, by generating 10 candidate partial sequences using back-translation, and then selecting the sequence with the highest minimum free energy (MFE). A higher MFE is indicative of a less stable mRNA secondary structure, which correlates with higher expression levels (Jia & Li, 2005, FEBS Letters, 579(24), 5333-5337). The MFE is calculated using the open-source ‘seqfold’ package, developed by JJTimons (pypi.org/project/seqfold/). The optimized 5′ start will then be used by FALCON as a starting point to generate 10 full candidate sequences. Subsequently, a step termed “Tournament style selection” follows.

In “Tournament style selection” the utilization of a weighted, yet random, codon selection approach is used such that if a gene is optimized multiple times, virtually every resulting sequence will differ to a degree from each other. This characteristic gives FALCON the advantage of generating multiple contesting full sequences, which can then be evaluated according to specific quality criteria. For each gene, FALCON creates 10 candidates, and the highest-ranking gene is selelected according to the following three parameters:
i) Codon Adaptation Index (CAI) (Sharp & Li, 1987, Nucleic Acids Research, 15(3), 1281-1295) (equation 3; FIG. 33),
ii) GC % (equation 4; FIG. 33) and,
iii) CpG dinucleotide content (equation 5; FIG. 33).

The CAI is a measure widely used to quantify codon preferences toward most common codons. A sequence that only uses the most common codons for a particular organism will have a CAI=1. Deviations from this usage will result in lower CAI values. FALCON first calculates the codon relative adaptiveness (CRA) of each candidate sequence (equation 2; FIG. 33), and then uses a slight modification to the CAI equation (Sharp & Li, 1987, Nucleic Acids Research, 15(3), 1281-1295), expressing the final result in % (equation 3; FIG. 33). The final score is calculated according to equation 5 (FIG. 33).

Therefore, in some embodiments, the minigene library comprises sequences that have been optimized for expression using the FALCON system comprising the steps of:
a) back-translating a target sequence to be expressed,
b) codon optimizing the target sequence,
c) optimizing the GC content of the target sequence to be within a pre-determined or set range,
d) eliminating restriction sites or other unwanted sequences, and
e) optimizing the 5′ start of the first 20 codons to have a high MFE, and selecting an optimized sequence based on the determination of the CAI, GC % and CpG dinucleotide content.

### Transmembrane Domains for Enhance Presentation

In some embodiments, the invention is based, in part, on the development of transmembrane domain for enhanced antigen presentation. In some embodiments, the transmembrane domains of the invention enhance the level or stability of class II MHC presentation of antigens. Exemplary enhanced presentation transmembrane domains include, but are not limited to, SEQ ID NO: 114, SEQ ID NO: 116, SEQ ID NO: 118, SEQ ID NO:120, SEQ ID NO:121, SEQ ID NO: 122, SEQ ID NO:123, SEQ ID NO:124, SEQ ID NO: 125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, and SEQ ID NO:135.

In some embodiments, the invention relates to fusion molecules comprising an enhanced transmembrane domain of the invention linked to an antigen for presentation on a cell surface. In some embodiments, the invention relates to nucleic acid molecules encoding fusion molecules comprising an enhanced transmembrane domain of the invention linked to an antigen for presentation on a cell surface.

In some embodiments, the invention relates to compositions comprising fusion molecules comprising an enhanced transmembrane domain of the invention linked to an antigen for presentation on a cell surface. In some embodiments, the invention relates to compositions comprising nucleic acid molecules encoding fusion molecules comprising an enhanced transmembrane domain of the invention linked to an antigen for presentation on a cell surface.

For example, in some embodiments, the invention provides compositions comprising a viral antigen linked to a transmembrane domain for presentation on the surface of a cell. Such an embodiment can be used to induce an immune response against the displayed antigen or to increase the efficacy of a vaccine. However, the invention is not limited by to any specific antigen or use, as any antigen or library of antigens can be linked to a transmembrane domain of the invention for enhanced display.

### Co-Culture of APC and T Cells

In some embodiments, invention provides a method of co-culturing at least one T cell of the invention with an APC library expressing antigens encoded by the minigene library invention, to identify the antigenic polypeptide target of the TCR of the T cell. The co-culture can be performed using any co-culture system including, but not limited to, cell culture plate-based systems, slide based systems, emulsion droplet based systems or other systems for co-culture of cells. In some embodiments, the co-culture is performed using a microfluidic co-culture device. An exemplary microfluidic co-culture device that can be used to co-culture an APC library of the invention and a T cell of the invention is described below, however, the invention should not be seen as being limited to the use of the described microfluidic co-culture device.

### Microfluidic Co-Encapsulation Device

Referring now to FIG. 42, an exemplary microfluidic device 100 is depicted. Device 100 comprises a proximal end 102 and a distal end 104, one or more inlets 106 at proximal end 102, wherein each inlet 106 is fluidly connected to one or more outlets 116 at distal end 104 by microchannels extending through a series of filters 108, fluid resistors 110, focusing loops 112, and nozzles 114. Device 100 further comprises one or more isolation fluid inlets 118 fluidly connected to nozzles 114, wherein each isolation fluid inlet 118 may further comprise a filter 108 or fluid resistor 110.

Device 100 can be fabricated as recesses in a substrate and sealed with a cover to form fluidly connected channels and structures. Contemplated substrate and cover materials include but are not limited to: silicon, glass, ceramic, metals, composites, inorganic materials and organic polymers, such as polydimethylsiloxane (PDMS), thermoset polyester, polystyrene, polycarbonate, poly-methyl methacrylate (PMMA), poly-ethylene glycol diacrylate (PEGDA), perfluorinated polymers (e.g., fluorinated ethylene propylene, perfluoroalkoxy, perfluoropolyether, etc.), polyurethane, and the like. In some embodiments, substrate materials may be selected based on a fabrication method of device 100. For example, in some embodiments a PDMS or thermoset polymer substrate material may be selected to fabricate a microfluidic device through casting with a mold, and in some embodiments a silicon, glass, or ceramic substrate material may be selected to fabricate a microfluidic device through lithography or etching. In some embodiments, certain components or portions of certain components can be constructed from a transparent or translucent material.

Referring now to FIG. 43 and FIG. 44, the features of device 100 are now described in detail. The top image in FIG. 43 depicts a magnified view of filter 108 positioned downstream from an inlet 106. Filter 108 can comprise an expanded width relative to inlet 106 such that a suspension of particles flowing in through inlet 106 has an increased space to separate into evenly spaced individual particles (such as individual cells). Contemplated widths include but are not limited to a width between about 10 μm and 10000 μm. Filter 108 can comprise one or more stream separators 120 extending from inlet 106, wherein each stream separator 120 is an elongated barrier substantially in parallel alignment with a channel fluidly connecting an inlet 106 to a filter 108 (such that each stream separator 120 is in alignment with a direction of fluid flow) and is configured to evenly distribute fluid flow from inlet 106 in a distal direction.

Downstream from stream separators 120, filter 108 can further comprise a filtering region comprising a plurality of pillars 122. Pillars 122 are spaced apart from each other such that individual particles are encouraged to separate from each other while minimizing the likelihood of particles clumping and clogging between each pillar 122. While pillars in microfluidic devices are generally understood as filtering elements, wherein decreasing spacing between pillars increases particle separation, the present invention is based in part on the surprising and unexpected discovery that increasing spacing between pillars achieves effective particle separation while minimizing the probability or occurrences of clogging. Pillars 122 can be spaced apart by any desired distance, such as between about 50 μm and 5000 μm. Spacing can be regular or irregular. Pillars 122 can have any desired cross-sectional shape, such as a circle, oval, triangle, square, rectangle, diamond, polygon, V-shape, U-shape, and the like. Pillars 122 can have any desired size, such as a width or diameter between about 1 μm and 100 μm.

The bottom image in FIG. 43 depicts a magnified view of focusing loops 112. Device 100 comprises a series of arcuate channel lengths forming individual focusing loops 112 positioned downstream from filter 108, wherein focusing loops 112 are configured to encourage individual particles to evenly space apart both laterally and longitudinally within the flow of fluid. The interconnected focusing loops 112 curve in alternating directions about a central axis (depicted as a dotted line) such that a series of focusing loops 112 has an undulating, serpentine, or wave-like shape. In some embodiments, focusing loops 112 are asymmetrical, such that a first side (relative to the central axis) comprises loops 112a of a larger size and a second opposing side comprises loops 112b of a smaller size. In some embodiments, the larger loops 112a and the smaller loops 112b have the same channel width 113 between about 20 μm and 5000 μm. In some embodiments, the larger loops 112a comprise a larger width 113a between about 50 μm and 10000 μm, and the smaller loops 112b comprise a smaller width 113b between about 20 μm and 5000 μm.

While the focusing loops 112 are depicted in FIG. 43 as comprising smaller loops 112b having a constant width 113b and larger loops 112a having a graded width 113a that narrows to match width 113b where a larger loop 112a and a smaller loop 112b connect, it should be understood that alternatives are also contemplated, such as constant width 113b and graded widths 113a that widen to match width 113b where a larger loop 112a and a smaller loop 112b connect, or such as graded widths 113a and graded widths 113b that respectively narrow and widen to match each other where a larger loop 112a and a smaller loop 112b connect.

While the focusing loops 112 are depicted in FIG. 43 as comprising larger loops 112a and smaller loops 112b each curving for about 1800 of a substantially circular path, it should be understood that each of the larger loops 112a and smaller loops 112b can have any desired curvature. For example, in some embodiments, a curvature of a loop 112 can follow any desired path, such as a substantially circular path, ovoid path, parabolic path, hyperbolic path, and the like. In some embodiments, a curvature of a loop 112 is defined by a degree of a substantially circular path, wherein the degree of curvature can be between about 5° and 355°. In some embodiments, a curvature of a loop 112 is defined by a diameter of a substantially circular path, wherein the diameter is between about 20 μm and 1000 μm. In some embodiments, a curvature of a loop 112 is defined by both a degree of curvature and a diameter of a substantially circular path. For example, a larger loop 112a depicted in FIG. 43 can be described as curving for about 1800 of a substantially circular path having a diameter of 110 μm and a smaller loop 112b depicted in FIG. 43 can be described as curving for about 180° of a substantially circular path having a diameter of 50 μm. In some embodiments, each individual loop 112 can have a different width 113 and curvature. A series of focusing loops 112 can form a substantially linear path as shown in FIG. 43 or one or more curved paths. A series of focusing loops 112 can extend for any desired length, such as a length between about 1000 μm and 20000 μm.

FIG. 44 depicts a magnified view of nozzle 114. Nozzle 114 comprises at least three converging microchannels, wherein a first microchannel is fluidly connected to one or more inlets 106, a second microchannel is fluidly connected to one or more isolation fluid inlets 118, and a third microchannel is fluidly connected to one or more outlets 116. The microchannels extending from proximal end 102 are each fluidly connected to an inlet 106 and are downstream from their respective filters 108 and focusing loops 112. Each microchannel extending from an inlet 106 converges into a single microchannel (the abovementioned first microchannel), such that individual particles flow from each inlet 106 combine into a single stream of individual particles, pass through nozzle 114, and flow into the microchannel extending towards distal end 104. In some embodiments, each microchannel extending from an inlet 106 tapers in width as they approach a convergence towards the single first microchannel. The microchannel extending towards distal end 104 is fluidly connected to an outlet 116 (the abovementioned third microchannel). In some embodiments, the microchannel extending towards distal end 104 expands in width as it approaches outlet 116. In some embodiments, the microchannel extending towards distal end 104 is aligned in-line with the single first microchannel. The microchannels extending vertically towards nozzle 114 are each fluidly connected to an isolation fluid inlet 118 (the abovementioned second microchannel) and are configured to form an isolation fluid interface in nozzle 114 through which the combined particle stream flows through. The combined particle stream is segmented as it passes through the isolation fluid interface, such that the resulting stream of fluid flowing towards outlet 116 comprises segments of fluid comprising co-encapsulated individual particles separated by segments of isolation fluid.

In various embodiments, device 100 can comprise one or more additional structures configured to augment its function. For example, device 100 can comprise one or more flow modulators configured to alter fluid flow within device 100. Contemplated flow modulators include but are not limited to valves, fluid resistors, expandable elements, contractible elements, pumps, membranes, and the like. Exemplary fluid resistors 110 are depicted in FIG. 42 through FIG. 45 and can be positioned along any of the fluid pathways described above. Fluid resistors 110 are formed from extended lengths of microchannel that are wound within a compact area for space efficiency and are configured to fine tune fluid flow rates within device 100 without significantly affecting fluid pressures. While device 100 is depicted in FIG. 42 as having two sets of inlet 106, filter 108, and focusing loops 112 that converge towards a nozzle 114, it should be understood that there is no limit to the number of inlets 106, filters 108, and focusing loops 112 that can converge towards a nozzle 114. For example, in some embodiments, device 100 can comprise a set of inlets 106, filter 108, and focusing loops 112 for each individual particle that is desired to be co-encapsulated.

In some embodiments, device 100 can further comprise one or more additional inlets, such as inlet 106b shown in FIG. 45. The one or more additional inlets can be used for any desired purpose. For example, in some embodiments the additional inlets may be configured to introduce one or more reagents into a particle stream. For example, inlet 106b being connected to nozzle 114a upstream from nozzle 114b is configured to introduce a reagent prior to co-encapsulation of individual particles at nozzle 114b. Certain particle suspensions may require an additive that facilitates particle separation but may not be ideal for a co-encapsulation study, or an additive that is beneficial in a co-encapsulation study but may not be ideal for particle separation, or some other similar scenario. Accordingly, one or more inlets 106b may be provided to introduce one or more reagents into a particle stream before or after the particles have been separated into a stream of individual particles. The one or more inlets 106b may fluidly connect to any location in device 100 where a reagent's effect is desired. The reagent can be selected based on the desired effect, e.g., in the previous examples, to lessen or neutralize the effect of the additive that facilitates particle separation but is not ideal for a co-encapsulation study, or to promote or enhance a co-encapsulation study. In some embodiments, the additional inlets may be configured to provide one or more additional layers of encapsulation. For example, inlet 106b can be configured to encapsulate individual particles at a first level at nozzle 114a, followed by a second encapsulation on a second level at nozzle 114b.

Device 100 can have any suitable dimensions. For example, device 100 can have an overall length between about 5000 μm to about 50000 μm. The fluid passageways of device 100 can have a channel height of between about 10 μm and 100 μm, or about 35 μm. The fluid passageways of device 100 can have a channel width of between about 5 μm and 100 μm. Channel height and channel width can be constant throughout device 100 or different in certain sections of device 100.

The microfluidic devices of the present invention can be fabricated using any suitable method known in the art. The method of making may vary depending on the materials used. For example, devices substantially comprising a metal may be milled from a larger block of metal or may be cast from molten metal. Likewise, components substantially comprising a plastic or polymer may be milled from a larger block, cast, or injection molded. In some embodiments, the devices may be made using 3D printing or other additive manufacturing techniques commonly used in the art. In some embodiments, the methods can embed additional components, such as circuitry, electrodes, magnets, diodes, and the like, such that the resulting device can be electrified to support electroporation, photoporation, magnetic fields, and the like. In various embodiments, coatings, patterns, and other finely detailed features can be applied using techniques such as etching, lithography, deposition, spin coating, dip coating, and the like. In some embodiments, coatings and patterns can include one or more capture agents immobilized on an inner surface of the device. Capture agents have an affinity to analytes of interest and can be used for a number of purposes, including but not limited to detecting analyte presence, quantifying analyte amount, and filtering analytes from a solution. In some embodiments, certain capture agents employ nonspecific binding. In some embodiments, certain capture agents employ size-specific filtration and do not rely on binding. Contemplated capture agents include at least antibodies, antigens, aptamers, affibodies, proteins, peptides, nucleic acids, carbon nanotubes, nanowires, magnetic beads, and fragments thereof. The capture molecules can be provided in a uniform coating, in an array pattern, or in any shape or form desired.

In certain embodiments, the devices are cast from molds. For example, a positive mold can be fabricated through patterning with SU-8 photoresist and soft lithography. For an exemplary device, a positive mold can comprise a channel layer having an exemplary 35 μm height. Curing a polymer with the mold would result in a substrate having a top surface with corresponding embedded channels having an exemplary 35 μm depth be included in the mold. Inlets, outlets, reservoirs, and the like can be added by punching or milling. The top surface of the substrate can be sealed using a layer of metal, glass, or polymer. In various embodiments, the device can be cleaned or sterilized using any sterilization techniques, including but not limited to autoclaving, gamma ray sterilization, electron beam sterilization, and the application of any sterilizing gas, plasma, or solution, such as ethylene oxide, chlorine dioxide, hydrogen peroxide, oxygen plasma, and the like.

In some embodiments, the devices are fabricated in the form of an array on a microfluidic chip. Referring now to FIG. 46A and FIG. 46B, exemplary arrays of microfluidic device 100 are depicted. As described elsewhere herein, the dimensions and placement of each of the components of a microfluidic device 100 enhance particle co-encapsulation while maintaining high space efficiency. In some embodiments, an array of devices 100 can arrange each device 100 in alternating orientations to increase space efficiency. Accordingly, a high density of devices 100 are configured to fit on a microfluidic chip. In some embodiments, a microfluidic chip can be in the form of a microscope slide. Microscope slides refer not only to the commonly used 75×25 mm slide, but also to any other slide as would be understood by persons having skill in the art, for example 76×26 mm, 75×38 mm, 76×51 mm, 76×52 mm, 46×27 mm, 48×28 mm, 22×22 mm, 24×50 mm, and the like. In some embodiments, a microfluidic chip can be in the form of a wafer. Wafers refer not only to the commonly used 300 mm diameter water, but also to any other wafer as would be understood by persons having skill in the art, for example 25 mm, 51 mm, 76 mm, 100 mm, 125 mm, 130 mm, 150 mm, 200 mm, 450 mm, and the like. In various embodiments, a microfluidic chip can comprise 1, 2, 3, 4, 5, 6, 7, 8, 9, 10, or more devices 100. In some embodiments, a microfluidic chip can be described in terms of chip density, wherein a chip comprises about 10, 20, 30, 40, 50, 60, 70, 80, 90, 100, or more devices 100 per 10 cm².

In some embodiments, the present invention relates to particle suspension reservoirs configured to supply input fluids to or receive output fluids from microfluidic devices described elsewhere herein. Referring now to FIG. 47, an exemplary reservoir 200 is depicted, wherein reservoir 200 is configured to fluidly connect to a device 100 via flexible tubing. Reservoir 200 can generally comprise a container 202 and a cap 204. A fluid conduit 208 can be provided to direct a sample solution 210 into an inlet 106 or to receive a sample solution 210 from an outlet 116. In some embodiments, a pressure conduit 206 can be provided to apply a positive pressure and induce a sample solution 210 to exit reservoir 200 through fluid conduit 208, to apply a negative pressure to induce a sample solution 210 to enter reservoir 200 through fluid conduit 208, or to act as a passive pressure relief conduit. In some embodiments, container 202 can receive a particle suspension that is in need of regular or semi-regular stirring or mixing. Accordingly, a stirring magnet 212 and a stirring plate 214 may be provided to effect the stirring or mixing. In some embodiments, container 202 may be placed on a shaking platform or rocking platform. In certain embodiments, container 202 may comprise a conical bottom, such that a stand 216 may be required to hold container 202 upright. In such a conical container 202, a stirring magnet 212 does not have a sufficiently wide bottom surface on which to spin. Reservoir 200 may accordingly further comprise an inner container 218 (such as a round bottom tube) having a wider bottom surface, wherein a sample solution 210 is placed within the inner container 218.

In some embodiments, the microfluidic device of the invention is used to co-culture an APC of the invention and a T cell of the invention. In such an embodiment, at least one T cell of the invention is applied to one inlet 106 of the microfluidic device 100 and at least one APC of the invention is applied to a second inlet 106 of the microfluidic device 100. The APC and the T cell of the invention then are flowed through the microfluidic device and combined at the nozzle of the microfluidic device such that each emulsion droplet comprises a number (n) of APCs, where n is 0, 1, 2, 3, 4 or 5, and a number (m) of T cells where m is 0, 1, 2, 3, 4 or 5.

In one embodiment, the APC applied to the microfluidic device comprises a B cell. In one embodiment, a B cell suspension applied to the microfluidic device comprises EDTA.

In one embodiment, the T cell suspension applied to the microfluidic device comprises Ca²⁺. In one embodiment, the Ca²⁺ is present in the T cell suspension at least at an equimolar concentration as that of the EDTA present in the B cell suspension.

### Methods of Use

The present invention also includes methods of using microfluidic devices for the co-encapsulation of individual particles (such as individual cells). As described elsewhere herein, the microfluidic devices of the present invention are capable of generating longitudinal flows of individual particles from fluid suspensions, combining two or more individual particle flows into a single stream of individual particles, and segmenting the single stream of individual particles using an isolation fluid such that each stream segment co-encapsulates an individual particle from each fluid suspension.

Contemplated particles can include but are not limited to: cells, viruses, bacteria, amoeba, protozoa, paramecium, microparticles, nanoparticles, beads, microorganisms, vesicles, nucleic acid oligonucleotides, proteins, polypeptides, carbohydrates, and fragments thereof. The particles can be provided in any desired suspension fluid, including but not limited to water, cell growth media, serum, plasma, oil, and the like. Isolation fluids can be any desired fluid, including but not limited to oils, gels, liquid metals, liquid polymers, glues, and the like. Isolation fluids and can be selected such that the isolation fluid is immiscible with the particle suspension fluids. For example, oil-based isolation fluids can be selected for aqueous suspension fluids, and vice versa (where aqueous isolation fluids can be selected for oil-based suspension fluids).

As described elsewhere herein, particle suspensions can include one or more additives that enhance particle separation into an individual particle flow. The microfluidic devices can also accept one or more reagents that are added into an individual particle flow or a combined individual particle stream for co-encapsulation. In various embodiments, the additives and reagents can include natural or synthetic drugs, including but not limited to: analgesics, anesthetics, antifungals, antibiotics, anti-inflammatories, nonsteroidal anti-inflammatory drugs (NSAIDs), anthelmintics, antidotes, antiemetics, antihistamines, anti-cancer drugs, antihypertensives, antimalarials, antimicrobials, antipsychotics, antipyretics, antiseptics, antiarthritics, antituberculotics, antitussives, antivirals, cardioactive drugs, cathartics, chemotherapeutic agents, a colored or fluorescent imaging agent, corticoids (such as steroids), antidepressants, depressants, diagnostic aids, diuretics, enzymes, expectorants, hormones, hypnotics, minerals, nutritional supplements, parasympathomimetics, potassium supplements, radiation sensitizers, a radioisotope, fluorescent nanoparticles such as nanodiamonds, sedatives, sulfonamides, stimulants, sympathomimetics, tranquilizers, urinary anti-infectives, vasoconstrictors, vasodilators, vitamins, xanthine derivatives, and the like. The therapeutic agent may also be other small organic molecules, naturally isolated entities or their analogs, organometallic agents, chelated metals or metal salts, peptide-based drugs, or peptidic or non-peptidic receptor targeting or binding agents. The therapeutic agent may also be a large molecule, such as a monoclonal antibody or other recombinant protein.

In some embodiments, the additives include a chelating agent. For example, B cells have a natural tendency to clump together in suspension, which impedes the generation of a flow of individual B cells and increases the likelihood of clogging. A chelator, such as a calcium ion chelator, can be added to a B-cell suspension to inhibit clumping of B cells. Contemplated chelating agents include but are not limited to EDTA and EGTA. In some embodiments, the additives include an ion solution to compensate for chelated ions. For example, T-cell activation signaling relies on extracellular calcium. However, co-encapsulating a B-cell suspension having a calcium ion chelator with a T-cell suspension would inhibit T-cell activation. Accordingly, calcium can be added to the T-cell suspension to compensate, thereby restoring a baseline calcium concentration of a resultant co-encapsulated B cell and T cell.

In various embodiments, the additives and reagents can include natural peptides, such as glycyl-arginyl-glycyl-aspartyl-serine (GRGDS), arginylglycylaspartic acid (RGD), and amelogenin. In some embodiments, the surface treatments can include sucrose, fructose, cellulose, or mannitol. In some embodiments, the surface treatments can include nutrients, such as bovine serum albumin. In some embodiments, the surface treatments can include vitamins, such as vitamin B2, vitamin Ad, Vitamin D, Vitamin E, and Vitamin K. In some embodiments, the surface treatments can include nucleic acids, such as mRNA and DNA. In some embodiments, the surface treatments can include natural or synthetic steroids and hormones, such as dexamethasone, hydrocortisone, estrogens, and its derivatives. In some embodiments, the surface treatments can include growth factors, such as fibroblast growth factor (FGF), transforming growth factor beta (TGF-β), and epidermal growth factor (EGF). In some embodiments, the surface treatments can include a delivery vehicle, such as nanoparticles, microparticles, liposomes, viral and non-viral transfection systems.

Additional contemplated additives and reagents include but are not limited to: excipients; surface active agents; dispersing agents; inert diluents; granulating and disintegrating agents; binding agents; lubricating agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; stabilizing agents; enzymes; nucleic acids; and the like.

### Methods for Identifying T Cell Receptor Antigens

The present invention relates, in part, to methods of identifying binding partners of T cell receptors (TCRs). In one embodiment, the method comprises identifying an antigenic polypeptide-HLA complex that specifically binds to a TCR of interest. In one embodiment, the method comprises identifying novel TCR:antigenic peptide-HLA complex interactions.

In one embodiment, the invention relates to a screening method for TCR-antigen interactions, wherein the method comprises generating a library of polypeptides that are then screened for interactions with at least one TCR. Therefore, in one embodiment, the invention relates to a polypeptide display library and methods of use thereof for screening for TCR:antigenic peptide-HLA complex interactions.

The present invention provides, in part, a method of identifying TCR-antigen interactions that are associated with a disease or disorder. In one embodiment, the APC library of the invention comprises a plurality of APCs purified from a biological sample obtained from a subject having a disease or disorder, and transfected with a minigene library of the invention. In one embodiment, the TCR is a recombinant TCR generated based on genetic information from a TCR of a subject having a disease or disorder. In one embodiment, the method comprises contacting one or more cells of an APC library of the invention with a T cell expressing a TCR. In some embodiments, the step of contacting one or more cells of an APC library of the invention with a T cell is performed using a plate-based co-culture system, a slide-based co-culture system, an emulsion based co-culture system, a chip-based co-culture system, a microfluidic co-culture device, or any appropriate device or system for co-culturing an APC and T cell of the invention. For example, in one embodiment, the contacting of an APC and a T cell is performed using the microfluidic co-culture device described elsewhere herein. However, the present invention is not limited to the device described herein. In one embodiment, the contacting of an APC and a T cell is performed in a single-well or multi-well cell culture plate.

In certain embodiments, the TCR expressed by the T ell is derived from a subject. In one embodiment, the subject has been diagnosed as having a disease or disorder. In one embodiment, the APC library is generated from immortalized B cells derived from a subject having a disease or disorder. In one embodiment, the disease or disorder is selected from an autoimmune disease or disorder, cancer, an inflammatory disease or disorder, a metabolic disease or disorder, a neurodegenerative disease or disorder, a disease or disorder associated with an infectious agent, or any combination thereof.

In one embodiment, the TCR is identified to be reactive with an antigen expressed by an APC based on expression of a marker operably linked to a TCR responsive promoter. For example, in some embodiments, the TCR is identified to be reactive with an antigen expressed by an APC based on the induction of expression of a fluorescent marker (e.g. GFP) operably linked to a TCR responsive promoter. In some embodiments, the TCR is identified to be reactive with an antigen expressed by an APC based on the induction of expression of an APC tagging molecule operably linked to a TCR responsive promoter.

In one embodiment, the TCR is identified to be reactive with an antigen expressed by an APC when the APC is tagged with an APC tagging molecule of the invention (e.g., a CD19 or CD20 scFv molecule expressed by the T cell).

In one embodiment, an APC bound by an APC tag of the invention (e.g. a CD19 or CD20 scFv) is identified using any appropriate sorting or selection method. Exemplary sorting and selection methods include, but are not limited to, affinity-based selection methods, pull down assays using a molecule having affinity to a tag (e.g., Flag or HA tags), biotinylated labeled anti-immunoglobulin antibody, fluorescence activated cell sorting (FACS), fluorescently labeled anti-immunoglobulin antibody, magnetic bead-based selection, magnetic bead conjugated to an anti-immunoglobulin antibody, or any combination thereof. In one embodiment, the method comprises isolating and enriching at least one antibody-bound APC.

In one embodiment, the method comprises amplifying the barcoded recombinant nucleic acid molecule (minigene) of the antibody-bound APC. In one embodiment, the method comprises sequencing the barcoded recombinant nucleic acid molecule (minigene) to identify the antigen associated with the TCR. In one embodiment, the screening methods of the invention include a step of isolating and sequencing barcoded nucleic acid molecules from a plurality of antibody-bound (tagged) APCs.

In one embodiment, the methods described herein can utilize next-generation sequencing technologies that allow multiple samples to be sequenced individually as genomic molecules (i.e., singleplex sequencing) or as pooled samples comprising indexed genomic molecules (e.g., multiplex sequencing) on a single sequencing run. These methods can generate up to several hundred million reads of DNA sequences. In various embodiments, the sequences of nucleic acid sequence barcodes, and thus the associated antigen sequence(s) can be determined using, for example, the next generation sequencing technologies described herein. In various embodiments, analysis of the massive amount of sequence data obtained using next-generation sequencing can be performed using one or more processors.

In some embodiments, the nucleic acid product can be sequenced by next generation sequencing methods. In some embodiments, the next generation sequencing method comprises a method selected from the group consisting of Ion Torrent, Illumina, SOLiD, 454; Massively Parallel Signature Sequencing, solid phase reversible dye terminator sequencing; and DNA nanoball sequencing may be included. In some embodiments, the first and second sequencing primers are compatible with the selected next generation sequencing method.

In some embodiments, sequencing can be performed by next generation sequencing methods. As used herein, “next generation sequencing” refers to methods that were developed to increase the speed, capacity and accuracy of generating sequence data over those that were possible with conventional sequencing methods (e.g., Sanger sequencing) by reading thousands of millions of sequencing reactions simultaneously. Non-limiting examples of next generation sequencing methods/platforms include Massively Parallel Signature Sequencing (Lynx Therapeutics); pyrophosphate sequencing/454; 454 Life Sciences/Roche Diagnostics; Solid Phase Reversible Dye Terminator Sequencing (Solexa/illumina): SOLiD technology (Applied Biosystems); ion semiconductor sequencing (ION Torrent.); DNA nanoball sequencing (Complete Genomics); and technologies available from Pacific Biosciences, Intelligen Bio-systems, Oxford Nanopore Technologies, and Helicos Biosciences. In some embodiments, the sequencing primer may comprise a moiety that is compatible with the selected next generation sequencing method.

Next generation sequencing techniques and related sequencing primer constraints and design parameters are well known in the art (e.g., Shendure et al., 2008, Nature, 26:1135-1145; Mardis, 2007, Trends in Genetics, 24:133-141; Su et al., 2011, Expert. Rev. Mol. Diagn., 11:333-43; Zhang et al., 2011, J. Genet. Genomics, 38:95-109; Nyren P et al. 1993, Anal. Biochem., 208:17175; Bentley et al., 2006, Curr. Opin. Genet. Dev., 16:545-552; Strausberg et al., 2008, Drug Disc. Today, 13:569-577; U.S. Pat. Nos. 7,282,337; 7,279,563; 7,226,720; 7,220,549; 7,169,560; U.S. Patent Application Publication No. 20070070349; U.S. Pat. Nos. 6,818,395; 6,911,345; U.S. Patent Application Publication No. 2006/0252077; No. 2007/0070349).

Several targeted next generation sequencing methods are described in the literature (for review see e.g., Teer and Mullikin, 2010, Human Mol. Genet. 19:R145-151), all of which can be used in conjunction with the present invention. Many of these methods (described e.g. as genome capture, genome partitioning, genome enrichment etc.) use hybridization techniques and include array-based (e.g., Hodges et al., 2007, Nat. Genet., 39:1522-1527) and liquid based (e.g., Choi et al., 2009, Proc. Natl. Acad. Sci USA, 106:19096-19101) hybridization approaches. Commercial kits for DNA sample preparation are also available: for example, Illumina Inc. (San Diego, California) offers the TruSeq™ DNA Sample Preparation Kit and the Exome Enrichment Kit TruSeq™ Exome Enrichment Kit.

There are many methods known in the art for the detection, identification, and quantification of specific nucleic acid sequences (e.g., nucleic acid sequence barcodes) and new methods are continually reported. A great majority of the known specific nucleic acid detection, identification, and quantification methods utilize nucleic acid probes in specific hybridization reactions. Many methods useful for the detection and quantification of nucleic acid take advantage of the polymerase chain reaction (PCR). The PCR process is well known in the art (U.S. Pat. Nos. 4,683,195, 4,683,202, and 4,800,159). To briefly summarize PCR, nucleic acid primers, complementary to opposite strands of a nucleic acid amplification target sequence, are permitted to anneal to the denatured sample. A DNA polymerase (typically heat stable) extends the DNA duplex from the hybridized primer. The process is repeated to amplify the nucleic acid target. If the nucleic acid primers do not hybridize to the sample, then there is no corresponding amplified PCR product. In this case, the PCR primer acts as a hybridization probe. In one embodiment, the method of sequencing the minigene comprises one or more PCR step.

### Kits of the Invention

The invention also includes a kit comprising components useful within the methods of the invention and an instructional material that describes, for instance, an APC library of the invention, a modified T cell of the invention, a microfluidic co-encapsulation devices, and methods of use of the APC library, the T cell and/or methods of using the microfluidic co-encapsulation device as described elsewhere herein. The kit may comprise components and materials useful for performing a co-culture assay of the invention. The kit may also comprise a premade microfluidic co-encapsulation device suitable for separating and co-encapsulating a T cell and APC cell of the invention.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically illustrate the preferred embodiments of the present invention, and are not to be construed as limiting in any way the remainder of the disclosure.

### Example 1: High-Throughput Functional Deconvolution of Autoreactive T Cell Receptors

This invention outlines the establishment of a functional, high-throughput TCR screening platform. A system to de-orphanize autoreactive TCRs needed to be able to de-orphanize TCRs that are patient-specific (autologous HLA haplotypes), detect both class I and class II-presented peptides, not be restricted to known autoantigenic peptides, detect post-translationally modified peptides, and be agnostic to the presence of autoantibodies.

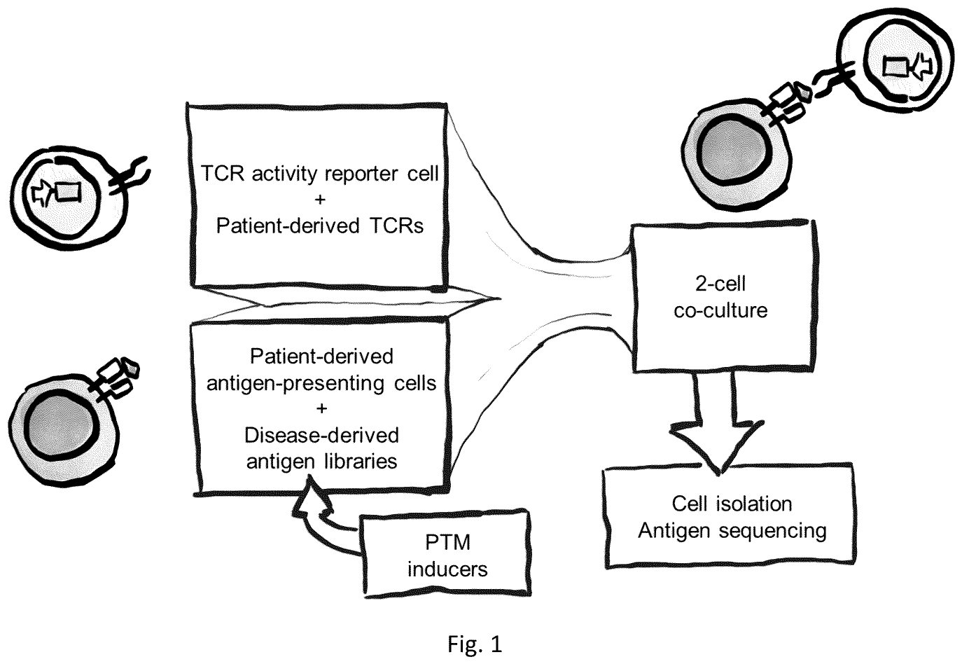

The system of the invention provides a cellular interaction screening method in which a TCR activity reporter cell expressing a patient-derived TCR is co-cultured with a patient-derived antigen-presenting cell expressing a disease-derived antigen library. The interacting cells are then isolated and the TCR-interacting antigen is sequenced (FIG. 1).

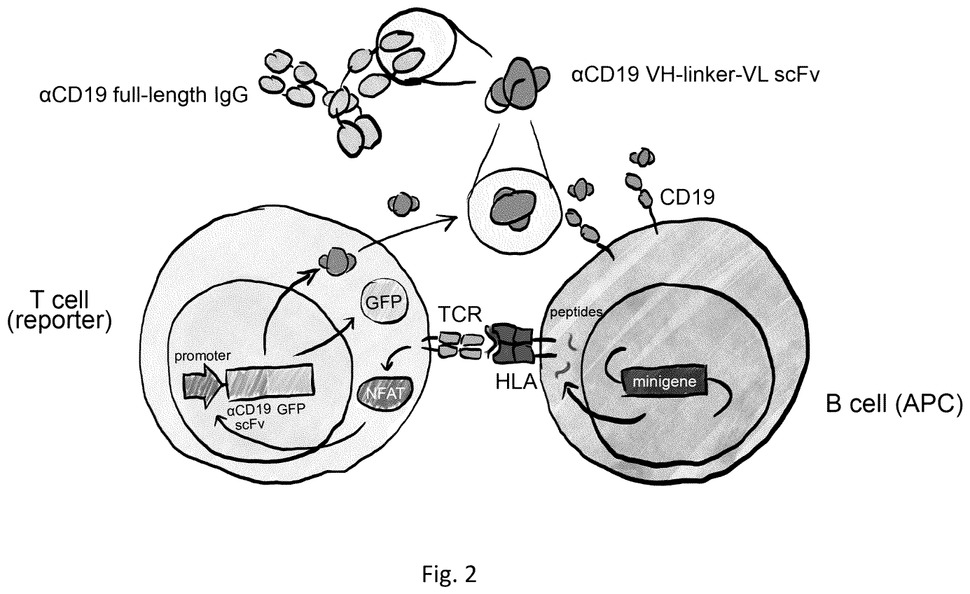

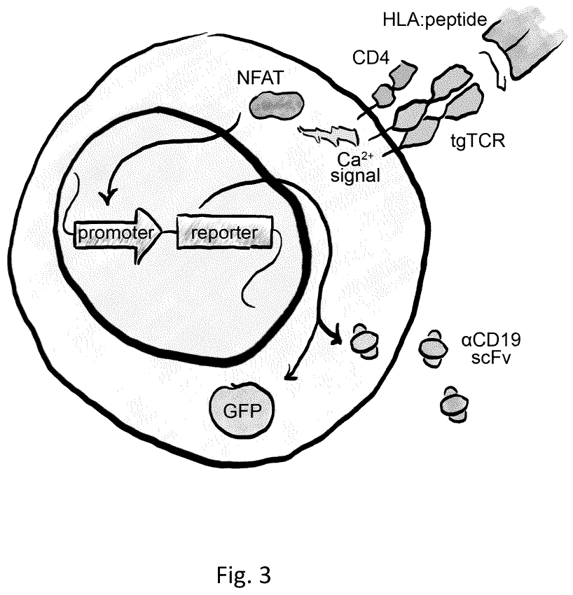

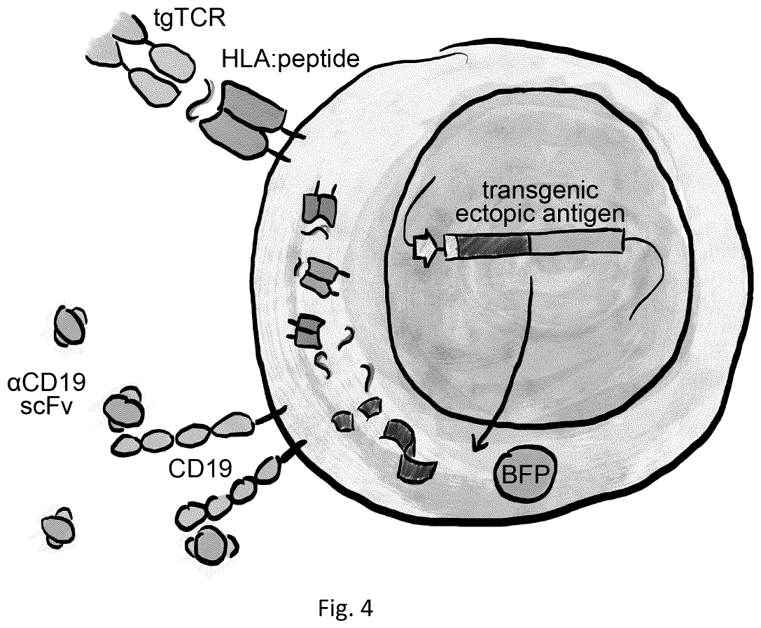

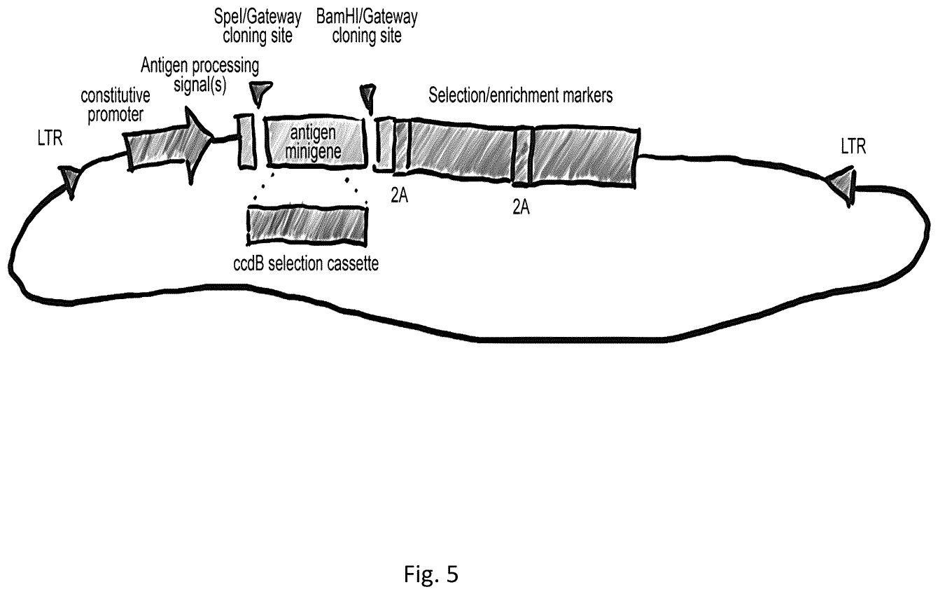

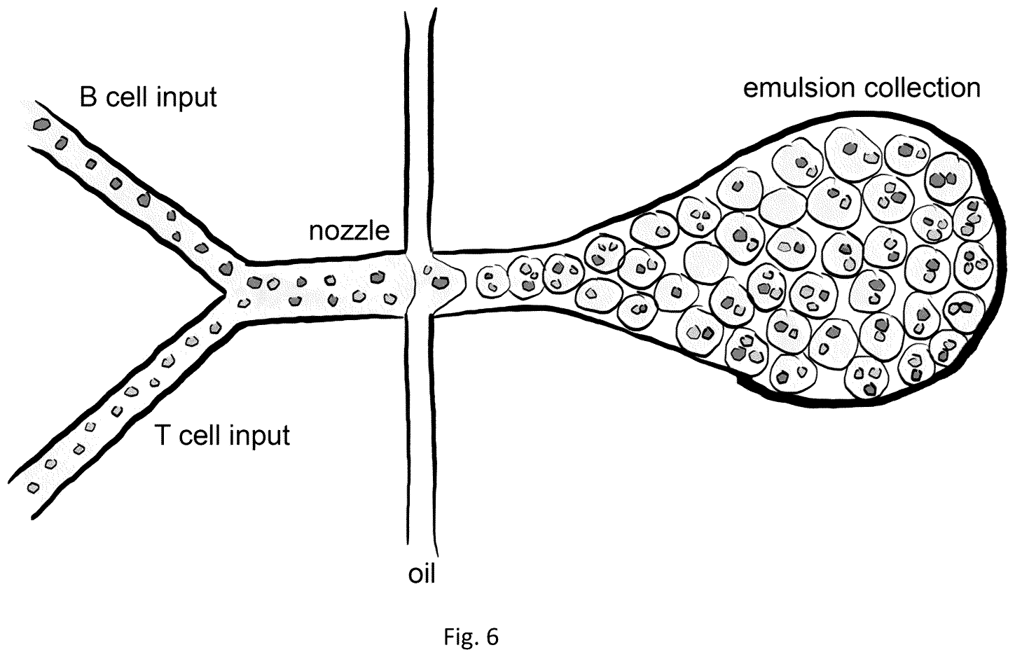

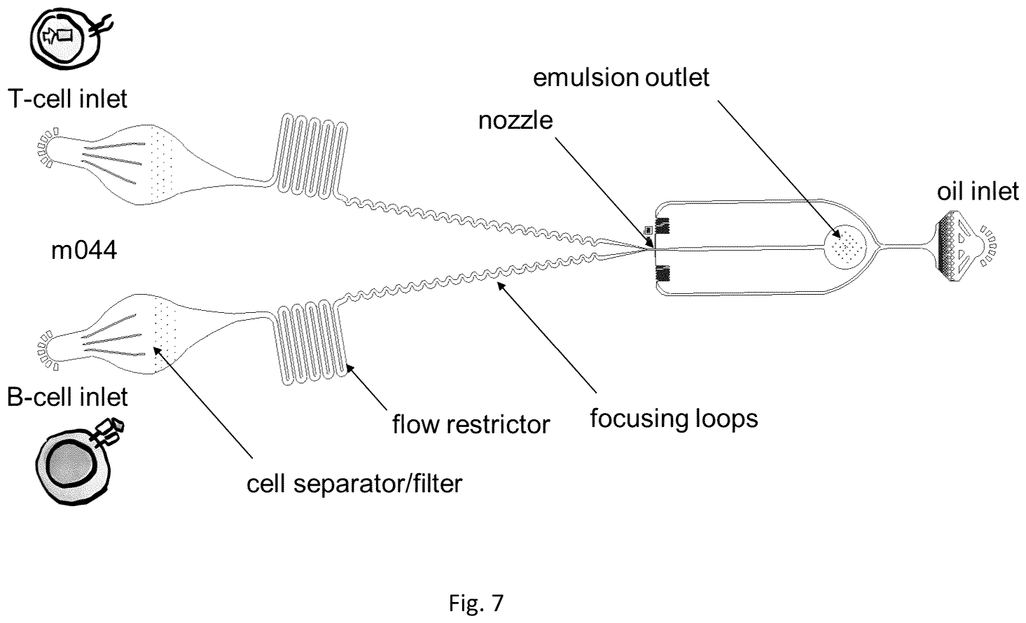

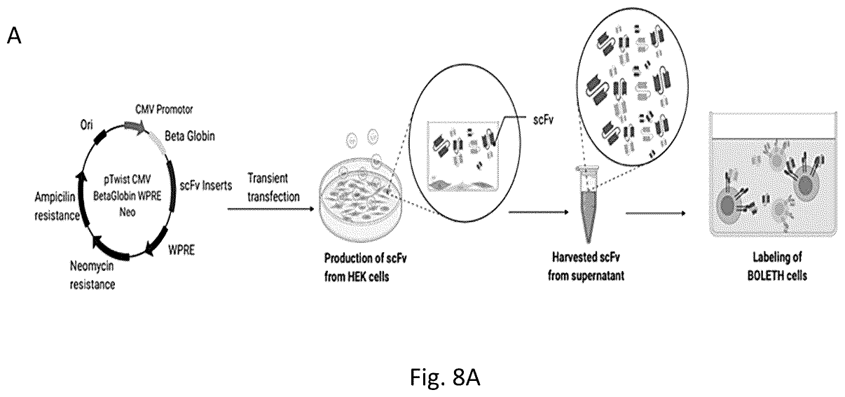

The screening method includes multiple components including a T cell secreted αCD19 scFv as a B cell targeting reporter (FIG. 2, described in more detail in Example 2), TCR reporter cell lines which express the B cell targeting reporter under the regulation of elements that are responsive to interactions between the TCR and an antigen-presenting cell (FIG. 3, described in more detail in Example 3) and antigen libraries presented by immortalized B cells (FIG. 4) which carry a universal antigen expression vector for expression of minigene libraries from multiple sources (FIG. 5, described in more detail in Example 4). Finally, the system employs a microfluidic co-culture system for high-throughput cellular interaction screening (FIG. 6 and FIG. 7, described in more detail in Example 5.)

### Example 2: In Vitro Labels for TCR Signaling Reporters

T lymphocytes comprise approximately 20% of all leukocytes in human peripheral blood and are a major component of the adaptive immune system. A unique feature of each newly formed T cell is its T cell receptor (TCR), which is formed during T cell genesis in the bone marrow and thymus via somatic recombination of genomic elements. As such, each TCR has a unique ligand-binding site, forming a receptor repertoire with the potential to bind up to 10²² distinct ligands. In order to aid in the diagnosis, prevention, and treatment of infectious disease, autoimmune disorders, and cancer, it is crucial to catalog functional relationships between TCRs and their ligands.

A platform was developed for the rapid deconvolution of orphaned TCR sequences. The basis of the system is a cellular interaction screen between T cell lines transgenically modified to express TCRs of interest, as well as genetic response elements (“promoters”) which are inducible upon TCR:cognate ligand binding, and antigen-presenting cells (APCs) which have been transgenically modified to ectopically express a potential cognate ligand. Induction of the TCR signal upon ligand binding indicates a functional “match” between the interrogated TCR sequence and the ectopic antigen expressed by the APC. However, the APC is the cell containing the genetic information required to identify the cognate antigen; therefore, it is necessary to transmit the induced T cell signal onto the APC for isolation.

The invention is based in part on the development of a secreted protein that specifically binds to the APCs being used. In this platform, the APCs are Epstein-Barr virally immortalized B cells, which specifically express the proteins CD19 and CD20 on their surface. Single-chain variable fragments (scFv) from CD19- and CD20-binding monoclonal antibodies have been engineered to be produced by activated T cells, which subsequently bind to and label the interacting B cell during co-culture. Because the scFv expression is under the control of a TCR-signaling induced response element, non-cognate B cells will not elicit secreting of the signal from interacting T cells, and will therefore not be labeled. B cells in the co-culture can then be isolated from the T cells and sorted based on whether or not the labeling antibody fragments have bound to their surface, providing an enriched population of B cells which express cognate antigens.

The experimental results are now described.

### Secreted αCD19 and αCD20 scFvs for B Cell Labeling

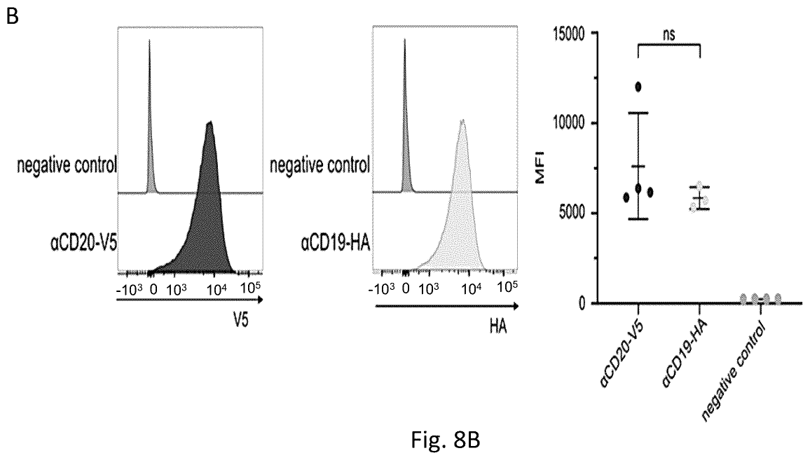

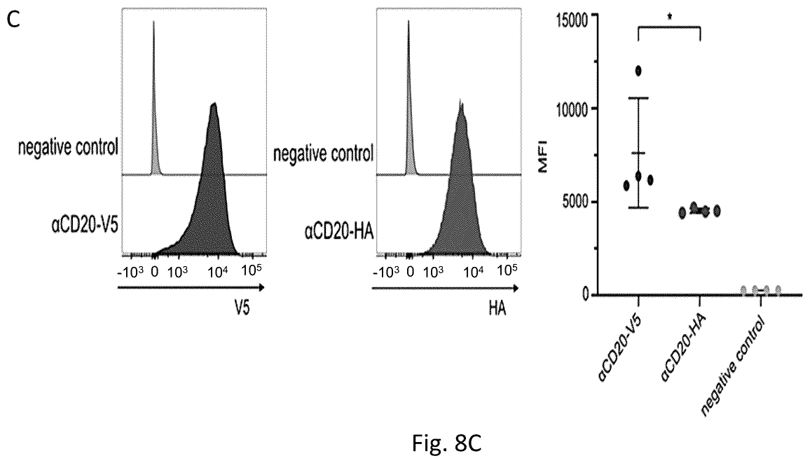

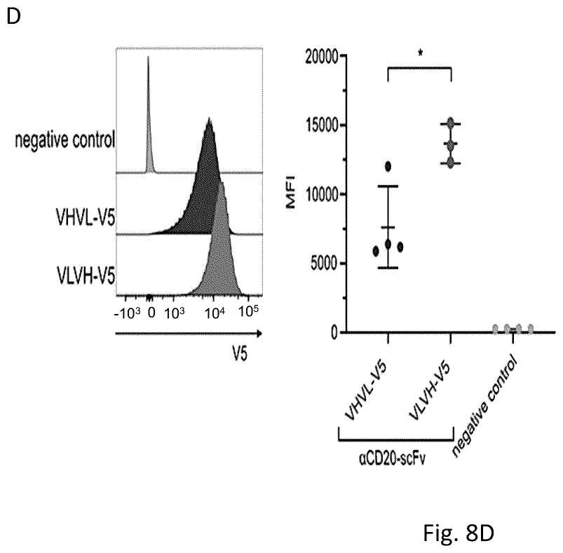

Current generation T cell reporter systems consists of a cis-regulatory element comprising multiple copies of a synthetic promoter sequence (‘NBV’). This promoter was observed to sensitively respond to T cell receptor engagement and drives the expression of downstream reporter elements consisting of a CD19-specific secreted single-chain variable fragment and cytoplasmic sfGFP. The scFv is derived from a monoclonal αCD19-antibody (clone B43) binding to the B cell-specific transmembrane protein CD19, which is commonly used in monoclonal antibody-mediated immunotherapy (Uckun et al., 1988, Blood, 71(1):13-29). It is tagged with six HA epitope tags at its C-terminus, which facilitates its detection by flow cytometry. To optimize the current reporter and B cell labeling strategy, the potential of additional scFvs was tested. In particular, an αCD20-scFv derived from a human monoclonal antibody was evaluated that recognizes an epitope encompassing the membrane-proximal small-loop on the CD20 molecule, known as ofatumumab (clone 2F2) (Teeling et al., 2004, Blood, 104(6):1793-800). In order to test its functionality, scFv constructs were assembled in both the VH-VL and VL-VH configuration. The heavy and light chain domains were separated by a flexible polypeptide linker (G4S)3. To confirm that ofatumumab, as an scFv tagged with four copies of the V5 label, a small epitope-tag found on P and V proteins of the paramyxovirus, can efficiently label BOLETH cells and be detected by flow cytometry, the coding region was inserted into a CMV expression plasmid. HEK293 cells were transfected with the construct using TurboFect. As a negative control, HEK293 cells were transfected with a sfGFP-containing construct. Medium was exchanged 24 hours post-transfection. The scFv-containing supernatant (SN) was collected 72 hours post-transfection and was used to label BOLETH cells. Using fluorescent dye-conjugated antibodies recognizing the tagged scFvs, the amount of B cell bound scFv was measured by flow cytometry (FIG. 8A).

In comparison to the previously used αCD19-scFv (clone B43), the ofatumumab derived αCD20-scFv (clone 2F2) labels BOLETH cells equally well and detection by flow cytometry provides similar signal strength measured by median fluorescence intensity (MFI) (FIG. 8B).

To further optimize the potential use of the αCD20-scFv, various peptide tags and configurations were tested. First, the quadruple V5-tag was exchanged with the 6×HA from the αCD19-scFv using InFusion cloning. Determination of signal strength by flow cytometry showed that the MFI obtained with V5-tagged αCD20-scFv is moderately higher than detection of the six-times HA-tagged scFv (FIG. 8C). Thus, future versions of ofatumumab αCD20-scFv were all V5-tagged.

Since these changes did not meet the anticipated signal optimization, the configuration of αCD20-scFv (clone 2F2) was changed in the expression plasmid from heavy-chain linker light-chain (VHVL) to light-chain followed by linker and heavy chain (VLVH). This configuration change to VLVH leads to the highest signal strength detected across all tested scFvs (FIG. 8D). Compared to the previously used αCD19-scFv (B43), VLVH-V5-scFv of ofatumumab increases the MFI by 300%.

### Tandem-scFv Reporters for Dual Labeling of B Cells

The current reporter only allows for one-dimensional sorting of αCD19-HA labeled B cells. To improve the reporter to achieve a dual labeling of B cells that present a cognate antigen and to allow sorting of the labeled B cells in two dimensions, tandem scFv reporters were designed and cloned and their functionality was tested with the labeling protocol described above. Both scFvs were connected by T2A peptide sequences that induce ribosomal skipping during translation to produce two independent proteins. Adding both αCD19-HA (B43) and αCD20-V5 (2F2) into such expression constructs drastically reduces the signal strength coming from the αCD20 scFv (FIG. 9A). Relative to single scFv constructs, the signal strength detected from αCD19-HA remains unchanged, while αCD20-V5 signal strength is clearly reduced. Further changing the order in the expression construct to αCD20-V5 directly after the promoter followed by αCD19-HA does not rescue the low signal from αCD20-V5 (FIG. 9A) but leads to decreased median fluorescence intensity detected from αCD19-scFv. Introducing the so-far strongest tested scFv-αCD20-V5 in VLVH configuration—into these tandem constructs significantly improves the detected V5 signal. However, compared to the HA signal from αCD19-scFv, it still is still reduced (FIG. 9B). To introduce both scFvs in a tandem reporter construct for B cell labeling and subsequent sorting of the dual labeled cells, they should perform equally good.

To investigate the reason behind the significantly reduced signal from αCD20-V5 scFv in tandem constructs compared to single scFv constructs, such as expression of the scFvs or steric hindrance during binding to the receptors, an experiment was performed in which the SN from HEK cells transfected with either single constructs, a tandem construct or single constructs in a co-transfection was used to label BOLETH cells. An additional condition in which the SN of single transfected single-scFv constructs was mixed was performed to provide information about the level in which the signal from the αCD20 is inhibited. Again, it was observed that the αCD19-scFv signal is not affected by any of the conditions and constructs used (FIG. 9C). Both conditions, co-transfection and SN mixed, results in reduction of MFI from the V5 signal, however not as drastic as observed in the double construct. This suggests that both scFvs compete are therefore sterically hindered which mainly affects the αCD20-scFv. The even further reduced MFIs using the double constructs also indicates that other factors such as reduced expression or folding of the αCD20-scFv are affected in presence of the αCD19-scFv. To conclude, the usage of tandem constructs is only recommended if both scFv are equally good in labeling BOLETH cells, which was not observed for the tested scFvs, due to low MFIs detected in the αCD20-scFv channel in presence of αCD19-scFv. Thus, tandem constructs in the tested conditions are not a robust option to improve the sorting strategy of labeled BOLETH cells.

To design a reporter that allows for a double labeling and therefore for a 2-dimensional sorting of scFv-bound B cells, a bispecific diabody consisting of both, the αCD19- and αCD20-scFv, connected by a flexible-rigid-flexible linker sequence (GGGGSAEAAAKEAAAKAGGGGS; SEQ ID NO:49) was tested for its ability to label B cells. The bispecific diabody was N-terminally HA- and C-terminally V5-tagged (FIG. 10A). Similar to previously described experiments, the coding region was inserted into a CMV-expression plasmid and transfected it into HEK cells to recover the SN for BOLETH cell labeling. Comparted to both, single and tandem scFv expression constructs, the diabody provides extremely low MFIs for HA and V5, indicating that this diabody is not a functional option for usage in T cell reporter constructs (FIG. 10A). Several reasons for the low signal strength in both channels are possible: The size of the construct could lead to folding and expression complications; the HA-tag at the N-terminus is located in the binding groove of the αCD19-scFv and could potentially sterically hinder the antigen recognition.

In summary, the experiments demonstrate that using the ofatumumab-derived αCD20-scFv in VLVH configuration could significantly improve the signal detected from B cell-bound tagged-scFvs.

### Optimized Leader Sequence for Ofatumumab-Derived αCD20-scFv

Finally, to further increase the signal strength of the previously tested reporters, the leader sequence of ofatumumab-αCD20 scFv was exchanged with the leader sequence of αCD19 (B43) (FIG. 11A). The leader sequence is characterized as the upstream sequence motif responsible for translocation across the plasma membrane and secretion into the extracellular space. The αCD19-scFv contains an optimized leader sequence for optimal and efficient secretion of antibodies. Each of the above tested constructs containing ofatumumab αCD20-scFv was equipped with the B43 leader sequence and expressed in HEK293 cells and used to subsequently label BOLETH cells with the scFv-containing SN. FIG. 11B shows the comparison of MFIs for each of the tested scFvs with the physiological leader sequence of ofatumumab-clone 2F2 or with the optimized B43 leader sequence. The HA-tagged scFv shows a significantly improved signal intensity, while all other αCD20-scFvs show no significantly different MFIs. Therefore, the αCD20-scFv was used with its physiological leader sequence. Taken together, the αCD20 scFv (clone 2F2) derived from the monoclonal antibody ofatumumab in the VLVH configuration was identified to further increase the signal. This offers a more sensitive detection of labeled B cells that present a cognate peptide by flow cytometry.

Testing of the Optimized scFv-Reporter Constructs Secreted by T Cells

Since previous experiments only represent an optimal setting of B cell labeling in the presence of saturating scFv concentrations, certain tested scFvs were cloned into lenti-viral reporter constructs (pLVX) consisting of a double NBV promoter element and a downstream T2A sequence connecting sfGFP. αCD19- (clone B43) and αCD20-scFv (clone 2F2, VLVH) both, HA and V5-tagged, as well as a tandem construct containing αCD19-HA and αCD20-V5 and the bispecific diabody were cloned into pLVX using InFusion cloning (FIG. 12A). Jurkat T cells expressing a tetanus-toxin derived TCR, TT2, were transduced with viral SN from HEK293 cells transfected with the mentioned reporter constructs. Jurkat TT2 reporter cells were selected with antibiotics for a week before testing them in a coculture with BOLETH cells in presence of increasing concentrations of the cognate TT2-TCR peptide (1 nM-10 μM). The current and best-performing generation of reporter cells (referred to as clone E3) were generated using Sleeping Beauty transposon-based DNA constructs based on their higher signal-to-noise ratio and unlimited size restriction. Here, lenti-viral constructs were chosen due to their ability to rapidly generate and test reporter cell lines.

FIG. 12B and FIG. 12C show the performance of the reporter constructs with respect to GFP and scFv signal strength at 1 μM peptide concentration. Similar to the observation that was made in the artificial setting of BOLETH labeling, also in cocultures of cognate peptide with reporter T cells, the αCD20-V5 (VLVH) shows the highest signal intensity. Even compared to the best reporter so far (E3) it shows almost a 2-fold increase in scFv signal strength. Surprisingly, not only the scFv signal is stronger but also the GFP MFI is very high. Consistent with previous results, the tandem constructs, independent of αCD20-scFv configuration, lead to reduced V5 signal detection while the αCD19-derived HA signal is stable.

To conclusively answer whether the αCD20-scFv (clone 2F2) or αCD19-scFv (clone B43) or the tags (HA vs. V5) recognized by the fluorophore labeled antibodies are responsible for the variation in signal intensity, six-times HA was added to the αCD20-scFv (VLVH), while four-times V5 was cloned onto αCD19-scFv (FIG. 9A) and the constructs were tested again in co-culture with increasing concentrations (0.1 nM-10 μM) of cognate peptide. Consistent with previous observations, the highest MFI was detected from V5-tagged αCD20-scFv (FIG. 13A). Surprisingly, αCD19-scFv V5-tagged shows about 5-fold reduction in MFI, indicating that the αCD20-scFv in VLVH configuration is superior. Both scFvs tagged with 6-times HA behave similar in terms of scFv signal strength, overall indicating that detection of the V5 tag on VLVH is most sensitive. Additionally, expression of the downstream sfGFP seems again increased in αCD20-V5 scFv reporter cells (FIG. 13B), further underlining the effect of T2A upstream genes on the downstream gene expression. In conclusion it is shown that αCD20-scFv in VLVH configuration tagged with four-times V5 consistently leads to the strongest signal detected on labeled BOLETH cells and of sfGFP in activated Jurkat cells.

Optimization of scFv Design

ScFv inserts αCD20-Rituximab (C2B8) and Ofatumumab (2F2) were cloned into pLVX-PGK-BSD containing αCD19-HA-scFv (B43) and were tested with two different tags (V5 and FLAG) (FIG. 14). FIG. 15 demonstrates the ability of Rituximab-FLAG(A), Rituximab-V5(B) and Ofatumumab to label B cells in a co-culture. Ofatumumab-scFv with four-times V5 is able to label B cells when secreted from an activated T cell.

Transient expression plasmids were tested for optimizing B cell scFv labeling. ScFv inserts αCD20-Ofatumumab (2F2) or αCD19 (B43) were cloned into pTwist CMV BetaGlobin WPRE Neo (FIG. 16A).

A comparison between HA-tag on αCD19-scFv (B43) and αCD20-scFv (2F2) was performed. HA signal was evaluated on B cells stained with the supernatant of transfected HEK cells secreting the respective scFvs (FIG. 16B and FIG. 16C). Supernatant of sfGFP transfected HEK cells was used as negative control. HA-tagged αCD19 (B43) scFv leads to higher MFIs compared to HA-tagged αCD20-scFV(Ofatumumab).

A comparison between HA- and V5-tag on αCD20-scFv(2F2) was performed. HA and V5 signals were evaluated on B cells stained with the supernatant of transfected HEK cells secreting the respective scFvs (FIG. 16D and FIG. 16E). Supernatant of sfGFP transfected HEK cells was used as negative control. V5-tag on αCD20-scFv(Ofatumumab) leads to higher signal intensity compared to the HA-tagged αCD20-scFV version.

A comparison between the intensity resulting from single scFv constructs and double scFv constructs was performed. HA and V5 signals were evaluated on B cells stained with the supernatant of transfected HEK cells secreting the respective scFvs (FIG. 16F and FIG. 16G). Supernatant of sfGFP transfected HEK cells was used as negative control. In tandem constructs of an αCD19-T2A-αCD20 scFv the αCD20 scFv shows reduced signal strength compared to single constructs. The αCD19 scFv retains similar binding capacity in single and double constructs.

A comparison of locating the scFvs upstream or downstream of the T2A sequence was performed. HA and V5 signals were evaluated on B cells stained with the supernatant of transfected HEK cells secreting the respective scFvs (FIG. 16H and FIG. 16I). Changing the order of scFv in tandem scFv constructs does not improve the signal strength of the αCD20-scFv. αCD20-scFv signal seems to be affected by the presence of an αCD19-scFv expressed from the same construct.

A comparison of αCD20-V5 scFv light and heavy chain arrangement was performed. The V5 signal was evaluated on B cells stained with the supernatant transfected HEK cells secreting the respective scFvs (FIG. 16J and FIG. 16K). Supernatant of sfGFP transfected HEK cells was used as negative control. Changing the domain order from VH-VL to VL-VH of the αCD20-scFv (Ofatumumab) increases the signal strength of the detected V5 signal.

FIG. 17 depicts the HA tag signal on B cells co-cultured with 1 μM cognate or mismatched peptide and Jurkat cells transgenically modified with synthetic promoters via lentiviral integration as well as on B cells co cultured with 1 μM cognate or mismatched peptide and T cells expressing different synthetic promoters in the lentiviral reporter. B cells that present cognate peptide activated the T cells and led to expression of anti-CD19 scFVs via synthetic promoters. All promoters except P1 and P2 led to positive HA signal and increased median fluorescence intensity of HA from CD20 positive B cells. Better the signal obtained from HA represents better activated synthetic promoter.

B43 Variable Heavy domain:
CDR1: GYAFSSYW (SEQ ID NO: 1)
CDR2: IWPGDSDT (SEQ ID NO: 2)
CDR3: ARRETTTVGRYYYAMDY (SEQ ID NO: 3)
Full-length variable heavy chain:
EVQLVQSGAEVKKPGSSVKVSCKASGYAFSSYWMNWVRQAPGQ
GLEWMGQIWPGDSDTNYAQKFQGRVTITADESTSTAYMELSSLRSEDTAVYYC
ARRETTTVGRYYYAMDYWGQGTTVTVSS (SEQ ID NO: 4)
B43 Variable Light domain:
CDR1: QSVDYSGDSY (SEQ ID NO: 5)
CDR2: DAS (SEQ ID NO: 6)
CDR3: QQSTENPWT (SEQ ID NO: 7)
Full-length variable light chain:
DIQLTQSPSFLSASVGDRVTITCKASQSVDYSGDSYLNWYQQKPG
KAPKLLIYDASNLVSGVPSRFSGSGSGTEFTLTISSLQPEDFATYYCQQSTENPWT
FGGGTKLEIK (SEQ ID NO: 8)
FMC63 Variable Heavy domain:
CDR1: GVSLPDYG (SEQ ID NO: 9)
CDR2: IWGSETT (SEQ ID NO: 10)
CDR3: AKHYYYGGSYAMDY (SEQ ID NO: 11)
Full-length variable heavy chain:
EVKLQESGPGLVAPSQSLSVTCTVSGVSLPDYGVSWIRQPPRKGLE
WLGVIWGSETTYYNSALKSRLTIIKDNSKSQVFLKMNSLQTDDTAIYYCAKHYY
YGGSYAMDYWGQGTSVTVSS (SEQ ID NO: 12)
FMC63 Variable Light domain:
CDR1: QDISKY (SEQ ID NO: 13)
CDR2: HTS (SEQ ID NO: 14)
CDR3: QQGNTLPYT (SEQ ID NO: 15)
Full-length variable light chain:
DIQMTQTTSSLSASLGDRVTISCRASQDISKYLNWYQQKPDGTVKL
LIYHTSRLHSGVPSRFSGSGSGTDYSLTISNLEQEDIATYFCQQGNTLPYTFGGGT
KLEIT (SEQ ID NO: 16)
2F2 Variable Heavy domain:
CDR1: GFTFNDYA (SEQ ID NO: 17)
CDR2: ISWNSGSI (SEQ ID NO: 18)
CDR3: AKDIQYGNYYYGMDV (SEQ ID NO: 19)
Full-length variable heavy chain:
EVOLVESGGGLVQPGRSLRLSCAASGFTFNDYAMHWVRQAPGKG
LEWVSTISWNSGSIGYADSVKGRFTISRDNAKKSLYLQMNSLRAEDTALYYCAK
DIQYGNYYYGMDVWGQGTTVTVSS (SEQ ID NO: 20)
2F2 Variable Light domain:
CDR1: QSVSSY (SEQ ID NO: 21)
CDR2: DAS (SEQ ID NO: 22)
CDR3: QQRSNWPIT (SEQ ID NO: 23)
Full-length variable light chain:
EIVLTQSPATLSLSPGERATLSCRASQSVSSYLAWYQQKPGQAPRL
LIYDASNRATGIPARFSGSGSGTDFTLTISSLEPEDFAVYYCQQRSNWPITFGQGT
RLEIK (SEQ ID NO: 24)
C2B8 Variable Heavy domain:
CDR1: GYAFSSYW (SEQ ID NO: 25)
CDR2: IWPGDSDT (SEQ ID NO: 26)
CDR3: ARRETTTVGRYYYAMDY (SEQ ID NO: 27)
Full-length variable heavy chain:
QVQLQQPGAELVKPGASVKMSCKASGYTFTSYNMHWVKQTPGR
GLEWIGAIYPGNGDTSYNQKFKGKATLTADKSSSTAYMQLSSLTSEDSAVYYCA
RSTYYGGDWYFNVWGAGTTVTVSA (SEQ ID NO: 28)
C2B8 Variable Light domain:
CDR1: QSVDYSGDSY (SEQ ID NO: 29)
CDR2: DAS (SEQ ID NO: 30)
CDR3: QQSTENPWT (SEQ ID NO: 31)
Full-length variable light chain:
QIVLSQSPAILSASPGEKVTMTCRASSSVSYIHWFQQKPGSSPKPWI
YATSNLASGVPVRFSGSGSGTSYSLTISRVEAEDAATYYCQQWTSNPPTFGGGTK
LEIK (SEQ ID NO: 32)
B43/2F2 Diabody
EVQLVQSGAEVKKPGSSVKVSCKASGYAFSSYWMNWVRQAPGQ
GLEWMGQIWPGDSDTNYAQKFQGRVTITADESTSTAYMELSSLRSEDTAVYYC
ARRETTTVGRYYYAMDYWGQGTTVTVSSGSTSGSGKPGSGEGSTKGDIQLTQSP
SFLSASVGDRVTITCKASQSVDYSGDSYLNWYQQKPGKAPKLLIYDASNLVSGV
PSRFSGSGSGTEFTLTISSLQPEDFATYYCQQSTENPWTFGGGTKLEIKGGGGSAE
AAAKEAAAKAGGGGSEIVLTQSPATLSLSPGERATLSCRASQSVSSYLAWYQQK
PGQAPRLLIYDASNRATGIPARFSGSGSGTDFTLTISSLEPEDFAVYYCQQRSNWPI
TFGQGTRLEIKGGGGSGGGGSGGGGSEVQLVESGGGLVQPGRSLRLSCAASGFT
FNDYAMHWVRQAPGKGLEWVSTISWNSGSIGYADSVKGRFTISRDNAKKSLYL
QMNSLRAEDTALYYCAKDIQYGNYYYGMDVWGQGTTVTVSS (SEQ ID NO: 33)
aCD20 (ofatumumab, clone 2F2), VHVL:
GAAGTCCAATTAGTGGAAAGCGGGGGGGGACTGGTCCAGCCCGGGAGATCC
CTGCGACTCTCCTGTGCCGCCTCTGGCTTTACCTTTAACGACTACGCTATGCA
CTGGGTGAGACAGGCCCCTGGTAAGGGGCTTGAGTGGGTATCAACTATTAGC
TGGAACTCTGGTTCCATAGGGTATGCAGACAGCGTCAAGGGCAGATTCACAA
TTTCACGAGATAATGCGAAAAAGTCATTGTATCTTCAAATGAACTCACTCAG
AGCCGAAGACACCGCCCTGTACTACTGCGCAAAGGACATTCAATACGGAAAT
TATTACTATGGAATGGATGTATGGGGACAAGGCACCACGGTGACAGTTTCCT
CCGGAGGGGGAGGCTCTGGGGGAGGAGGAAGTGGCGGCGGAGGGTCCGAAA
TCGTGCTGACACAGTCTCCAGCTACCCTGAGCCTTTCTCCCGGCGAAAGGGCC
ACCCTGAGCTGTCGAGCTTCTCAATCGGTGTCGAGTTACCTCGCTTGGTATCA
GCAGAAACCGGGCCAAGCACCACGGCTCTTGATCTATGATGCTAGTAACCGC
GCAACCGGCATTCCGGCAAGGTTTTCGGGGTCAGGTTCTGGCACAGACTTTA
CATTGACTATCTCTTCACTCGAACCCGAGGATTTTGCAGTCTACTACTGCCAG
CAGAGGAGTAATTGGCCAATTACATTCGGTCAAGGAACCCGGCTTGAAATAA
AG (SEQ ID NO: 34)
EVOLVESGGGLVQPGRSLRLSCAASGFTFNDYAMHWVRQAPGKGLEWVSTISW
NSGSIGYADSVKGRFTISRDNAKKSLYLQMNSLRAEDTALYYCAKDIQYGNYYY
GMDVWGQGTTVTVSSGGGGSGGGGSGGGGSEIVLTQSPATLSLSPGERATLSCR
ASQSVSSYLAWYQQKPGQAPRLLIYDASNRATGIPARFSGSGSGTDFTLTISSLEP
EDFAVYYCQQRSNWPITFGQGTRLEIK (SEQ ID NO: 35)
aCD20 (ofatumumab, clone 2F2), VLVH:
GAAATCGTGCTGACACAGTCTCCAGCTACCCTGAGCCTTTCTCCCGGCGAAA
GGGCCACCCTGAGCTGTCGAGCTTCTCAATCGGTGTCGAGTTACCTCGCTTGG
TATCAGCAGAAACCGGGCCAAGCACCACGGCTCTTGATCTATGATGCTAGTA
ACCGCGCAACCGGCATTCCGGCAAGGTTTTCGGGGTCAGGTTCTGGCACAGA
CTTTACATTGACTATCTCTTCACTCGAACCCGAGGATTTTGCAGTCTACTACT
GCCAGCAGAGGAGTAATTGGCCAATTACATTCGGTCAAGGAACCCGGCTTGA
AATAAAGGGAGGGGGAGGCTCTGGGGGAGGAGGAAGTGGCGGCGGAGGGTC
CGAAGTCCAATTAGTGGAAAGCGGGGGGGGACTGGTCCAGCCCGGGAGATC
CCTGCGACTCTCCTGTGCCGCCTCTGGCTTTACCTTTAACGACTACGCTATGC
ACTGGGTGAGACAGGCCCCTGGTAAGGGGCTTGAGTGGGTATCAACTATTAG
CTGGAACTCTGGTTCCATAGGGTATGCAGACAGCGTCAAGGGCAGATTCACA
ATTTCACGAGATAATGCGAAAAAGTCATTGTATCTTCAAATGAACTCACTCA
GAGCCGAAGACACCGCCCTGTACTACTGCGCAAAGGACATTCAATACGGAAA
TTATTACTATGGAATGGATGTATGGGGACAAGGCACCACGGTGACAGTTTCC
TCC (SEQ ID NO: 36)
EIVLTQSPATLSLSPGERATLSCRASQSVSSYLAWYQQKPGQAPRLLIYDASNRA
TGIPARFSGSGSGTDFTLTISSLEPEDFAVYYCQQRSNWPITFGQGTRLEIKGGGGS
GGGGGGGGSEVQLVESGGGLVQPGRSLRLSCAASGFTFNDYAMHWVRQAPG
KGLEWVSTISWNSGSIGYADSVKGRFTISRDNAKKSLYLQMNSLRAEDTALYYC
AKDIQYGNYYYGMDVWGQGTTVTVSS (SEQ ID NO: 37)
aCD19 (B43), VHVL:
GAGGTGCAGCTGGTCCAGTCAGGAGCAGAGGTCAAGAAGCCAGGCTCCTCTG
TCAAAGTGTCCTGCAAAGCCTCAGGCTATGCCTTCTCCTCCTACTGGATGAAC
TGGGTGCGCCAGGCCCCTGGGCAGGGCCTGGAGTGGATGGGACAGATCTGGC
CAGGAGATTCAGACACCAACTACGCCCAGAAGTTCCAGGGAAGGGTCACCAT
CACAGCAGACGAGAGCACCTCCACAGCCTACATGGAGCTGTCTTCACTCAGG
TCAGAGGACACAGCTGTGTATTACTGTGCCAGAAGGGAGACCACCACTGTGG
GCCGCTACTATTATGCCATGGACTACTGGGGCCAGGGGACCACAGTTACTGT
TTCAAGTGGGGGTGGTGGCTCAGGAGGAGGAGGAAGTGGGGGTGGGGGCTC
AGACATCCAGCTCACCCAGAGCCCCAGCTTCCTCTCAGCCTCTGTGGGAGAC
AGAGTCACTATCACCTGCAAGGCCAGCCAGAGTGTGGACTACTCAGGGGATT
CATACCTCAACTGGTACCAGCAAAAACCTGGCAAAGCTCCCAAGCTGCTCAT
CTACGATGCCAGCAACCTGGTTTCTGGTGTGCCTTCCAGATTTTCTGGCTCTG
GAAGTGGCACAGAGTTCACCCTTACCATTTCTTCCCTGCAGCCAGAAGATTTT
GCCACCTACTACTGCCAGCAGAGCACAGAAAACCCCTGGACATTTGGAGGGG
GAACCAAACTGGAAATCAAA (SEQ ID NO: 38)
EVQLVQSGAEVKKPGSSVKVSCKASGYAFSSYWMNWVRQAPGQGLEWMGQI
WPGDSDTNYAQKFQGRVTITADESTSTAYMELSSLRSEDTAVYYCARRETTTVG
RYYYAMDYWGQGTTVTVSSGGGGSGGGGSGGGGSDIQLTQSPSFLSASVGDRV
TITCKASQSVDYSGDSYLNWYQQKPGKAPKLLIYDASNLVSGVPSRFSGSGSGTE
FTLTISSLQPEDFATYYCQQSTENPWTFGGGTKLEIK (SEQ ID NO: 39)
aCD20 (rituximab), VHVL:
GGAGTCCAGTGTCAGGTGCAGCTGCAGCAGCCAGGGGCAGAGCTGGTCAAA
CCTGGTGCCAGTGTCAAGATGTCCTGCAAGGCTTCAGGCTACACCTTCACCTC
TTACAACATGCACTGGGTGAAGCAGACCCCAGGAAGAGGCCTGGAGTGGATT
GGTGCTATTTATCCTGGAAATGGAGACACCAGCTACAATCAGAAGTTCAAGG
GCAAGGCCACACTCACAGCAGACAAGAGCTCCAGCACAGCCTACATGCAGCT
CAGCAGCCTTACTTCAGAGGATTCTGCTGTGTACTATTGTGCCAGGAGCACCT
ATTATGGAGGGGACTGGTACTTCAATGTGTGGGGAGCTGGCACCACAGTCAC
TGTGTCTGCAGGGGGTGGGGGCAGTGGAGGTGGTGGCTCAGGTGGAGGAGG
CAGCCAGATTGTGCTGTCCCAGAGCCCAGCCATCCTGTCTGCCAGCCCTGGG
GAGAAGGTCACCATGACCTGCAGGGCCTCCTCCTCTGTGTCCTACATCCACTG
GTTCCAGCAAAAGCCAGGCTCCTCTCCCAAGCCCTGGATCTATGCCACTTCCA
ACCTGGCCTCAGGAGTGCCTGTCAGATTTTCTGGAAGTGGCTCTGGCACAAGT
TACAGCCTCACCATCAGCAGAGTGGAGGCAGAAGATGCTGCCACCTACTACT
GCCAGCAGTGGACAAGCAACCCCCCCACCTTTGGAGGGGGGACCAAGCTGG
AAATCAAG (SEQ ID NO: 40)
GVQCQVQLQQPGAELVKPGASVKMSCKASGYTFTSYNMHWVKQTPGRGLEWI
GAIYPGNGDTSYNQKFKGKATLTADKSSSTAYMQLSSLTSEDSAVYYCARSTYY
GGDWYFNVWGAGTTVTVSAGGGGSGGGGSGGGGSQIVLSQSPAILSASPGEKV
TMTCRASSSVSYIHWFQQKPGSSPKPWIYATSNLASGVPVRFSGSGSGTSYSLTIS
RVEAEDAATYYCQQWTSNPPTFGGGTKLEIK (SEQ ID NO: 41)

The materials and methods are now described.

Sequences encoding anti-CD19 [clone FMC63 (Nicholson, et al., 1997, Mol Immunol, 34(16-17):1157-65) and clone B43 (Uckun et al., 1988, Blood, 71(1):13-29)] and anti-CD20 [clone 2F2 (Ofatumumab, Teeling et al., 2004, Blood, 104(6):1793-800) and clone C2B8 (Rituximab, Reff et al., 1994, Blood, 83(2):435-45)] variable heavy and variable light chain domains were identified and isolated. All amino acid sequences were back-translated to nucleotide sequences and synthesized by Twist Biosciences.

In order to test functionality, scFv constructs were assembled in both VH-VL and VL-VH configurations. The heavy and light domains were separated by a flexible polypeptide linker, e.g. (G4S)3 or GSTSGSGKPGSGEGSTKG (SEQ ID NO:42) (for the latter, cf Whitlow et al., 1993, Protein Eng, 6(8):989-95). An immunoglobulin domain signal peptide for the secretory pathway was included at the N-terminus of each construct (MEFGLSWLFLVAILKGVQC; SEQ ID NO:43). For identification of labeled cells with dye-conjugated antibodies, amino acid tags were included at either the N-terminus or the C-terminus of each construct. Examples of these tags are the 4×V5

(GKPIPNPLLGLDSTGAGSGGKPIPNPLLGLDSTGAGSGGKPIPNPLLG
LDSTGGAGSGGKPIPNPLLGLDSTGSGAGGSSL; SEQ ID NO: 44),
the 3xHA
(GGGGSGGGAYPYDVPDYAGGGSGYPYDVPDYAGGGSGYPYDVPDYAAS
GPS; SEQ ID NO: 45),
the 6xHA
(GAGSYPYDVPDYAGAGSYPYDVPDYAGGSYPYDVPDYAGGAGSYPYDV
PDYAGAGSYPYDVPDYAGAGSYPYDVPDYAG; SEQ ID NO: 46)
and
the 6xFLAG
(GSGAGGSGSGAGGSGDYKDDDDKGAGSGDYKDDDDKGAGSDYKDDDDK
GGAGSDYKDDDDKGSGDYKDDDDKGAGSGDYKDDDDKGSGAGGSS;
SEQ ID NO: 47) sequences.

For a tandem scFv, fragments for CD19 and CD20 were separated by the flexible-rigid-flexible linker sequence GGGGSAEAAAKEAAAKAGGGGS (SEQ ID NO:49).

Co-culture experiments were performed using round-bottom 96-well plates. Jurkat TT2 T cells expressing the scFv reporter constructs and B cells are seeded at a 3-to-1 ratio into each well. Either 1 μM of cognate or negative control peptide was added to each well and incubated for 16 hours. Subsequently, cells are spun down and stained for flow cytometry.

For optimizing scFv reporter constructs, constructs containing combinations of B43 and 2F2 tagged with either HA or V5 were cloned in pTwist CMV WPRE Neo BetaGlobin. For testing, HEK cells were transiently transfected with the plasmids using TurboFect (Fermentas). At 24 hours post-transfection, medium was replaced with normal HEK culture medium. At 72 hours post-transfection, the supernatant was recovered, centrifuged, and used to stain BOLETH cells. Subsequently, cells were centrifuged and stained with fluorescent dye-conjugated antibodies for flow cytometric analysis.

### Example 3: Synthetic T Cell Receptor Signaling Response Elements for Cellular Assays

A T cell reporter system has been developed that is activated upon TCR engagement by its cognate HLA:peptide complex. This system is based on a potent transgenic cassette introduced into reporter T cell lines using the Sleeping Beauty transposon system, a nonviral DNA tool that can stably integrate DNA sequences into the eukaryotic cells.

The T cell reporter system developed and shown here has several important components: the cis-regulatory nucleotide sequences for recruiting transcription factors in response to T cell activation herein called “promoters”, a synthetic transcriptional co-activator to amplify endogenous signal transduction, tagged signaling accessory molecules CD19 single-chain variable fragment tagged with HA, and sfGFP.

Upon TCR engagement by its cognate HLA:peptide complex, endogenous NFAT and other transcription factors are activated, as well as the synthetic transcriptional co-activator (NBV eTF), and recruited to the promoters that contains their binding sites. The T cell reporter system's promoters drive the expression of CD-19 scFV with the 3 HA tags, and sfGFP. Expressed sfGFP marks the T cell that is being activated; whereas the secreted CD19-scFV with 3 HA tags can bind to the activating B cell, the antigen-presenting cell, which have the cognate peptide on its HLAs.

The ectopic transcription factor NBV (‘NFAT-Bob1-VP16’) is a fusion protein consisting of the following elements: the cytoplasmic retention and DNA-binding domains from the N′-terminus of the nuclear factor in activated T cells (NFAT), the octamer motif (‘ATGCAAAT’)-binding domain from the transcriptional co-activator Bob1, and the C′-terminal transactivation domain (TAD) from the herpesvirus VP16 protein. The functional principle is that this transcription factor is recruited to the nucleus in response to calcium-induced signaling from the TCR by virtue of its NFAT-derived N′-terminus, upon which it can bind to NFAT and octamer motifs in synthetic promoters. It can then recruit the transcription factors Oct1/2 through its central Bob1-derived domain, as well as drive transcription on its own via the C′-terminal VP16 TAD.

When introducing transgenic constructs via transposable elements, construct size is not as absolute a constraint as when working with other systems (e.g., lentiviral particles). Therefore, several additional design features into the construct in order to optimize its TCR signaling-dependent response. These features include:
Insulator elements flanking the signaling-dependent portion of the transgene to avoid spreading of activated chromatin from adjacent genetic elements or from the constitutively expressed downstream selection cassette,
A 3′-untranslated region (UTR, derived from the human IgG1 heavy chain locus) to increase transcript stability and provide a dedicated polyadenylation sequence to the inducible cassette, and
Unique restriction enzyme sites for the incorporation of signal-responsive intron elements, which also serve to increase transcript stability, and downstream cis-regulatory regions (‘enhancers’).

T cell activation is defined primarily by the interaction between the TCR itself and its the peptide-binding HLA molecule on the surface of the APC. However, this interaction and its downstream signaling cascades are mediated by additional surface molecules found on both the T cell and the APC. The strongest and best-studied interaction is that of the CD28 surface molecule on the T cells with members of the B7 family expressed on the surface of the APCs.

As a means to further potentiate minigene-based T cell activation in T cell lines used, surface receptors known to be involved in T cell activation were ectopically expressed and screened for functional synergy with the TCR-mediated signal. Constitutive expression of CD2, CD226, CD40L, ICOS, OX40, and 41BB was thus induced in TCR-expressing Jurkat and SKW-3 cells and then they were co-cultured with cognate minigene-transduced B cells. These were instrumental in potentiating T cell activation.

The materials and methods are now described:

### Cell Lines

BOLETH cells were used as B cells for antigen-presenting cells and they are maintained in RPMI 1640 medium supplemented with 10% FCS, Pen/Strep, Kanamycin, 1 mM sodium pyruvate, MEM non-essential amino acids, and 50 uM β-mercaptoethanol. T cell lines used were Jurkat and SKW-3 cells, maintained in RPMI 1640 medium supplemented with 10% FCS and Pen/Strep.

### Generation of TCR-Transgenic T Cells

T cell receptors in lentiviral constructs are used to transfect HEK cells using the TransIT protocol. After 2 days, virus is collected and T cells are transduced. T cell medium is changed 3 days later and medium with 1 ug/ul puromycin is used for selection.

Transfection of T Cells with Reporters

Sleeping beauty transposon reporter constructs were transfected together with transposases into T cells, 5 to 1 ratio, using the Neon transfection system following the manufacturer's protocol. The voltages used for Jurkat cells were 1350V/10 W/3 pulse, and for SKW-3 cells 1500V, 30 W and 1 pulse were used as settings. 48-72 hours after transfection, the cells were selected using 10 μg/μl Blasticidin for Jurkat and 6 μg/μl Blasticidin for SKW-3 cells.

### Minigene Vector Cloning

In order to induce minigene expression and subsequent presentation on HLA molecules, lentiviral particles driving expression of the respective minigene from a constitutive promoter were generated. Specifically, cognate or mismatch minigene were restriction-based cloned into the pLVX-EF1a-IRES-Puro vector (TAKARA). This vector was further modified to include a T2A polycistronic cassette in frame with the minigene, directly followed by a TagBFP2 fluorescent reporter. Moreover, a CMA sequence was introduced immediately upstream of the minigene's coding sequence to facilitate chaperon-mediated autophagy and subsequent peptide presentation on ClassII HLA molecules.

### B Cell Culture and Minigene Transduction

BOLETH cells (Sigma, 88052031-1VL) were cultured in 6-well cell culture plates in RMPI medium (Gibco, #61870143) supplemented with 10% FBS (HyClone), 1% penicillin-streptomycin, 1% kanamycin, 50 μM β-mercaptoethanol, 1 mM sodium pyruvate, and 1 mM NEAA (Gibco, #11140050) at 37° C. humidified incubator with 5% CO₂. Cells were passaged every two to three days at a ratio of 1:2.

In order to induce minigene expression and presentation, lentiviral particles were generated via co-transfection into HEK 293FT cells in 6-well plates of 1.33 μg of the transfer vector alongside 1 ug of psPAX2 and 0.67 ug of pMD2.G accessory vectors using the TransIT VirusGEN transfection reagent (Mirus). Following 48 hours of incubation, supernatants were collected, filtered through a 0.45 μm PES filter. BOLETH cells were subsequently transduced by resuspending 1.5×10⁶/6 well plate in a 1:1 viral supernatant dilution in the presence of 8 μg/ml of Polybrene (Millipore) and 2 mM BX795 (Sigma). After 24 hours of incubation cells were washed from virus-containing medium and resuspended with full B cell medium. Functional experiments were conducted 4-5 days following transduction.

### Overexpression of Cross-Activation Surface Molecules in T Cell Lines

The endogenous coding region of the human surface molecules CD2, CD226, CD40L, ICOS, OX40 and 41BB was ordered from TWIST bioscience directly cloned into the pTwist Lenti SFFV lentiviral vector. In these vectors, expression of each candidate is controlled by the SFFV-derived promoter sequence. Lentiviral supernatants were generated via co-transfection into HEK 293FT cells in 6-well plates of 1.33 μg of the transfer vector alongside 1 ug of psPAX2 and 0.67 μg of pMD2.G vector using the TransIT VirusGEN transfection reagent (Mirus). Following 48 h of incubation, supernatants were collected, filtered through a 0.45 μm PES filter. TCR-expressing Jurkat cells were then transduced in a 1:4 supernatant dilution ratio and TCR-expressing SKW3 cells were comparably transduced in a 1:2 dilution ratio. After 24 hours of incubation, cells were washed from virus-containing media and resuspended in full RPMI medium. Expression of above-mentioned surface receptors was then validated using flow-cytometry grade fluorescent dye-conjugated antibodies. Functional experiments were conducted 1 week post-transduction.

### Functional Co-Culture Experiments

Co-culture experiments were performed using round-bottom 96-well plates. P10-NBV Jurkat and SKW-3 reporter cells and B cells were seeded at a 1:1 ratio into each well. For peptide co-culture experiments, either cognate peptide or mismatched peptide is added to each well and incubated for 16 hours. For minigene-based co-culture experiments, minigene-transduced B cells harboring either the cognate of mismatch minigenes were used. Next day the cells are spun down and stained for Flow cytometry.

The experimental results are now described.

Sleeping beauty-based transposon constructs were designed with TCR inducible reporter genes cassettes upstream of a constitutively expressed blasticidin resistance gene (FIG. 18A). Sleeping beauty constructs with TCR signaling-inducible reporter gene cassette flanked by insulator sequences with a downstream, constitutively expressed puromycin resistance gene and the co-stimulatory molecule CD28 (FIG. 18B). NBVp and the P10 promoter drive high GFP and HA reporter gene expression in sleeping beauty constructs. Synthetic promoter sequences 5′ of P10 or NBV sequences were cloned (FIG. 18C), and there was synergy between synthetic promoter sequences (FIG. 18D). Adding additional synthetic promoter sequences upstream of P10 or NBV sequences further improves reporter sensitivity in co-culture of Jurkat-P10NBV-TT2 cells with BOLETH cells loaded with 0.1 μM cognate or negative control peptides.

The sensitivity of the Jurkat-P10NBV reporter line with various TCRs targeting DR4-presented peptides from the tetanus toxin protein is shown in FIG. 19.

The GFP and HA median intensities of B cells co cultured with 1 μM cognate or mismatched peptide and SKW-3 cells expressing sleeping beauty transposon system reporters is shown in FIG. 20. The different synthetic promoters were compared to 4×NFAT promoter in the sleeping beauty transposon reporter. GFP median fluorescence intensity signal obtained from all synthetic promoters were greater than 4×NFAT promoter which leads to much easier identification of the activated T cells. Both HA and GFP fluorescence intensities were doubled upon using 2 copies of NBV promoter instead of 1.

A 4×NFAT and NBV promoter comparison in sleeping beauty transposon system via co-culturing with B cells using different concentrations of cognate peptide is shown in FIG. 21. NBV promoter leads to higher intensity of both HA and GFP signals compared to NFAT. Even with lower concentrations of peptide NBV promoter leads to a positive signal which could potentially identify an activated T cell, whereas signal levels obtained from NFAT are lower. Higher the concentration of peptide used NBV promoter response reaches a plateau, and the activation pattern is similar to an ON and OFF switch.

The GFP and HA median intensities of B cells co cultured with 1 μM cognate or mismatched peptide and Jurkat cells expressing sleeping beauty transposon system reporters is shown in FIG. 22. Many other synthetic promoters are depicted in the figure lead to better signal than NFAT promoters; however, the best signal is still obtained from NBV promoter.

The ectopic expression of co-stimulatory molecules from lentiviral constructs can further increase reporter gene expression in Jurkat-NBV58-TT11 cells (FIG. 23).

FIG. 24 provides a diagram of the principle of function for the NBV synthetic transcription factor.

The TCR responsive promoter-driven synthetic transcription factor NBVtf can further increase reporter gene expression (FIG. 25). The synthetic transcription factor NBVtf can bind to the synthetic NBV promoter and activate reporter gene expression. FIG. 25B provides a schematic design of inducibly-expressed NBVtf in sleeping beauty constructs with P32-driven NBVtf expression and constitutively expressed neomycin resistance gene and co-stimulatory molecules CD226 and 4-1BB. Sleeping beauty-expressed NBVtf further improves reporter gene expression in plate co-culture of Jurkat-P10NBV-TT2 cells with BOLETH cells loaded with 0.01 μM cognate peptide (FIG. 25C). Sleeping beauty-expressed NBVtf further improves reporter gene expression in plate co-culture of Jurkat-P10NBV-TT11 cells with BOLETH cells loaded with. 0.1 μM cognate peptide (FIG. 25D).

Surface expression of co-stimulatory proteins potentiates minigene-based T cell activation. TT2-expressing P10-NBV reporter Jurkat and SKW3 co-culture experiment summary depicting CD69 and GFP upregulation in T cells and anti CD19 scFv marking on cognate minigene-transduced B cells (FIG. 26).

FIG. 23-26 demonstrate the positive effects of two trans-elements in improving TCR reporter cell function.

Element 1: synthetic transcription factor NBVtf, which is TCR signaling responsive expressed under the promoter P32.

Element 2: co-stimulatory molecules, which are constitutively expressed under commercial/published promoter PGK.

With the sleeping beauty construct shown in FIG. 25, two elements were combined. Expression of this construct further improved the reporter gene GFP/HA expression.

SEQUENCES

NBV promoter nucleotide sequence: (SEQ ID NO: 50)
GAATTCAAGTACCGTGCAGGAGGAAAAACTGTTTCATACAGAA
GGCGTGGGCTATGCAAATCGAAACGCTGGAAACACTGAGTAATCAGATCAAG
TCATGTTTCCATTTGAATGCAAATGGATACAGAAGGCGTGGAGGAAAAACTG
TTTCCTGTTCCAATAGACGCCCTTTTTGAATGCAAATGGCGAGCTCATGGGTT
TCTCCACCAAGGTGACGAGTGGAAAATCTGACTCAGAATTATCCCTTGTTATG
CAAATCGCCCAAAGAGGAAAATTTGTTTCATAGAACATACTGTCTCAAATAG
ACGCCTCCTCGAGCGTTTCCCGGGGTACC
P1 promoter nucleotide sequence: (SEQ ID NO: 51)
GAATTCAAGTACCGTGCATTGAAGTTAAATAATTTACAGCATAT
GGAAAATAAATTTCAGATGGATTACGAGGCTGCAAGAGCTCCCTGGCCTTGG
AAAACCAACTCACACACCTTGACTGAGGAAACTGCACAAATATTGTGTTTTC
CATCTGCCCTTGGTTTAAAAAACTGTGTATCTTCTCTCCACTTCATTTTCCATA
TCTGCAAAATGGGAACGAGCTGGGTGGTGGCTACATACAGATGGAAAAATTC
ATCAGCTGTACACTGCAAGGGCCATGGGTGAGCACCAGCTGGAAAATTACTG
CTTCAACTTTCATATTTTGGTAACTAATTCATATTTTTGGAAAACATACTGGCC
ACACAAAATTTAAAGAGGAAGTTATAGTTCAGTTTTTCCAAGTTAGCATTTCC
TGCTTTGTAGTTTTATGACAAAGAAAATTTTCTGAGTTACTTTTGTATCCCCAC
CCCCTTAAAGAAAGGAGGAAAAACTGTTTCATACAGAAGGCGTTAATTGCAA
ACATACTGTCTCAAATAGACGCCTCCTCGAGCGTTTCCCGGGGTACC
P2 promoter nucleotide sequence (SEQ ID NO: 52):
GAATTCAAGTACCGTGCATCCAGGGGTCTCTTATATGCACACTT
TTTCCAAACAAACGGGGATCAAAATGTTGGAACCTGCTGTTGGCCTGCCTGG
AAAATTCTTCCCTCCTTACCTTGAGCTGGGTGGTGGCTACATACAGATGGAAA
AATTCATCAGCTGTACACTCGTCCTCCACCCTCCAAGTCCGCGCTGGAAAATC
ACCCGCTGCGGGCTCCCACCAGTGAAATGGGATATTAGATATTTTCCAGCTA
GGATACGACAGCTCCTATATGTGGGACAGAGCCTTGGTCTGGAAAACACTTT
CAAGCCCACTGTGAGACATTTGGGAATATTGGGGGAGTTTTCCAAAAGAGAA
ATCCAGGTGAGGGTCCAAAGGGACTGATTATGGGCTGGAAAATCAAACTGGC
ACCTGCAATGCATGCTGACGTATGATTTTTATTTGGAAAAACCTCAAAATAGT
AAATGTCCCCTATTAGCTGTTTTGGGAATCCTTTTCCAGTCATGACCACATTTT
GCAGAGATGGGCTAACAGGTATGAGCATGGGAAAAGCATGTTTCAAGAATTT
GAGATGTATTTCCCAGAAAAGGAACATGATGAAAATGGTCAGAAAAGGCAA
ACATACTGTCTCAAATAGACGCCTCCTCGAGCGTTTCCCGGGGTACC
P3 promoter nucleotide sequence (SEQ ID NO: 53):
GAATTCAAGTACCGTGCATTAGCGGGTCGGTATAATCCCCGACT
GGAAAACCGCCCTAAGCCTTATGAGAGCTGGGTGGTGGCTACATACAGATGG
AAAAATTCATCAGCTGTACACTTGAGTCAGGCATCTATACCTGTTAATGGAA
AAAGATGGAAGGGGGTATTGAGATGCCCTATGTAAGTCAAGTGTCTTTTCCA
GAGGAAGTTTGCATCCGATGATATGACATTATTCAATCTGTCATGGAAAAGT
GCTGCGTAGCCTACCCAGAATAGACATATTTTTCATTCATTTGGAAAACACCC
ACTTCCCTGTTTCATTTATTTGAGGAACAAACTTGCCGTTTTCCATAACAGCT
GCACTATTTTTTATATATAATGTGTGTAACACAGCTTTTCCAGTGCCTGGTAC
AAGGTAAATAGAGTTTTGGGTAAGAAAACCCTTTTTCCACTTTCTAAATCTTC
AAGGAAATACAAAATTCTGCTAGTTCATATTTTCCATTTCATTTATTCAGCAA
ATTTTTTAAAAAACAGTAAGTCTTGGCAAGTCCATCTCCCCCATCTGACATCT
TGAAACAAAGTTTCCTCTCTGGGTTCTCAGTACCAAGAACAAACATACTGTCT
CAAATAGACGCCTCCTCGAGCGTTTCCCGGGGTACC
P4 promoter nucleotide sequence (SEQ ID NO: 54):
GAATTCAAGTACCGTGCATCGCAGACATCATCTTTGATGCTCTT
TTTCCACTGTTTCGGTGCTTTAATTCAGTGGATCAATAGACAGTTCCTGTTTTC
CACACAACTGAAAGGGTGGAACAGGCCAAGAGGCAGAGTGGGAGATTTTCC
AGAGAGAAAGAAATAGAAGCAACATCCTGTGTGCACAGCAAGTGTGGAAAA
CTGAAATAGTGATCTGAGAAGAGATTGGCCACAGAAATCCAGGTGGAAAAC
CTGCAGAAGAGTGGGTTTACCAACCCTGCCCATAGTTGACATTTTTCCACCAC
TCCCCCCTTCCCAGGGATATTATTTAAAAAGGAAACACTTGGAAAAAATTATT
TCATTTGTTCAGCCCTGGATGTGGACAGCGGCCCCCTGGAAAACGTGTATGA
GAGCATCCGTCCAGGGGTCTCTTATATGCACACTTTTTCCAAACAAACGGGG
ATCAAAATGGGAGGCCAAGGTGGGAGAATTTTTTTTCCAAGTGTGCAGTTAA
TATTTAATAATTTGCCTGAATCATCCTGGTAGTCACAGAGGTAAACGTTTTTG
AAACAATTGTTCCAAATTGTGGGTACAACTTACAACTAATTTTTCCCATTAAA
ACATACTGTCTCAAATAGACGCCTCCTCGAGCGTTTCCCGGGGTACC
P5 promoter nucleotide sequence (SEQ ID NO: 55):
GAATTCAAGTACCGTGCAAAGGCTTCAGTTTCAAATTGAATACA
TTTTCCATCCATGGATTGGCTTGTTGCCTCCTGCGTGAACCAGCACTGGTTTTT
CCAGTAACTGGAGCCACAAGCTGTTGGAACCTGCTGTTGGCCTGCCTGGAAA
ATTCTTCCCTCCTTACCTTCTTCAGCTGAGGTCATCTGCAGACATTTTCCATAG
TTGGGAGCTATTTTTAAAGTGTAAAATGGAGCAGCCACTTTGGAAAACAGTT
TAGCAATTCATAATTTCAGTCTAACATGCAATTTAATTTTTTCCATTTAACAA
GTTTTATGGAACACTGATAATCTGTTGCCACCAAATGGAAAACGTAAACAAG
ATATTCTACACATGTATGCCAATGCAGATTCACTGGAAAAATATTGAAAATG
AAAACTCCCCTCCCCTTCGCATCGCCGGGGTTTTTCCAGCCGACCGTCGGCCA
CTTAACACAAAATTGTATTCTTTTGATTTTTTCCAATCATTTAAAAATGTCAAG
CCAGGCCAGATTTCCTGTGGCCCCGCCTGAATGATGAAACACGGGATGGCCA
TTGCACTCCCTGGCTTTTCCAGAGATCTCGGTCCTCGGTCCTGATGCAACCGT
CTGGAACATACTGTCTCAAATAGACGCCTCCTCGAGCGTTTCCCGGGGTACC
P6 promoter nucleotide sequence (SEQ ID NO: 56):
GAATTCAAGTACCGTGCATCATGTTGGGAATGGCACAACTTTTT
GGAAAAAAATACATTTATAAAATAAGAACAGATGCCTTCTGATGCTAGATTT
TCCAAAACTCCTACTGTTATGCTTTGAGGAGGCTATATAAATATTAATTTTCC
AGTTCTGGAATTAAGAGAACGGACTACAACCCACAGCTGGTGAGTGGAAAA
GAGGACATTCTTCTAAGAAAATAGATTGTTGGGAAGGTAACATTTTTCCATG
GTTTTGATTTTTCCCAAAAGGTCATTCTTTGCGGGTACATTTTCCAGGGTCCA
GCTCCGCAACAGGGCACAGCATCCGGAATTTTGCGTTTTTCCAGCGGGAAAA
CCAAGACCACACAGTAAGGTTTCTCATCTGCCTGGAAAAATGTGGAATTAGC
TCTTTTGGTAAATTGAGACCCCTTTTACAATGGAAAAATAACTGGTTATAACG
GTCGTGAGCCACCGCGCCCGGCCAACCTGGAAAATTTTGCAAGGAAATACAG
TTACCCAAGCCTCACCTCACTGATATTTTCCAACCAAAATGGGGGCTAAAAG
GGAGAGCGAGACATGCAGGGAGATGGAAAAAGAATCTTTTAGAGAAAACCC
ACCCCACCTTGGGAGCTGTGTCTGGAAAAGAGACTTTTCCTGAGCCGAATATT
CTAAATGTGCTTGTAATCATTTTCCACACTTGTTTATTTTTTGAGCTGGGTGGT
GGCTACATACAGATGGAAAAATTCATCAGCTGTACACTAACATACTGTCTCA
AATAGACGCCTCCTCGAGCGTTTCCCGGGGTACC
P7 promoter nucleotide sequence (SEQ ID NO: 57):
GAATTCAAGTACCGTGCATTTCCTCTTGTGCAACTGAAGAACAT
GGAAAAAATGATCCCTAAAGTCCTGTGTTTTTAAACGTGCGCTTTGGGATTTT
CCACACTGAAGGGTGGTGGCCTTTATTTCAAGATAGCTTTGGAAGTTTTCCAT
CAATGTAACCAGTTTCAAAAAGGATAGCACAAAACTGCTGTGTGGAAAAACC
TATAATGAACAACTGTTCACCCCCAACCCATTTGCTGTTTTTTTCCATGTGAG
CTTGAGAAGGCAACAGATTACCTAACCAACATGTGTGGAAAAGGAGTTAAAA
TCTGACAGGAGCTGGGTGGTGGCTACATACAGATGGAAAAATTCATCAGCTG
TACACTCCTAGTCAGAATTTGTACCCCAGACTGGAAAACTGGGGAATTTCCCT
GTAAATCCTTCTCCAATTGGGTCCCTTGCTTTTCCACCTCCTGTGCCTCTGGTT
TACTATTATTATCTTAGATGAGTTGGAAAACTGTGCTCAGAAGGGGTCAAAG
GCAGTTGTTGGATTGAGCATTTTCCAAGGAGACTACTTTGAACCCATTTCTCT
TTTTATGGCTTCATAATTTTCCACTATGTAGACATACTATAAAGTGTCATAAG
GAAGCAATATTTTTTCCAATTTGCATGAAGTTGCCTTGAGCACAGTCGGGGTA
TCGCAATGGAAAACTCTTGGCAAACTGTAAATTCCTGTTTTCCTTTCAGATGG
TGTTGGAAAACATTGCAGGAAAACCATGAACATACTGTCTCAAATAGACGCC
TCCTCGAGCGTTTCCCGGGGTACC
P8 promoter nucleotide sequence (SEQ ID NO: 58):
GAATTCAAGTACCGTGCATTGGAACCCATTGATTTATTCTGGAT
TTTCCACTGCTTTCTACCATTTTAACTTTTAAATTATGCTGACAGGGTTTTCCA
TAATTAATAATGTTTAAAGATTCTGCATGGTCTCTGCCTTCTTTTCCATCATGT
CTTCCTGTTCCTATCTGTTCCCTAACGCGTCTGACCGTGGAAAAGTGGATCAG
CAGGAGTGGTGAGAAAAATCCTTAAAAAGTCACGTTTTCCATTTGATTGGGT
CAAAACTCATTTGAAGCAATGTCCCTCCTTTTTTCCACTGTGGTCTGTGTGTCT
GGCTTGGGTTTTCCATTCAAGTCATTTGGAAAATATCCACCCCCCGGGGATAA
GTACTTACTGAGCTGGGTGGATTTTCCATTCATGGTTCCCTGGGGCTGCTCAC
CCTAGGGCAGTATGAAGTTTTTCCATCTCCAGCAGAGGACTCTTTTATCCAAT
CACATTTGTTTACAGTTTTCCATGTTGTAATCATGTCTGCTATCTTTTGTGCTT
TGGTGGTGAGCTGGAAAATCCTCAGTTCTGGCTGTCGCCTCCTGCGTGAACCA
GCACTGGTTTTTCCAGTAACTGGAGCCACAAGCCTTAGATCCTAAAAGCGTTC
CTTGGATGGAAAACCAACTCTTCCACTGGCTTCCAGGGGTCTCTTATATGCAC
ACTTTTTCCAAACAAACGGGGATCAAAAGGGAAAAGATATGTTTTTAACAGC
TTTTTCCATTCCTTCTCTGTTTCCAACATACTGTCTCAAATAGACGCCTCCTCG
AGCGTTTCCCGGGGTACC
P9 promoter nucleotide sequence (SEQ ID NO: 59):
GAATTCAAGTACCGTGCAACAAAACAGAGATTTTGCACAGGGC
TTTTCCAGTTAAATTTTAGGAAAGCAGAGCTGCCACGTTGGTACAGAAGTTG
GAAAACACCATACGCTTCAACTGCCCTAATTTTAGTGTCATGGCAACATTTTC
CAAGGGGATGGAATTGGGTATGCTATGTACGTCTATAAATTTTAATGGAAAA
ACCAAATAACTTTATCGTCACGCGCCACCATGTACTGATTTTGGAAAATGGTG
ATTTTCTAACTCCCTAAAGAGGGTTTATGTAGACTTTTTTTTCCATTTTTGACA
ACTTAAAAGAGGACTCAATAGATGCATCTGCATGGAAAACATCCTCCCCTCT
ACCAGTATGTACGTGCAAATATTCCAAAATTGGAAAAAAAATCTGAAATTTG
AAAACATAAAATGTCTATTAAAAGGGCTTTTTCCAATTCCTCCACACAAGTGG
TTTAGTAACGTAATGGGAACTTTCCTTTTCCATAAAACTGGGGAATCAAGGCA
TCGGTCAACAGAAAAGGCCCAGTTTTCCATGACAATGTCCAACCACTCGGGA
TGGCCATTGCACTCCCTGGCTTTTCCAGAGATCTCGGTCCTCGGTGCTCTTCTG
AGGGGCCAGGAAGTTCTGGAAAACTTCCCCTCAGCCGAGCTAGGAGATTTTA
AGGTGCACGCAAGGTGGAAAACCACTGCTGAAGCAGATTAGGCAAATGCTA
AATCTACCTCATTTTCCATACCTGTCTTTTGATTAACATACTGTCTCAAATAGA
CGCCTCCTCGAGCGTTTCCCGGGGTACC
P10 promoter nucleotide sequence (SEQ ID NO: 60):
AGAGATGGGCTAACAGGTATGAGCATGGGAAAAGCATGTTTCA
AGAATTTGAGATGTATTTCCCAGAAAAGGAACATGATGAAAATGGTCAGAAA
AGGCATTCTTAGGGTCCCCAGATGTCTGCCTTTTCCATTGATTTTCTTTCCTGG
TTAATTGCGAGGCTTGCCTCAAGGTGGAAAACAGTATCGGTTTCCACTGGATC
ACTAGGTCACAGGGTTTGCCTTTTTCCAAACCTGCATCTCAATTTTACCTATG
ATTTGCATTAAATAACTTTGGAAAAATTGAAGTTCTTGAGAAGCCACCGCGC
CCGGCCTCCTCTCCTTTTCCAAATTTATACATTTTGATTGGTGGGGAGATTCCA
TTATAGGGGCTGGAAAACCCCTTTACCATACCATGAATGTAACTGTACTGTTA
CAAAGTGGAAAATAACAGTTTCCACTTTTCGCAGCTCTTTGAGAGCAGGACT
CTATTTTCCAGTCTCTTTATAAGCTTCATTCATAGTGAAGCTAACAGAAGTTTT
TCCATGGTTAGATTGTGCACCGGGCTGTTTTTAAATGTAATATGATTTTCCATT
TACTTATTTATCTTTGTAGTTTTATGACAAAGAAAATTTTCTGAGTTACTTTTG
TATCCCCACCCCCTTAAAGAAAGGAGGAAAAACTGTTTCATACAGAAGGCGT
TAATTGCA
P14 promoter nucleotide sequence (SEQ ID NO: 61):
GAATTCAAGTACCGTGCAATATGAGAGCCCAGCACTTTACCTCC
TGACCACAGAGCTGGGTGGTGGCTACATACAGATGGAAAAATTCATCAGCTG
TACACTAAATACTTTATATTTTATAACTATATTTTCCAAGTTTGTGCTTTAACT
TTGTGATGGTTTAGCGGCAGCTGCTTGTAGGGGTATTTTTGGTTTCCTGCTTCT
TTTTTCCCTTCTAAGTAAAGACAAAATAACATAAGTGTTTGACTAATGATCAA
ATGGAAAATAGAATCTGGTGAAGGTGTCCAAACGCAGAAAGGAAGGTGTTT
ACAGGGAGGACATAGATCCCTCCACCCCACCACCCCATCATTTCGTTGTTGCC
AGCTGGCTGATGTTAACAACATTGGAAAATATAGATCTGCGGGAGCATTTTG
GTTTGCAGAATATGCTGTATTTTTCCATAGCTGTTAGAAATGCTAAAACCCCT
GGCACACGGTTGGCAGTAGTTCTCCCCGACTCTCCTAGGAATGTTTTTCCTCT
GTGCTGAGCTGTTGTCACTGCTCTTTGCAACACTTTTGCTCCACGGAGGGGGA
GGATAACATACTGTCTCAAATAGACGCCTCCTCGAGCGTTTCCCGGGGTACC
P15 promoter nucleotide sequence (SEQ ID NO: 62):
GAATTCAAGTACCGTGCAAGACTTGTTCTTCTTCTGCAGTATCT
GGAAAATCTGGAGAAATTAATGTTAAAAGACATTATTATTAAGTCCCTGGAA
AAACACATGGCCATTTTCAATAAAACCCCTGGCACACGGTTGGCAGTAGTTC
TCCCCGACTCTCCTAGGAATGTTTTTCCTCTGTGCTGAGCTGTTGTCACTGCTC
TTTGCAACACTTTTGCTCCACGGAGGGGGAGGATTCAACTGTGTATCTGCTTG
CAGTGTTTTTCCATTATAATAGTTTTTAAAATATGACTCCATCCCAAGAAATT
GGTTTTAAAGATTTGAGGAAAAACTGCTTTTAGATTTCAGAGAAAGAAAAAG
AGTGTGTTTCTACTTCCCCCTTTTAAATCTGATAACTTTTATTTGATCACTAGG
TCACAGGGTTTGCCTTTTTCCAAACCTGCATCTCAATTTTCATTAACTACTTAA
AGCATAAGCTATTTTCCAGGAGAGGCAGCAAGTGCATATTCAGATAATCGGG
TCCCAGGTCTGGAAAAGCAGCCTTTTCCCCACGTAACATACTGTCTCAAATAG
ACGCCTCCTCGAGCGTTTCCCGGGGTACC
P32 promoter nucleotide sequence (SEQ ID NO: 63):
TGTTTTTGATGGGAAAATTGAGATGCAGGTTTGGAAAAAGGCA
AACCCTGTGACCTAGTGATCATAAACAACAGGCACCCCCTTAAAGAAAGGAG
GAAAAACTGTTTCATACAGAAGGCGTTAATTGCATGAATTAGAACCCATATG
ACTGATGGCATATTCAGATAATCGGGTCCCAGGTCTGGAAAAGCAGCCTTTT
CCCCACGTTTCTTTCCCCACCTAAGAATGATTTCTATATAAGCATAACAGTAG
GAGTTTTGGAAAATCTAGCATCAGAAGGCATCTGTTCTTCACTTTTATGTCTG
TACACTCGCTGTCTGCTGTCGGCTCAGGAAAAGTCTCTTTTCCAGACACAGCT
CCCAAGGTGGGGTGGGCGCAGCTGAGAAGCCGTAATCAGATCAAGTCATGTT
TCCATTTGAATGCAAATGGATACAGAAGGCGTGGAGGAAAAACTGTTTCCTG
TTCCAATAGACGCCCTTCAGTTTCTGGCCCACTTGTTTGTCTAATAAGTGTTTG
ACTAATGATCAAATGGAAAATAGAATCTGGTGAAGGTGTCCAAACGCAGAA
AGGAAGGTGTTTACAGGGAGGACAATATGAGAGCCCAGCACTTTACCTCCTG
ACCACACAGTAGCAGGTGTTTCCTTAAGGCCTCAGCTGAAGAGTGAGTCGGT
TAAAGGGAGAGCGAGACATGCAGGGAGATGGAAAAAGAATCTTTTAGAGAA
AAAGTAAAAGCCCTGTAGTTCCTTTGTTGGTAGCTTCTTAGAAGAATGTCCTC
TTTTCCACTCACCAGCTGTGGGTTGTAGTCCGGGAAGTGGGAAGATGATCAA
TAAACTTCTTCCAGTAACCCTTTCAAGTTACATCCTCCCAATATGCAAATCCT
CCGTGGAGCAAAAGTGTTGCAAAGAGCAGTGACAACAGCTCAGCACAGAGG
AAAAACATTCCTAGGAGAGTCGGGGAGAACTACTGCCAACCGTGTGCCAGGG
GTTTTAGTGTAAATTATCTTTTTGTTACCCAAGCCTCACCTCACTGATATTTTC
CAACCAAAATGGGGGCTAAATGGTCTGATTACAGGCGTGTGTGACTCATAGC
TTTTCCTCACCGCGCCCGGCCTCCTCTCCTTTTCCAAATTTTCCAATACATTTT
GATTTTCTTAAATGGATGTGTCATGTTTTCCAATTTGAAATGCAAATGTCAGG
AAAGTCCCCCAGCTCACCETTATAACCAGTTATAGGGGACTTTCCATCTATGC
ATGGGGATTCCCCTTTTCCATTGTAAAAATTTGAAATGCAAATAGGAAAGTCC
CGGGGTCTCAAAAGATGATGCAATTTTACCATTATACCCCACAGCT
P45 promoter nucleotide sequence (SEQ ID NO: 64):
CACCCCCTTAAAGAAAGGAGGAAAAACTGTTTCATACAGAAGG
CGTTAATTGCATGAATTAGACTAAAAAGGTAGTGCATTCTTAGGGTCCCCAG
ATGTCTGCCTTTTCCATTGATTTTCTTTCCTGCCCGCTCCTTAGGACACCTCTT
AAGTGTACAGCTGATGAATTTTTCCATCTGTATGTAGCCACCACCCAGCTCCA
CGTATTTTCAGGTCTCTTTTACTCATTTTTAATTGAAAATGGCCATGTGTTTTT
CCAGGGACTTAATAATAATGTCTTTTAAAAATAAAGGATTGAATGCAAATGG
CGAGCTCATGGGTTTCTCCACCAAGGTGACGAGTGGAAAAGGTGAAGGTAAC
AATCTTTTCCTTGACTCAGAATTATCCCTTGTTATGCAAATCGCCCTGCTGAA
GTCCTTTCAAAAGGTCATTCTTTGCGGGTACATTTTCCAGGGTCCAGCTCCGC
AACAAATGTGGACCCTGTCATTGAATGCCGCAGATGTACATGCTCCCGCAGA
TCTATATTTTCCAATGTTGTTAACATCAGCCAGCTGGCAATCTACAATTTCCA
ACCTGTCTCTTCGCATATTTTAGTTTTCCAAATTTCCAAATTCTTCCAACTTTA
TATTTTATAAAATATGAAATCTATATTTTCCAAGTTTGTGCTTTAACTTTAATT
TTCTTATAAACCCATATGACTGATGGCATATTCAGATAATCGGGTCCCAGGTC
TGGAAAAGCAGCCTTTTCCCCACGTTTCTTTCCCCACCTAAGGAAAGTCCCAT
TTGAAATGCAAATATGGGGTCCCAGCTCACCGTTATAACCAGTTATTTTTCCA
TTGTAAAAGGGGTCTCAATTTACCATTATACCCCACAGCTCAGCTGAAGAGT
GAGTCGGTTAAAGGGAGAGCGAGACATGCAGGGAGATATTTGAAATGCAAA
TTCTTCCAGGAAAAATTTCCAAGAATCTTTTAGAGAAAAAGTAAAAGCCCTG
TAGTTAGAAAAGAGGAAGGAAATTGCCTTTTCTGACCATTTTCATCATGTTGC
TTTTCTGGGAAATACATCTCAAATTCTTGAAACATGCTTTTCCCATGCTCATA
CCTGTTAGCCCATCTCTCCCAGTTTGAATCTCTGATAAACTATGGGTCTCTGT
AAAATAGATTGTTGGGAAGGTAACATTTTTCCATGGTTTTGATTTTTCCCAAT
TTGAAATGCAAATAGGAAAGTCCCAAAGTAAGGTGATGTCAATATTTAAGGT
GAAGGTAACATGTCTTCCATATTGATTTA
P46 promoter sequence (SEQ ID NO: 65):
AACCCATATGACTGATGGCATATTCAGATAATCGGGTCCCAGGT
CTGGAAAAGCAGCCTTTTCCCCACGTTTCTTTCCCCACCTAAATGACAATCCA
CATTAACTACTTAAAGCATAAGCTATTTTCCAGGAGAGGCAGCAAGTGCATT
CTACTCCCATGAGAGAAGTCGGGCTGTTTTTAAATGTAATATGATTTTCCATT
TACTTATTTATCTTTAAAATGAGGTTTTTTGTTACCCAAGCCTCACCTCACTGA
TATTTTCCAACCAAAATGGGGGCTAAATGGTCTGAGGAAAGATTCTATTGCA
TTTCTAACAGCTATGGAAAAATACAGCATATTCTGCAAACCAAAATGTAGAA
CGAAGGACAGCTGAAGAGTGAGTCGGTTAAAGGGAGAGCGAGACATGCAGG
GAGATGGAAAAAGAATCTTTTAGAGAAAAAGTAAAAGCCCTGTAGTGGGGA
TTCCCCAAACTGTTTCCTGTTCCAATAGACGCCCTTTTTGAATGCAAATGGCG
AGCTCATGGGTTTCTCCACCAAGGTGACGAGTGGAAAATCTGACACTCGCTG
TCTGCTGTCGGCTCAGGAATGTGAGTCATAAGTTTTTCCACTCTTTTCCAGAC
ACAGCTCCCAAGGTGGGGTGGGCGCAGCTGAGAAGCCAGGAAAGTCCCAGT
GGAAAATCTGACTCAGAATTATCCCTTGTTATGCAAATCGCCCAAAGAGGAA
AATTTGTTTCATAGAACATACTGTCTCAAATAGACGCCTCTATGAGATGACCT
ATGATTTGCATTAAATAACTTTGGATTTTCCAAAAAATTCCATTGAAGTTCTT
GAAATATGCAAATGAAACGTTGACGCATCTGAGGTCCCAAAATGTGATGGTT
TAGCGGCAGCTGCTTGTAGGGGTATTTTTGGTTTCCTGCTTCTTTTTTCCCTTC
TAAGTAAAGACAAAATAACTAGATCCCTCCACCCCACCACCCCATCATTTCG
TTGTTGTTTAACAAGTGTTGAGTAAAGGGGACTTTCCAGGGGACTTTCCACTA
AAAAGGTAGTGCATTCTTAGGGTCCCCAGATGTCTGCCTTTTCCATTGATTTT
CTTTCCTGCCCGCTCCTTAGAGGAAAGTCCCATTTGAAATGCAAATAGGGGA
CTTTCCCTATTCACATGTTCAGTGTAGTTTTATGACAAAGAAAATTTTCTGAG
TTACTTTTGTATCCCCTGTGAGTCATACCTTTTCCACCTTTTCCACTTATTCCA
ATTTGAAATGCAAATAAAGAAAGGAGGAAAAACTGTTTCATACAGAAGGCG
TTAATTGCATGAATTAGAGCTA
P47 promoter nucleotide sequence (SEQ ID NO: 66):
CTTCGCATATTTTAGAAATACTTTATATTTTATAACTATATTTTC
CAAGTTTGTGCTTTAACTTTAATTTTCTTATATGTTTTTGATGGGAAAATTGAG
ATGCAGGTTTGGAAAAAGGCAAACCCTGTGACCTAGTGATCATAAACAACAG
GTACAGAAGGCGTGGGCTATGCAAATCGAAACGCTGGAAACACTGAGTAATC
AGATCAAGTCATGTTTCCATTTGAATGCAAATGGATACAAATCTGTGTGGTGC
ACAATCTAACCATGGAAAAACTTCTGTTAGCTTCACTATGAATGGCATTAAG
ATATGAGCCCAGGGCCCACGCTCCCTCCGGGCACAGCATCCGGAATTTTGCG
TTTTTCCAGCGGGAAAACCAAGACGTGTTTTCCCGTTTCCCACAAGGAAAACT
TCCTTGGTGGAGAAACCCATGAGCTCATCTGGAGGGCATCTGAGGTCCCAAA
ATGTGATGGTTTAGCGGCAGCTGCTTGTAGGGGTATTTTTGGTTTCCTGCTTCT
TTTTTCCCTTCTAAGTAAAGACAAAATAACTAGATCCCTCCACCCCACCACCC
CATCATTTCGTTGTTGTTTAACAAGTGTTGAGTAAATGGGGTCCCAGCTCACC
GTTATAACCAGTTATTTTTCCATTGTAAAAGGGGTCTCAATTTACCATTATAC
CCCACAGCTATTTCCAAGGAAAGTCCCATAATATGTTCTAGAAAAGTGGAAA
CTGTTATTTTCCACTTTGTAACAGTACAGTTACATTCATCTTTTTGGTAGTAGG
GGACTTTCCTGTGACTCATTTTTCCAAACCCATATGACTGATGGCATATTCAG
ATAATCGGGTCCCAGGTCTGGAAAAGCAGCCTTTTCCCCACGTTTCTTTCCCC
ACCTAAATATGCAAATAGGAAAGTCCCATTTGAAATGCAAATCAGTTTCTGG
CCCACTTGTTTGTCTAATAAGTGTTTGACTAATGATCAAATGGAAAATAGAAT
CTGGTGAAGGTGTCCAAACGCAGAAAGGAAGGTGTTTACAGGGAGGACAAT
ATGAGAGCCCAGCACTTTACCTCCTGACCACACAGTAGCAGGTGTTTCCTTAA
GGCCTATTTGAAATGCAAATAGGAAAGTCCCTCTTCCAATTTCCAAGGGGAC
TTTCCCTATTCACATGTTCAGTGTAGTTTTATGACAAAGAAAATTTTCTGAGTT
ACTTTTGTATCCCCACCCCCTTAAAGAAAGGAGGAAAAACTGTTTCATACAG
AAGGCGTTAATTGCATGAATTAGAGCTAAGGGGACTTTCCATTTGAAATGCA
AATAGGGGACTTTCCAAGATGATGCAATAAATCTTCCAAGGGGACTTTCCGA
GAGAAGTCGGGCTGTTTTTAAATGTAATATGATTTTCCATTTACTTATTTATCT
TTAAAATGAGGT
P53 promoter nucleotide sequence (SEQ ID NO: 67):
AGTGCTGGGTCAGCAGCTCTTTGAGAGCAGGACTCTATTTTCCA
GTCTCTTTATAAGCTTTGATGCAGTGTCAGAGAGAAGTCGGGCTGTTTTTAAA
TGTAATATGATTTTCCATTTACTTATTTATCTTTAAAATGAGGTATAAACTATG
GGTCTCTGTAAAATAGATTGTTGGGAAGGTAACATTTTTCCATGGTTTTGATT
TTTCCCAAAAGTATTTATGTATTGATTTAAGGAGTGTAGCTGTTAATTGCGAG
GCTTGCCTCAAGGTGGAAAACAGTATCGGTTTCCACTGCCACCCCAGAGGAA
AAACTGTTTCATACAGAAGGCGTGGGCTATGCAAATCGAAACGCTGGAAACA
CTGAGTAATCAGATCAAGTCATGTTTCCATTTGAACTTCGCATATTTTAGAAA
TACTTTATATTTTATAACTATATTTTCCAAGTTTGTGCTTTAACTTTAATTTTCT
TATATTTTGATGGGAAAATTGAGATGCAGGTTTGGAAAAAGGCAAACCCTGT
GACCTAGTGATCATAAACAACAGGAGCTCATGGGTTTCTCCACCAAGGTGAC
GAGTGGAAAATCTGACTCAGAATTATCCCTTGTTATGCAAATCGCCCAAAGA
GGAAAATTTAGGAAAGTCCCTTTCCAGAAGCATCTGAGGTCCCAAAATGTGA
TGGTTTAGCGGCAGCTGCTTGTAGGGGTATTTTTGGTTTCCTGCTTCTTTTTTC
CCTTCTAAGTAAAGACAAAATAACTAGATCCCTCCACCCCACCACCCCATCA
TTTCGTTGTTGTTTAACAAGTGTTGAGATTTGAAATGCAAATTAACCCTTTCA
AGTTACATCCTCCCCCTCCGTGGAGCAAAAGTGTTGCAAAGAGCAGTGACAA
CAGCTCAGCACAGAGGAAAAACATTCCTAGGAGAGTCGGGGAGAACTACTG
CCAACCGTGTGCCAGGGGTTTTAGTGTAAATTATCAGGGGACTTTCCAATATG
CAAATATGCAAATGGATACAGAAGGCGTGGAGGAAAAACTGTTTCCTGTTCC
AATAGACGCCCTTTTTGAATGCAAATGGCGAGCTCATGGGTTTATTTGAAATG
CAAATGGGGATTCCCCAGGGGACTTTCCAAGATGATGCAATGCATTATCTTAT
TCTAAGGTGAGGTAACTATCCTAGCTATCTTCCAAGGAAAGTCCCATTTGAAA
TGCAAATTAGAAAAGAGGAAGGAAATTGCCTTTTCTGACCATTTTCATCATGT
TGCTTTTCTGGGAAATACATCTCAAATTCTTGAAACATGCTTTTCCCATGCTC
ATACCTGTTAGCCCATCTCTCCCAGTTTGAATCTCTGATTTGAAATGCAAATT
CTTCCAATTTCCATTTTTTATGAGATGACCTATGATTTGCATTAAATAACTTTG
GAAAAATTGAAGTTCTTGAGAAACGTTGAC
P54 promoter nucleotide sequence (SEQ ID NO: 68):
TATGAGATGACCTATGATTTGCATTAAATAACTTTGGAAAAATT
GAAGTTCTTGAGAAACGTTGACTTTTGATGGGAAAATTGAGATGCAGGTTTG
GAAAAAGGCAAACCCTGTGACCTAGTGATCATAAACAACAGATAAACTATGG
GTCTCTGTAAAATAGATTGTTGGGAAGGTAACATTTTTCCATGGTTTTGATTT
TTCCCAAAAGTATTTATGTATTGATTTAACTAAAAAGGTAGTGCATTCTTAGG
GTCCCCAGATGTCTGCCTTTTCCATTGATTTTCTTTCCTGCCCGCTCCTTAGAT
AATATGTTCTAGAAAAGTGGAAACTGTTATTTTCCACTTTGTAACAGTACAGT
TACATTCATCTTTTTGGTAGTGAGCTCATGGGTTTCTCCACCAAGGTGACGAG
TGGAAAATCTGACTCAGAATTATCCCTTGTTATGCAAATCGCCCAAAGAGGA
AAATTTTCTTCCATGATCGTAGTTTTCCAAGTGCTGGGTCAGCAGCTCTTTGA
GAGCAGGACTCTATTTTCCAGTCTCTTTATAAGCTTTGATGCAGTGTCACACT
CGCTGTCTGCTGTCGGCTCAGGAAAAGTCTCTTTTCCAGACACAGCTCCCAAG
GTGGGGTGGGCGCAGCTGAGAAGCCAGAACCATCCAATATTCATGTTGGGAA
TGGCACAACTTTTTGGAAAAAAATACATTTATAAAATAAAACATACATTTTCT
ATGTATGTGATATATGTAGATGCATGATTTCCAGACACCTCTTAAGTGTACAG
CTGATGAATTTTTCCATCTGTATGTAGCCACCACCCAGCTCCACGTATTTTCA
GGATTTGAAATGCAAATAGGAAAGTCCCCTGTCAGTTTCTGGCCCACTTGTTT
GTCTAATAAGTGTTTGACTAATGATCAAATGGAAAATAGAATCTGGTGAAGG
TGTCCAAACGCAGAAAGGAAGGTGTTTACAGGGAGGACAATATGAGAGCCC
AGCACTTTACCTCCTGACCACACAGTAGCAGGTGTTTCTTTTCCAAAGATGAT
GTCATAGGGGACTTTCCATTTCCAATTTGAAATGCAAATAACCCATATGACTG
ATGGCATATTCAGATAATCGGGTCCCAGGTCTGGAAAAGCAGCCTTTTCCCC
ACGTTTCTTTCCCCACCTAGGGGATTCCCCAGGGGACTTTCCTGTGACTCATT
CGTGCAGCTTCTTCCAATTTCCGCTCGTTTTTCCTAGGGGACTTTCCATTTGAA
ATGCAAATGCATATTTCTACTAAAAATATGACTCCATCCCAAGAAATTGGTTT
TAAAGATTTGAGGAAAAACTGCTTTTAGATTTCAGAGAAAGAAAAAGAGTGT
GTTTCTACTTCCCCCTTTTAAATCTGATAACTTTTATTTTAATCAGGTTGACAT
T
P69 promoter nucleotide sequence (SEQ ID NO: 69):
GAGAGAAGTCGGGCTGTTTTTAAATGTAATATGATTTTCCATTT
ACTTATTTATCTTTAAAATGAGGTTGAGCCCAGGGCCCACGCTCCCTCCGGGC
ACAGCATCCGGAATTTTGCGTTTTTCCAGCGGGAAAACCAAGACGTGTTTTCC
CGTTTCCCACAACACTCGCTGTCTGCTGTCGGCTCAGGAAAAGTCTCTTTTCC
AGACACAGCTCCCAAGGTGGGGTGGGCGCAGCTGAGAAGCCTGATGAGGGA
TGTGGTATGGTAAAGGGGTTTTCCAGCCCCTATAATGGAATCTCCCCACCCTC
TTTCAGTAGATTTGCATTAAAAAAATAAACAAGTGTGGAAAATGATTACAAG
CACATTTAGAATATTTTGTATGCCACAGGGGGATTCCCCTGTTTTTGATGGGA
AAATTGAGATGCAGGTTTGGAAAAAGGCAAACCCTGTGACCTAGTGATCATA
AACAACAGGTTTTCCAATTTGAAATGCAAATCAGTTTCTGGCCCACTTGTTTG
TCTAATAAGTGTTTGACTAATGATCAAATGGAAAATAGAATCTGGTGAAGGT
GTCCAAACGCAGAAAGGAAGGTGTTTACAGGGAGGACAATATGAGAGCCCA
GCACTTTACCTCCTGACCACACAGTAGCAGGTGTTTCCTTAAGGCCTAAGATG
ATGCAATATTTCCAGCATATTTCTACTAAAAATATGACTCCATCCCAAGAAAT
TGGTTTTAAAGATTTGAGGAAAAACTGCTTTTAGATTTCAGAGAAAGAAAAA
GAGTGTGTTTCTACTTCCCCCTTTTAAATCTGATAACTTTTATTTTAATCAGGT
TGACATTTGTGACTCATTTTTCCTGAGGGATTAGTAACCCTTTCAAGTTACAT
CCTCCCCCTCCGTGGAGCAAAAGTGTTGCAAAGAGCAGTGACAACAGCTCAG
CACAGAGGAAAAACATTCCTAGGAGAGTCGGGGAGAACTACTGCCAACCGT
GTGCCAGGGGTTTTAGTGTAAATTATCTCACGCATTCCATTTGAAATGCAAAT
TCTCTTTTACTCATTTTTAATTGAAAATGGCCATGTGTTTTTCCAGGGACTTAA
TAATAATGTCTTTTAAAAATAAAGGATTTTCCATTTTCCAAAGGTGAGGTAAC
ATTTGAAATGCAAATATAATATGTTCTAGAAAAGTGGAAACTGTTATTTTCCA
CTTTGTAACAGTACAGTTACATTCATCTTTTTGGTAGTGGGGATTCCCCATTTG
AAATGCAAATTTACAGGCGTGAGCCACCGCGCCCGGCCTCCTCTCCTTTTCCA
AATTTATACATTTTGATTTTCTTAAAGGGGACTTTCCTTTTCCAAAGATGATGC
AATAGGAAAGTCCCATTTGAAATGCAAATAGGAAAGTCCCAGAACCATCCAA
TATTCATGTTGGGAATGGCACAACTTTTTGGAAAAAAATACATTTATAAAATA
AAACATACATTTTCTATGTA
P78 promoter nucleotide sequence (SEQ ID NO: 70):
GGAAAGATTCTATTGCATTTCTAACAGCTATGGAAAAATACAG
CATATTCTGCAAACCAAAATGTAGAACGAAGGAAGAATGATTTCTATATAAG
CATAACAGTAGGAGTTTTGGAAAATCTAGCATCAGAAGGCATCTGTTCTTCA
CTTTTATGTCTGTACAGCTGAAGAGTGAGTCGGTTAAAGGGAGAGCGAGACA
TGCAGGGAGATGGAAAAAGAATCTTTTAGAGAAAAAGTAAAAGCCCTGTAG
TATTTCCAAATGCTAGCAGGGGAATCCCCATAATATGTTCTAGAAAAGTGGA
AACTGTTATTTTCCACTTTGTAACAGTACAGTTACATTCATCTTTTTGGTAGTC
ACTCGCTGTCTGCTGTCGGCTCAGGAAAAGTCTCTTTTCCAGACACAGCTCCC
AAGGTGGGGTGGGCGCAGCTGAGAAGCCGGGGATTCCCCATCGAGCTATATT
TCCATTCGAGCTGATTTTTCCTAATCTGTGTGGTGCACAATCTAACCATGGAA
AAACTTCTGTTAGCTTCACTATGAATGGCATTAAGATACTATGCAAATCGAAA
CGCTGGAAACACTGAGTAATCAGATCAAGTCATGTTTCCATTTGAATGCAAA
TGGATACAGAAGGCGTGGAGGAATTTTGATGGGAAAATTGAGATGCAGGTTT
GGAAAAAGGCAAACCCTGTGACCTAGTGATCATAAACAACAGGACACCTCTT
AAGTGTACAGCTGATGAATTTTTCCATCTGTATGTAGCCACCACCCAGCTCCA
CGTATTTTCAGGGGGGATTCCCCATTTGAAATGCAAATGAATGCCGCAGATG
TACATGCTCCCGCAGATCTATATTTTCCAATGTTGTTAACATCAGCCAGCTGG
CAATCTACAACCTGTCTATTTGAAATGCAAATATTTCCAGCTATCATTGTCTT
CCAAGGAAAGTCCCTAGAAAAGAGGAAGGAAATTGCCTTTTCTGACCATTTT
CATCATGTTGCTTTTCTGGGAAATACATCTCAAATTCTTGAAACATGCTTTTCC
CATGCTCATACCTGTTAGCCCATCTCTCCCAGTTTGAATCTCTGTCTTCCAAGT
GCTGGGTCAGCAGCTCTTTGAGAGCAGGACTCTATTTTCCAGTCTCTTTATAA
GCTTTGATGCAGTGTCAATTTGAAATGCAAATAGGAAAGTCCCAAGGTGAGG
TAACATGTGACCGTAGCTTTTCCAAGGGGACTTTCCTATGAGATGACCTATGA
TTTGCATTAAATAACTTTGGAAAAATTGAAGTTCTTGAGAAACGTTGAC
P86 promoter nucleotide sequence (SEQ ID NO: 71):
ATGGGGTCCCAGCTCACCGTTATAACCAGTTATTTTTCCATTGT
AAAAGGGGTCTCAATTTACCATTATACCCCACAGCTGACACCTCTTAAGTGTA
CAGCTGATGAATTTTTCCATCTGTATGTAGCCACCACCCAGCTCCACGTATTT
TCAGGGAGCTCATGGGTTTCTCCACCAAGGTGACGAGTGGAAAATCTGACTC
AGAATTATCCCTTGTTATGCAAATCGCCCAAAGAGGAAAATTTCAGCTGAAG
AGTGAGTCGGTTAAAGGGAGAGCGAGACATGCAGGGAGATGGAAAAAGAAT
CTTTTAGAGAAAAAGTAAAAGCCCTGTAGTGGAAAACTTCCTTGGTGGAGAA
ACCCATGAGCTCATCTGGAGGAGAATGATTTCTATATAAGCATAACAGTAGG
AGTTTTGGAAAATCTAGCATCAGAAGGCATCTGTTCTTCACTTTTATGTCTGT
ACATGTTTCCATTTGAATGCAAATGGATACAGAAGGCGTGGAGGAAAAACTG
TTTCCTGTTCCAATAGACGCCCTTTTTGAATGCAAATGGGAGGGATTAGTAAC
CCTTTCAAGTTACATCCTCCCCCTCCGTGGAGCAAAAGTGTTGCAAAGAGCA
GTGACAACAGCTCAGCACAGAGGAAAAACATTCCTAGGAGAGTCGGGGAGA
ACTACTGCCAACCGTGTGCCAGGGGTTTTAGTGTAAATTATCTCACGCATTCC
TGTGACTCATATGCGTATAGCTGCATTTCCATGATCATATGCTATGCAATATG
CAAATTAGATTTGCATTAAAAAAATAAACAAGTGTGGAAAATGATTACAAGC
ACATTTAGAATATTTTGTATGCCACAGCTGTCAGTTTCTGGCCCACTTGTTTGT
CTAATAAGTGTTTGACTAATGATCAAATGGAAAATAGAATCTGGTGAAGGTG
TCCAAACGCAGAAAGGAAGGTGTTTACAGGGAGGACAATATGAGAGCCCAG
CACTTTACCTCCTGACCACACAGTAGCAGGTGTTTCTGTGACTCATAATGATG
CTCGCTTTTCCAATGATAGCCCTGAGGGGATTCCCCATAATATGTTCTAGAAA
AGTGGAAACTGTTATTTTCCACTTTGTAACAGTACAGTTACATTCATCTTTTTG
GTAGTAGGGGACTTTCCTCTTCCAAGGAAAGTCCCAAGATGATGCAATATGC
CTATCTATCTTCCAATTTGAAATGCAAATGCATCTGAGGTCCCAAAATGTGAT
GGTTTAGCGGCAGCTGCTTGTAGGGGTATTTTTGGTTTCCTGCTTCTTTTTTCC
CTTCTAAGTAAAGACAAAATAACTAGATCCCTCCACCCCACCACCCCATCATT
TCGTTGTTGTTTAACAAGTGTTGAGTAAATTTGAAATGCAAATTTTTCCATGA
TCTATTACTCTTCCAGTAGCTATATATTTCCATCTAGAAATCAACTGTGTATCT
GCTTGCAGTGTTTTTCCATTATAATAGTTTTTAATCCCTGA
P87 promoter nucleotide sequence (SEQ ID NO: 72):
GGAAAACTTCCTTGGTGGAGAAACCCATGAGCTCATCTGGAGG
ATGCAAATGGATACAGAAGGCGTGGAGGAAAAACTGTTTCCTGTTCCAATAG
ACGCCCTTTTTGAATGCAAATGGCGAGCTCATGGGTTTAGGTTACCCATTTCT
GTATTTCCTTGCAAAATTTTCCAGGTTGGCCGGGCGCGGTGGCTCACGCCTGT
AATCCCAGTGTTTTTGATGGGAAAATTGAGATGCAGGTTTGGAAAAAGGCAA
ACCCTGTGACCTAGTGATCATAAACAACAGGTGTGACTCATTCTAGAAATCA
ACTGTGTATCTGCTTGCAGTGTTTTTCCATTATAATAGTTTTTAATCCCTGATT
TTCCATCCACCAAGGTGACGAGTGGAAAATCTGACTCAGAATTATCCCTTGTT
ATGCAAATCGCCCAAAGAGGAAAATTTGTTTCATAGAACATAGGGGATTCCC
CCTGTCAGTTTCTGGCCCACTTGTTTGTCTAATAAGTGTTTGACTAATGATCA
AATGGAAAATAGAATCTGGTGAAGGTGTCCAAACGCAGAAAGGAAGGTGTT
TACAGGGAGGACAATATGAGAGCCCAGCACTTTACCTCCTGACCACACAGTA
GCAGGTGTTTCTTTTCCAATGCAGCATGCTAAGATGATGCAATGACACCTCTT
AAGTGTACAGCTGATGAATTTTTCCATCTGTATGTAGCCACCACCCAGCTCCA
CGTATTTTCAGGATTTGCATATTTTTTTGTTACCCAAGCCTCACCTCACTGATA
TTTTCCAACCAAAATGGGGGCTAAATGGTCTGAAGAATGATTTCTATATAAG
CATAACAGTAGGAGTTTTGGAAAATCTAGCATCAGAAGGCATCTGTTCTTCA
CTTTTATGTCTGTAAAGATGATGCAATATGCAAGCTAGGATTCTTCCAATGAC
GAGGATAATATGCAAATGAGAGAAGTCGGGCTGTTTTTAAATGTAATATGAT
TTTCCATTTACTTATTTATCTTTAAAATGAGGTAGGGGACTTTCCAGGGGACT
TTCCAAGGTGAGGTAACATGCATGCATGCTTTTCCTGGGGATTCCCCTATGAG
ATGACCTATGATTTGCATTAAATAACTTTGGAAAAATTGAAGTTCTTGAGAAA
CGTTGACAGGAAAGTCCCATTTGAAATGCAAATAGGAAAGTCCCTGTGACTC
ATAGGGGACTTTCCTTTTCCTATATCGATCTATCTTCCATGTGACTATATTTGA
AATGCAAATTCTCTTTTACTCATTTTTAATTGAAAATGGCCATGTGTTTTTCCA
GGGACTTAATAATAATGTCTTTTAAAAATAAAGGA
P88 promoter nucleotide sequence (SEQ ID NO: 73):
TAGAAAAGAGGAAGGAAATTGCCTTTTCTGACCATTTTCATCAT
GTTGCTTTTCTGGGAAATACATCTCAAATTCTTGAAACATGCTTTTCCCATGCT
CATACCTGTTAGCCCATCTCTCCCAGTTTGAATCTCTGAGGAGTGTAGCTGTT
AATTGCGAGGCTTGCCTCAAGGTGGAAAACAGTATCGGTTTCCACTGCCACC
CCAGAGATAAACTATGGGTCTCTGTAAAATAGATTGTTGGGAAGGTAACATT
TTTCCATGGTTTTGATTTTTCCCAAAAGTATTTATGTATTGATTTACAGTTTCT
GGCCCACTTGTTTGTCTAATAAGTGTTTGACTAATGATCAAATGGAAAATAGA
ATCTGGTGAAGGTGTCCAAACGCAGAAAGGAAGGTGTTTACAGGGAGGACA
ATATGAGAGCCCAGCACTTTACCTCCTGACCACACAGTAGCAGGTGTTTCCTT
AAGGCCTATAATATGTTCTAGAAAAGTGGAAACTGTTATTTTCCACTTTGTAA
CAGTACAGTTACATTCATCTTTTTGGTAGTGACACCTCTTAAGTGTACAGCTG
ATGAATTTTTCCATCTGTATGTAGCCACCACCCAGCTCCACGTATTTTCAGGA
TGCAAATGGATACAGAAGGCGTGGAGGAAAAACTGTTTCCTGTTCCAATAGA
CGCCCTTTTTGAATGCAAATGGCGAGCTCATGGGTTTTTTTTGTTACCCAAGC
CTCACCTCACTGATATTTTCCAACCAAAATGGGGGCTAAATGGTCTGAATTTC
CAGCATCTGAGGTCCCAAAATGTGATGGTTTAGCGGCAGCTGCTTGTAGGGG
TATTTTTGGTTTCCTGCTTCTTTTTTCCCTTCTAAGTAAAGACAAAATAACTAG
ATCCCTCCACCCCACCACCCCATCATTTCGTTGTTGTTTAACAAGTGTTGAGT
AAAAGATGATGCAATTTTTCCTAGGGGACTTTCCTGTTTTTGATGGGAAAATT
GAGATGCAGGTTTGGAAAAAGGCAAACCCTGTGACCTAGTGATCATAAACAA
CAGGATTTGAAATGCAAATACTAAAAAGGTAGTGCATTCTTAGGGTCCCCAG
ATGTCTGCCTTTTCCATTGATTTTCTTTCCTGCCCGCTCCTTAGAGGAAAGTCC
CAATCTGTGTGGTGCACAATCTAACCATGGAAAAACTTCTGTTAGCTTCACTA
TGAATGGCATTAAGATAAGGAAAGTCCCAAGGTGATGTCAATATAGAAGTCG
AGCTTTTCCATGCAAGGGGACTTTCCCAGTGCCATTTTCCAATTTGAAATGCA
AATCAGCTGAAGAGTGAGTCGGTTAAAGGGAGAGCGAGACATGCAGGGAGA
TGGAAAAAGAATCTTTTAGAGAAAAAGTAAAAGCCCTGTAGTATTTGAAATG
CAAAT
P93 promoter nucleotide sequence (SEQ ID NO: 74):
CGCTGGAAACACTGAGTAATCAGATCAAGTCATGTTTCCATTTG
AATGCAAATGGATACAGAAGGCGTGGAGGAAAAACTGTTTCCTGTTTCCTTT
GTTGGTAGCTTCTTAGAAGAATGTCCTCTTTTCCACTCACCAGCTGTGGGTTG
TAGTCCGGGAAGTGGGAAACTGTCCACCAAGGTGACGAGTGGAAAATCTGAC
TCAGAATTATCCCTTGTTATGCAAATCGCCCAAAGAGGAAAATTTGTTTCATA
GAACATATGAGCCCAGGGCCCACGCTCCCTCCGGGCACAGCATCCGGAATTT
TGCGTTTTTCCAGCGGGAAAACCAAGACGTGTTTTCCCGTTTCCCACAAGGGG
ATTCCCCAGAATGATTTCTATATAAGCATAACAGTAGGAGTTTTGGAAAATCT
AGCATCAGAAGGCATCTGTTCTTCACTTTTATGTCTGTAACCGGACTGCATTT
CTCAAAGAGCTAATTCCACATTTTTCCAGGCAGATGAGAAACCTTACTGTGTG
GTAAATCCCACAAACAGGAATATGCAAATTAGCTACTATCAGCGGGGATTCC
CCCTTCGCATATTTTAGAAATACTTTATATTTTATAACTATATTTTCCAAGTTT
GTGCTTTAACTTTAATTTTCTTATAGGAAAGATTCTATTGCATTTCTAACAGCT
ATGGAAAAATACAGCATATTCTGCAAACCAAAATGTAGAACGAAGGAAGGG
GACTTTCCAGATTTGCATTAAAAAAATAAACAAGTGTGGAAAATGATTACAA
GCACATTTAGAATATTTTGTATGCCACAGGGGGATTCCCCAGCTGAAGAGTG
AGTCGGTTAAAGGGAGAGCGAGACATGCAGGGAGATGGAAAAAGAATCTTT
TAGAGAAAAAGTAAAAGCCCTGTAGTAGGGGACTTTCCTTTTCCATAGCGAT
CGATATTTCCAATGCAGCTATTTTTCCTAGGAAAGTCCCTTACAGGCGTGAGC
CACCGCGCCCGGCCTCCTCTCCTTTTCCAAATTTATACATTTTGATTTTCTTAA
ATAAACTATGGGTCTCTGTAAAATAGATTGTTGGGAAGGTAACATTTTTCCAT
GGTTTTGATTTTTCCCAAAAGTATTTATGTATTGATTTAATTTGAAATGCAAAT
GGGGATTCCCCTGTGAGTCATATGCATCGATTAGTGCTAAGGTGAGGTAACT
ATGCAGTCAGTAGTTTTCCTTCATCGAGGAAAGTCCCATGCATATTTCCAATT
TGAAATGCAAATTGTTTTTGATGGGAAAATTGAGATGCAGGTTTGGAAAAAG
GCAAACCCTGTGACCTAGTGATCATAAACAACAGGATTTGAAATGCAAAT
NBV eTF nucleotide sequence (SEQ ID NO: 75):
ATGCCTTCCACCTCTTTCCCTGTGCCCTCCAAATTTCCACTGGGG
CCTGCAGCAGCTGTGTTTGGAAGAGGGGAGACCTTAGGACCTGCACCAAGAG
CAGGGGGCACCATGAAGAGTGCAGAAGAAGAGCACTATGGCTATGCCTCATC
CAATGTGAGCCCAGCCCTACCCCTGCCCACTGCCCACTCCACACTACCAGCTC
CCTGCCACAATCTGCAGACTTCTACACCAGGCATCATCCCCCCAGCAGACCA
CCCCAGTGGGTATGGAGCAGCTCTGGATGGAGGCCCAGCAGGCTACTTCCTG
TCCTCTGGCCACACTAGACCAGATGGTGCCCCTGCACTGGAGAGTCCTAGAA
TAGAAATCACTTCCTGCTTGGGCCTCTACCACAACAACAACCAGTTCTTCCAT
GATGTGGAGGTGGAGGATGTTCTCCCATCTAGTAAAAGATCCCCATCCACTG
CAACACTGTCACTTCCTTCACTGGAAGCCTACAGGGACCCATCCTGTCTTAGC
CCTGCTTCTTCTCTGTCCTCCAGATCTTGTAATTCAGAGGCCTCTAGTTATGAA
AGCAACTATAGTTACCCTTATGCAAGTCCACAGACATCACCCTGGCAGAGCC
CTTGTGTCAGCCCCAAGACCACTGACCCTGAGGAAGGCTTCCCCCGGGGGCT
TGGAGCCTGCACCCTCCTGGGCAGTCCAAGACATTCTCCCAGTACATCCCCA
AGGGCATCAGTAACTGAGGAGTCCTGGTTGGGTGCTAGGTCCTCAAGGCCTG
CCAGCCCATGCAACAAAAGAAAGTACTCCCTGAATGGGAGACAGCCTCCTTA
CTCCCCACACCACAGCCCCACCCCTAGTCCCCATGGCAGCCCCAGGGTTTCTG
TGACAGATGATAGCTGGCTTGGAAACACAACCCAGTACACATCTTCAGCAAT
AGTAGCTGCCATCAATGCCCTGACCACAGACAGCAGCTTGGACCTGGGTGAT
GGGGTGCCAGTGAAGAGCAGAAAAACCACCCTGGAGCAGCCCCCCTCAGTG
GCTTTGAAGGTGGAACCTGTGGGAGAAGACCTTGGTTCACCACCACCTCCAG
CTGACTTCGCCCCAGAGGACTACAGCTCCTTCCAGCACATCAGGAAAGGGGG
TTTCTGTGACCAGTATCTGGCTGTTCCCCAGCATCCTTATCAGTGGGCCAAGC
CCAAACCCCTCTCACCTACATCCTACATGTCACCTACACTCCCTGCTCTGGAC
TGGCAATTACCTGGTAGCACCTCAGGATCAGGCAAGCCAGGATCAGGGGAAG
GAAGCACCAAGGGCATGCTGTGGCAGAAACCAACAGCTCCAGAACAGGCAC
CTGCCCCCGCCAGACCATACCAGGGTGTGAGAGTAAAGGAGCCAGTGAAAG
AGCTGCTGAGGAGGAAGAGGGGCCATGCCAGTAGTGGTGCTGCACCAGCAC
CCACAGCAGTGGTGCTGCCTCACCAACCTCTGGCCACCTACACCACTGTGGG
CCCCAGCTGCCTGGATATGGAGGGCTCTGTCAGTGCAGTCACAGAGGAGGCA
GCCCTCTGTGCAGGCTGGCTGTCTCAGCCAGGTGGAGGGGGGAGTGGAGGTG
GGGGGTCTGGAGGGGGTGGGAGCACAGCCCCACCTACAGATGTAAGCCTGG
GAGATGAGCTTCATTTGGATGGTGAAGATGTGGCCATGGCCCATGCAGATGC
TTTAGATGACTTTGATCTTGACATGCTTGGTGATGGTGACTCACCTGGCCCTG
GATTCACACCTCATGACTCAGCTCCCTATGGGGCCCTTGACATGGCTGATTTT
GAATTTGAGCAGATGTTCACTGATGCCTTAGGGATTGATGAATATGGTGGCT
GA
NBV eTF protein sequence (SEQ ID NO: 76):
MPSTSFPVPSKFPLGPAAAVFGRGETLGPAPRAGGTMKSAEEEHY
GYASSNVSPALPLPTAHSTLPAPCHNLQTSTPGIIPPADHPSGYGAALDGGPAGYF
LSSGHTRPDGAPALESPRIEITSCLGLYHNNNQFFHDVEVEDVLPSSKRSPSTATLS
LPSLEAYRDPSCLSPASSLSSRSCNSEASSYESNYSYPYASPQTSPWQSPCVSPKTT
DPEEGFPRGLGACTLLGSPRHSPSTSPRASVTEESWLGARSSRPASPCNKRKYSLN
GRQPPYSPHHSPTPSPHGSPRVSVTDDSWLGNTTQYTSSAIVAAINALTTDSSLDL
GDGVPVKSRKTTLEQPPSVALKVEPVGEDLGSPPPPADFAPEDYSSFQHIRKGGF
CDQYLAVPQHPYQWAKPKPLSPTSYMSPTLPALDWQLPGSTSGSGKPGSGEGST
KGMLWQKPTAPEQAPAPARPYQGVRVKEPVKELLRRKRGHASSGAAPAPT
AVVLPHQPLATYTTVGPSCLDMEGSVSAVTEEAALCAGWLSQPGGGGSGGG
GSGGGGSTAPPTDVSLGDELHLDGEDVAMAHADALDDFDLDMLGDGDSPGPGF
TPHDSAPYGALDMADFEFEQMFTDALGIDEYGG

### Example 4: Design Principles for Ectopic Gene Fragment Expression

The identification of foreign, self-, and neo-antigens presented by cells is an essential step in the prevention, diagnosis, and therapy of not only infectious disease, but also cancer and autoimmunity. Here, new methodologies are presented for generating unbiased in-frame synthetic antigen-presenting libraries for application in high-throughput TCR: peptide screening approaches. The antigens will be processed by the intracellular machinery, while avoiding the potential restrictions on spliced peptides and haplotype-specific presentation events. Once established, these libraries can be exploited in high-throughput screens to functionally identify neo-antigens together with their corresponding T cell receptor. The antigen-presenting library design presented here has four major advantages compared to published methods: HLA-haplotype independent, genome-wide, employs natural antigen processing, and simultaneously includes MHC class I and class II antigens in the screening.

### Materials & Methods:

### Antigen-Presenting Cells

B cells purified from human peripheral blood are incubated with infectious supernatant from the Epstein-Barr virus-producing cell line B-95. After several weeks of continuous culture, outgrown cells are passaged and expanded. It has been demonstrated that these cells continue to express both class II and class I HLA molecules and are functionally professional antigen-presenting cells.

### Gene Fragment (“Minigene”) Library Design Techniques for Use in High-Throughput Screening

The main advantage of using a synthetic library is to minimize sequence-derived bias during library cloning and transduction. To solve these problems, selected genes will process to generate gene fragments with the same length and normalized GC content. Each gene fragment that resulted from this process is called ‘minigene’.

The first step in the minigene generation process is to break down the amino-acid sequence up to the maximum fragment length. Fragments derived from the same protein will have an overlap of a 30 amino-acid length. Fragments shorter than the maximum length will be linked together using ‘GSGSGG’ sequence. This iterative process of break down and build-up will ensure that generated fragment will have the same amino-acid length. In the remaining fragments, a protein domain will be linked to reach the maximum fragment length.

After the back-translation steps the minigene sequences are generated. A scheme of the process is illustrated in FIG. 27. A minigene can be a gene fragment, two short genes linked together, a short gene linked with a gene fragment, or a gene linked with a protein domain.

A peptide barcode is added at the 3′ end to enable library NGS analysis. In FIG. 28 are shown some possible minigene structures. Following the approach mentioned above, two synthetic libraries were generated starting from a subset of 1000 human proteins. The first library was designed with a maximum length of 330 amino-acid residues, which results in a minigene library with 2096 elements. The second libraries have a maximum length fragment of 400 amino-acid and a total number of 2096 minigenes. In FIG. 29 the number of minigene with different lengths in the two libraries are plotted.

To test the ability of minigene containing antigen to activated T cells, a co-culture experiment was performed comparing minigene sequence to natural open reading frame (ORF). The DMF5 TCR and MLANA gene were selected for the experiment. As shown in FIG. 30, the minigene processing does not affect the antigen presentation and T cell activation.

Because minigenes in a screening library are large (1200 bp) and diverse, full-amplicon sequencing for the identification of positive interactions can be difficult. In particular, sequencing and amplification biases negatively influence the experimenter's ability to isolate the correct sequence, even when the cellular interaction was present in the screen. In order to reduce this bias, each minigene is provided in a library with a unique molecular barcode using DNA. Nucleotide barcodes have been in use for some time to track DNA molecules; however, approaches that rely heavily on the sequence of the translated peptide, and using random nucleotide barcodes could introduce further unwanted effects. Therefore, a system was developed for barcoding individual members of a screening library that maintains an identical translated sequence across the entire library. Furthermore, the barcode generator ensures that a minimal Levenshtein distance between any two members of the library is maintained (as shown for example, FIGS. 36 and 37). This approach allows us to sequence a much smaller, more conserved amplicon after experimental enrichment of target cells, using the unique barcode sequence to identify the correct target from the library.

### Use of Tagged Signaling Accessory Molecules for the Identification of Minigene-Expressing Antigen-Presenting Cells

Expression of minigenes in EBV-transformed B cell lines is mediated by lentiviral-based overexpression. The number of copies of minigenes that subsequently integrate into the genome is defined by the viral multiplicity-of-infection (MOI), and occurs stochastically based on Poisson distributions. Thus, transduction of complex libraries is expected to result in either several integration events per cell when using maximal MOI, or result in few integration events alongside a large fraction of non-transduced cells upon diluting of viral supps, resulting in high inefficacy of any subsequent screen.

In order to minimize the amount of irrelevant integration events in cells containing the cognate minigene while at the same time conducting screens with a homogenously-transduced B cell populations, a method was developed in which a Flag-tagged surface receptor is expressed in tandem with the minigene. B cells are thus transduced to achieve 30% Flag⁺ cells, ensuring an average of a single integration event, and subsequently are enriched for Flag expression via magnetic-based cell enrichment.

Candidate surface receptors were chosen based on potential ability to further increase minigene-based T cell activation by TCR-cross activation. Thus, candidate surface receptors were screened both of ability to subsequently mediate successful enrichment of Flag⁺ minigene-expressing cells as well as their respective potency in mediating T cell activation. Combined, these were instrumental at highlighting OX40L-Flag and 41BBL-Flag as two surface receptors who are effectively expressed on transduced B cells, whose Flag-expression enables cell enrichment, and whose expression further potentiates minigene-based T cell activation.

### Minigene Vector Cloning

In order to induce minigene expression and subsequent presentation on HLA molecules, lentiviral particles driving expression of the respective minigene from a constitutive promoter were generated. Specifically, cognate or mismatch minigene were restriction-based cloned into the pLVX-EF1a-IRES-Puro vector (TAKARA). This vector was further modified to include a T2A polycistronic cassette in frame with the minigene, directly followed by an mTagBFP2 fluorescent reporter. Moreover, a CMA sequence was introduced immediately upstream of the minigene's coding sequence to facilitate chaperon-mediated autophagy and subsequent peptide presentation on Class II HLA molecules. This CMA-T2A-BFP backbone vector is thus named C2B (SEQ ID NO:77).

For the purpose of testing the different Flag-tagged surface receptors, InFusion-based cloning (TAKARA) was used to replace the mTagBFP2 reporter with the human codon optimized coding sequence of each candidate surface receptor, N-terminally or C-terminally tagged with a flexible 6× Flag to face the extracellular space. Specifically, the coding region of CD40, CD58, CD80, CD83, CD86, OX40L and 4-1BBL were cloned and tested.

### B Cell Culture and Minigene Transduction

BOLETH cells (Sigma, 88052031-1VL) were cultured in 6-well cell culture plates in RMPI medium (Gibco, #61870143) supplemented with 10% FBS (HyClone), 1% penicillin-streptomycin, 1% kanamycin, 50 μM β-mercaptoethanol, 1 mM sodium pyruvate, and 1 mM NEAA (Gibco, #11140050) at 37° C. humidified incubator with 5% CO₂. Cells were passaged every two to three days at a ratio of 1:2.

In order to induce minigene expression and presentation, lentiviral particles were generated via co-transfection into HEK 293FT cells in 6-well plates of 1.33 ug of the transfer vector alongside 1 ug of psPAX2 and 0.67 ug of pMD2.G accessory vectors using the TransIT VirusGEN transfection reagent (Mirus). Following 48 hours of incubation, supernatants were collected, filtered through a 0.45 μm PES filter. BOLETH cells were subsequently transduced by resuspending 1.5×10⁶/6 well plate in a 1:1 viral supernatant dilution in the presence of 8 μg/ml of Polybrene (Millipore) and 2 mM BX795 (Sigma). After 24 hours of incubation cells were washed from virus-containing medium and resuspended with full B cell medium. Functional experiments were conducted 4-5 days following transduction.

### Enrichment for Flag-Expressing B Cells

Minigene-transduced B cells were removed from culture plates and washed with cold Enrichment buffer (PBS+2% FBS+1 mM EDTA) to remove any residual media. Cell pellets were resuspended in same buffer supplemented with 0.05 μg of PE-conjugated aDYKDDDDK antibody (Biolegend) and 2 μl of human FcR blocker (Miltenyi) per 10⁶ total B cells. Following 20 minutes incubation, cells were washed with same buffer. Enrichment for PE-bound B cells was conducted using EasySep™ PE Positive Selection Kit II (STEMCELL) according to manufactures' instructions. Respective enrichment compared to the input cell fraction was subsequently determined using a FACS Aria II (BD) by probing for the PE signal.

### Functional Co-Culture Experiments

Co-culture experiments were done using round bottom 96 well plates. P10-NBV Jurkat and SKW3 reporter cells and B cells are seeded 1 to 1 ratio into each well. Next day the cells are spun down and stained for Flow cytometry.

### Algorithms for Computationally Rapid Codon Selection for High-Yield Protein Expression

The genetic code that dictates the translation of nucleotide triplets (i.e., codons) into amino acids is redundant in that most amino acids are coded for by several codons. Despite this apparent redundancy, non-random codon usage has been widely observed among different cell lines and organisms. The common and rare codons in a particular organism will vary from those of another (Novoa & Ribas de Pouplana, 2012, Trends in Genetics, Vol. 28, pp. 574-581). Moreover, not every codon composition will sustain the same expression level for a given protein (Plotkin & Kudla, 2011, Nature Reviews Genetics, Vol. 12, pp. 32-42). For this reason, codon optimization is used as a means to increase protein expression.

The Fast ALgorithm for Codon OptimizatioN (herein referred to as FALCON) is a tool written in Python that optimizes nucleotide sequences according to multiple parameters for a high-yield protein expression. A list of the features included in FALCON can be viewed in FIG. 34.

### FALCON Codon Optimization Process

FIG. 31 shows an overview of the FALCON algorithm.

The process starts with a list of amino acid sequences corresponding to the genes that will be optimized. The system in which the genes will be expressed is chosen (i.e., HEK293 cells), and the sequences are back-translated using a weighted random codon selection function. Multiple codon usage tables were developed for the expression systems that FALCON supports (see example in FIG. 32). These tables store the weights that reflect the single and bi-codon usage pattern in highly expressed genes of several tissues. Codons with a higher representation have higher weights. Additionally, the codon weights also reflect the abundance of their cognate tRNAs. Thus, more commonly used codons for which respective tRNAs abound are more likely to be selected. This supports efficient protein translation in cells, as it promotes the usage of tRNAs that are more readily available (Novoa & Ribas de Pouplana, 2012, Trends in Genetics, Vol. 28, pp. 574-581). Furthermore, before a codon is taken, their weights go through a series of adjustment layers that will modify their values according to the GC content of the growing nucleotide sequence and codon autocorrelation (i.e., the bias that exists in a number of codons that are correlated with themselves and other codons). The latter case means that, during the back-translation process, if a codon was already used by FALCON within the last 25 nucleotide triplets, its likelihood to be selected again will be proportionally higher to the distance of its last occurrence (distance in codons). An accepted model to explain the biological rationale for this phenomenon states that tRNA recycling supports efficient mRNA translation (Cannarrozzi et al., 2010, Cell, 141(2), 355-367; Godinic-Mikulcic et al., 2014, Nucleic Acids Research, 42(8), 5191-5201).

### GC-Content Correction According to a 4-Parameter Logistic Function

FALCON optimization allows for a desired GC content to be set by the user at the beginning of the optimization process. The GC content optimization is performed using a 4-parameter logistic function (4PL). In this function, the four parameters; A, B, C and D, and the independent variable ‘x’; are used to calculate the correction coefficient ‘y’, according to equation 1 (FIG. 33). The equation defines the change of the correction coefficient ‘y’ as a function of the GC content of the growing nucleotide sequence. The parameters are calibrated with the least squares method using the scipy.optimize library (Virtanen et al., 2020, Nature Methods, 17(3), 261-272), such that when ‘x’, the GC % of a growing sequence, is equal to the GC % specified by the user (GC-aim), the equation will result in a correction coefficient ‘y’=0 (i.e., no GC correction). Contrariwise, any deviation from the GC-aim will be countered by the correction ratio. The higher the deviation, the stronger the correction. If the GC %>GC-aim, the weights of the codons containing an A/T at the wobble position will be increased, and the codons containing a G/C will be disfavored proportionally. If the GC %<GC-aim, the opposite applies.

After 10 codons have been selected, the sequence is subjected to several motif avoidance steps. Restriction sites as well as other undesired patterns are eliminated.

The process of back-translation and motif avoidance is repeated continuously until the sequence is finished.

### Minimum Free Energy Optimization of the 5′ Start

One of the main rate-limiting steps in protein synthesis is at the translation initiation (Shah et al., 2013, Cell, 153(7), 1589). If the mRNA folds and forms a thermodynamically-stable structure, especially at the 5′ start, the coupling of the ribosome and the translation machinery can be hindered (Kudla et al., 2009, Science, 324(5924), 255-258). Thus, the potential for any stable structure forming at this position should be reduced. In order to promote an efficient translation initiation, FALCON optimizes the 5′ start of the first 20 codons, by generating 10 candidate partial sequences using the back-translation approach described above, and then selecting the sequence with the highest minimum free energy (MFE). A higher MFE is indicative of a less stable mRNA secondary structure, which correlates with higher expression levels (Jia & Li, 2005, FEBS Letters, 579(24), 5333-5337). The MFE is calculated using the open-source ‘seqfold’ package, developed by JJTimons (pypi.org/project/seqfold/). The optimized 5′ start will then be used by FALCON as a starting point, to generate 10 full candidate sequences. Subsequently, a step termed ‘Tournament style selection’ follows.

### Tournament-Style Selection.

The utilization of a weighted, yet random, codon selection approach in FALCON means that, if a gene is optimized multiple times, virtually every resulting sequence will differ to a degree from each other. This characteristic gives FALCON the advantage of generating multiple contesting full sequences, which can then be evaluated according to specific quality criteria. For each gene, FALCON creates 10 candidates, and selects the highest-ranking according to the following three parameters:
iv) Codon Adaptation Index (CAI) (Sharp & Li, 1987, Nucleic Acids Research, 15(3), 1281-1295) (equation 3; FIG. 33),
v) GC % (equation 4; FIG. 33) and,
vi) CpG dinucleotide content (equation 5; FIG. 33).

The CAI is a measure widely used to quantify codon preferences toward most common codons. A sequence that only uses the most common codons for a particular organism will have a CAI=1. Deviations from this usage will result in lower CAI values. FALCON first calculates the codon relative adaptiveness (CRA) of each candidate sequence (equation 2; FIG. 33), and then uses a slight modification to the CAI equation (Sharp & Li, 1987, Nucleic Acids Research, 15(3), 1281-1295), expressing the final result in % (equation 3; FIG. 33).

The final score is calculated according to equation 5 (FIG. 33).

The evaluation system has been tested and calibrated with a large set of tests such that, effectually in every case, the championed sequence has scored the highest in at least one or two of the assessed criteria, and it is among the top in the third. It is thus assured that the selected sequences will reliably be the most optimal from their batch.

### Multiprocessing and Algorithm Speed

A comparison of the FALCON algorithm is available in FIG. 35.

A last and important feature of FALCON algorithm is that it makes use of multiprocessing, allowing it to optimize several sequences concurrently. The adjustment-layers approach used by FALCON for sequence optimization is already in itself extremely fast. Other available algorithms such as the genetic algorithm used by COOL (Chin et al., 2014, Bioinformatics, 30(15), 2210-2212), or the simulated annealing and genetic algorithm used by EuGene (Gaspar et al., 2012, Bioinformatics, 28(20), 2683-2684), by contrast, take a considerably higher amount of time to find an optimal solution to the sequence optimization problem. For instance, Chin and colleagues report that COOL takes around 20 min to optimize a 500 amino acid sequence. Slightly faster, EuGene took over 24 minutes for a double-sized 1000 amino acid sequence. In the same machine in which the EuGene software was run, FALCON took over 4 seconds to optimize the same 1000 amino acid sequence. FALCON optimization was 360 times faster than EuGene, and included all of the parameters listed in FIG. 34. The improved speed that FALCON achieves using its unique approach and multiprocessing allows for the optimization of entire libraries consisting of thousands of sequences in less than a day.

The experimental results are now described

Minigene sequence libraries of different lengths were designed (FIG. 27-29). The minigene processing does not affect the antigen presentation and T cell activation (FIG. 30).

A FALCON algorithm, which employs an adjusted codon usage table for EBV immortalized B cells, was developed and optimized (FIG. 31-35).

FIG. 31 shows that a novel and extremely fast approach is used by the FALCON algorithm to generate codon-optimized nucleotide sequences. FALCON takes user-provided amino acid sequences and back-translates them in an iterative fashion. Each amino acid is back-translated to one or several codons, which are stored in a codon usage table along with their weights (i.e. selection probabilities). Before random weighted codon selection, the weights go through several modification layers according to GC-content and autocorrelation bias. Additionally, restriction sites and other motifs are controlled for. In this iterative optimization process, several candidates are built, among which, the one best fulfilling specific quality criteria is chosen.

FIG. 32 provides an example of an adjusted codon usage table used by the FALCON algorithm and designed for EBV immortalized B cells is shown. Amino acids are listed in their single-letter codes. Each codon has an assigned weight, which corresponds to its usage in highly expressed genes and the abundance of their tRNAs.

FIG. 33 provides equations used in the FALCON algorithm for sequence optimization are listed. Equation 1 is used to calculate the correction coefficient ‘y’, which modifies the weights of the codons as a function of the GC-content of the growing sequence and the user-specified GC target. Equation 2 is used to calculate the ratio of the occurrence of a given codon, relative to the occurrence of the most abundant codon for a given amino acid (Codon Relative Adaptiveness or C.R.A). In order to select an optimized nucleotide sequence from the 10 independently-generated candidates, their quality is evaluated according to equations 3-6. Equation 3 is used to calculate the codon adaptation index (C.A.I) based on the relative adaptiveness (RA) of each of its codons, equation 4 to calculate the GC-content score, equation 5 to calculate a score based on the number of CG dinucleotides, and equation 6 to calculate the final quality score.

FIG. 34 provides a summary of the most relevant characteristics featured by the FALCON algorithm is provided.

FIG. 35 provides a comparison between the characteristics of the FALCON algorithm and other commonly available tools. The FALCON algorithm takes more parameters into account and generates codon-optimized sequences over 250 times faster.

FIG. 36 shows the amino acid redundancy principle for peptide barcode sets.

A barcode generator that ensures that a minimal Levenshtein distance between any two members of the library is maintained was developed (FIG. 37).

CMA-Minigene-BFP (C2B) based constructs and modified, surface-receptor Flag-conjugated constructs (C2F) were designed (FIG. 38).

Flag-based surface receptor constructs can be used to successfully enrich for transduced B cells (FIG. 39A). B cells transduced with lentiviral-containing supernatants were subsequently enriched for Flag surface expression. Relative Flag surface expression was then determined using a flow cytometer (FIG. 39B).

OX40L and 4-1BBL overexpression potentiates minigene-based T cell activation (FIG. 40).

C2B vector sequence
(SEQ ID NO: 77)
ATGAAAGAGACCGCCGCAGCCAAGTTCGAAAGACAGCACATG
GACTCTAGCACTAGTGCTGGGATAGACGCTAACATCCTGAGCATAGGTG
GATCCGGAGGTGCGAGCGGCGAGGGGAGGGGCAGCCTTCTTACTTGCGG
AGATGTGGAAGAGAATCCAGGACCCGTGTCAAAAGGAGAGGAGCTTATC
AAAGAAAATATGCACATGAAGCTCTACATGGAGGGAACAGTGGACAACC
ACCACTTCAAATGTACCTCAGAAGGAGAAGGGAAGCCCTACGAGGGCAC
ACAGACCATGCGCATCAAGGTGGTGGAAGGTGGCCCCCTGCCCTTTGCC
TTCGACATCCTGGCCACCAGCTTCCTGTATGGCAGCAAAACCTTCATCA
ATCACACCCAGGGCATCCCCGACTTCTTCAAACAGAGCTTTCCAGAAGG
CTTCACCTGGGAGCGGGTCACCACCTATGAAGATGGAGGCGTGCTCACA
GCCACTCAGGACACCTCCCTGCAGGATGGCTGCCTCATCTACAACGTGA
AGATCCGCGGCGTCAACTTCACTTCCAATGGCCCTGTCATGCAGAAGAA
GACCTTGGGCTGGGAAGCCTTCACAGAGACCCTGTACCCAGCAGATGGG
GGCCTGGAGGGGCGGAACGACATGGCCCTGAAACTGGTGGGCGGCTCCC
ACCTCATTGCCAATGCCAAAACCACCTACAGAAGCAAGAAACCAGCCAA
GAACCTCAAGATGCCTGGGGTGTACTACGTGGATTACCGCCTGGAAAGA
ATCAAGGAGGCCAACAACGAGACGTATGTGGAGCAGCACGAGGTGGCTG
TGGCCCGCTACTGCGACCTGCCTTCCAAGCTGGGCCACAAGCTCAAC
Coding sequence of 6X flexible Flag construct
(SEQ ID NO: 78)
GGAAGCGGTGCAGGGGGCTCTGGCTCCGGTGCTGGCGGTTCAG
GTGATTACAAAGATGACGACGATAAGGGCGCCGGAAGCGGCGACTATAA
AGACGATGACGACAAAGGCGCAGGTAGCGACTATAAAGACGACGACGAT
AAGGGAGGAGCTGGTAGTGATTATAAAGACGATGACGATAAGGGGAGCG
GAGATTACAAGGATGACGACGATAAGGGAGCCGGAAGCGGAGACTACAA
AGACGATGATGACAAGGGCTCTGGCGCAGGGGGCTCTTCTTGA

Codon optimized candidate surface receptor accessory molecules with Flag flexible linker

CD40
(SEQ ID NO: 79)
GCGTGGATTCCTCTGCTTCTCCTGTTCCTGTCTCATTGCACAGGC
AGCCTCAGCGATTATAAGGATGATGATGACAAGGGTGCCGGCTCAGGCG
ATTACAAGGACGATGATGACAAGGGAGCCGGTAGTGACTACAAAGACGA
TGATGATAAAGGGGGTGCTGGAAGTGACTATAAAGACGACGACGACAAG
GGATCAGGCGACTATAAAGACGATGATGACAAAGGCGCCGGATCTGGGG
ACTATAAGGACGACGATGACAAGGGGAGCGGCGCAGGTGGCTCTGGGTC
CGGGGCCGGAGGCTCAGGCGAGCCCCCAACCGCATGTCGGGAGAAGCAG
TATCTGATTAATTCACAGTGCTGCTCCCTTTGCCAACCCGGGCAGAAAC
TTGTGTCCGACTGTACAGAGTTCACAGAGACAGAATGCCTGCCATGCGG
AGAATCTGAATTTCTGGACACATGGAACCGGGAGACGCATTGTCATCAG
CATAAGTATTGCGACCCAAACCTTGGACTTCGGGTGCAGCAGAAAGGCA
CATCTGAGACCGATACCATTTGTACGTGCGAAGAAGGATGGCATTGCAC
CTCCGAAGCGTGCGAGTCCTGTGTGCTTCACAGGTCCTGCTCACCTGGG
TTTGGCGTGAAGCAGATCGCCACCGGCGTGTCCGACACCATATGTGAGC
CGTGTCCAGTTGGATTCTTCAGCAATGTGTCTAGCGCTTTTGAGAAATG
CCATCCTTGGACGAGTTGCGAGACCAAGGATCTCGTGGTGCAGCAGGCC
GGAACCAATAAGACCGACGTGGTTTGTGGACCCCAGGATAGGTTACGCG
CCCTGGTAGTTATTCCTATTATTTTTGGCATCCTTTTCGCTATACTGTT
GGTCCTGGTCTTCATCAAAAAGGTTGCTAAAAAGCCTACCAACAAAGCT
CCTCATCCTAAGCAGGAGCCACAAGAGATTAACTTTCCAGACGACTTGC
CCGGATCTAACACCGCAGCACCAGTGCAGGAAACTCTTCATGGTTGTCA
ACCTGTCACCCAGGAAGATGGGAAAGAGAGCCGAATCAGTGTACAAGAA
CGCCAGTGATAA
CD58
(SEQ ID NO: 80)
GCGTGGATTCCTCTGCTTCTCCTGTTCCTGTCTCATTGCACAGGC
AGCCTCAGCGATTATAAGGATGATGATGACAAGGGTGCCGGCTCAGGCG
ATTACAAGGACGATGATGACAAGGGAGCCGGTAGTGACTACAAAGACGA
TGATGATAAAGGGGGTGCTGGAAGTGACTATAAAGACGACGACGACAAG
GGATCAGGCGACTATAAAGACGATGATGACAAAGGCGCCGGATCTGGGG
ACTATAAGGACGACGATGACAAGGGGAGCGGCGCAGGTGGCTCTGGGTC
CGGGGCCGGAGGCTCAGGCTTCTCCCAACAAATCTACGGCGTGGTGTAC
GGGAATGTAACATTCCATGTGCCGTCCAATGTTCCATTAAAGGAGGTGC
TTTGGAAGAAGCAGAAGGACAAAGTCGCTGAACTGGAGAACAGCGAATT
CAGGGCCTTCAGCTCTTTCAAAAATCGGGTTTATCTGGACACTGTGTCC
GGGTCACTGACAATCTATAACCTGACCAGCTCCGACGAAGACGAGTACG
AAATGGAATCGCCAAACATCACGGATACCATGAAGTTCTTTCTTTACGT
GCTCGAATCATTACCTTCACCCACCCTAACATGTGCACTGACAAATGGA
AGCATTGAGGTGCAGTGTATGATCCCCGAGCATTACAACTCGCATCGTG
GCTTAATTATGTATTCTTGGGATTGTCCGATGGAACAGTGCAAGAGGAA
CTCTACCTCAATTTACTTTAAGATGGAGAATGATTTACCGCAAAAAATC
CAGTGTACCCTGAGCAACCCCCTTTTCAATACCACATCTAGTATTATCC
TGACTACTTGTATTCCTTCCTCTGGACACAGCCGCCACCGGTACGCCCT
TATCCCCATCCCTCTGGCTGTCATTACCACCTGTATTGTCCTCTATATG
AATGGTATTCTGAAATGCGACCGGAAACCAGACCGGACTAATTCTAATT
GATAA
CD80
(SEQ ID NO: 81)
GCGTGGATTCCTCTGCTTCTCCTGTTCCTGTCTCATTGCACAGGC
AGCCTCAGCGATTATAAGGATGATGATGACAAGGGTGCCGGCTCAGGCG
ATTACAAGGACGATGATGACAAGGGAGCCGGTAGTGACTACAAAGACGA
TGATGATAAAGGGGGTGCTGGAAGTGACTATAAAGACGACGACGACAAG
GGATCAGGCGACTATAAAGACGATGATGACAAAGGCGCCGGATCTGGGG
ACTATAAGGACGACGATGACAAGGGGAGCGGCGCAGGTGGCTCTGGGTC
CGGGGCCGGAGGCTCAGGCGTCATTCATGTAACCAAAGAAGTTAAAGAG
GTAGCCACACTGAGCTGTGGGCACAATGTAAGTGTAGAAGAGCTTGCTC
AGACACGGATTTACTGGCAGAAAGAGAAGAAAATGGTGCTGACGATGAT
GTCTGGCGACATGAATATTTGGCCCGAGTACAAGAATAGAACTATTTTC
AATATAGCGAATAATCTTAGCATAGTAATTCTGGCCCTGAGGCCATCTG
ACGAGGGGACATATGAGTGCGTGGTCCTTAAGTACGAGAAGGATGCCTT
CAAGCGCGAGCATTTGGCTGAGGTGACTCTCTCGGTCAAGGCCGATTTT
CCTACGCCCTCGATATCAGACTTTGAAATCCCCACCAGCAATATCCGAC
GGATTATATGCAGTACCTCCGGGGGATTTCCGGAGCCACATCTGTCTTG
GCTGGAGAACGGGGAAGAGCTCAACGCTATTAACACCACTGTATCCCAG
GACCCTGAGACCGAGCTGTACGCTGTGTCCAGCAAACTTGACTTTAACA
TGACTACTAACCACTCCTTCATGTGTTTAATAAAGTACGGCCACTTGAG
AGTGAATCAGACCTTTAACTGGAATACCACCAAACAGGAGCACTTTCCT
GATAACCTGCTGCCTTCCTGGGCGATCACTCTCATTTCTGTGAATGGAA
TTTTTGTAATTTGCTGTCTCACTTACTGTTTCGCCCCTCGGTGCCGAGA
AAGAAGGCGCAACGAGCGCCTTAGGCGCGAGTCTGTCCGCCCCGTG
CD83
(SEQ ID NO: 82)
GCCTGGATTCCACTGCTCCTGCTCTTTCTTTCCCATTGCACAGGG
AGCCTGAGTGATTACAAAGACGACGATGACAAGGGTGCTGGAAGCGGGG
ATTATAAAGATGACGATGATAAGGGGGCCGGAAGTGACTACAAAGACGA
CGATGATAAAGGAGGAGCAGGAAGCGATTATAAAGATGACGACGACAAG
GGGAGTGGGGACTACAAGGACGACGATGATAAAGGGGCTGGCTCTGGAG
ACTATAAGGATGATGACGACAAAGGCAGCGGTGCAGGTGGGTCTGGCTC
TGGTGCCGGCGGATCTGGTACCCCAGAAGTAAAGGTGGCCTGTTCGGAA
GACGTAGATCTGCCTTGCACCGCTCCCTGGGACCCCCAGGTGCCCTATA
CGGTCTCGTGGGTAAAACTGCTCGAGGGCGGGGAGGAAAGGATGGAAAC
ACCTCAGGAAGACCACCTGCGCGGTCAGCATTACCACCAGAAGGGCCAG
AATGGATCATTCGACGCCCCAAACGAGAGACCTTACAGTCTGAAGATTC
GGAATACCACAAGCTGTAACAGTGGCACATATAGGTGTACCTTACAGGA
CCCGGACGGACAGCGCAATCTATCCGGCAAAGTGATACTGAGAGTCACG
GGGTGTCCTGCCCAGCGGAAGGAAGAAACGTTTAAGAAATACCGGGCAG
AAATTGTGTTGCTTCTTGCCCTGGTCATCTTCTACCTTACCCTGATTAT
CTTTACTTGTAAGTTCGCAAGGCTCCAAAGTATCTTCCCTGACTTCTCT
AAGGCCGGGATGGAGAGAGCCTTTCTGCCAGTCACCTCCCCTAATAAGC
ACCTGGGTCTAGTAACACCACACAAGACAGAACTGGTT
CD86
(SEQ ID NO: 83)
GCGTGGATTCCTCTGCTTCTCCTGTTCCTGTCTCATTGCACAGGC
AGCCTCAGCGATTATAAGGATGATGATGACAAGGGTGCCGGCTCAGGCG
ATTACAAGGACGATGATGACAAGGGAGCCGGTAGTGACTACAAAGACGA
TGATGATAAAGGGGGTGCTGGAAGTGACTATAAAGACGACGACGACAAG
GGATCAGGCGACTATAAAGACGATGATGACAAAGGCGCCGGATCTGGGG
ACTATAAGGACGACGATGACAAGGGGAGCGGCGCAGGTGGCTCTGGGTC
CGGGGCCGGAGGCTCAGGCGCACCACTAAAGATTCAAGCCTATTTCAAC
GAGACAGCAGATCTTCCCTGCCAATTCGCTAATTCACAGAACCAGTCCT
TGTCTGAGCTAGTAGTCTTCTGGCAGGACCAGGAAAACCTGGTCTTGAA
TGAGGTGTACCTGGGTAAAGAGAAGTTCGACAGCGTACACTCAAAGTAC
ATGGGCCGCACCTCTTTTGATTCTGACAGTTGGACACTACGCTTGCACA
ATCTACAAATTAAGGATAAAGGCCTCTACCAGTGTATTATCCACCATAA
AAAGCCAACTGGCATGATCCGTATTCATCAGATGAACTCTGAACTCTCT
GTGCTTGCTAACTTCTCACAACCAGAAATAGTGCCCATTTCTAATATCA
CAGAGAACGTGTACATTAATCTGACCTGTTCCTCAATCCACGGCTACCC
CGAACCCAAAAAGATGTCCGTTCTGTTGCGGACCAAGAACTCAACTATC
GAATATGACGGCGTGATGCAGAAATCCCAGGATAACGTAACCGAGCTGT
ATGATGTAAGCATATCCTTGAGTGTGAGTTTTCCTGATGTCACCTCTAA
CATGACGATCTTCTGTATCCTCGAAACTGACAAAACGCGGCTCCTGTCC
AGCCCATTTTCCATAGAGCTAGAAGATCCCCAGCCCCCACCCGATCACA
TCCCCTGGATTACAGCCGTCTTGCCGACAGTGATCATATGCGTGATGGT
TTTTTGTCTGATCTTGTGGAAATGGAAAAAGAAGAAGCGGCCTAGGAAT
AGCTATAAATGCGGGACTAACACAATGGAGAGGGAGGAAAGCGAACAAA
CAAAAAAGAGAGAAAAAATCCATATCCCGGAGAGAAGCGATGAGGCACA
GAGGGTTTTCAAGTCTTCCAAAACTTCCTCTTGCGACAAGAGTGACACC
TGCTTCTGATAA
OX40L
(SEQ ID NO: 84)
GAGCGGGTGCAGCCTTTGGAAGAGAATGTCGGTAACGCCGCCC
GTCCCCGCTTCGAGCGCAATAAGTTACTGCTTGTGGCCTCTGTTATCCA
AGGGCTCGGTTTGCTTTTATGTTTTACCTACATCTGCCTGCACTTCTCT
GCCCTGCAAGTTAGTCACCGGTATCCCCGGATACAGTCTATCAAGGTCC
AGTTCACGGAATACAAAAAGGAGAAGGGATTCATATTAACTTCACAGAA
GGAGGACGAAATCATGAAAGTGCAGAACAACTCCGTGATCATCAACTGC
GACGGTTTCTACCTCATTAGCTTGAAGGGCTACTTCAGCCAGGAGGTGA
ACATTTCCCTTCACTACCAGAAAGACGAGGAACCTCTTTTCCAGCTGAA
GAAGGTACGCTCGGTGAACTCTTTAATGGTGGCCTCTTTGACATATAAA
GACAAGGTTTATCTGAATGTGACCACAGATAATACCTCTCTGGACGATT
TCCATGTGAATGGTGGTGAGCTCATACTGATCCATCAAAACCCGGGCGA
ATTCTGCGTACTCGGAAGCGGTGCAGGGGGCTCTGGCTCCGGTGCTGGC
GGTTCAGGTGATTACAAAGATGACGACGATAAGGGCGCCGGAAGCGGCG
ACTATAAAGACGATGACGACAAAGGCGCAGGTAGCGACTATAAAGACGA
CGACGATAAGGGAGGAGCTGGTAGTGATTATAAAGACGATGACGATAAG
GGGAGCGGAGATTACAAGGATGACGACGATAAGGGAGCCGGAAGCGGAG
ACTACAAAGACGATGATGACAAGGGCTCTGGCGCAGGGGGCTCTTCT
41BBL
(SEQ ID NO: 85)
GAATACGCTTCAGATGCCTCACTCGACCCAGAGGCACCCTGGC
CCCCGGCCCCCCGAGCCCGGGCATGTCGCGTGCTACCCTGGGCTCTGGT
CGCCGGCTTACTGTTGCTCCTGCTCCTGGCCGCAGCTTGCGCAGTCTTT
CTGGCTTGTCCGTGGGCAGTGTCTGGCGCAAGGGCATCTCCCGGAAGTG
CCGCAAGCCCAAGGCTTCGAGAGGGTCCGGAGCTGTCCCCCGATGACCC
CGCAGGCTTGCTCGACCTGCGGCAGGGGATGTTCGCTCAGTTGGTTGCA
CAGAACGTCTTATTGATTGATGGCCCTCTGTCGTGGTACTCTGATCCGG
GACTTGCCGGCGTTAGTTTGACCGGCGGACTGTCATACAAGGAAGACAC
CAAGGAACTTGTCGTCGCTAAGGCCGGGGTGTACTATGTATTTTTCCAA
CTCGAGCTCCGCAGAGTCGTGGCGGGCGAAGGCTCGGGTAGCGTCAGCC
TTGCACTACACCTTCAACCTCTGCGGTCAGCTGCCGGCGCAGCAGCACT
CGCTTTGACAGTTGATCTCCCACCTGCTAGTAGCGAGGCTAGAAACAGT
GCCTTCGGCTTCCAAGGGCGCCTTCTCCATCTGTCAGCCGGACAGAGAC
TTGGCGTCCACCTGCACACCGAAGCACGGGCACGCCACGCCTGGCAGTT
AACTCAGGGTGCCACCGTCCTGGGTCTCTTCAGAGTAACCCCAGAGATA
CCCGCCGGACTCCCTAGCCCACGCTCCGAAGGGTCCGGCGCAGGGGGAT
CAGGTTCCGGTGCCGGTGGGTCTGGAGACTACAAAGACGACGATGATAA
AGGGGCTGGTTCTGGGGATTACAAAGATGACGACGACAAGGGTGCCGGA
TCAGACTACAAAGATGACGATGACAAAGGAGGCGCCGGTTCAGATTATA
AGGACGATGACGACAAAGGCTCAGGTGACTATAAAGATGACGACGACAA
AGGCGCAGGGTCCGGAGACTACAAGGACGACGATGATAAAGGATCAGGA
GCCGGCGGTTCCTCT

### Example 5: Testing of Constitutive Promoters

Expression of a transgene from different promoters was tested. FIG. 41 demonstrates the transgene expression level. Human GAPDH, TSMB4X, and other genome-derived regulatory elements serve as promising constitutively active promoters.

### Example 6: Design Principles for 2-Cell Microfluidic Co-Culture Screening Devices

Described herein is a new microfluidic co-encapsulation device that enables prolonged, clog-free encapsulation with eventual distributed cell flow and improved cell encapsulation ratios. The current study investigates the benefits of an increase of overall height from 25 μm to 35 μm, an inverted oil inlet, enlarged cell filter regions, stream separator elements that ensure even distribution of cell flow along the entire cell filter area, a novel cell filter shape, an overall shape that supports laminar flow, and the inclusion of asymmetrical focusing loops that lead to a more even flow of longitudinally aligned cells (FIG. 50, FIG. 51).

One of the physical limitations of the microfluidic system used for this screening method is the natural behavior of Epstein-Barr virally immortalized B cells to form clumps, which are not amenable to single-cell encapsulation and are impeded by the narrow structure of the device channels. To co-encapsulate B and T cells in droplets, a microfluidic PDMS device is used (FIG. 42). Both cell types enter the microfluidic chip separately though punches in the PDMS. Upon entry, the cellular flow is dispersed to a larger area where the cells progress through a filter element consisting of regularly interspaced, diamond-shaped elements. This cell filter element should ideally dissociate cell clumps or block them from flowing into the narrower areas of the chip, where they would subsequently clog the channels. As the channels around the nozzle of the chip have a diameter of only 40 μm, clumps consisting of only small numbers of cells can block the chip entirely. For T cells, this does not present a major problem, as they tend to enter the device as single cells. B cells, however, are naturally prone to clumping. Accordingly, it has been a continuous problem that larger clumps of B cells enter the microfluidic chip and clog the narrow filter region or channels. Those which do manage to reach the nozzle area tend to do so as multi-cell clumps, compromising the quality of the screens. Therefore, a protocol was developed herein to inhibit B-cell clumping and thereby improve the flow of B cells through a microfluidic co-encapsulation device and improve single B-cell encapsulation ratios. Several reagents were tested to prevent the clumping of B cells. EDTA was found to successfully inhibit the clumping of B cells and highly improved cell flow in microfluidic devices at concentrations >1 mM (FIG. 48, FIG. 49A through FIG. 49C). However, as EDTA chelates calcium ions from the media, T-cell activation is inhibited, as extracellular calcium is essential for T-cell activation signaling. Therefore, a workflow was devised to include inhibition of B-cell clumping via introducing EDTA only to the B-cell fraction while restoring the baseline calcium concentration in the formed droplets by adding calcium to the T-cell fraction.

To enable a constant flow of single cells into the microfluidic device, it is necessary to keep the cells in suspension. Therefore, magnetic stirring is needed to avoid cellular aggregation and uneven distribution. In an OB-1 microfluidic flow controller device (ELVEFLOW), pressure is applied to a fluid suspension of cells through a tube which has an adaptor for 15 ml centrifuge tubes (specifically FALCON 352095). Due to the V-bottom shape of the centrifuge tube, it is not possible to keep the cells in suspension via magnetic stirring. To overcome this problem, a 5 ml round bottom polystyrene test tube (FALCON 352052) was placed inside the 15 ml centrifuge tube. (FIG. 47, FIG. 52). With this setup, it is possible to have the required connection with the OB-1 and ensure the pressure flow while keeping the cells in suspension without adding any further chemicals or reagents to the cells.

The materials and methods are now described.

### Film Photomask Design

Computer-aided photomask designs were printed on high-resolution photolithography film photomasks.

### Wafer Fabrication

Depending on the desired channel depth, a specific SU-8 photoresist was used. All soft lithography work was conducted in clean room facilities. For a channel depth of 25 μm or 35 μm, SU-8 2025 photoresist was used. A specific guideline can be found online (https://kayakuam.com/products/su-8-2000/). Coating of the silicon wafer with photoresist was conducted by centering the wafer on a spin coater (Spin150 wafer spinner) and dispensing approximately 3 ml of photoresist onto the wafer. To equally disperse the photoresist along the wafer, the spin coater's rotational speed was set to 500 rpm (at an acceleration rate of 500 rpm/s) for 15 s, followed by an increase of the rotational speed to 3000 rpm for achieving a 25 μm coating, or 2000 rpm for achieving a 35 μm coating (at an acceleration rate of 500 rpm/s) for 30 s. Afterwards, the wafer was removed from the spin coater and baked at 65° C. for 1 min and then at 95° C. for 3 min (pre-bake) on a hotplate. Once the wafer cooled to RT, it was illuminated to constant ultraviolet (UV) light using a SUSS MICROTEC Mask Aligner MA6 with the following settings: mask aligner pressure: check if 0.8 bar or higher, wedge error compensation (WEC) pressure: adjustment to 0.15 bar (as indicated on the chuck), alignment gap: 30 μm, exposure type: hard, work type: cont (contact), upset offset: 10 s, HC wait: 10 s, exposure time: 20 s.

The film photomask was taped to a soda-lime glass plate. This was then brought in contact with the wafer and the UV source of the mask aligner was turned on. Following UV exposure, the wafer was subsequently baked at 65° C. for 1 min, then at 95° C. for 3 min (post-bake) on a hotplate. For developing the design and removing residual (non-exposed) photoresist, the wafer was submerged in PGMEA (propylene glycol monomethyl ether acetate) in a crystallizing dish and gently agitated for 4 min. The wafer was then removed from the crystallizing dish, rinsed with isopropanol and dried using a nitrogen gun. After developing, the wafer was baked at 150° C. for 10-30 min on a hotplate to fully resolve the features and remove tension-based cracks in the design.

### Production of the PDMS Device

The master wafer was rinsed with isopropanol and dried with nitrogen, then subsequently placed in the center of a plastic petri dish. PDMS base and curing agent were mixed in a 9:1 ratio (w/w) (Dow SYLGARD™ 184 Silicone Elastomer Kit), mixed well, and depressurized in a vacuum chamber to remove trapped air bubbles. Per PDMS mold, approximately 30 g of mixture were applied. After removal of a majority of air bubbles, the PDMS was poured onto the wafer in a plastic petri dish. The PDMS was cured overnight at 65° C. The next day, the PDMS (including the wafer) was cut out of the petri dish using a scalpel and carefully peeled off the wafer. The wafer was cleaned with isopropanol and stored at room temperature for future use. Using a 0.75 μm diameter biopsy punch, holes were created at each fluid inlet or outlet of the PDMS mold. 75 mm×50 mm glass slides were pre-cleaned in parallel by submerging in isopropanol and sonicating for 10 min. The PDMS mold was then positioned on the glass slide and hybridized into it using oxygen plasma with the following settings: plasma duration: 12 s, plasma power: 90%, process pressure: 0.3 mbar, gas supply duration: 60 s, gas type: O₂, flushing duration: 10 s, venting duration: 30 s. Following the oxygen plasma treatment of the PDMS (channel side facing up) and the glass slide, the two parts become irreversibly attached.

### AQUAPEL Treatment of Microfluidic Devices

For AQUAPEL treatment, the microfluidic chip containing the droplet encapsulation design was connected to a pressure control unit (ELVEFLOW OB1mk3 Flow Controller) which in turn is attached to a pressure pump. This allows flushing the channels with the desired fluid at a pre-defined pressure. The chip was placed on a microscope to enable visualization of the different processes. In order to treat the glass surface and facilitate laminar flow of aqueous fractions, the different channels were flashed with AQUAPEL prior to conducting encapsulation experiments. Specifically, AQUAPEL was filtered through a 0.22 μm filter and then flushed into all inlets and channels of the device using a syringe. Subsequently, the chip is similarly flushed with oil (3M NOVEC 7500) to remove the AQUAPEL agent. This was followed by drying of the design by flushing it with air. The chip is then left to dry overnight at room temperature prior to conducting encapsulation experiments.

### Droplet Encapsulation

For droplet encapsulation, Aquapel-treated microfluidic chips were used. The aqueous cell fractions containing the B and T cell fractions were each connected to the aqueous phase cell inlets using flexible PTFE tubing with an average diameter of 0.32 μm (ADTECH). Additionally, PICO-SURF detergent (SPHERE FLUIDICS) reconstituted to a 1% final concentration in 3M Novec 7500 oil was connected to the oil inlet using PTFE tubing. These were then connected to individual inlets of the pressure control unit. The chip was placed on a microscope to record droplet encapsulation. During encapsulation, cells were constantly mixed using a 5 mm×2 mm magnetic stir bar. Pressure was turned on for all inlets at the same time (detergent: 1000 mbar, aqueous inlets: 600 mbar). Depending on the filter design and height on the design, the pressure was adjusted to generate droplets of equal shape and diameter. Droplets were then collected in a microcentrifuge tube pre-filled with 400 μl oil and sampled after various encapsulation durations.

### Cell Culture

BOLETH cells (SIGMA, 88052031-1VL) were cultured in 6-well cell culture plates in B cell medium (RPMI 1640 (GIBCO, #61870143) supplemented with 10% FBS (HyClone), 1% penicillin-streptomycin, 1% kanamycin, 50 μM β-mercaptoethanol, 1 mM sodium pyruvate, and 1 mM NEAA (GIBCO, #11140050)) at 37° C. humidified incubator with 5% CO₂. Cells were passaged every two to three days at a ratio of 1:2. Reductions in B-cell clumping were tested with the following treatments: Gibco Anti-Clumping agent (GIBCO 0010057AE); EDTA (INVITROGEN #15575020); Opti-prep density gradient medium (STEMCELL TECHNOLGIES #07820); Cell clumping was evaluated in plates by time-resolved imaging using an INCUCYTE automated microscopy system (SARTORIUS). Jurkat cells were cultured in RPMI 1640+10% heat-inactivated FBS (HYCLONE) and 1% penicillin-streptomycin at 37° C. in a humidified incubator with 5% CO₂. Cells were split every two to three days at a ratio of 1:4.

### Plate Co-Culture of B and T Cells

For functional assays involving presentation of soluble peptides from the tetanus toxin (tetanospasmin, tetX), BOLETH cells were incubated overnight in the presence of 1 μM peptide. BOLETH and transgenic T-cell receptor-expressing Jurkat cells were collected and centrifuged at 300 g for 5 min. BOLETH cells were then resuspended in B-cell medium to a concentration of 1×10⁶ cells/ml and the respective peptide was added at a final concentration of 1 μM. Jurkat cells were comparably resuspended in B-cell medium to a final concentration of 3×10⁶ cells/ml. For the co-culture experiment, 50 μl of each cell suspension were mixed in a round-bottom 96-well plate to obtain a 3:1 ratio of T to B cells. Cells were incubated for 7 h and subsequently analyzed.

### Droplet Co-Culture of B and T Cells

One day prior to encapsulation, BOLETH cells were cultured in the presence of cognate or negative control peptides at a final concentration of 1 μM. Cells were collected the next day, centrifuged at 300 g for 5 min, then resuspended in B-cell medium at a final concentration of 1.4×10⁷ cells/ml. These were further supplemented with same peptide and with or without EDTA. TCR-expressing Jurkat cells were centrifuged at 300 g and resuspended in B-cell medium at a concentration of 4×10⁷ cells/ml. These were then further supplemented with respective concentrations of calcium. BOLETH, Jurkat, and detergent-containing fractions were connected to the microfluidic chip as described. Pressures were applied using an OB1 MK3+ and the droplet emulsion was collected into a 1.5 ml reaction tube containing oil.

Droplets were incubated at 37° C. in a 5% CO₂ humidified incubator. Following overnight incubation, droplet emulsions were disrupted by adding two volumes of Pico-Break 1 (SPHERE FLUIDICS). Tubes were quickly inverted several times and centrifuged for 15 s at 300 rpm to facilitate separation of the cell-containing fraction. This was then transferred into new tubes containing 1 ml FACS buffer (PBS+10% FBS+5 mM EDTA). Tubes were centrifuged for 5 min at 300 g and cell pellets were subsequently resuspended in 100 μl of FACS buffer and were analyzed via flow cytometry.

### Quantitation of B and T Cells in Droplets

To quantitate the ratios of encapsulated B and T cells in each design or upon addition of EDTA, cells were stained using membrane-penetrating fluorescent dyes. Specifically, BOLETH cells were stained with a red dye (AAT BIOQUEST CYTOTRADE RED) while Jurkat cells were stained with CFSE according to manufacturer protocols. Cells were subsequently washed in B-cell medium to remove any residual unbound dye. Cell pellets were then resuspended in B-cell medium at concentrations as described above and processed for droplet generation. Following encapsulation, 3 μl of droplet emulsion was then overlayed with 5 μl of 3M NOVEC 7500 oil on standard glass microscopy slides. Representative images were taken in the brightfield, Texas Red, and FITC microscope channels. Total droplets, B cell-containing droplets, T cell-containing droplets, and droplets containing both at least one T and one B cell were then counted from the images.

### Flow Cytometry

At the conclusion of a co-culture experiment, cells were first washed in cold FACS buffer and centrifuged at 300 g for 5 min. Supernatants were removed and pellets were then resuspended in FACS buffer supplemented with fluorescent dye-conjugated antibodies and incubated for 20 minutes at 4° C. in the dark. Antibodies were used at the following dilutions:

| Antibody | Fluorophore | Dilution | Product |
|---|---|---|---|
| α-CD20 | AF488 | 1:200 | BIOLEGEND #302316 |
| α-CD20 | PE/Cy7 | 1:200 | BIOLEGEND #302312 |
| α-CD3 | APC/Cy7 | 1:150 | BIOLEGEND #300318 |
| α-CD69 | PE | 1:200 | BIOLEGEND #310906 |
| α-HA | APC | 1:400 | BIOLEGEND # 901524 |

Following incubation, cells were washed again with cold FACS buffer and resuspended in 100 ul cold buffer prior to analysis. Flow cytometry analysis was conducted with a FACS ARIA II (BD BIOSCIENCES). Data analysis was subsequently performed using FLOWJO (BD BIOSCIENCES).

### Example 7: Transmembrane Domains for Class II Presentation

The identification of foreign, self-, and neo-antigens presented by cells is an essential step in the prevention, diagnosis, and therapy of a spectrum of disorders, including infectious disease, cancer, and autoimmunity. Here, new methodologies are presented for generating unbiased in-frame antigen presenting libraries for application in high-throughput TCR: peptide screening approaches. This simultaneously addresses class I and class II HLA (generally, MHC) complexes by non-professional and professional antigen-presenting cells. Furthermore, the antigens will be processed by the intracellular machinery, while avoiding potential issues with spliced peptides and haplotype-specific presentation events. Once established, these libraries can be exploited in high-throughput screens to functionally identify neo-antigens together with their cognate T-cell receptor. The antigen-presenting library design presented here has three major advantages compared to published methodologies: HLA-haplotype independence, MHC class II antigen inclusion, and genome-wide screening capability (Kula et al., 2019, Cell, 178(4):1016-1028.e13).

### Antigen Processing Attenuation is a Class II Antigen Specific

The platform aims to present both class II and class I antigens encoded by the same transgenic construct. The construct contains a constitutively active EF1α promoter which drives expression of the open reading frame (ORF) containing the minigene linked via a 2A viral peptide linker to mTagBFP2. The ORF is transcribed as a single mRNA but two separate proteins will be produced at a stoichiometric ratio; therefore mTagBFP2 is used as a fluorescence marker for transduction control as well as verifying the reading frame of the upstream ORF. The minigene is flanked by SpeI and BamHI restriction sites to facilitate synthetic library cloning.

A MLANA A27L transgene and DMF5 TCR (Abdel-Wahab et al., 2003, Cell Immunol, 224(2):86-97) were used as positive controls for class I presentation, while for class II presentation a fragment of the tetanospasmin protein, (TetX_400_1) was used together with a cognate TCR (TT2). Both minigenes were cloned in a lentiviral construct for B-cell transduction, cells were used in a co-culture experiment with their cognate TCRs. In both samples, clear TCR activation was detected, even for the class II positive control which was not expected, because class II presentation is generally associated with extracellular antigens (FIG. 54). Most interestingly, the BOLETH cells transduced with MLANA A27L can activate the T cells also 8 and 12 days after the transduction. However, for class II presentation, the T-cell activation potential decreases over time following APC transgenesis.

Constant mTagBFP2 expression levels in the B cells excluded the possibility of transduction biases and transgene silencing. Presentation biases were tested with a co-culture experiment in which freshly peptide-pulsed BOLETH cells were compared to re-pulsed transgenic BOLETH cells cultured over time (FIG. 55); both conditions led to strong T-cell activation, indicating that the ability of the cell to present loaded peptides is not affected by sustained transgenic expression of that epitope. With the previous results, transduction and presentation were excluded to be the cause of this phenomenon; thus it was concluded that the class II processing pathway itself plays a key role in this process, which was named “antigen processing attenuation”.

Lysosomal Targeting does not Improve Class II Antigen Processing

Class II antigens are processed and loaded on HLA molecules in the lysosome. To improve class II processing, three strategies were used to target the lysosome. The chaperone-mediated autophagy (CMA) pathway was targeted using the KFERQ sequence (Koga et al., 2011, Nat Commun 2, 386), the binding motif for the HSC70 chaperone protein that will translocate the minigenes through LAMP2. DC-LAMP is another lysosomal membrane proteins, N-term and C-term domains were used to anchor the minigenes in the membrane and localize it in the lysosomal lumen (Dominguez-Bautista et al., 2015, Eur J Cell Biol. 2015 March-April; 94(3-4):148-61). LIR domains are found in cargo proteins and interact with LC3 at the level of the phagophore formation; LIR domains were used to localize the minigenes to the lysosome at this step (Johansen et al., 2020, J Mol Biol, 432(1):80-103). (FIG. 56) Three LIR sequences were tested separately, ATG14 sequence was selected for comparison with other lysosomal constructs (FIG. 57). None of the mentioned strategies improved the T-cell activation profile over time with class II minigenes (FIG. 58).

### BCAP31 TM1 Sequence Induces a Strong and Stable T-Cells Activation

In a recent publication, a class II antigen screening experiment was performed cloning the ORF into a CD74 fusion protein instead of the CLIP peptide. When testing this construct, two key observations were made: the minigene activation was as strong as that with pulsed peptide and T-cell activation did not decrease over time (FIG. 59). This experiment confirmed that antigen processing attenuation does not involve the presentation machinery at the penultimate step before HLA loading. However, CD74 construct has the limitation of only being suitable for minigenes shorter than 100 AA (FIG. 60), which limits the possibility of generating a full-proteome library that can be screened at sufficient depth.

CD74 binds the HLA class II molecule in the ER, then the complex is translocated to the lysosome, where Cathepsin S cleaves CD74, leaving the CLIP peptide bound on class II HLA. HLA-DM releases the CLIP peptide and allows another peptide to bind class II HLA. To target the antigen presentation pathway, minigenes were cloned into a fusion with HLA-DM; this construct similarly failed to improve T-cell activation in a co-culture experiment (FIG. 61).

Bcap31 is an ER chaperone membrane protein, which is also involved in the ER-associated degradation (ERAD) pathway. The protein contains three transmembrane domains. Protein fragments were cloned and added to the N-terminus of the minigene in transgenic expression construct (FIG. 62). The first fragment used Bcap31_104_1 (length 104 AA and starting point 1 AA), which contains two transmembrane domains and an ER lumenal tail. Small-fragment fusions Bcap31_TM1_21_6 (BT1) and Bcap31_TM2_21_44 contain the first and second transmembrane domains, respectively.

Constructs were tested with a 100 AA tetanospasmin fragment containing the target of the TT2 TCR. When minigene-expressing cells and T-cells were co-cultured, the BT1 construct induced a strong T-cell activation, similar to the positive control. Moreover, the construct overcomes antigen processing attenuation and demonstrates stable T-cell activation over time (FIG. 63). The Bcap31_TM1 construct was tested with a 400 AA minigene containing another fragment of the tetanospasmin protein recognized by the TT7 TCR. BT1 induces strong T-cell activation even with a 400 AA minigene, and this activation is stable over time. (FIG. 64) The BT1 fusion construct can strongly and stably present a 100 AA minigene. An important step in the characterization of this construct is to test whether longer minigenes can also be processed. For this, the Tet93-containing minigenes, from 100 to 400 AA long, were subcloned into the BT1 constructs. The results are depicted as the ratio of T-cell activation with the BT1 construct to the 2B construct. For each construct, the results from three time points are shown. The BT1 construct greatly improves antigen presentation for the TetX_100_50 minigene; overall, the BT1 construct also improves T-cell activation for the shorter TetX_125_31 and TetX_150_22 minigenes (FIG. 65A). With respect to increasing minigene length, BT1 performed better than the CD74 fusion (which failed to present epitopes from minigenes longer than 175 AA).

However, the BT1 construct did not improve the presentation of the TetX_400_1 minigene. In this case, T-cell activation was lower in the BT1 construct relative to 2B. The experiment was expanded to include the TT7 and TT11 TCRs and their 400 AA cognate minigenes. TetX_400_916 (which contains the TT7 target at its C-terminus) was included in the experiment. In this co-culture series, the BT1 construct outperformed the 2B constructs and improved antigen presentation (FIG. 65B).

### Screening of Natural and Synthetic Transmembrane Domain

The BT1 construct significantly increases T-cell activation, but still has several limitations. These observations focused our attention on ER membrane proteins as a potential source of TM-spanning sequences able to target minigenes to the class II processing and presentation pathway. Here a functional screening strategy is presented to identify additional TM sequences that can further improve the BT1 construct results.

Transmembrane domains from ER protein sequences were downloaded from UniProt. The list contains 1410 sequences with lengths between 8 and 45 AA, with a modal length (n=977) of 21 AA. 800 sequences with a length of 21 AA were randomly selected for the screening. These were extended by positions −2, −1, +1, +2 from the TM to provide cytoplasmic/luminal anchors, giving a final sequence length of AA. The amino acid composition of these TM domains was analyzed; as expected, they show a clear enrichment of hydrophobic residues. Anchor positions, in contrast, were strongly enriched in hydrophilic and charged amino acids. With this data, an R script was written to generate synthetic TM sequences which maintain this composition. From the synthetic TM domain batch, 400 sequences were randomly selected for screening. The final library is composed of 400 synthetic and 800 natural TM domains. The sequences were back-translated and ordered as an oligonucleotide pool with the restriction sites required for cloning.

To subsample the library and assess individual sequences, a microscope-based screen was performed with 1152 individually picked transformants following bulk cloning of the entire library. The transduced BOLETH cells were co-cultured with T-cells expressing the cognate TT7 TCR, and T-cell activation was measured via image-based sfGFP intensity.

In all the three independent replicates, approximately 20 transmembrane sequences induced T-cell activation in two technical replicates, meaning 10% of the library sequences were able to induce synthetic class II presentation. These sequences outperformed BT1 construct, which induced a weaker T-cell activation (data not shown). Of these, fewer TM sequences induced a positive signal at both time points, suggesting not all presentation-promoting sequences could overcome antigen processing attenuation. In an initial round of screening, 576 transformants were tested and 5 of them were able to strongly activate T-cells at both time points (FIG. 66). These sequences will be tested in a co-culture validation experiment, and the best performing will be further tested with a larger TCR:antigen pair set. In a second round, an additional 576 transformants were screened for processing-promoting activity, of which one of the sequences from the first round was re-identified (DERL3 TM3) along with five more constructs which overcome antigen processing attenuation reasonably well for both the TT7 and TT11 TCRs (see FIGS. 67-75).

CMA
(SEQ ID NO: 86)
MKETAAAKFERQHMDSS
(SEQ ID NO: 87)
ATGAAAGAGACCGCCGCAGCCAAGTTCGAAAGACAGCACATGGACTCTA
GCA
DC LAMP
N-term
(SEQ ID NO: 88)
MPRQLRAAAALFARLAVILH
(SEQ ID NO: 89)
ATGCCGAGGCAGTTGAGGGCGGCGGCGGCGTTGTTTGCGAGGTTGGC
GGTGATATTGCAT
C-term
(SEQ ID NO: 90)
SSDYTIVLPVIGAIVVGLCLMGMGVYKIRLRCQSSGYQRI
(SEQ ID NO: 91)
AGCAGCGATTATACGATAGTGTTGCCGGTGATAGGGGCGATAGTGGTG
GGGTTGTGTTTGATGGGGATGGGGGTGTATAAGATAAGGTTGAGGTGT
CAGAGCAGCGGGTATCAGAGGATA
LIR-ATG4B
(SEQ ID NO: 92)
DSEDEDFEILSL
(SEQ ID NO: 93)
GATTCAGAAGATGAGGACTTTGAAATTCTGTCCCTG
LIR-ATG14
(SEQ ID NO: 94)
TDLGTDWENLPSPRFC
(SEQ ID NO: 95)
ACCGACTTAGGCACCGACTGGGAAAATTTACCCTCCCCCAGATTTTGT
LIR-ULK2
(SEQ ID NO: 96)
SCDTDDFVLVPHNISS
(SEQ ID NO: 97)
TCTTGTGATACTGATGACTTTGTTCTGGTGCCACATAACATATCATCC
CD74
N-term
(SEQ ID NO: 98)
MHRRRSRSCREDQKPVMDDQRDLISNNEQLPMLGRRPGAPESKCSRGAL
YTGFSILVTLLLAGQATTAYFLYQQQGRLDKLTVTSQNLQLENLRMKLP
KPPKPVSK
(SEQ ID NO: 99)
ATGCACCGTAGGCGGTCAAGATCATGCAGGGAAGATCAGAAGCCGGTAA
TGGACGACCAGCGGGACCTGATTAGCAACAATGAGCAGCTGCCCATGCT
CGGCAGACGACCCGGGGCTCCAGAGAGTAAATGTTCTAGGGGCGCTCTT
TACACCGGGTTTAGTATTCTTGTGACTTTATTACTGGCTGGGCAGGCTA
CTACCGCATATTTCCTGTACCAACAGCAGGGCCGTCTGGATAAGCTGAC
AGTGACATCACAGAACTTGCAGCTCGAGAACCTGAGAATGAAGCTCCCG
AAGCCTCCGAAGCCTGTTTCGAAA
C-term
(SEQ ID NO: 100)
QALPMGALPQGPMQNATKYGNMTEDHVMHLLQNADPLKVYPPLKGSFPE
NLRHLKNTMETIDWKVFESWMHHWLLFEMSRHSLEQKPTDAPPKESLEL
EDPSSGLGVTKQDLGPVPM
(SEQ ID NO: 101)
CAGGCCCTGCCCATGGGAGCCCTCCCCCAGGGCCCCATGCAGAACGCCA
CCAAGTACGGCAACATGACCGAGGACCATGTGATGCATCTGCTGCAGAA
CGCCGATCCTCTGAAGGTGTACCCACCGCTGAAGGGGTCCTTCCCAGAG
AACCTTAGACATTTGAAGAACACGATGGAGACCATCGATTGGAAGGTCT
TTGAATCCTGGATGCATCACTGGCTGCTGTTCGAGATGTCCAGGCACTC
CCTTGAGCAGAAGCCCACAGACGCCCCGCCTAAAGAGTCCTTGGAGTTA
GAGGACCCATCCTCTGGTCTTGGAGTCACGAAGCAGGATCTCGGACCTG
TGCCAATG
HLADMA_1_2
N-term
(SEQ ID NO: 102)
MGHEQNQGAALLQMLPLLWLLPHSWAVPEAPTPMWPDDLQNHTFLHTVY
CQDGSPSVGLSEAYDEDQLFFFDFSQNTRVPRLPEF
(SEQ ID NO: 103)
ATGGGACATGAGCAGAACCAGGGCGCTGCTTTACTGCAGATGCTGCCTT
TACTTTGGCTCCTGCCTCACAGTTGGGCTGTGCCCGAAGCCCCCACTCC
CATGTGGCCCGACGATCTGCAGAATCATACTTTTCTGCATACAGTGTAC
TGCCAGGATGGAAGCCCTAGCGTTGGCCTCTCTGAGGCCTACGATGAAG
ACCAGCTGTTCTTCTTTGACTTCTCACAGAATACCAGAGTTCCAAGATT
GCCCGAATTT
C-term
(SEQ ID NO: 104)
ADWAQEQGDAPAILFDKEFCEWMIQQIGPKLDGKIPVSRGFPIAEVFTL
KPLEFGKPNTLVCFVSNLFPPMLTVNWHDHSVPVEGFGPTFVSAVDGLS
FQAFSYLNFTPEPSDIFSCIVTHEIDRYTAIAYWVPRNALPSDLLENVL
CGVAFGLGVLGIIVGIVLIIYFRKPCSGD
(SEQ ID NO: 105)
GCCGACTGGGCCCAGGAGCAGGGAGACGCCCCAGCCATTTTGTTCGACA
AGGAGTTCTGTGAGTGGATGATTCAGCAGATTGGCCCCAAATTGGACGG
GAAGATTCCCGTATCACGCGGATTCCCGATTGCGGAGGTATTTACCCTG
AAACCACTGGAATTTGGAAAGCCTAACACACTGGTGTGTTTCGTGAGCA
ACCTGTTCCCTCCCATGCTGACGGTGAACTGGCACGATCACTCCGTGCC
TGTGGAAGGCTTCGGCCCTACATTTGTGTCTGCAGTGGACGGGCTATCT
TTCCAGGCTTTCAGCTATCTGAATTTCACGCCCGAGCCCAGCGACATTT
TCTCCTGCATTGTAACACACGAGATCGACCGGTATACTGCTATAGCCTA
CTGGGTGCCCCGCAACGCTCTGCCGAGTGATCTGTTGGAGAACGTGTTG
TGCGGGGTAGCTTTCGGGTTGGGGGTGCTTGGAATTATTGTGGGCATCG
TGCTGATTATCTATTTTAGGAAACCTTGCTCAGGGGAC
HLADMA_2_3
C-term
(SEQ ID NO: 106)
MGHEQNQGAALLQMLPLLWLLPHSWAVPEAPTPMWPDDLQNHTFLHTVY
CQDGSPSVGLSEAYDEDQLFFFDFSQNTRVPRLPEFADWA
(SEQ ID NO: 107)
ATGGGACATGAGCAGAACCAGGGCGCTGCTTTACTGCAGATGCTGCCTT
TACTTTGGCTCCTGCCTCACAGTTGGGCTGTGCCCGAAGCCCCCACTCC
CATGTGGCCCGACGATCTGCAGAATCATACTTTTCTGCATACAGTGTAC
TGCCAGGATGGAAGCCCTAGCGTTGGCCTCTCTGAGGCCTACGATGAAG
ACCAGCTGTTCTTCTTTGACTTCTCACAGAATACCAGAGTTCCAAGATT
GCCCGAATTTGCCGACTGGGCC
N-term
(SEQ ID NO: 108)
QEQGDAPAILFDKEFCEWMIQQIGPKLDGKIPVSRGFPIAEVFTLKPLE
FGKPNTLVCFVSNLFPPMLTVNWHDHSVPVEGFGPTFVSAVDGLSFQAF
SYLNFTPEPSDIFSCIVTHEIDRYTAIAYWVPRNALPSDLLENVLCGVA
FGLGVLGIIVGIVLIIYFRKPCSGD
(SEQ ID NO: 109)
CAGGAGCAGGGAGACGCCCCAGCCATTTTGTTCGACAAGGAGTTCTGTG
AGTGGATGATTCAGCAGATTGGCCCCAAATTGGACGGGAAGATTCCCGT
ATCACGCGGATTCCCGATTGCGGAGGTATTTACCCTGAAACCACTGGAA
TTTGGAAAGCCTAACACACTGGTGTGTTTCGTGAGCAACCTGTTCCCTC
CCATGCTGACGGTGAACTGGCACGATCACTCCGTGCCTGTGGAAGGCTT
CGGCCCTACATTTGTGTCTGCAGTGGACGGGCTATCTTTCCAGGCTTTC
AGCTATCTGAATTTCACGCCCGAGCCCAGCGACATTTTCTCCTGCATTG
TAACACACGAGATCGACCGGTATACTGCTATAGCCTACTGGGTGCCCCG
CAACGCTCTGCCGAGTGATCTGTTGGAGAACGTGTTGTGCGGGGTAGCT
TTCGGGTTGGGGGTGCTTGGAATTATTGTGGGCATCGTGCTGATTATCT
ATTTTAGGAAACCTTGCTCAGGGGAC
HLA_DMB_2_3
C-term
(SEQ ID NO: 110)
MITFLPLLLGLSLGCTGAGGFVAHVESTCLLDDAGTPKDFTYCISFNKD
LLTCWDPEENKMAPCEFGVLNSLANVLSQHLNQKDTLMQRLR
(SEQ ID NO: 111)
ATGATCACTTTCCTGCCCCTGCTGTTAGGGTTAAGCCTGGGGTGTACCG
GGGCAGGAGGCTTCGTTGCACATGTTGAGAGCACTTGTCTGCTGGACGA
CGCAGGAACACCAAAGGATTTCACGTACTGCATTAGCTTCAACAAAGAC
CTGCTTACATGCTGGGACCCTGAGGAGAACAAGATGGCCCCTTGCGAGT
TCGGCGTGTTAAACAGCCTGGCTAACGTGCTGTCACAGCACCTGAATCA
GAAGGACACCCTCATGCAGAGACTGCGG
N-term
(SEQ ID NO: 112)
NGLQNCATHTQPFWGSLTNRTRPPSVQVAKTTPFNTREPVMLACYVWGF
YPAEVTITWRKNGKLVMPHSSAHKTAQPNGDWTYQTLSHLALTPSYGDT
YTCVVEHIGAPEPILRDWTPGLSPMQTLKVSVSAVTLGLGLIIFSLGVI
SWRRAGHSSYTPLPGSNYSEGWHIS
(SEQ ID NO: 113)
AACGGCCTTCAGAATTGCGCTACTCACACTCAGCCGTTCTGGGGGTCAT
TGACCAACCGGACTCGCCCTCCATCTGTACAGGTGGCGAAAACTACCCC
TTTCAACACCCGGGAGCCAGTCATGCTGGCTTGCTATGTTTGGGGGTTT
TACCCAGCTGAGGTTACTATAACGTGGCGCAAAAACGGGAAGCTCGTAA
TGCCTCATTCGTCCGCCCATAAGACGGCTCAACCCAACGGAGACTGGAC
ATATCAGACCCTTTCCCACCTTGCACTGACTCCATCTTATGGGGACACT
TACACCTGCGTGGTGGAGCACATTGGGGCCCCAGAACCAATCCTGCGAG
ACTGGACCCCAGGGCTCTCACCAATGCAGACGCTGAAGGTATCAGTGAG
TGCTGTGACTCTGGGCCTGGGACTGATCATTTTCAGCCTGGGGGTAATT
AGTTGGAGAAGGGCTGGACACAGCAGTTACACACCCCTGCCTGGCAGCA
ATTACTCTGAAGGCTGGCATATTAGC
Bcap31_104_1
(SEQ ID NO: 114)
MSLQWTAVATFLYAEVFVVLLLCIPFISPKRWQKIFKSRLVELLVSYGN
TFFVVLIVILVLLVIDAVREIRKYDDVTEKVNLQNNPGAMEHFHMKLFR
AQRN
(SEQ ID NO: 115)
ATGAGTCTGCAGTGGACTGCAGTTGCCACCTTCCTCTATGCGGAGGTCT
TTGTTGTGTTGCTTCTCTGCATTCCCTTCATTTCTCCTAAAAGATGGCA
GAAGATTTTCAAGTCCCGGCTGGTGGAGTTGTTAGTGTCCTATGGCAAC
ACCTTCTTTGTGGTTCTCATTGTCATCCTTGTGCTGTTGGTCATCGATG
CCGTGCGCGAAATTCGGAAGTATGATGATGTGACGGAAAAGGTGAACCT
CCAGAACAATCCCGGGGCCATGGAGCACTTCCACATGAAGCTTTTCCGT
GCCCAGAGGAAT
Bcap31_TM1_21_7
(SEQ ID NO: 116)
AVATFLYAEVFVVLLLCIPFI
(SEQ ID NO: 117)
GCAGTTGCCACCTTCCTCTATGCGGAGGTCTTTGTTGTGTTGCTTCTCT
GCATTCCCTTCATT
Bcap31_TM2_21_44
(SEQ ID NO: 118)
LVSYGNTFFVVLIVILVLLVI
(SEQ ID NO: 119)
TTAGTGTCCTATGGCAACACCTTCTTTGTGGTTCTCATTGTCATCCTTG
TGCTGTTGGTCATC
Sequences identified in the TM_ER screens
840_ESYT1_HUMAN
(SEQ ID NO: 120)
SGGQPAGPGAAGEALAVLTSFGRR
960_PIGZ_HUMAN
(SEQ ID NO: 121)
PRLLLTALSFALDGAVYHLAPPMGA
944_PIGG_HUMAN
(SEQ ID NO: 122)
KDISKGIIEARFVYVFVLGILFTGT
390_synthetic
(SEQ ID NO: 123)
RPFCIILLISFFFVIVVGFFIYDQS
1121_DERL3_HUMAN
(SEQ ID NO: 124)
FVFMFLFGGVLMTLLGLLGSLFFLG
489_DAD1_HUMAN
(SEQ ID NO: 125)
FPFNSFLSGFISCVGSFILAVCLRI
451_ABCB9_HUMAN_2BT
(SEQ ID NO: 126)
SWLVITLVCLFVGIYAMVKLLLFSE
108_synthetic
(SEQ ID NO: 127)
PTMYLALIFTLVCILIVLGCILLLN
289_synthetic
(SEQ ID NO: 128)
QQLIVSLWCILLGGLCLLVGILLTK
205_synthetic_2BT
(SEQ ID NO: 129)
DLTLWLLASSILFLVVYSLLVINR
Region Containing the BT1 TM domain
(SEQ ID NO: 130)
WTAVATFLYAEVFVVLLLCIPFISP
1008_RER1_HUMAN
(SEQ ID NO: 131)
FDAFNVPVFWPILVMYFIMLFCITM
270_synthetic
(SEQ ID NO: 132)
FNLCLLIAGGLLVFTLLMVLGVSGV
7_synthetic
(SEQ ID NO: 133)
YKLYSLVSLSGWVLVKAFGMFGHRV
417_PIEZ1_HUMAN
(SEQ ID NO: 134)
PCLDLGAMLLYTLTFWLLLRQFVKE
427_SYT6_HUMAN
(SEQ ID NO: 135)
VSLLAVVVIVCGVALVAVFLFLFWK

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of identifying an antigenic polypeptide as a ligand for binding by a T cell receptor (TCR), the method comprising:
a) co-culturing at least one cell comprising:
i) at least one TCR and
ii) a nucleic acid molecule comprising a TCR responsive promoter operably linked to an expression cassette comprising a nucleotide sequence encoding a marker,
with one or more antigen presenting cell (APC) of an APC library comprising a plurality of APCs, wherein each APC comprises a nucleic acid molecule of a minigene library comprising a plurality of nucleic acid molecules wherein each nucleic acid molecule comprises a nucleotide sequence encoding at least one antigenic polypeptide for presentation as an antigenic polypeptide-human leukocyte antigen (HLA) complex, wherein the sequence encoding the at least one antigenic polypeptide is of a predetermined length;
wherein binding of the TCR to at least one antigenic polypeptide-HLA complex of the APC induces expression of the nucleic acid molecule encoding the marker;
b) isolating the APC cell bound to the cell comprising the TCR; and
c) sequencing the nucleic acid molecule of the minigene library of the APC cell to identify the antigenic polypeptide as a ligand for binding by the TCR.

2. The method of claim 1, wherein the expression cassette comprises a nucleotide sequence encoding a marker selected from the group consisting of a fluorescent marker and an antibody, or fragment thereof, specific for binding to an APC marker.

3.-4. (canceled)

5. The method of claim 1, wherein the method of co-culturing comprises applying the at least one cell expressing a TCR to a first inlet of a microfluidic co-culture device comprising two inlets and applying the APC library to a second inlet of the microfluidic co-culture device.

6.-8. (canceled)

9. The method of claim 1, wherein the TCR responsive promoter comprises a sequence selected from the group consisting of SEQ ID NO:50-SEQ ID NO:74.

10.-21. (canceled)

22. The method of claim 1, wherein the nucleic acid molecule of the minigene library further comprises a sequence encoding at least one transmembrane domain, or fragment thereof.

23. The method of claim 22, wherein the transmembrane domain is selected from the group consisting of SEQ ID NO:114, SEQ ID NO:116, SEQ ID NO:118, SEQ ID NO:120, SEQ ID NO:121, SEQ ID NO:122, SEQ ID NO:123, SEQ ID NO: 124, SEQ ID NO:125, SEQ ID NO:126, SEQ ID NO:127, SEQ ID NO:128, SEQ ID NO:129, SEQ ID NO:131, SEQ ID NO:132, SEQ ID NO:133, SEQ ID NO:134, and SEQ ID NO:135.

24.-67. (canceled)

68. A microfluidic device for co-encapsulation of individual cells, comprising:
two or more proximal inlets fluidly connected to one or more distal outlets;
a filter positioned downstream from each proximal inlet;
a series of asymmetrical focusing loops positioned downstream from each filter;
a nozzle positioned upstream from the one or more distal outlets; and
one or more isolation fluid inlets connected to the nozzle;
wherein each of the series of asymmetrical focusing loops comprise arcuate channel lengths curving in alternating directions about a central axis, such that arcuate channel lengths on a first side of the central axis are larger than arcuate channel lengths on an opposing second side of the central axis; and
wherein each of the series of asymmetrical focusing loops converge into a single microchannel fluidly connected to the nozzle.

69.-93. (canceled)

94. The device of claim 68, wherein the device further comprises one or more additional inlets fluidly connected directly to or to a position between one or more of the proximal inlets, filters, focusing loops, nozzle, and outlet.

95. (canceled)

96. A method of forming co-encapsulated particles, comprising the steps of:
providing the device of claim 68;
providing a first suspension of a particle;
providing at least one second suspension of a particle;
flowing each of the suspensions through a proximal inlet of the device; and
flowing an isolation fluid through an isolation fluid inlet of the device.

97. The method of claim 96, wherein the particle of the first suspension and the at least one second suspension is selected from the group consisting of: cells, viruses, bacteria, amoeba, protozoa, paramecium, microparticles, nanoparticles, beads, microorganisms, vesicles, nucleic acid oligonucleotides, proteins, polypeptides, carbohydrates, and fragments thereof.

98. The method of claim 97, wherein the first suspension and the at least one second suspension comprises a suspension fluid selected from the group consisting of: water, cell growth media, serum, plasma, and oil.

99. The method of claim 98, wherein the isolation fluid is immiscible with the suspension fluid.

100. The method of claim 97, wherein the particle of the first suspension is a B cell.

101.-104. (canceled)

105. The method of claim 97, wherein the particle of the at least one second suspension is a T cell.

106.-109. (canceled)

110. A nucleic acid molecule encoding an antibody or fragment thereof specific for binding to an antigen-presenting cell (APC) marker selected from the group consisting of CD19 and CD20, wherein the nucleic acid molecule comprises at least one selected from the group consisting of:
a) a nucleotide sequence encoding a heavy chain variable region of an anti-CD19 synthetic antibody selected from the group consisting of SEQ ID NO:4 and SEQ ID NO:12;
b) a nucleotide sequence encoding a light chain variable region of an anti-CD19 synthetic antibody selected from the group consisting of SEQ ID NO:8 and SEQ ID NO:16;
c) a nucleotide sequence encoding a heavy chain variable region of an anti-CD20 synthetic antibody selected from the group consisting of SEQ ID NO:20 and SEQ ID NO:28; and
d) a nucleotide sequence encoding a light chain variable region of an anti-CD20 synthetic antibody selected from the group consisting of SEQ ID NO:24 and SEQ ID NO:32.

111. The nucleic acid molecule of claim 110, encoding an scFV antibody fragment selected from the group consisting of:
a) a nucleotide sequence encoding a heavy chain variable region of an anti-CD19 synthetic antibody comprising SEQ ID NO:4 and a light chain variable region of an anti-CD19 synthetic antibody comprising SEQ ID NO:8;
b) a nucleotide sequence encoding a heavy chain variable region of an anti-CD19 synthetic antibody comprising SEQ ID NO:12 and a light chain variable region of an anti-CD19 synthetic antibody comprising SEQ ID NO:16;
c) a nucleotide sequence encoding a heavy chain variable region of an anti-CD20 synthetic antibody comprising SEQ ID NO:20 and a light chain variable region of an anti-CD20 synthetic antibody comprising SEQ ID NO:24; and
d) a nucleotide sequence encoding a heavy chain variable region of an anti-CD20 synthetic antibody comprising SEQ ID NO:28 and a light chain variable region of an anti-CD20 synthetic antibody comprising SEQ ID NO:32.

112. (canceled)

113. The nucleic acid molecule of claim 110, wherein the nucleic acid molecule comprises a nucleotide sequence encoding at least two tandem scFv molecules separated by a linker sequence.

114. A nucleic acid molecule comprising a nucleotide sequence encoding at least one antigenic polypeptide for presentation at a cell surface, and further comprising at least one transmembrane sequence for enhanced presentation, or fragment thereof.

115. The nucleic acid molecule of claim 114, wherein the transmembrane domain is selected from the group consisting of SEQ ID NO:114, SEQ ID NO:116, SEQ ID NO:118, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO: 128, SEQ ID NO:129, SEQ ID NO:131, SEQ ID NO:132, SEQ ID NO:133, SEQ ID NO:134, and SEQ ID NO:135.

116.-117. (canceled)

118. An immunogenic composition comprising at least one nucleic acid molecule comprising a nucleotide sequence encoding at least one antigenic polypeptide for presentation at a cell surface, and further comprising at least one transmembrane sequence for enhanced presentation of claim 114.

119.-121. (canceled)


SEQUENCE LISTING (140 sequences)

SEQ ID NO: 1 - protein, 8 aa, synthetic construct
GYAFSSYW

SEQ ID NO: 2 - protein, 8 aa, synthetic construct
IWPGDSDT

SEQ ID NO: 3 - protein, 17 aa, synthetic construct
ARRETTTVGRYYYAMDY

SEQ ID NO: 4 - protein, 124 aa, synthetic construct
EVQLVQSGAEVKKPGSSVKVSCKASGYAFSSYWMNWVRQAPGQGLEWMGQIWPGDSDTNYAQKFQGRVTITADESTSTAYMELSSLRSEDTAVYYCARRETTTVGRYYYAMDYWGQGTTVTVSS

SEQ ID NO: 5 - protein, 10 aa, synthetic construct
QSVDYSGDSY

SEQ ID NO: 6 - skipped

SEQ ID NO: 7 - protein, 9 aa, synthetic construct
QQSTENPWT

SEQ ID NO: 8 - protein, 111 aa, synthetic construct
DIQLTQSPSFLSASVGDRVTITCKASQSVDYSGDSYLNWYQQKPGKAPKLLIYDASNLVSGVPSRFSGSGSGTEFTLTISSLQPEDFATYYCQQSTENPWTFGGGTKLEIK

SEQ ID NO: 9 - protein, 8 aa, synthetic construct
GVSLPDYG

SEQ ID NO: 10 - protein, 7 aa, synthetic construct
IWGSETT

SEQ ID NO: 11 - protein, 14 aa, synthetic construct
AKHYYYGGSYAMDY

SEQ ID NO: 12 - protein, 120 aa, synthetic construct
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 13 - protein, 6 aa, synthetic construct
QDISKY

SEQ ID NO: 14 - skipped

SEQ ID NO: 15 - protein, 9 aa, synthetic construct
QQGNTLPYT

SEQ ID NO: 16 - protein, 107 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 17 - protein, 8 aa, synthetic construct
GFTFNDYA

SEQ ID NO: 18 - protein, 8 aa, synthetic construct
ISWNSGSI

SEQ ID NO: 19 - protein, 15 aa, synthetic construct
AKDIQYGNYYYGMDV

SEQ ID NO: 20 - protein, 122 aa, synthetic construct
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSS

SEQ ID NO: 21 - protein, 6 aa, synthetic construct
QSVSSY

SEQ ID NO: 22 - skipped

SEQ ID NO: 23 - protein, 9 aa, synthetic construct
QQRSNWPIT

SEQ ID NO: 24 - protein, 107 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIK

SEQ ID NO: 25 - protein, 8 aa, synthetic construct
GYAFSSYW

SEQ ID NO: 26 - protein, 8 aa, synthetic construct
IWPGDSDT

SEQ ID NO: 27 - protein, 17 aa, synthetic construct
ARRETTTVGRYYYAMDY

SEQ ID NO: 28 - protein, 121 aa, synthetic construct [sequence omitted]

SEQ ID NO: 29 - protein, 10 aa, synthetic construct
QSVDYSGDSY

SEQ ID NO: 30 - skipped

SEQ ID NO: 31 - protein, 9 aa, synthetic construct
QQSTENPWT

SEQ ID NO: 32 - protein, 106 aa, synthetic construct [sequence omitted]

SEQ ID NO: 33 - protein, 519 aa, synthetic construct
EVQLVQSGAEVKKPGSSVKVSCKASGYAFSSYWMNWVRQAPGQGLEWMGQIWPGDSDTNYAQKFQGRVTITADESTSTAYMELSSLRSEDTAVYYCARRETTTVGRYYYAMDYWGQGTTVTVSSGSTSGSGKPGSGEGSTKGDIQLTQSPSFLSASVGDRVTITCKASQSVDYSGDSYLNWYQQKPGKAPKLLIYDASNLVSGVPSRFSGSGSGTEFTLTISSLQPEDFATYYCQQSTENPWTFGGGTKLEIKGGGGSAEAAAKEAAAKAGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKGGGGSGGGGSGGGGSEVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSS

SEQ ID NO: 34 - DNA, 732 bp, synthetic construct
  source 1..732: mol_type = other DNA
gaagtccaattagtggaaagcggggggggactggtccagcccgggagatccctgcgactctcctgtgccgcctctggctttacctttaacgactacgctatgcactgggtgagacaggcccctggtaaggggcttgagtgggtatcaactattagctggaactctggttccatagggtatgcagacagcgtcaagggcagattcacaatttcacgagataatgcgaaaaagtcattgtatcttcaaatgaactcactcagagccgaagacaccgccctgtactactgcgcaaaggacattcaatacggaaattattactatggaatggatgtatggggacaaggcaccacggtgacagtttcctccggagggggaggctctgggggaggaggaagtggcggcggagggtccgaaatcgtgctgacacagtctccagctaccctgagcctttctcccggcgaaagggccaccctgagctgtcgagcttctcaatcggtgtcgagttacctcgcttggtatcagcagaaaccgggccaagcaccacggctcttgatctatgatgctagtaaccgcgcaaccggcattccggcaaggttttcggggtcaggttctggcacagactttacattgactatctcttcactcgaacccgaggattttgcagtctactactgccagcagaggagtaattggccaattacattcggtcaaggaacccggcttgaaataaag

SEQ ID NO: 35 - protein, 244 aa, synthetic construct
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIK

SEQ ID NO: 36 - DNA, 732 bp, synthetic construct
  source 1..732: mol_type = other DNA
gaaatcgtgctgacacagtctccagctaccctgagcctttctcccggcgaaagggccaccctgagctgtcgagcttctcaatcggtgtcgagttacctcgcttggtatcagcagaaaccgggccaagcaccacggctcttgatctatgatgctagtaaccgcgcaaccggcattccggcaaggttttcggggtcaggttctggcacagactttacattgactatctcttcactcgaacccgaggattttgcagtctactactgccagcagaggagtaattggccaattacattcggtcaaggaacccggcttgaaataaagggagggggaggctctgggggaggaggaagtggcggcggagggtccgaagtccaattagtggaaagcggggggggactggtccagcccgggagatccctgcgactctcctgtgccgcctctggctttacctttaacgactacgctatgcactgggtgagacaggcccctggtaaggggcttgagtgggtatcaactattagctggaactctggttccatagggtatgcagacagcgtcaagggcagattcacaatttcacgagataatgcgaaaaagtcattgtatcttcaaatgaactcactcagagccgaagacaccgccctgtactactgcgcaaaggacattcaatacggaaattattactatggaatggatgtatggggacaaggcaccacggtgacagtttcctcc

SEQ ID NO: 37 - protein, 244 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKGGGGSGGGGSGGGGSEVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSS

SEQ ID NO: 38 - DNA, 750 bp, synthetic construct
  source 1..750: mol_type = other DNA
gaggtgcagctggtccagtcaggagcagaggtcaagaagccaggctcctctgtcaaagtgtcctgcaaagcctcaggctatgccttctcctcctactggatgaactgggtgcgccaggcccctgggcagggcctggagtggatgggacagatctggccaggagattcagacaccaactacgcccagaagttccagggaagggtcaccatcacagcagacgagagcacctccacagcctacatggagctgtcttcactcaggtcagaggacacagctgtgtattactgtgccagaagggagaccaccactgtgggccgctactattatgccatggactactggggccaggggaccacagttactgtttcaagtgggggtggtggctcaggaggaggaggaagtgggggtgggggctcagacatccagctcacccagagccccagcttcctctcagcctctgtgggagacagagtcactatcacctgcaaggccagccagagtgtggactactcaggggattcatacctcaactggtaccagcaaaaacctggcaaagctcccaagctgctcatctacgatgccagcaacctggtttctggtgtgccttccagattttctggctctggaagtggcacagagttcacccttaccatttcttccctgcagccagaagattttgccacctactactgccagcagagcacagaaaacccctggacatttggagggggaaccaaactggaaatcaaa

SEQ ID NO: 39 - protein, 250 aa, synthetic construct
EVQLVQSGAEVKKPGSSVKVSCKASGYAFSSYWMNWVRQAPGQGLEWMGQIWPGDSDTNYAQKFQGRVTITADESTSTAYMELSSLRSEDTAVYYCARRETTTVGRYYYAMDYWGQGTTVTVSSGGGGSGGGGSGGGGSDIQLTQSPSFLSASVGDRVTITCKASQSVDYSGDSYLNWYQQKPGKAPKLLIYDASNLVSGVPSRFSGSGSGTEFTLTISSLQPEDFATYYCQQSTENPWTFGGGTKLEIK

SEQ ID NO: 40 - DNA, 738 bp, synthetic construct
  source 1..738: mol_type = other DNA
ggagtccagtgtcaggtgcagctgcagcagccaggggcagagctggtcaaacctggtgccagtgtcaagatgtcctgcaaggcttcaggctacaccttcacctcttacaacatgcactgggtgaagcagaccccaggaagaggcctggagtggattggtgctatttatcctggaaatggagacaccagctacaatcagaagttcaagggcaaggccacactcacagcagacaagagctccagcacagcctacatgcagctcagcagccttacttcagaggattctgctgtgtactattgtgccaggagcacctattatggaggggactggtacttcaatgtgtggggagctggcaccacagtcactgtgtctgcagggggtgggggcagtggaggtggtggctcaggtggaggaggcagccagattgtgctgtcccagagcccagccatcctgtctgccagccctggggagaaggtcaccatgacctgcagggcctcctcctctgtgtcctacatccactggttccagcaaaagccaggctcctctcccaagccctggatctatgccacttccaacctggcctcaggagtgcctgtcagattttctggaagtggctctggcacaagttacagcctcaccatcagcagagtggaggcagaagatgctgccacctactactgccagcagtggacaagcaacccccccacctttggaggggggaccaagctggaaatcaag

SEQ ID NO: 41 - protein, 246 aa, synthetic construct
GVQCQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAGGGGSGGGGSGGGGSQIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIK

SEQ ID NO: 42 - protein, 18 aa, synthetic construct
GSTSGSGKPGSGEGSTKG

SEQ ID NO: 43 - protein, 19 aa, synthetic construct
MEFGLSWLFLVAILKGVQC

SEQ ID NO: 44 - protein, 81 aa, synthetic construct
GKPIPNPLLGLDSTGAGSGGKPIPNPLLGLDSTGAGSGGKPIPNPLLGLDSTGGAGSGGKPIPNPLLGLDSTGSGAGGSSL

SEQ ID NO: 45 - protein, 51 aa, synthetic construct
GGGGSGGGAYPYDVPDYAGGGSGYPYDVPDYAGGGSGYPYDVPDYAASGPS

SEQ ID NO: 46 - protein, 79 aa, synthetic construct
GAGSYPYDVPDYAGAGSYPYDVPDYAGGSYPYDVPDYAGGAGSYPYDVPDYAGAGSYPYDVPDYAGAGSYPYDVPDYAG

SEQ ID NO: 47 - protein, 93 aa, synthetic construct
GSGAGGSGSGAGGSGDYKDDDDKGAGSGDYKDDDDKGAGSDYKDDDDKGGAGSDYKDDDDKGSGDYKDDDDKGAGSGDYKDDDDKGSGAGGSS

SEQ ID NO: 48 - protein, 8 aa, synthetic construct
DYKDDDDK

SEQ ID NO: 49 - protein, 22 aa, synthetic construct
GGGGSAEAAAKEAAAKAGGGGS

SEQ ID NO: 50 - DNA, 334 bp, synthetic construct
  source 1..334: mol_type = other DNA
gaattcaagtaccgtgcaggaggaaaaactgtttcatacagaaggcgtgggctatgcaaatcgaaacgctggaaacactgagtaatcagatcaagtcatgtttccatttgaatgcaaatggatacagaaggcgtggaggaaaaactgtttcctgttccaatagacgccctttttgaatgcaaatggcgagctcatgggtttctccaccaaggtgacgagtggaaaatctgactcagaattatcccttgttatgcaaatcgcccaaagaggaaaatttgtttcatagaacatactgtctcaaatagacgcctcctcgagcgtttcccggggtacc

SEQ ID NO: 51 - DNA, 566 bp, synthetic construct
  source 1..566: mol_type = other DNA
gaattcaagtaccgtgcattgaagttaaataatttacagcatatggaaaataaatttcagatggattacgaggctgcaagagctccctggccttggaaaaccaactcacacaccttgactgaggaaactgcacaaatattgtgttttccatctgcccttggtttaaaaaactgtgtatcttctctccacttcattttccatatctgcaaaatgggaacgagctgggtggtggctacatacagatggaaaaattcatcagctgtacactgcaagggccatgggtgagcaccagctggaaaattactgcttcaactttcatattttggtaactaattcatatttttggaaaacatactggccacacaaaatttaaagaggaagttatagttcagtttttccaagttagcatttcctgctttgtagttttatgacaaagaaaattttctgagttacttttgtatccccacccccttaaagaaaggaggaaaaactgtttcatacagaaggcgttaattgcaaacatactgtctcaaatagacgcctcctcgagcgtttcccggggtacc

SEQ ID NO: 52 - DNA, 667 bp, synthetic construct
  source 1..667: mol_type = other DNA
gaattcaagtaccgtgcatccaggggtctcttatatgcacactttttccaaacaaacggggatcaaaatgttggaacctgctgttggcctgcctggaaaattcttccctccttaccttgagctgggtggtggctacatacagatggaaaaattcatcagctgtacactcgtcctccaccctccaagtccgcgctggaaaatcacccgctgcgggctcccaccagtgaaatgggatattagatattttccagctaggatacgacagctcctatatgtgggacagagccttggtctggaaaacactttcaagcccactgtgagacatttgggaatattgggggagttttccaaaagagaaatccaggtgagggtccaaagggactgattatgggctggaaaatcaaactggcacctgcaatgcatgctgacgtatgatttttatttggaaaaacctcaaaatagtaaatgtcccctattagctgttttgggaatccttttccagtcatgaccacattttgcagagatgggctaacaggtatgagcatgggaaaagcatgtttcaagaatttgagatgtatttcccagaaaaggaacatgatgaaaatggtcagaaaaggcaaacatactgtctcaaatagacgcctcctcgagcgtttcccggggtacc

SEQ ID NO: 53 - DNA, 659 bp, synthetic construct
  source 1..659: mol_type = other DNA
gaattcaagtaccgtgcattagcgggtcggtataatccccgactggaaaaccgccctaagccttatgagagctgggtggtggctacatacagatggaaaaattcatcagctgtacacttgagtcaggcatctatacctgttaatggaaaaagatggaagggggtattgagatgccctatgtaagtcaagtgtcttttccagaggaagtttgcatccgatgatatgacattattcaatctgtcatggaaaagtgctgcgtagcctacccagaatagacatatttttcattcatttggaaaacacccacttccctgtttcatttatttgaggaacaaacttgccgttttccataacagctgcactattttttatatataatgtgtgtaacacagcttttccagtgcctggtacaaggtaaatagagttttgggtaagaaaaccctttttccactttctaaatcttcaaggaaatacaaaattctgctagttcatattttccatttcatttattcagcaaattttttaaaaaacagtaagtcttggcaagtccatctcccccatctgacatcttgaaacaaagtttcctctctgggttctcagtaccaagaacaaacatactgtctcaaatagacgcctcctcgagcgtttcccggggtacc

SEQ ID NO: 54 - DNA, 666 bp, synthetic construct
  source 1..666: mol_type = other DNA
gaattcaagtaccgtgcatcgcagacatcatctttgatgctctttttccactgtttcggtgctttaattcagtggatcaatagacagttcctgttttccacacaactgaaagggtggaacaggccaagaggcagagtgggagattttccagagagaaagaaatagaagcaacatcctgtgtgcacagcaagtgtggaaaactgaaatagtgatctgagaagagattggccacagaaatccaggtggaaaacctgcagaagagtgggtttaccaaccctgcccatagttgacatttttccaccactccccccttcccagggatattatttaaaaaggaaacacttggaaaaaattatttcatttgttcagccctggatgtggacagcggccccctggaaaacgtgtatgagagcatccgtccaggggtctcttatatgcacactttttccaaacaaacggggatcaaaatgggaggccaaggtgggagaattttttttccaagtgtgcagttaatatttaataatttgcctgaatcatcctggtagtcacagaggtaaacgtttttgaaacaattgttccaaattgtgggtacaacttacaactaatttttcccattaaaacatactgtctcaaatagacgcctcctcgagcgtttcccggggtacc

SEQ ID NO: 55 - DNA, 677 bp, synthetic construct
  source 1..677: mol_type = other DNA
gaattcaagtaccgtgcaaaggcttcagtttcaaattgaatacattttccatccatggattggcttgttgcctcctgcgtgaaccagcactggtttttccagtaactggagccacaagctgttggaacctgctgttggcctgcctggaaaattcttccctccttaccttcttcagctgaggtcatctgcagacattttccatagttgggagctatttttaaagtgtaaaatggagcagccactttggaaaacagtttagcaattcataatttcagtctaacatgcaatttaattttttccatttaacaagttttatggaacactgataatctgttgccaccaaatggaaaacgtaaacaagatattctacacatgtatgccaatgcagattcactggaaaaatattgaaaatgaaaactcccctccccttcgcatcgccggggtttttccagccgaccgtcggccacttaacacaaaattgtattcttttgattttttccaatcatttaaaaatgtcaagccaggccagatttcctgtggccccgcctgaatgatgaaacacgggatggccattgcactccctggcttttccagagatctcggtcctcggtcctgatgcaaccgtctggaacatactgtctcaaatagacgcctcctcgagcgtttcccggggtacc

SEQ ID NO: 56 - DNA, 810 bp, synthetic construct
  source 1..810: mol_type = other DNA
gaattcaagtaccgtgcatcatgttgggaatggcacaactttttggaaaaaaatacatttataaaataagaacagatgccttctgatgctagattttccaaaactcctactgttatgctttgaggaggctatataaatattaattttccagttctggaattaagagaacggactacaacccacagctggtgagtggaaaagaggacattcttctaagaaaatagattgttgggaaggtaacatttttccatggttttgatttttcccaaaaggtcattctttgcgggtacattttccagggtccagctccgcaacagggcacagcatccggaattttgcgtttttccagcgggaaaaccaagaccacacagtaaggtttctcatctgcctggaaaaatgtggaattagctcttttggtaaattgagaccccttttacaatggaaaaataactggttataacggtcgtgagccaccgcgcccggccaacctggaaaattttgcaaggaaatacagttacccaagcctcacctcactgatattttccaaccaaaatgggggctaaaagggagagcgagacatgcagggagatggaaaaagaatcttttagagaaaacccaccccaccttgggagctgtgtctggaaaagagacttttcctgagccgaatattctaaatgtgcttgtaatcattttccacacttgtttattttttgagctgggtggtggctacatacagatggaaaaattcatcagctgtacactaacatactgtctcaaatagacgcctcctcgagcgtttcccggggtacc

SEQ ID NO: 57 - DNA, 806 bp, synthetic construct
  source 1..806: mol_type = other DNA
gaattcaagtaccgtgcatttcctcttgtgcaactgaagaacatggaaaaaatgatccctaaagtcctgtgtttttaaacgtgcgctttgggattttccacactgaagggtggtggcctttatttcaagatagctttggaagttttccatcaatgtaaccagtttcaaaaaggatagcacaaaactgctgtgtggaaaaacctataatgaacaactgttcacccccaacccatttgctgtttttttccatgtgagcttgagaaggcaacagattacctaaccaacatgtgtggaaaaggagttaaaatctgacaggagctgggtggtggctacatacagatggaaaaattcatcagctgtacactcctagtcagaatttgtaccccagactggaaaactggggaatttccctgtaaatccttctccaattgggtcccttgcttttccacctcctgtgcctctggtttactattattatcttagatgagttggaaaactgtgctcagaaggggtcaaaggcagttgttggattgagcattttccaaggagactactttgaacccatttctctttttatggcttcataattttccactatgtagacatactataaagtgtcataaggaagcaatattttttccaatttgcatgaagttgccttgagcacagtcggggtatcgcaatggaaaactcttggcaaactgtaaattcctgttttcctttcagatggtgttggaaaacattgcaggaaaaccatgaacatactgtctcaaatagacgcctcctcgagcgtttcccggggtacc

SEQ ID NO: 58 - DNA, 807 bp, synthetic construct
  source 1..807: mol_type = other DNA
gaattcaagtaccgtgcattggaacccattgatttattctggattttccactgctttctaccattttaacttttaaattatgctgacagggttttccataattaataatgtttaaagattctgcatggtctctgccttcttttccatcatgtcttcctgttcctatctgttccctaacgcgtctgaccgtggaaaagtggatcagcaggagtggtgagaaaaatccttaaaaagtcacgttttccatttgattgggtcaaaactcatttgaagcaatgtccctccttttttccactgtggtctgtgtgtctggcttgggttttccattcaagtcatttggaaaatatccaccccccggggataagtacttactgagctgggtggattttccattcatggttccctggggctgctcaccctagggcagtatgaagtttttccatctccagcagaggactcttttatccaatcacatttgtttacagttttccatgttgtaatcatgtctgctatcttttgtgctttggtggtgagctggaaaatcctcagttctggctgtcgcctcctgcgtgaaccagcactggtttttccagtaactggagccacaagccttagatcctaaaagcgttccttggatggaaaaccaactcttccactggcttccaggggtctcttatatgcacactttttccaaacaaacggggatcaaaagggaaaagatatgtttttaacagctttttccattccttctctgtttccaacatactgtctcaaatagacgcctcctcgagcgtttcccggggtacc

SEQ ID NO: 59 - DNA, 808 bp, synthetic construct
  source 1..808: mol_type = other DNA
gaattcaagtaccgtgcaacaaaacagagattttgcacagggcttttccagttaaattttaggaaagcagagctgccacgttggtacagaagttggaaaacaccatacgcttcaactgccctaattttagtgtcatggcaacattttccaaggggatggaattgggtatgctatgtacgtctataaattttaatggaaaaaccaaataactttatcgtcacgcgccaccatgtactgattttggaaaatggtgattttctaactccctaaagagggtttatgtagacttttttttccatttttgacaacttaaaagaggactcaatagatgcatctgcatggaaaacatcctcccctctaccagtatgtacgtgcaaatattccaaaattggaaaaaaaatctgaaatttgaaaacataaaatgtctattaaaagggctttttccaattcctccacacaagtggtttagtaacgtaatgggaactttccttttccataaaactggggaatcaaggcatcggtcaacagaaaaggcccagttttccatgacaatgtccaaccactcgggatggccattgcactccctggcttttccagagatctcggtcctcggtgctcttctgaggggccaggaagttctggaaaacttcccctcagccgagctaggagattttaaggtgcacgcaaggtggaaaaccactgctgaagcagattaggcaaatgctaaatctacctcattttccatacctgtcttttgattaacatactgtctcaaatagacgcctcctcgagcgtttcccggggtacc

SEQ ID NO: 60 - DNA, 688 bp, synthetic construct
  source 1..688: mol_type = other DNA
agagatgggctaacaggtatgagcatgggaaaagcatgtttcaagaatttgagatgtatttcccagaaaaggaacatgatgaaaatggtcagaaaaggcattcttagggtccccagatgtctgccttttccattgattttctttcctggttaattgcgaggcttgcctcaaggtggaaaacagtatcggtttccactggatcactaggtcacagggtttgcctttttccaaacctgcatctcaattttacctatgatttgcattaaataactttggaaaaattgaagttcttgagaagccaccgcgcccggcctcctctccttttccaaatttatacattttgattggtggggagattccattataggggctggaaaacccctttaccataccatgaatgtaactgtactgttacaaagtggaaaataacagtttccacttttcgcagctctttgagagcaggactctattttccagtctctttataagcttcattcatagtgaagctaacagaagtttttccatggttagattgtgcaccgggctgtttttaaatgtaatatgattttccatttacttatttatctttgtagttttatgacaaagaaaattttctgagttacttttgtatccccacccccttaaagaaaggaggaaaaactgtttcatacagaaggcgttaattgca

SEQ ID NO: 61 - DNA, 624 bp, synthetic construct
  source 1..624: mol_type = other DNA
gaattcaagtaccgtgcaatatgagagcccagcactttacctcctgaccacagagctgggtggtggctacatacagatggaaaaattcatcagctgtacactaaatactttatattttataactatattttccaagtttgtgctttaactttgtgatggtttagcggcagctgcttgtaggggtatttttggtttcctgcttcttttttcccttctaagtaaagacaaaataacataagtgtttgactaatgatcaaatggaaaatagaatctggtgaaggtgtccaaacgcagaaaggaaggtgtttacagggaggacatagatccctccaccccaccaccccatcatttcgttgttgccagctggctgatgttaacaacattggaaaatatagatctgcgggagcattttggtttgcagaatatgctgtatttttccatagctgttagaaatgctaaaacccctggcacacggttggcagtagttctccccgactctcctaggaatgtttttcctctgtgctgagctgttgtcactgctctttgcaacacttttgctccacggagggggaggataacatactgtctcaaatagacgcctcctcgagcgtttcccggggtacc

SEQ ID NO: 62 - DNA, 602 bp, synthetic construct
  source 1..602: mol_type = other DNA
gaattcaagtaccgtgcaagacttgttcttcttctgcagtatctggaaaatctggagaaattaatgttaaaagacattattattaagtccctggaaaaacacatggccattttcaataaaacccctggcacacggttggcagtagttctccccgactctcctaggaatgtttttcctctgtgctgagctgttgtcactgctctttgcaacacttttgctccacggagggggaggattcaactgtgtatctgcttgcagtgtttttccattataatagtttttaaaatatgactccatcccaagaaattggttttaaagatttgaggaaaaactgcttttagatttcagagaaagaaaaagagtgtgtttctacttcccccttttaaatctgataacttttatttgatcactaggtcacagggtttgcctttttccaaacctgcatctcaattttcattaactacttaaagcataagctattttccaggagaggcagcaagtgcatattcagataatcgggtcccaggtctggaaaagcagccttttccccacgtaacatactgtctcaaatagacgcctcctcgagcgtttcccggggtacc

SEQ ID NO: 63 - DNA, 1348 bp, synthetic construct
  source 1..1348: mol_type = other DNA
tgtttttgatgggaaaattgagatgcaggtttggaaaaaggcaaaccctgtgacctagtgatcataaacaacaggcacccccttaaagaaaggaggaaaaactgtttcatacagaaggcgttaattgcatgaattagaacccatatgactgatggcatattcagataatcgggtcccaggtctggaaaagcagccttttccccacgtttctttccccacctaagaatgatttctatataagcataacagtaggagttttggaaaatctagcatcagaaggcatctgttcttcacttttatgtctgtacactcgctgtctgctgtcggctcaggaaaagtctcttttccagacacagctcccaaggtggggtgggcgcagctgagaagccgtaatcagatcaagtcatgtttccatttgaatgcaaatggatacagaaggcgtggaggaaaaactgtttcctgttccaatagacgcccttcagtttctggcccacttgtttgtctaataagtgtttgactaatgatcaaatggaaaatagaatctggtgaaggtgtccaaacgcagaaaggaaggtgtttacagggaggacaatatgagagcccagcactttacctcctgaccacacagtagcaggtgtttccttaaggcctcagctgaagagtgagtcggttaaagggagagcgagacatgcagggagatggaaaaagaatcttttagagaaaaagtaaaagccctgtagttcctttgttggtagcttcttagaagaatgtcctcttttccactcaccagctgtgggttgtagtccgggaagtgggaagatgatcaataaacttcttccagtaaccctttcaagttacatcctcccaatatgcaaatcctccgtggagcaaaagtgttgcaaagagcagtgacaacagctcagcacagaggaaaaacattcctaggagagtcggggagaactactgccaaccgtgtgccaggggttttagtgtaaattatctttttgttacccaagcctcacctcactgatattttccaaccaaaatgggggctaaatggtctgattacaggcgtgtgtgactcatagcttttcctcaccgcgcccggcctcctctccttttccaaattttccaatacattttgattttcttaaatggatgtgtcatgttttccaatttgaaatgcaaatgtcaggaaagtcccccagctcaccgttataaccagttataggggactttccatctatgcatggggattccccttttccattgtaaaaatttgaaatgcaaataggaaagtcccggggtctcaaaagatgatgcaattttaccattataccccacagct

SEQ ID NO: 64 - DNA, 1286 bp, synthetic construct
  source 1..1286: mol_type = other DNA
cacccccttaaagaaaggaggaaaaactgtttcatacagaaggcgttaattgcatgaattagactaaaaaggtagtgcattcttagggtccccagatgtctgccttttccattgattttctttcctgcccgctccttaggacacctcttaagtgtacagctgatgaatttttccatctgtatgtagccaccacccagctccacgtattttcaggtctcttttactcatttttaattgaaaatggccatgtgtttttccagggacttaataataatgtcttttaaaaataaaggattgaatgcaaatggcgagctcatgggtttctccaccaaggtgacgagtggaaaaggtgaaggtaacaatcttttccttgactcagaattatcccttgttatgcaaatcgccctgctgaagtcctttcaaaaggtcattctttgcgggtacattttccagggtccagctccgcaacaaatgtggaccctgtcattgaatgccgcagatgtacatgctcccgcagatctatattttccaatgttgttaacatcagccagctggcaatctacaatttccaacctgtctcttcgcatattttagttttccaaatttccaaattcttccaactttatattttataaaatatgaaatctatattttccaagtttgtgctttaactttaattttcttataaacccatatgactgatggcatattcagataatcgggtcccaggtctggaaaagcagccttttccccacgtttctttccccacctaaggaaagtcccatttgaaatgcaaatatggggtcccagctcaccgttataaccagttatttttccattgtaaaaggggtctcaatttaccattataccccacagctcagctgaagagtgagtcggttaaagggagagcgagacatgcagggagatatttgaaatgcaaattcttccaggaaaaatttccaagaatcttttagagaaaaagtaaaagccctgtagttagaaaagaggaaggaaattgccttttctgaccattttcatcatgttgcttttctgggaaatacatctcaaattcttgaaacatgcttttcccatgctcatacctgttagcccatctctcccagtttgaatctctgataaactatgggtctctgtaaaatagattgttgggaaggtaacatttttccatggttttgatttttcccaatttgaaatgcaaataggaaagtcccaaagtaaggtgatgtcaatatttaaggtgaaggtaacatgtcttccatattgattta

SEQ ID NO: 65 - DNA, 1325 bp, synthetic construct
  source 1..1325: mol_type = other DNA
aacccatatgactgatggcatattcagataatcgggtcccaggtctggaaaagcagccttttccccacgtttctttccccacctaaatgacaatccacattaactacttaaagcataagctattttccaggagaggcagcaagtgcattctactcccatgagagaagtcgggctgtttttaaatgtaatatgattttccatttacttatttatctttaaaatgaggttttttgttacccaagcctcacctcactgatattttccaaccaaaatgggggctaaatggtctgaggaaagattctattgcatttctaacagctatggaaaaatacagcatattctgcaaaccaaaatgtagaacgaaggacagctgaagagtgagtcggttaaagggagagcgagacatgcagggagatggaaaaagaatcttttagagaaaaagtaaaagccctgtagtggggattccccaaactgtttcctgttccaatagacgccctttttgaatgcaaatggcgagctcatgggtttctccaccaaggtgacgagtggaaaatctgacactcgctgtctgctgtcggctcaggaatgtgagtcataagtttttccactcttttccagacacagctcccaaggtggggtgggcgcagctgagaagccaggaaagtcccagtggaaaatctgactcagaattatcccttgttatgcaaatcgcccaaagaggaaaatttgtttcatagaacatactgtctcaaatagacgcctctatgagatgacctatgatttgcattaaataactttggattttccaaaaaattccattgaagttcttgaaatatgcaaatgaaacgttgacgcatctgaggtcccaaaatgtgatggtttagcggcagctgcttgtaggggtatttttggtttcctgcttcttttttcccttctaagtaaagacaaaataactagatccctccaccccaccaccccatcatttcgttgttgtttaacaagtgttgagtaaaggggactttccaggggactttccactaaaaaggtagtgcattcttagggtccccagatgtctgccttttccattgattttctttcctgcccgctccttagaggaaagtcccatttgaaatgcaaataggggactttccctattcacatgttcagtgtagttttatgacaaagaaaattttctgagttacttttgtatcccctgtgagtcataccttttccaccttttccacttattccaatttgaaatgcaaataaagaaaggaggaaaaactgtttcatacagaaggcgttaattgcatgaattagagcta

SEQ ID NO: 66 - DNA, 1427 bp, synthetic construct
  source 1..1427: mol_type = other DNA
cttcgcatattttagaaatactttatattttataactatattttccaagtttgtgctttaactttaattttcttatatgtttttgatgggaaaattgagatgcaggtttggaaaaaggcaaaccctgtgacctagtgatcataaacaacaggtacagaaggcgtgggctatgcaaatcgaaacgctggaaacactgagtaatcagatcaagtcatgtttccatttgaatgcaaatggatacaaatctgtgtggtgcacaatctaaccatggaaaaacttctgttagcttcactatgaatggcattaagatatgagcccagggcccacgctccctccgggcacagcatccggaattttgcgtttttccagcgggaaaaccaagacgtgttttcccgtttcccacaaggaaaacttccttggtggagaaacccatgagctcatctggagggcatctgaggtcccaaaatgtgatggtttagcggcagctgcttgtaggggtatttttggtttcctgcttcttttttcccttctaagtaaagacaaaataactagatccctccaccccaccaccccatcatttcgttgttgtttaacaagtgttgagtaaatggggtcccagctcaccgttataaccagttatttttccattgtaaaaggggtctcaatttaccattataccccacagctatttccaaggaaagtcccataatatgttctagaaaagtggaaactgttattttccactttgtaacagtacagttacattcatctttttggtagtaggggactttcctgtgactcatttttccaaacccatatgactgatggcatattcagataatcgggtcccaggtctggaaaagcagccttttccccacgtttctttccccacctaaatatgcaaataggaaagtcccatttgaaatgcaaatcagtttctggcccacttgtttgtctaataagtgtttgactaatgatcaaatggaaaatagaatctggtgaaggtgtccaaacgcagaaaggaaggtgtttacagggaggacaatatgagagcccagcactttacctcctgaccacacagtagcaggtgtttccttaaggcctatttgaaatgcaaataggaaagtccctcttccaatttccaaggggactttccctattcacatgttcagtgtagttttatgacaaagaaaattttctgagttacttttgtatccccacccccttaaagaaaggaggaaaaactgtttcatacagaaggcgttaattgcatgaattagagctaaggggactttccatttgaaatgcaaataggggactttccaagatgatgcaataaatcttccaaggggactttccgagagaagtcgggctgtttttaaatgtaatatgattttccatttacttatttatctttaaaatgaggt

SEQ ID NO: 67 - DNA, 1446 bp, synthetic construct
  source 1..1446: mol_type = other DNA
agtgctgggtcagcagctctttgagagcaggactctattttccagtctctttataagctttgatgcagtgtcagagagaagtcgggctgtttttaaatgtaatatgattttccatttacttatttatctttaaaatgaggtataaactatgggtctctgtaaaatagattgttgggaaggtaacatttttccatggttttgatttttcccaaaagtatttatgtattgatttaaggagtgtagctgttaattgcgaggcttgcctcaaggtggaaaacagtatcggtttccactgccaccccagaggaaaaactgtttcatacagaaggcgtgggctatgcaaatcgaaacgctggaaacactgagtaatcagatcaagtcatgtttccatttgaacttcgcatattttagaaatactttatattttataactatattttccaagtttgtgctttaactttaattttcttatattttgatgggaaaattgagatgcaggtttggaaaaaggcaaaccctgtgacctagtgatcataaacaacaggagctcatgggtttctccaccaaggtgacgagtggaaaatctgactcagaattatcccttgttatgcaaatcgcccaaagaggaaaatttaggaaagtccctttccagaagcatctgaggtcccaaaatgtgatggtttagcggcagctgcttgtaggggtatttttggtttcctgcttcttttttcccttctaagtaaagacaaaataactagatccctccaccccaccaccccatcatttcgttgttgtttaacaagtgttgagatttgaaatgcaaattaaccctttcaagttacatcctccccctccgtggagcaaaagtgttgcaaagagcagtgacaacagctcagcacagaggaaaaacattcctaggagagtcggggagaactactgccaaccgtgtgccaggggttttagtgtaaattatcaggggactttccaatatgcaaatatgcaaatggatacagaaggcgtggaggaaaaactgtttcctgttccaatagacgccctttttgaatgcaaatggcgagctcatgggtttatttgaaatgcaaatggggattccccaggggactttccaagatgatgcaatgcattatcttattctaaggtgaggtaactatcctagctatcttccaaggaaagtcccatttgaaatgcaaattagaaaagaggaaggaaattgccttttctgaccattttcatcatgttgcttttctgggaaatacatctcaaattcttgaaacatgcttttcccatgctcatacctgttagcccatctctcccagtttgaatctctgatttgaaatgcaaattcttccaatttccattttttatgagatgacctatgatttgcattaaataactttggaaaaattgaagttcttgagaaacgttgac

SEQ ID NO: 68 - DNA, 1417 bp, synthetic construct
  source 1..1417: mol_type = other DNA
tatgagatgacctatgatttgcattaaataactttggaaaaattgaagttcttgagaaacgttgacttttgatgggaaaattgagatgcaggtttggaaaaaggcaaaccctgtgacctagtgatcataaacaacagataaactatgggtctctgtaaaatagattgttgggaaggtaacatttttccatggttttgatttttcccaaaagtatttatgtattgatttaactaaaaaggtagtgcattcttagggtccccagatgtctgccttttccattgattttctttcctgcccgctccttagataatatgttctagaaaagtggaaactgttattttccactttgtaacagtacagttacattcatctttttggtagtgagctcatgggtttctccaccaaggtgacgagtggaaaatctgactcagaattatcccttgttatgcaaatcgcccaaagaggaaaattttcttccatgatcgtagttttccaagtgctgggtcagcagctctttgagagcaggactctattttccagtctctttataagctttgatgcagtgtcacactcgctgtctgctgtcggctcaggaaaagtctcttttccagacacagctcccaaggtggggtgggcgcagctgagaagccagaaccatccaatattcatgttgggaatggcacaactttttggaaaaaaatacatttataaaataaaacatacattttctatgtatgtgatatatgtagatgcatgatttccagacacctcttaagtgtacagctgatgaatttttccatctgtatgtagccaccacccagctccacgtattttcaggatttgaaatgcaaataggaaagtcccctgtcagtttctggcccacttgtttgtctaataagtgtttgactaatgatcaaatggaaaatagaatctggtgaaggtgtccaaacgcagaaaggaaggtgtttacagggaggacaatatgagagcccagcactttacctcctgaccacacagtagcaggtgtttcttttccaaagatgatgtcataggggactttccatttccaatttgaaatgcaaataacccatatgactgatggcatattcagataatcgggtcccaggtctggaaaagcagccttttccccacgtttctttccccacctaggggattccccaggggactttcctgtgactcattcgtgcagcttcttccaatttccgctcgtttttcctaggggactttccatttgaaatgcaaatgcatatttctactaaaaatatgactccatcccaagaaattggttttaaagatttgaggaaaaactgcttttagatttcagagaaagaaaaagagtgtgtttctacttcccccttttaaatctgataacttttattttaatcaggttgacatt

SEQ ID NO: 69 - DNA, 1487 bp, synthetic construct
  source 1..1487: mol_type = other DNA
gagagaagtcgggctgtttttaaatgtaatatgattttccatttacttatttatctttaaaatgaggttgagcccagggcccacgctccctccgggcacagcatccggaattttgcgtttttccagcgggaaaaccaagacgtgttttcccgtttcccacaacactcgctgtctgctgtcggctcaggaaaagtctcttttccagacacagctcccaaggtggggtgggcgcagctgagaagcctgatgagggatgtggtatggtaaaggggttttccagcccctataatggaatctccccaccctctttcagtagatttgcattaaaaaaataaacaagtgtggaaaatgattacaagcacatttagaatattttgtatgccacagggggattcccctgtttttgatgggaaaattgagatgcaggtttggaaaaaggcaaaccctgtgacctagtgatcataaacaacaggttttccaatttgaaatgcaaatcagtttctggcccacttgtttgtctaataagtgtttgactaatgatcaaatggaaaatagaatctggtgaaggtgtccaaacgcagaaaggaaggtgtttacagggaggacaatatgagagcccagcactttacctcctgaccacacagtagcaggtgtttccttaaggcctaagatgatgcaatatttccagcatatttctactaaaaatatgactccatcccaagaaattggttttaaagatttgaggaaaaactgcttttagatttcagagaaagaaaaagagtgtgtttctacttcccccttttaaatctgataacttttattttaatcaggttgacatttgtgactcatttttcctgagggattagtaaccctttcaagttacatcctccccctccgtggagcaaaagtgttgcaaagagcagtgacaacagctcagcacagaggaaaaacattcctaggagagtcggggagaactactgccaaccgtgtgccaggggttttagtgtaaattatctcacgcattccatttgaaatgcaaattctcttttactcatttttaattgaaaatggccatgtgtttttccagggacttaataataatgtcttttaaaaataaaggattttccattttccaaaggtgaggtaacatttgaaatgcaaatataatatgttctagaaaagtggaaactgttattttccactttgtaacagtacagttacattcatctttttggtagtggggattccccatttgaaatgcaaatttacaggcgtgagccaccgcgcccggcctcctctccttttccaaatttatacattttgattttcttaaaggggactttccttttccaaagatgatgcaataggaaagtcccatttgaaatgcaaataggaaagtcccagaaccatccaatattcatgttgggaatggcacaactttttggaaaaaaatacatttataaaataaaacatacattttctatgta

SEQ ID NO: 70 - DNA, 1301 bp, synthetic construct
  source 1..1301: mol_type = other DNA
ggaaagattctattgcatttctaacagctatggaaaaatacagcatattctgcaaaccaaaatgtagaacgaaggaagaatgatttctatataagcataacagtaggagttttggaaaatctagcatcagaaggcatctgttcttcacttttatgtctgtacagctgaagagtgagtcggttaaagggagagcgagacatgcagggagatggaaaaagaatcttttagagaaaaagtaaaagccctgtagtatttccaaatgctagcaggggaatccccataatatgttctagaaaagtggaaactgttattttccactttgtaacagtacagttacattcatctttttggtagtcactcgctgtctgctgtcggctcaggaaaagtctcttttccagacacagctcccaaggtggggtgggcgcagctgagaagccggggattccccatcgagctatatttccattcgagctgatttttcctaatctgtgtggtgcacaatctaaccatggaaaaacttctgttagcttcactatgaatggcattaagatactatgcaaatcgaaacgctggaaacactgagtaatcagatcaagtcatgtttccatttgaatgcaaatggatacagaaggcgtggaggaattttgatgggaaaattgagatgcaggtttggaaaaaggcaaaccctgtgacctagtgatcataaacaacaggacacctcttaagtgtacagctgatgaatttttccatctgtatgtagccaccacccagctccacgtattttcaggggggattccccatttgaaatgcaaatgaatgccgcagatgtacatgctcccgcagatctatattttccaatgttgttaacatcagccagctggcaatctacaacctgtctatttgaaatgcaaatatttccagctatcattgtcttccaaggaaagtccctagaaaagaggaaggaaattgccttttctgaccattttcatcatgttgcttttctgggaaatacatctcaaattcttgaaacatgcttttcccatgctcatacctgttagcccatctctcccagtttgaatctctgtcttccaagtgctgggtcagcagctctttgagagcaggactctattttccagtctctttataagctttgatgcagtgtcaatttgaaatgcaaataggaaagtcccaaggtgaggtaacatgtgaccgtagcttttccaaggggactttcctatgagatgacctatgatttgcattaaataactttggaaaaattgaagttcttgagaaacgttgac

SEQ ID NO: 71 - DNA, 1505 bp, synthetic construct
  source 1..1505: mol_type = other DNA
atggggtcccagctcaccgttataaccagttatttttccattgtaaaaggggtctcaatttaccattataccccacagctgacacctcttaagtgtacagctgatgaatttttccatctgtatgtagccaccacccagctccacgtattttcagggagctcatgggtttctccaccaaggtgacgagtggaaaatctgactcagaattatcccttgttatgcaaatcgcccaaagaggaaaatttcagctgaagagtgagtcggttaaagggagagcgagacatgcagggagatggaaaaagaatcttttagagaaaaagtaaaagccctgtagtggaaaacttccttggtggagaaacccatgagctcatctggaggagaatgatttctatataagcataacagtaggagttttggaaaatctagcatcagaaggcatctgttcttcacttttatgtctgtacatgtttccatttgaatgcaaatggatacagaaggcgtggaggaaaaactgtttcctgttccaatagacgccctttttgaatgcaaatgggagggattagtaaccctttcaagttacatcctccccctccgtggagcaaaagtgttgcaaagagcagtgacaacagctcagcacagaggaaaaacattcctaggagagtcggggagaactactgccaaccgtgtgccaggggttttagtgtaaattatctcacgcattcctgtgactcatatgcgtatagctgcatttccatgatcatatgctatgcaatatgcaaattagatttgcattaaaaaaataaacaagtgtggaaaatgattacaagcacatttagaatattttgtatgccacagctgtcagtttctggcccacttgtttgtctaataagtgtttgactaatgatcaaatggaaaatagaatctggtgaaggtgtccaaacgcagaaaggaaggtgtttacagggaggacaatatgagagcccagcactttacctcctgaccacacagtagcaggtgtttctgtgactcataatgatgctcgcttttccaatgatagccctgaggggattccccataatatgttctagaaaagtggaaactgttattttccactttgtaacagtacagttacattcatctttttggtagtaggggactttcctcttccaaggaaagtcccaagatgatgcaatatgcctatctatcttccaatttgaaatgcaaatgcatctgaggtcccaaaatgtgatggtttagcggcagctgcttgtaggggtatttttggtttcctgcttcttttttcccttctaagtaaagacaaaataactagatccctccaccccaccaccccatcatttcgttgttgtttaacaagtgttgagtaaatttgaaatgcaaatttttccatgatctattactcttccagtagctatatatttccatctagaaatcaactgtgtatctgcttgcagtgtttttccattataatagtttttaatccctga

SEQ ID NO: 72 - DNA, 1343 bp, synthetic construct
  source 1..1343: mol_type = other DNA
ggaaaacttccttggtggagaaacccatgagctcatctggaggatgcaaatggatacagaaggcgtggaggaaaaactgtttcctgttccaatagacgccctttttgaatgcaaatggcgagctcatgggtttaggttacccatttctgtatttccttgcaaaattttccaggttggccgggcgcggtggctcacgcctgtaatcccagtgtttttgatgggaaaattgagatgcaggtttggaaaaaggcaaaccctgtgacctagtgatcataaacaacaggtgtgactcattctagaaatcaactgtgtatctgcttgcagtgtttttccattataatagtttttaatccctgattttccatccaccaaggtgacgagtggaaaatctgactcagaattatcccttgttatgcaaatcgcccaaagaggaaaatttgtttcatagaacataggggattccccctgtcagtttctggcccacttgtttgtctaataagtgtttgactaatgatcaaatggaaaatagaatctggtgaaggtgtccaaacgcagaaaggaaggtgtttacagggaggacaatatgagagcccagcactttacctcctgaccacacagtagcaggtgtttcttttccaatgcagcatgctaagatgatgcaatgacacctcttaagtgtacagctgatgaatttttccatctgtatgtagccaccacccagctccacgtattttcaggatttgcatatttttttgttacccaagcctcacctcactgatattttccaaccaaaatgggggctaaatggtctgaagaatgatttctatataagcataacagtaggagttttggaaaatctagcatcagaaggcatctgttcttcacttttatgtctgtaaagatgatgcaatatgcaagctaggattcttccaatgacgaggataatatgcaaatgagagaagtcgggctgtttttaaatgtaatatgattttccatttacttatttatctttaaaatgaggtaggggactttccaggggactttccaaggtgaggtaacatgcatgcatgcttttcctggggattcccctatgagatgacctatgatttgcattaaataactttggaaaaattgaagttcttgagaaacgttgacaggaaagtcccatttgaaatgcaaataggaaagtccctgtgactcataggggactttccttttcctatatcgatctatcttccatgtgactatatttgaaatgcaaattctcttttactcatttttaattgaaaatggccatgtgtttttccagggacttaataataatgtcttttaaaaataaagga

SEQ ID NO: 73 - DNA, 1419 bp, synthetic construct
  source 1..1419: mol_type = other DNA
tagaaaagaggaaggaaattgccttttctgaccattttcatcatgttgcttttctgggaaatacatctcaaattcttgaaacatgcttttcccatgctcatacctgttagcccatctctcccagtttgaatctctgaggagtgtagctgttaattgcgaggcttgcctcaaggtggaaaacagtatcggtttccactgccaccccagagataaactatgggtctctgtaaaatagattgttgggaaggtaacatttttccatggttttgatttttcccaaaagtatttatgtattgatttacagtttctggcccacttgtttgtctaataagtgtttgactaatgatcaaatggaaaatagaatctggtgaaggtgtccaaacgcagaaaggaaggtgtttacagggaggacaatatgagagcccagcactttacctcctgaccacacagtagcaggtgtttccttaaggcctataatatgttctagaaaagtggaaactgttattttccactttgtaacagtacagttacattcatctttttggtagtgacacctcttaagtgtacagctgatgaatttttccatctgtatgtagccaccacccagctccacgtattttcaggatgcaaatggatacagaaggcgtggaggaaaaactgtttcctgttccaatagacgccctttttgaatgcaaatggcgagctcatgggttttttttgttacccaagcctcacctcactgatattttccaaccaaaatgggggctaaatggtctgaatttccagcatctgaggtcccaaaatgtgatggtttagcggcagctgcttgtaggggtatttttggtttcctgcttcttttttcccttctaagtaaagacaaaataactagatccctccaccccaccaccccatcatttcgttgttgtttaacaagtgttgagtaaaagatgatgcaatttttcctaggggactttcctgtttttgatgggaaaattgagatgcaggtttggaaaaaggcaaaccctgtgacctagtgatcataaacaacaggatttgaaatgcaaatactaaaaaggtagtgcattcttagggtccccagatgtctgccttttccattgattttctttcctgcccgctccttagaggaaagtcccaatctgtgtggtgcacaatctaaccatggaaaaacttctgttagcttcactatgaatggcattaagataaggaaagtcccaaggtgatgtcaatatagaagtcgagcttttccatgcaaggggactttcccagtgccattttccaatttgaaatgcaaatcagctgaagagtgagtcggttaaagggagagcgagacatgcagggagatggaaaaagaatcttttagagaaaaagtaaaagccctgtagtatttgaaatgcaaat

SEQ ID NO: 74 - DNA, 1357 bp, synthetic construct
  source 1..1357: mol_type = other DNA
cgctggaaacactgagtaatcagatcaagtcatgtttccatttgaatgcaaatggatacagaaggcgtggaggaaaaactgtttcctgtttcctttgttggtagcttcttagaagaatgtcctcttttccactcaccagctgtgggttgtagtccgggaagtgggaaactgtccaccaaggtgacgagtggaaaatctgactcagaattatcccttgttatgcaaatcgcccaaagaggaaaatttgtttcatagaacatatgagcccagggcccacgctccctccgggcacagcatccggaattttgcgtttttccagcgggaaaaccaagacgtgttttcccgtttcccacaaggggattccccagaatgatttctatataagcataacagtaggagttttggaaaatctagcatcagaaggcatctgttcttcacttttatgtctgtaaccggactgcatttctcaaagagctaattccacatttttccaggcagatgagaaaccttactgtgtggtaaatcccacaaacaggaatatgcaaattagctactatcagcggggattcccccttcgcatattttagaaatactttatattttataactatattttccaagtttgtgctttaactttaattttcttataggaaagattctattgcatttctaacagctatggaaaaatacagcatattctgcaaaccaaaatgtagaacgaaggaaggggactttccagatttgcattaaaaaaataaacaagtgtggaaaatgattacaagcacatttagaatattttgtatgccacagggggattccccagctgaagagtgagtcggttaaagggagagcgagacatgcagggagatggaaaaagaatcttttagagaaaaagtaaaagccctgtagtaggggactttccttttccatagcgatcgatatttccaatgcagctatttttcctaggaaagtcccttacaggcgtgagccaccgcgcccggcctcctctccttttccaaatttatacattttgattttcttaaataaactatgggtctctgtaaaatagattgttgggaaggtaacatttttccatggttttgatttttcccaaaagtatttatgtattgatttaatttgaaatgcaaatggggattcccctgtgagtcatatgcatcgattagtgctaaggtgaggtaactatgcagtcagtagttttccttcatcgaggaaagtcccatgcatatttccaatttgaaatgcaaattgtttttgatgggaaaattgagatgcaggtttggaaaaaggcaaaccctgtgacctagtgatcataaacaacaggatttgaaatgcaaat

SEQ ID NO: 75 - DNA, 1872 bp, synthetic construct
  source 1..1872: mol_type = other DNA
atgccttccacctctttccctgtgccctccaaatttccactggggcctgcagcagctgtgtttggaagaggggagaccttaggacctgcaccaagagcagggggcaccatgaagagtgcagaagaagagcactatggctatgcctcatccaatgtgagcccagccctacccctgcccactgcccactccacactaccagctccctgccacaatctgcagacttctacaccaggcatcatccccccagcagaccaccccagtgggtatggagcagctctggatggaggcccagcaggctacttcctgtcctctggccacactagaccagatggtgcccctgcactggagagtcctagaatagaaatcacttcctgcttgggcctctaccacaacaacaaccagttcttccatgatgtggaggtggaggatgttctcccatctagtaaaagatccccatccactgcaacactgtcacttccttcactggaagcctacagggacccatcctgtcttagccctgcttcttctctgtcctccagatcttgtaattcagaggcctctagttatgaaagcaactatagttacccttatgcaagtccacagacatcaccctggcagagcccttgtgtcagccccaagaccactgaccctgaggaaggcttcccccgggggcttggagcctgcaccctcctgggcagtccaagacattctcccagtacatccccaagggcatcagtaactgaggagtcctggttgggtgctaggtcctcaaggcctgccagcccatgcaacaaaagaaagtactccctgaatgggagacagcctccttactccccacaccacagccccacccctagtccccatggcagccccagggtttctgtgacagatgatagctggcttggaaacacaacccagtacacatcttcagcaatagtagctgccatcaatgccctgaccacagacagcagcttggacctgggtgatggggtgccagtgaagagcagaaaaaccaccctggagcagcccccctcagtggctttgaaggtggaacctgtgggagaagaccttggttcaccaccacctccagctgacttcgccccagaggactacagctccttccagcacatcaggaaagggggtttctgtgaccagtatctggctgttccccagcatccttatcagtgggccaagcccaaacccctctcacctacatcctacatgtcacctacactccctgctctggactggcaattacctggtagcacctcaggatcaggcaagccaggatcaggggaaggaagcaccaagggcatgctgtggcagaaaccaacagctccagaacaggcacctgcccccgccagaccataccagggtgtgagagtaaaggagccagtgaaagagctgctgaggaggaagaggggccatgccagtagtggtgctgcaccagcacccacagcagtggtgctgcctcaccaacctctggccacctacaccactgtgggccccagctgcctggatatggagggctctgtcagtgcagtcacagaggaggcagccctctgtgcaggctggctgtctcagccaggtggaggggggagtggaggtggggggtctggagggggtgggagcacagccccacctacagatgtaagcctgggagatgagcttcatttggatggtgaagatgtggccatggcccatgcagatgctttagatgactttgatcttgacatgcttggtgatggtgactcacctggccctggattcacacctcatgactcagctccctatggggcccttgacatggctgattttgaatttgagcagatgttcactgatgccttagggattgatgaatatggtggctga

SEQ ID NO: 76 - protein, 623 aa, synthetic construct
MPSTSFPVPSKFPLGPAAAVFGRGETLGPAPRAGGTMKSAEEEHYGYASSNVSPALPLPTAHSTLPAPCHNLQTSTPGIIPPADHPSGYGAALDGGPAGYFLSSGHTRPDGAPALESPRIEITSCLGLYHNNNQFFHDVEVEDVLPSSKRSPSTATLSLPSLEAYRDPSCLSPASSLSSRSCNSEASSYESNYSYPYASPQTSPWQSPCVSPKTTDPEEGFPRGLGACTLLGSPRHSPSTSPRASVTEESWLGARSSRPASPCNKRKYSLNGRQPPYSPHHSPTPSPHGSPRVSVTDDSWLGNTTQYTSSAIVAAINALTTDSSLDLGDGVPVKSRKTTLEQPPSVALKVEPVGEDLGSPPPPADFAPEDYSSFQHIRKGGFCDQYLAVPQHPYQWAKPKPLSPTSYMSPTLPALDWQLPGSTSGSGKPGSGEGSTKGMLWQKPTAPEQAPAPARPYQGVRVKEPVKELLRRKRGHASSGAAPAPTAVVLPHQPLATYTTVGPSCLDMEGSVSAVTEEAALCAGWLSQPGGGGSGGGGSGGGGSTAPPTDVSLGDELHLDGEDVAMAHADALDDFDLDMLGDGDSPGPGFTPHDSAPYGALDMADFEFEQMFTDALGIDEYGG

SEQ ID NO: 77 - DNA, 873 bp, synthetic construct
  source 1..873: mol_type = other DNA
atgaaagagaccgccgcagccaagttcgaaagacagcacatggactctagcactagtgctgggatagacgctaacatcctgagcataggtggatccggaggtgcgagcggcgaggggaggggcagccttcttacttgcggagatgtggaagagaatccaggacccgtgtcaaaaggagaggagcttatcaaagaaaatatgcacatgaagctctacatggagggaacagtggacaaccaccacttcaaatgtacctcagaaggagaagggaagccctacgagggcacacagaccatgcgcatcaaggtggtggaaggtggccccctgccctttgccttcgacatcctggccaccagcttcctgtatggcagcaaaaccttcatcaatcacacccagggcatccccgacttcttcaaacagagctttccagaaggcttcacctgggagcgggtcaccacctatgaagatggaggcgtgctcacagccactcaggacacctccctgcaggatggctgcctcatctacaacgtgaagatccgcggcgtcaacttcacttccaatggccctgtcatgcagaagaagaccttgggctgggaagccttcacagagaccctgtacccagcagatgggggcctggaggggcggaacgacatggccctgaaactggtgggcggctcccacctcattgccaatgccaaaaccacctacagaagcaagaaaccagccaagaacctcaagatgcctggggtgtactacgtggattaccgcctggaaagaatcaaggaggccaacaacgagacgtatgtggagcagcacgaggtggctgtggcccgctactgcgacctgccttccaagctgggccacaagctcaac

SEQ ID NO: 78 - DNA, 282 bp, synthetic construct
  source 1..282: mol_type = other DNA
ggaagcggtgcagggggctctggctccggtgctggcggttcaggtgattacaaagatgacgacgataagggcgccggaagcggcgactataaagacgatgacgacaaaggcgcaggtagcgactataaagacgacgacgataagggaggagctggtagtgattataaagacgatgacgataaggggagcggagattacaaggatgacgacgataagggagccggaagcggagactacaaagacgatgatgacaagggctctggcgcagggggctcttcttga

SEQ ID NO: 79 - DNA, 1086 bp, synthetic construct
  source 1..1086: mol_type = other DNA
gcgtggattcctctgcttctcctgttcctgtctcattgcacaggcagcctcagcgattataaggatgatgatgacaagggtgccggctcaggcgattacaaggacgatgatgacaagggagccggtagtgactacaaagacgatgatgataaagggggtgctggaagtgactataaagacgacgacgacaagggatcaggcgactataaagacgatgatgacaaaggcgccggatctggggactataaggacgacgatgacaaggggagcggcgcaggtggctctgggtccggggccggaggctcaggcgagcccccaaccgcatgtcgggagaagcagtatctgattaattcacagtgctgctccctttgccaacccgggcagaaacttgtgtccgactgtacagagttcacagagacagaatgcctgccatgcggagaatctgaatttctggacacatggaaccgggagacgcattgtcatcagcataagtattgcgacccaaaccttggacttcgggtgcagcagaaaggcacatctgagaccgataccatttgtacgtgcgaagaaggatggcattgcacctccgaagcgtgcgagtcctgtgtgcttcacaggtcctgctcacctgggtttggcgtgaagcagatcgccaccggcgtgtccgacaccatatgtgagccgtgtccagttggattcttcagcaatgtgtctagcgcttttgagaaatgccatccttggacgagttgcgagaccaaggatctcgtggtgcagcaggccggaaccaataagaccgacgtggtttgtggaccccaggataggttacgcgccctggtagttattcctattatttttggcatccttttcgctatactgttggtcctggtcttcatcaaaaaggttgctaaaaagcctaccaacaaagctcctcatcctaagcaggagccacaagagattaactttccagacgacttgcccggatctaacaccgcagcaccagtgcaggaaactcttcatggttgtcaacctgtcacccaggaagatgggaaagagagccgaatcagtgtacaagaacgccagtgataa

SEQ ID NO: 80 - DNA, 981 bp, synthetic construct
  source 1..981: mol_type = other DNA
gcgtggattcctctgcttctcctgttcctgtctcattgcacaggcagcctcagcgattataaggatgatgatgacaagggtgccggctcaggcgattacaaggacgatgatgacaagggagccggtagtgactacaaagacgatgatgataaagggggtgctggaagtgactataaagacgacgacgacaagggatcaggcgactataaagacgatgatgacaaaggcgccggatctggggactataaggacgacgatgacaaggggagcggcgcaggtggctctgggtccggggccggaggctcaggcttctcccaacaaatctacggcgtggtgtacgggaatgtaacattccatgtgccgtccaatgttccattaaaggaggtgctttggaagaagcagaaggacaaagtcgctgaactggagaacagcgaattcagggccttcagctctttcaaaaatcgggtttatctggacactgtgtccgggtcactgacaatctataacctgaccagctccgacgaagacgagtacgaaatggaatcgccaaacatcacggataccatgaagttctttctttacgtgctcgaatcattaccttcacccaccctaacatgtgcactgacaaatggaagcattgaggtgcagtgtatgatccccgagcattacaactcgcatcgtggcttaattatgtattcttgggattgtccgatggaacagtgcaagaggaactctacctcaatttactttaagatggagaatgatttaccgcaaaaaatccagtgtaccctgagcaacccccttttcaataccacatctagtattatcctgactacttgtattccttcctctggacacagccgccaccggtacgcccttatccccatccctctggctgtcattaccacctgtattgtcctctatatgaatggtattctgaaatgcgaccggaaaccagaccggactaattctaattgataa

SEQ ID NO: 81 - DNA, 1071 bp, synthetic construct
  source 1..1071: mol_type = other DNA
gcgtggattcctctgcttctcctgttcctgtctcattgcacaggcagcctcagcgattataaggatgatgatgacaagggtgccggctcaggcgattacaaggacgatgatgacaagggagccggtagtgactacaaagacgatgatgataaagggggtgctggaagtgactataaagacgacgacgacaagggatcaggcgactataaagacgatgatgacaaaggcgccggatctggggactataaggacgacgatgacaaggggagcggcgcaggtggctctgggtccggggccggaggctcaggcgtcattcatgtaaccaaagaagttaaagaggtagccacactgagctgtgggcacaatgtaagtgtagaagagcttgctcagacacggatttactggcagaaagagaagaaaatggtgctgacgatgatgtctggcgacatgaatatttggcccgagtacaagaatagaactattttcaatatagcgaataatcttagcatagtaattctggccctgaggccatctgacgaggggacatatgagtgcgtggtccttaagtacgagaaggatgccttcaagcgcgagcatttggctgaggtgactctctcggtcaaggccgattttcctacgccctcgatatcagactttgaaatccccaccagcaatatccgacggattatatgcagtacctccgggggatttccggagccacatctgtcttggctggagaacggggaagagctcaacgctattaacaccactgtatcccaggaccctgagaccgagctgtacgctgtgtccagcaaacttgactttaacatgactactaaccactccttcatgtgtttaataaagtacggccacttgagagtgaatcagacctttaactggaataccaccaaacaggagcactttcctgataacctgctgccttcctgggcgatcactctcatttctgtgaatggaatttttgtaatttgctgtctcacttactgtttcgcccctcggtgccgagaaagaaggcgcaacgagcgccttaggcgcgagtctgtccgccccgtg

SEQ ID NO: 82 - DNA, 867 bp, synthetic construct
  source 1..867: mol_type = other DNA
gcctggattccactgctcctgctctttctttcccattgcacagggagcctgagtgattacaaagacgacgatgacaagggtgctggaagcggggattataaagatgacgatgataagggggccggaagtgactacaaagacgacgatgataaaggaggagcaggaagcgattataaagatgacgacgacaaggggagtggggactacaaggacgacgatgataaaggggctggctctggagactataaggatgatgacgacaaaggcagcggtgcaggtgggtctggctctggtgccggcggatctggtaccccagaagtaaaggtggcctgttcggaagacgtagatctgccttgcaccgctccctgggacccccaggtgccctatacggtctcgtgggtaaaactgctcgagggcggggaggaaaggatggaaacacctcaggaagaccacctgcgcggtcagcattaccaccagaagggccagaatggatcattcgacgccccaaacgagagaccttacagtctgaagattcggaataccacaagctgtaacagtggcacatataggtgtaccttacaggacccggacggacagcgcaatctatccggcaaagtgatactgagagtcacggggtgtcctgcccagcggaaggaagaaacgtttaagaaataccgggcagaaattgtgttgcttcttgccctggtcatcttctaccttaccctgattatctttacttgtaagttcgcaaggctccaaagtatcttccctgacttctctaaggccgggatggagagagcctttctgccagtcacctcccctaataagcacctgggtctagtaacaccacacaagacagaactggtt

SEQ ID NO: 83 - DNA, 1233 bp, synthetic construct
  source 1..1233: mol_type = other DNA
gcgtggattcctctgcttctcctgttcctgtctcattgcacaggcagcctcagcgattataaggatgatgatgacaagggtgccggctcaggcgattacaaggacgatgatgacaagggagccggtagtgactacaaagacgatgatgataaagggggtgctggaagtgactataaagacgacgacgacaagggatcaggcgactataaagacgatgatgacaaaggcgccggatctggggactataaggacgacgatgacaaggggagcggcgcaggtggctctgggtccggggccggaggctcaggcgcaccactaaagattcaagcctatttcaacgagacagcagatcttccctgccaattcgctaattcacagaaccagtccttgtctgagctagtagtcttctggcaggaccaggaaaacctggtcttgaatgaggtgtacctgggtaaagagaagttcgacagcgtacactcaaagtacatgggccgcacctcttttgattctgacagttggacactacgcttgcacaatctacaaattaaggataaaggcctctaccagtgtattatccaccataaaaagccaactggcatgatccgtattcatcagatgaactctgaactctctgtgcttgctaacttctcacaaccagaaatagtgcccatttctaatatcacagagaacgtgtacattaatctgacctgttcctcaatccacggctaccccgaacccaaaaagatgtccgttctgttgcggaccaagaactcaactatcgaatatgacggcgtgatgcagaaatcccaggataacgtaaccgagctgtatgatgtaagcatatccttgagtgtgagttttcctgatgtcacctctaacatgacgatcttctgtatcctcgaaactgacaaaacgcggctcctgtccagcccattttccatagagctagaagatccccagcccccacccgatcacatcccctggattacagccgtcttgccgacagtgatcatatgcgtgatggttttttgtctgatcttgtggaaatggaaaaagaagaagcggcctaggaatagctataaatgcgggactaacacaatggagagggaggaaagcgaacaaacaaaaaagagagaaaaaatccatatcccggagagaagcgatgaggcacagagggttttcaagtcttccaaaacttcctcttgcgacaagagtgacacctgcttctgataa

SEQ ID NO: 84 - DNA, 825 bp, synthetic construct
  source 1..825: mol_type = other DNA
gagcgggtgcagcctttggaagagaatgtcggtaacgccgcccgtccccgcttcgagcgcaataagttactgcttgtggcctctgttatccaagggctcggtttgcttttatgttttacctacatctgcctgcacttctctgccctgcaagttagtcaccggtatccccggatacagtctatcaaggtccagttcacggaatacaaaaaggagaagggattcatattaacttcacagaaggaggacgaaatcatgaaagtgcagaacaactccgtgatcatcaactgcgacggtttctacctcattagcttgaagggctacttcagccaggaggtgaacatttcccttcactaccagaaagacgaggaacctcttttccagctgaagaaggtacgctcggtgaactctttaatggtggcctctttgacatataaagacaaggtttatctgaatgtgaccacagataatacctctctggacgatttccatgtgaatggtggtgagctcatactgatccatcaaaacccgggcgaattctgcgtactcggaagcggtgcagggggctctggctccggtgctggcggttcaggtgattacaaagatgacgacgataagggcgccggaagcggcgactataaagacgatgacgacaaaggcgcaggtagcgactataaagacgacgacgataagggaggagctggtagtgattataaagacgatgacgataaggggagcggagattacaaggatgacgacgataagggagccggaagcggagactacaaagacgatgatgacaagggctctggcgcagggggctcttct

SEQ ID NO: 85 - DNA, 1038 bp, synthetic construct
  source 1..1038: mol_type = other DNA
gaatacgcttcagatgcctcactcgacccagaggcaccctggcccccggccccccgagcccgggcatgtcgcgtgctaccctgggctctggtcgccggcttactgttgctcctgctcctggccgcagcttgcgcagtctttctggcttgtccgtgggcagtgtctggcgcaagggcatctcccggaagtgccgcaagcccaaggcttcgagagggtccggagctgtcccccgatgaccccgcaggcttgctcgacctgcggcaggggatgttcgctcagttggttgcacagaacgtcttattgattgatggccctctgtcgtggtactctgatccgggacttgccggcgttagtttgaccggcggactgtcatacaaggaagacaccaaggaacttgtcgtcgctaaggccggggtgtactatgtatttttccaactcgagctccgcagagtcgtggcgggcgaaggctcgggtagcgtcagccttgcactacaccttcaacctctgcggtcagctgccggcgcagcagcactcgctttgacagttgatctcccacctgctagtagcgaggctagaaacagtgccttcggcttccaagggcgccttctccatctgtcagccggacagagacttggcgtccacctgcacaccgaagcacgggcacgccacgcctggcagttaactcagggtgccaccgtcctgggtctcttcagagtaaccccagagatacccgccggactccctagcccacgctccgaagggtccggcgcagggggatcaggttccggtgccggtgggtctggagactacaaagacgacgatgataaaggggctggttctggggattacaaagatgacgacgacaagggtgccggatcagactacaaagatgacgatgacaaaggaggcgccggttcagattataaggacgatgacgacaaaggctcaggtgactataaagatgacgacgacaaaggcgcagggtccggagactacaaggacgacgatgataaaggatcaggagccggcggttcctct

SEQ ID NO: 86 - protein, 17 aa, synthetic construct
MKETAAAKFERQHMDSS

SEQ ID NO: 87 - DNA, 52 bp, synthetic construct
  source 1..52: mol_type = other DNA
atgaaagagaccgccgcagccaagttcgaaagacagcacatggactctagca

SEQ ID NO: 88 - protein, 20 aa, synthetic construct
MPRQLRAAAALFARLAVILH

SEQ ID NO: 89 - DNA, 60 bp, synthetic construct
  source 1..60: mol_type = other DNA
atgccgaggcagttgagggcggcggcggcgttgtttgcgaggttggcggtgatattgcat

SEQ ID NO: 90 - protein, 40 aa, synthetic construct
SSDYTIVLPVIGAIVVGLCLMGMGVYKIRLRCQSSGYQRI

SEQ ID NO: 91 - DNA, 120 bp, synthetic construct
  source 1..120: mol_type = other DNA
agcagcgattatacgatagtgttgccggtgataggggcgatagtggtggggttgtgtttgatggggatgggggtgtataagataaggttgaggtgtcagagcagcgggtatcagaggata

SEQ ID NO: 92 - protein, 12 aa, synthetic construct
DSEDEDFEILSL

SEQ ID NO: 93 - DNA, 36 bp, synthetic construct
  source 1..36: mol_type = other DNA
gattcagaagatgaggactttgaaattctgtccctg

SEQ ID NO: 94 - protein, 16 aa, synthetic construct
TDLGTDWENLPSPRFC

SEQ ID NO: 95 - DNA, 48 bp, synthetic construct
  source 1..48: mol_type = other DNA
accgacttaggcaccgactgggaaaatttaccctcccccagattttgt

SEQ ID NO: 96 - protein, 16 aa, synthetic construct
SCDTDDFVLVPHNISS

SEQ ID NO: 97 - DNA, 48 bp, synthetic construct
  source 1..48: mol_type = other DNA
tcttgtgatactgatgactttgttctggtgccacataacatatcatcc

SEQ ID NO: 98 - protein, 106 aa, synthetic construct
MHRRRSRSCREDQKPVMDDQRDLISNNEQLPMLGRRPGAPESKCSRGALYTGFSILVTLLLAGQATTAYFLYQQQGRLDKLTVTSQNLQLENLRMKLPKPPKPVSK

SEQ ID NO: 99 - DNA, 318 bp, synthetic construct
  source 1..318: mol_type = other DNA
atgcaccgtaggcggtcaagatcatgcagggaagatcagaagccggtaatggacgaccagcgggacctgattagcaacaatgagcagctgcccatgctcggcagacgacccggggctccagagagtaaatgttctaggggcgctctttacaccgggtttagtattcttgtgactttattactggctgggcaggctactaccgcatatttcctgtaccaacagcagggccgtctggataagctgacagtgacatcacagaacttgcagctcgagaacctgagaatgaagctcccgaagcctccgaagcctgtttcgaaa

SEQ ID NO: 100 - protein, 117 aa, synthetic construct
QALPMGALPQGPMQNATKYGNMTEDHVMHLLQNADPLKVYPPLKGSFPENLRHLKNTMETIDWKVFESWMHHWLLFEMSRHSLEQKPTDAPPKESLELEDPSSGLGVTKQDLGPVPM

SEQ ID NO: 101 - DNA, 351 bp, synthetic construct
  source 1..351: mol_type = other DNA
caggccctgcccatgggagccctcccccagggccccatgcagaacgccaccaagtacggcaacatgaccgaggaccatgtgatgcatctgctgcagaacgccgatcctctgaaggtgtacccaccgctgaaggggtccttcccagagaaccttagacatttgaagaacacgatggagaccatcgattggaaggtctttgaatcctggatgcatcactggctgctgttcgagatgtccaggcactcccttgagcagaagcccacagacgccccgcctaaagagtccttggagttagaggacccatcctctggtcttggagtcacgaagcaggatctcggacctgtgccaatg

SEQ ID NO: 102 - protein, 85 aa, synthetic construct
MGHEQNQGAALLQMLPLLWLLPHSWAVPEAPTPMWPDDLQNHTFLHTVYCQDGSPSVGLSEAYDEDQLFFFDFSQNTRVPRLPEF

SEQ ID NO: 103 - DNA, 255 bp, synthetic construct
  source 1..255: mol_type = other DNA
atgggacatgagcagaaccagggcgctgctttactgcagatgctgcctttactttggctcctgcctcacagttgggctgtgcccgaagcccccactcccatgtggcccgacgatctgcagaatcatacttttctgcatacagtgtactgccaggatggaagccctagcgttggcctctctgaggcctacgatgaagaccagctgttcttctttgacttctcacagaataccagagttccaagattgcccgaattt

SEQ ID NO: 104 - protein, 176 aa, synthetic construct
ADWAQEQGDAPAILFDKEFCEWMIQQIGPKLDGKIPVSRGFPIAEVFTLKPLEFGKPNTLVCFVSNLFPPMLTVNWHDHSVPVEGFGPTFVSAVDGLSFQAFSYLNFTPEPSDIFSCIVTHEIDRYTAIAYWVPRNALPSDLLENVLCGVAFGLGVLGIIVGIVLIIYFRKPCSGD

SEQ ID NO: 105 - DNA, 528 bp, synthetic construct
  source 1..528: mol_type = other DNA
gccgactgggcccaggagcagggagacgccccagccattttgttcgacaaggagttctgtgagtggatgattcagcagattggccccaaattggacgggaagattcccgtatcacgcggattcccgattgcggaggtatttaccctgaaaccactggaatttggaaagcctaacacactggtgtgtttcgtgagcaacctgttccctcccatgctgacggtgaactggcacgatcactccgtgcctgtggaaggcttcggccctacatttgtgtctgcagtggacgggctatctttccaggctttcagctatctgaatttcacgcccgagcccagcgacattttctcctgcattgtaacacacgagatcgaccggtatactgctatagcctactgggtgccccgcaacgctctgccgagtgatctgttggagaacgtgttgtgcggggtagctttcgggttgggggtgcttggaattattgtgggcatcgtgctgattatctattttaggaaaccttgctcaggggac

SEQ ID NO: 106 - protein, 89 aa, synthetic construct
MGHEQNQGAALLQMLPLLWLLPHSWAVPEAPTPMWPDDLQNHTFLHTVYCQDGSPSVGLSEAYDEDQLFFFDFSQNTRVPRLPEFADWA

SEQ ID NO: 107 - DNA, 267 bp, synthetic construct
  source 1..267: mol_type = other DNA
atgggacatgagcagaaccagggcgctgctttactgcagatgctgcctttactttggctcctgcctcacagttgggctgtgcccgaagcccccactcccatgtggcccgacgatctgcagaatcatacttttctgcatacagtgtactgccaggatggaagccctagcgttggcctctctgaggcctacgatgaagaccagctgttcttctttgacttctcacagaataccagagttccaagattgcccgaatttgccgactgggcc

SEQ ID NO: 108 - protein, 172 aa, synthetic construct
QEQGDAPAILFDKEFCEWMIQQIGPKLDGKIPVSRGFPIAEVFTLKPLEFGKPNTLVCFVSNLFPPMLTVNWHDHSVPVEGFGPTFVSAVDGLSFQAFSYLNFTPEPSDIFSCIVTHEIDRYTAIAYWVPRNALPSDLLENVLCGVAFGLGVLGIIVGIVLIIYFRKPCSGD

SEQ ID NO: 109 - DNA, 516 bp, synthetic construct
  source 1..516: mol_type = other DNA
caggagcagggagacgccccagccattttgttcgacaaggagttctgtgagtggatgattcagcagattggccccaaattggacgggaagattcccgtatcacgcggattcccgattgcggaggtatttaccctgaaaccactggaatttggaaagcctaacacactggtgtgtttcgtgagcaacctgttccctcccatgctgacggtgaactggcacgatcactccgtgcctgtggaaggcttcggccctacatttgtgtctgcagtggacgggctatctttccaggctttcagctatctgaatttcacgcccgagcccagcgacattttctcctgcattgtaacacacgagatcgaccggtatactgctatagcctactgggtgccccgcaacgctctgccgagtgatctgttggagaacgtgttgtgcggggtagctttcgggttgggggtgcttggaattattgtgggcatcgtgctgattatctattttaggaaaccttgctcaggggac

SEQ ID NO: 110 - protein, 91 aa, synthetic construct
MITFLPLLLGLSLGCTGAGGFVAHVESTCLLDDAGTPKDFTYCISFNKDLLTCWDPEENKMAPCEFGVLNSLANVLSQHLNQKDTLMQRLR

SEQ ID NO: 111 - DNA, 273 bp, synthetic construct
  source 1..273: mol_type = other DNA
atgatcactttcctgcccctgctgttagggttaagcctggggtgtaccggggcaggaggcttcgttgcacatgttgagagcacttgtctgctggacgacgcaggaacaccaaaggatttcacgtactgcattagcttcaacaaagacctgcttacatgctgggaccctgaggagaacaagatggccccttgcgagttcggcgtgttaaacagcctggctaacgtgctgtcacagcacctgaatcagaaggacaccctcatgcagagactgcgg

SEQ ID NO: 112 - protein, 172 aa, synthetic construct
NGLQNCATHTQPFWGSLTNRTRPPSVQVAKTTPFNTREPVMLACYVWGFYPAEVTITWRKNGKLVMPHSSAHKTAQPNGDWTYQTLSHLALTPSYGDTYTCVVEHIGAPEPILRDWTPGLSPMQTLKVSVSAVTLGLGLIIFSLGVISWRRAGHSSYTPLPGSNYSEGWHIS

SEQ ID NO: 113 - DNA, 516 bp, synthetic construct
  source 1..516: mol_type = other DNA
aacggccttcagaattgcgctactcacactcagccgttctgggggtcattgaccaaccggactcgccctccatctgtacaggtggcgaaaactacccctttcaacacccgggagccagtcatgctggcttgctatgtttgggggttttacccagctgaggttactataacgtggcgcaaaaacgggaagctcgtaatgcctcattcgtccgcccataagacggctcaacccaacggagactggacatatcagaccctttcccaccttgcactgactccatcttatggggacacttacacctgcgtggtggagcacattggggccccagaaccaatcctgcgagactggaccccagggctctcaccaatgcagacgctgaaggtatcagtgagtgctgtgactctgggcctgggactgatcattttcagcctgggggtaattagttggagaagggctggacacagcagttacacacccctgcctggcagcaattactctgaaggctggcatattagc

SEQ ID NO: 114 - protein, 102 aa, synthetic construct
MSLQWTAVATFLYAEVFVVLLLCIPFISPKRWQKIFKSRLVELLVSYGNTFFVVLIVILVLLVIDAVREIRKYDDVTEKVNLQNNPGAMEHFHMKLFRAQRN

SEQ ID NO: 115 - DNA, 306 bp, synthetic construct
  source 1..306: mol_type = other DNA
atgagtctgcagtggactgcagttgccaccttcctctatgcggaggtctttgttgtgttgcttctctgcattcccttcatttctcctaaaagatggcagaagattttcaagtcccggctggtggagttgttagtgtcctatggcaacaccttctttgtggttctcattgtcatccttgtgctgttggtcatcgatgccgtgcgcgaaattcggaagtatgatgatgtgacggaaaaggtgaacctccagaacaatcccggggccatggagcacttccacatgaagcttttccgtgcccagaggaat

SEQ ID NO: 116 - protein, 21 aa, synthetic construct
AVATFLYAEVFVVLLLCIPFI

SEQ ID NO: 117 - DNA, 63 bp, synthetic construct
  source 1..63: mol_type = other DNA
gcagttgccaccttcctctatgcggaggtctttgttgtgttgcttctctgcattcccttcatt

SEQ ID NO: 118 - protein, 21 aa, synthetic construct
LVSYGNTFFVVLIVILVLLVI

SEQ ID NO: 119 - DNA, 63 bp, synthetic construct
  source 1..63: mol_type = other DNA
ttagtgtcctatggcaacaccttctttgtggttctcattgtcatccttgtgctgttggtcatc

SEQ ID NO: 120 - protein, 24 aa, synthetic construct
SGGQPAGPGAAGEALAVLTSFGRR

SEQ ID NO: 121 - protein, 25 aa, synthetic construct
PRLLLTALSFALDGAVYHLAPPMGA

SEQ ID NO: 122 - protein, 25 aa, synthetic construct
KDISKGIIEARFVYVFVLGILFTGT

SEQ ID NO: 123 - protein, 25 aa, synthetic construct
RPFCIILLISFFFVIVVGFFIYDQS

SEQ ID NO: 124 - protein, 25 aa, synthetic construct
FVFMFLFGGVLMTLLGLLGSLFFLG

SEQ ID NO: 125 - protein, 25 aa, synthetic construct
FPFNSFLSGFISCVGSFILAVCLRI

SEQ ID NO: 126 - protein, 25 aa, synthetic construct
SWLVITLVCLFVGIYAMVKLLLFSE

SEQ ID NO: 127 - protein, 25 aa, synthetic construct
PTMYLALIFTLVCILIVLGCILLLN

SEQ ID NO: 128 - protein, 25 aa, synthetic construct
QQLIVSLWCILLGGLCLLVGILLTK

SEQ ID NO: 129 - protein, 24 aa, synthetic construct
DLTLWLLASSILFLVVYSLLVINR

SEQ ID NO: 130 - protein, 25 aa, synthetic construct
WTAVATFLYAEVFVVLLLCIPFISP

SEQ ID NO: 131 - protein, 25 aa, synthetic construct
FDAFNVPVFWPILVMYFIMLFCITM

SEQ ID NO: 132 - protein, 25 aa, synthetic construct
FNLCLLIAGGLLVFTLLMVLGVSGV

SEQ ID NO: 133 - protein, 25 aa, synthetic construct
YKLYSLVSLSGWVLVKAFGMFGHRV

SEQ ID NO: 134 - protein, 25 aa, synthetic construct
PCLDLGAMLLYTLTFWLLLRQFVKE

SEQ ID NO: 135 - protein, 25 aa, synthetic construct
VSLLAVVVIVCGVALVAVFLFLFWK

SEQ ID NO: 136 - protein, 24 aa, synthetic construct
MEFGLSWLFLVAILKGVQCEVQLV

SEQ ID NO: 137 - protein, 24 aa, synthetic construct
MAWIPLLLLFLSHCTGSLSEVQLV

SEQ ID NO: 138 - DNA, 27 bp, synthetic construct
  source 1..27: mol_type = other DNA
agtatcctgatggcccgaattctgagc

SEQ ID NO: 139 - DNA, 27 bp, synthetic construct
  source 1..27: mol_type = other DNA
tccatgttcatggcaaggatcttatca

SEQ ID NO: 140 - protein, 13 aa, synthetic construct
SGAGSGGGSGAGS